(19) United States
(12) Patent Application Publication
(10) Pub. No.: US 2013/0243789 A1
(43) Pub. Date: Sep. 19, 2013

(54) Multiple Gene Expression Including sORF Constructs and Methods with Polyproteins, Pro-Proteins and Proteolysis

(71) Applicants: Gerald R. Carson, Belmont, MA (US); Jochen G. Salfeld, North Grafton, MA (US); Dean A. Regier, Upton, MA (US); Jijie Gu, Shrewsbury, MA (US); Wendy Gion, Charlton, MA (US); Yune Z. Kunes, Winchester, MA (US)

(72) Inventors: Gerald R. Carson, Belmont, MA (US); Jochen G. Salfeld, North Grafton, MA (US); Dean A. Regier, Upton, MA (US); Jijie Gu, Shrewsbury, MA (US); Wendy Gion, Charlton, MA (US); Yune Z. Kunes, Winchester, MA (US)

(73) Assignee: Abbott Laboratories, Abbott Park, IL (US)

(21) Appl. No.: 13/739,304

(22) Filed: Jan. 11, 2013

Related U.S. Application Data
(63) Continuation of application No. 12/822,598, filed on Jun. 24, 2010, now abandoned.
Application No. 12/822,598 is a continuation of application No. 11/459,098, filed on Jul. 21, 2006, now abandoned.
(60) Provisional application No. 60/701,855, filed on Jul. 21, 2005.

(51) Int. Cl. C12N 15/67

(52) U.S. Cl. CPC C12N 15/67
USPC 424/158.1; 435/69.1; 435/69.6; 435/325; 435/348; 435/349; 435/358; 435/365; 435/369; 435/419; 435/252.33; 435/254.11; 435/254.2; 435/254.21; 435/320.1; 530/350; 530/387.1; 530/389.2; 530/389.8; 424/172.1

(57) ABSTRACT

Disclosed are useful constructs and methods for the expression of proteins using primary translation products that are processed within a recombinant host cell. Constructs comprising a single open reading frame (sORF) are described for protein expression including expression of multiple polypeptides. A primary translation product (a pro-protein or a polyprotein) contains polypeptides such as inteins or hedgehog family auto-processing domains, or variants thereof, inserted in frame between multiple protein subunits of interest. Also disclosed are independent aspects of conducting efficient expression, secretion, and/or multimeric assembly of proteins such as immunoglobulins.

### CROSS-REFERENCES TO RELATED APPLICATIONS

This application is a continuation of U.S. patent application Ser. No. 12/822,598, filed Jun. 24, 2010, which is a continuation of U.S. patent application Ser. No. 11/459,098, filed Jul. 21, 2006, which claims the benefit of U.S. Provisional Application Ser. No. 60/701,855, filed Jul. 21, 2005; all of the foregoing are incorporated herein by reference in entirety.

### STATEMENT ON FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISK APPENDIX

Not Applicable (sequence listing provided but not as compact disk appendix).

### BACKGROUND OF THE INVENTION

The field of the present invention is molecular biology, especially as generally related to the area of recombinant protein expression, and the expression and processing, including post-translational processing, of recombinant polyproteins or pre-proteins in particular.

The use of antibodies as diagnostic tools and therapeutic modalities has found increasing use in recent years. The first FDA-approved monoclonal antibody, OKT3 (Johnson and Johnson) was approved for the treatment of patients with kidney transplant rejection. Herceptin (trademark of Genentech Inc., South San Francisco, Calif.), a humanized monoclonal antibody for treatment of patients with metastatic breast cancer, was approved in 1998. Numerous antibody-based therapies are showing promise in various stages of clinical development. One limitation in widespread clinical application of antibody technology is that typically large amounts of antibody are required for therapeutic efficacy and the costs associated with sufficient production are significant. Chinese Hamster Ovary (CHO) cells and NS0 myeloma cells are the most commonly used mammalian cell lines for commercial scale production of glycosylated human proteins such as antibodies and other biotherapeutics (Humphreys and Glover 2001. Curr. Opin. Drug Discov. Devel. 4:172-85). Mammalian cell line production yields typically range from 50-250 mg/L for 5-7 day culture in a batch fermentor or 300-600 mg/L in 7-12 days in fed batch fermentors. Non-glycosylated immunoglobulin proteins can be successfully produced in yeast or E. coli (see, e.g., Humphreys D P, et al., 2000, Protein Expr Purif. 20(2):252-64), however most successes in bacterial expression systems have been with antibody fragments (Humphreys, D. P. 2003. Curr. Opin. Drug Discov. Devel. 2003 6:188-96).

An important development in the field of expressing multiple gene segments or genes has been the discovery of inteins (see, e.g., Hirata, R et al., 1990, J. Biol. Chem. 265:6726-6733; Kane, P M et al., 1990, Science 250, 651-657; Xu, M-Q and Perler, F B, 1996, EMBO Journal 15(19):5146-5153). Inteins are considered the protein equivalent of gene introns and facilitate protein splicing. As noted in U.S. Pat. No. 7,026,526 by Snell K., protein splicing is a process in which an interior region of a precursor protein (an intein) is excised and the flanking regions of the protein (exteins) are ligated to form the mature protein. This process has been observed in numerous proteins from both prokaryotes and eukaryotes (Perler, F. B., Xu, M. Q., Paulus, H. Current Opinion in Chemical Biology 1997, 1, 292-299; Perler, F. B. Nucleic Acids Research 1999, 27, 346-347). The intein unit contains the necessary components needed to catalyze protein splicing and often contains an endonuclease domain that participates in intein mobility (Perler, F. B., et al., Nucleic Acids Research 1994, 22, 1127-1127).

While the main focus of intein-based systems has been on the generation of purification technologies and new fusion proteins from expressing gene segments, U.S. Pat. No. 7,026,526 reports DNA constructs with modified inteins for expression of multiple gene products as separate proteins to achieve stacked traits in plants. Still lacking, however, is an indication that those systems can be successfully used for expression of separate proteins that assemble into functional multimeric proteins, extracellularly secreted proteins, mammalian proteins, or proteins produced in eukaryotic host cells. It is noteworthy that immunoglobulins fall into all of these categories.

Compounding the difficulty of extending the modified intein approach of U.S. Pat. No. 7,026,526 to other genes or purposes is the recognition of the potential importance of the contributions of the desired extein gene segments relative to the intein system that is involved. Paulus reports, “Indeed, protein splicing, even though catalyzed entirely by the intein, can be strikingly influenced by extein sequences. This influence is shown by the fact that the expression of chimeric protein splicing systems, in which intein sequences are inserted in-frame between foreign coding sequences, often leads to substantial side reactions, such as cleavage at the upstream or downstream splice junctions (Xu M-Q, et al., 1993, Cell 75:1371-77; and Shingledecker K, et al., 1998, Gene 207:187-95). This suggests that the ability of inteins to assume a structure optimal for protein splicing without side reactions has evolved in the context of specific exteins.” See Paulus H, 2000, Protein splicing and related forms of protein autoprocessing, Annu. Rev. Biochem. 69:447-96. Another commentator states: “Although it is possible to introduce desirable properties and activities into proteins using rational design, subtle changes necessary to make an engineered product efficient and practical are often still beyond our predictive capacity (Shao, Z. and Arnold, F. H. 1996. Curr. Opin. Struct. Biol. 6, 513-518) . . . . Nevertheless, the regions immediately flanking inteins have been found to affect the efficiency of splicing (Chong, S. et al., 1998, Nucleic Acids Res. 26, 5109-5115; Southworth, M. W. et al., 199, Biotechniques 27, 110-114) and some protein hosts might be incompatible with intein activity. Although high expression and product purity are important considerations, they are moot if the final product is inactive.” See Amitai G and Pietrokovski, 1999, Nature Biotechnology 17:854-855.

Therefore, in a modified intein system where a preferred outcome is cleavage without re-ligation, the presence of a foreign extein relative to a given intein sequence may affect a practically efficient combination of precise cleavages, absence of re-ligation, and absence of side reactions. Clearly the adaptation of a modified intein approach for recombinant production of certain proteins that retain functional activity as final product, e.g., immunoglobulins and other biotherapeutics, represents a substantial challenge for innovation.

In the present invention this challenge has been taken up not only for intein-based systems but also has been explored in a pioneering sense for useful applications regarding hedgehog domains. Proteins in the hedgehog family are intercellular signaling molecules essential for patterning in vertebrate embryos. See, e.g., Mann, R. K. and Beachy, P. A. (2000) Biochim. Biophys. Acta. 1529, 188-202; Beachy, P A, (1997) Cold Spring Harb Symp Quant Biol 62: 191-204. Native hedgehog precursor proteins are cleaved into C-terminal (Hh-C) and N-terminal fragments (Hh-N) by an autoprocessing reaction that has similarity to protein splicing. The hedgehog system presents an untested opportunity for the creative development of systems including modified versions suitable for expression of multiple separate protein segments.

Previous attempts to express a full length antibody/immunoglobulin molecule via recombinant DNA technology using a single vector have met with limited success, typically resulting in significantly dissimilar levels of expression of the heavy and light chains of the antibody/immunoglobulin molecule, and more particularly, a lower level of expression for the second gene. Other factors may require relatively higher expression levels of one chain compared to the other for optimal production of a properly assembled, multimeric antibody or functional fragment thereof. Thus one problem is a suboptimal stoichiometry of expression of heavy and light chains within the cell which results in an overall low yield of assembled, multimeric antibody. Fang et al. indicate that in order to express high levels of a fully biological functional antibody from a single vector, equimolar expression of the heavy and light chains is required (see Fang et al., 2005, Nature Biotechnology 23:584-590; US Patent Publication 2004/0265955A1). Additionally, conventional expression systems relying on vector systems that independently express multiple polypeptides are significantly affected by such factors as promoter interactions (e.g., promoter interference). These interactions may compromise efficient expression of the genes and/or assembly of the expressed chains, or require the use of more than one vector (see, e.g., U.S. Pat. No. 6,331,415, Cabilly et al.). The requirement of multiple vectors is disadvantageous due to potential complications such as loss of one or more of the individual vectors in addition to generally needing additional manipulations.

Other factors that limit the ability to express two or more coding sequences from a single vector include the packaging capacity of the vector itself. For example, in considering the appropriate vector/coding sequence, factors to be considered include the packaging capacity of the vector (e.g., approx. 4,500 bp for adeno-associated virus, AAV); the duration of in vitro/in vivo expression of the recombinant protein by a vector-transfected cell or organ (e.g., short term expression for adenoviral vectors); the cell types supporting efficient infection by the vector if a viral vector is used; and the desired expression level of the gene product(s). The requirement for controlled expression of two or more gene products together with the packaging limitations of viral vectors such as adenovirus and AAV limits the choices with respect to vector construction and systems for expression of certain genes such as immunoglobulins or fragments thereof.

In further approaches to express two or more protein or polypeptide sequences from a single vector, two or more promoters or a single promoter and an internal ribosome entry site (IRES) sequence between the coding sequences of interest are used to drive expression of individual coding sequences. The use of two promoters within a single vector can result in low protein expression due to promoter interference. When two coding sequences are separated by an IRES sequence, the translational expression of the second coding sequence is often significantly weaker than that of the first (Furler et al. 2001. Gene Therapy 8:864-873). US Patent Publication 2004/0241821 describes flavivirus vectors in which a heterologous coding sequence is incorporated downstream of the virus polyprotein coding sequence, and separated therefrom by an IRES. A nuclear-anchored vector strategy for recombinant gene expression, including fusion proteins in which segments are separated by protease recognition sites, is described in US Patent Publication 2005/0026137.

The linking of proteins in the form of polyproteins in a single open reading frame (sORF) is a strategy observed in the replication of many natural viruses including the picornaviridae. Upon translation, virus-encoded proteinases mediate rapid intramolecular (cis) cleavage of a polyprotein to yield discrete mature protein products. Foot and Mouth Disease viruses (FMDV) are a group within the picornaviridae which express a single, long open reading frame encoding a polyprotein of approximately 225 kD. The full length translation product undergoes rapid intramolecular (cis) cleavage at the C-terminus of a 2A region occurring between the capsid protein precursor (P1-2A) and replicative domains of the polyprotein 2BC and P3, and this cleavage is mediated by the 2A region itself via a ribosomal stutter mechanism (Ryan et al. 1991. J. Gen. Virol. 72:2727-2732); Vakharia et al. 1987. J. Virol. 61:3199-3207). The essential amino acid residues for expression of the cleavage activity by the FMDV 2A region have been identified. The 2A and similar domains have also been characterized from aphthoviridae and cardioviridae of the picornavirus family (Donnelly et al. 1997. J. Gen. Virol. 78:13-21).

In still other attempts to use proteolytic processing techniques, early descriptions of recombinant insulin production include, e.g., EP055945 (Genentech); and EP037723 (The Regents of the University of California). It is a tremendous leap, however, to be able to apply such efforts in the context of exploiting recombinant expression of much larger and more complex functional proteins such as immunoglobulins. Examples of functional antibody molecules can involve heteromultimers requiring assembly of four or more chains (e.g., two immunoglobulin heavy chains and two light chains).

There remains a need for alternative and/or improved expression systems for generating recombinant proteins. A particular need is reflected in the area of efficient and/or correct expression of full length immunoglobulins and antigen-binding fragments thereof which provide advantages relative to currently available technology. The present invention addresses these needs by providing single vector constructs using a variety of strategies such as inteins, hedgehog autoprocessing segments, autocatalytic viral proteases, and variations thereof respectively. Independently, the need of efficient multimeric (e.g., immunoglobulin) assembly is addressed by adjusting the stoichiometric relationship of the subunits (e.g., heavy and light chains or fragments thereof). In embodiments, the constructs in a sORF encode a self-processing peptide component for expression of an industrially or biologically functional polypeptide, such as an enzyme, immunoglobulin, cytokine, chemokine, receptor, hormone, components of a two hybrid system, or other multi-subunit proteins of interest.

### BRIEF SUMMARY OF THE INVENTION

The present invention provides expression cassettes, vectors, recombinant host cells and methods for the recombinant expression and processing, including post-translational processing, of recombinant polyproteins and pre-proteins.

In an embodiment, the invention provides an expression vector for generating one or more recombinant protein products comprising a sORF insert; said sORF insert comprising a first nucleic acid sequence encoding a first polypeptide, an intervening nucleic acid sequence encoding a first protein cleavage site, and a second nucleic acid sequence encoding a second polypeptide; wherein said intervening nucleic acid sequence encoding said first protein cleavage site is operably positioned between said first nucleic acid sequence and said second nucleic acid sequence; and wherein said expression vector is capable of expressing a sORF polypeptide cleavable at said first protein cleavage site. In an embodiment, the first protein cleavage site comprises a self-processing cleavage site. In an embodiment, the self-processing cleavage site comprises an intein segment or modified intein segment, wherein the modified (or unmodified) intein segment permits cleavage but not complete ligation of expressed first polypeptides to expressed second polypeptides. In an embodiment, the self-processing cleavage site comprises a hedgehog segment or modified hedgehog segment, wherein the modified (or unmodified) hedgehog segment permits cleavage of expressed first polypeptides and expressed second polypeptides. In an embodiment, multiple separate proteins (e.g., first polypeptides, second polypeptides, third polypeptides, etc.) are expressed. In an embodiment, the first polypeptide and second polypeptide are capable of multimeric assembly. In an embodiment, at least one of said first polypeptide and second polypeptide are capable of extracellular secretion. In an embodiment, at least one of said first polypeptide and second polypeptide are of mammalian origin. In an embodiment, vectors and methods generating assembled antibodies are provided.

In embodiments, the invention provides constructs and methods for recombinant expression of multiple separate proteins. In particular embodiments, the proteins are capable of extracellular secretion. In particular embodiments, the proteins are of mammalian origin. In particular embodiments, the proteins are capable of multimeric assembly. In particular embodiments, the proteins are immunoglobulins.

In an embodiment, the incorporation of a protease recognition site, cleavable signal peptide or an autoprocessing polypeptide sequence (including an intein, a C-terminal auto-processing domain of hedgehog from drosophila, mouse, human, and other species (Dassa et al, Trends in Genetics, Vol. 20 No. 11 Nov., 2004, 538-542; Ibrahim et al, Biochimica et Biophysics Acta 1760 (2006) 347-355). We note that in some cases an autoprocessing polypeptide sequence can be referred to as a proteolytic site in connection with proteolytic processing. The C-terminal auto-processing domains of warthog, groundhog, and other hog-containing gene from nematodes such as Caenorhabditis elegans (Snell E A et al, Proc. R. Soc. B (2006) 273, 401-407; Aspock et al, Genome Research, 1999, 9:909-923); and Hoglet-C autoprocessing domain from choanoflagellate (Aspock et al, Genome Research, 1999, 9:909-923) are used. A-type bacterial intein-like (BIL) domains such as those from bacteria such as Clostridium thermocellum, and B-type BIL domains from bacteria such as Rhodobacter sphaeroides (Dassa et al, Journal of Biological Chemistry, Vol. 279, No. 31, July 30, 32001-32007), in wild type, truncated, or otherwise modified forms) into a recombinant pre-protein sequence allows efficient expression and cleavage of a pro-protein such that the bioactive portion is released or so that desired proteins expressed within a polyprotein are released. This embodiment eliminates the need for co-expression of the pro-protein's natural proteolytic processing enzymes. Alternatively, a protease cognate to the particular recognition site can be expressed coextensively with the pre-protein sequence, with a protease recognition site there between such that the protease can be released via proteolytic action and the precursor portion of the pre-protein is then released by subsequent proteolytic cleavage, such that the active portion of the pre-protein is released. In a still further embodiment, the 2A autoproteolytic processing peptide sequence can be engineered into the pre-protein between the mature (bioactive) portion and the precursor protein so that there is a self-processing of the engineered recombinant protein after expression.

In another embodiment of the invention, the present invention provides a method for efficient expression of recombinant immunoglobulin molecules, by recombinantly expressing a polyprotein comprising at least one heavy chain region and at least one light chain regions, wherein said regions are separated by one or more protease recognition sites, signal peptides, intein sequences which mediate cleavage but not joining of polypeptides, hedgehog sequence, other intein-like or hedgehog-like autoprocessing sequence or variation thereof, or by sequences such as as the 2A peptide that separate the flanking peptides during translation. In a further embodiment, a protease can be expressed as part of the polyprotein, separated from the remainder of the polyprotein by protease recognition sites, and wherein each protease recognition site is cognate to the concomitantly expressed protease. Then proteolytic or signal peptidase action releases the protease and the other individual proteins from the primary translation product. The above described methods for separating protein subunits in a poly protein can also be used in combination to achieve desired cleavage and protein expression outcomes.

In the case of an embodiment of immunoglobulin expression, the duplication of the light chain coding region allows for improved assembly and/or expression of the complete immunoglobulin molecule over the situation where the light chain coding regions are present in the expression cassette and/or expression vector at a 1:1 ratio with the heavy chain coding region. In the context of the present invention, heavy and light chain proteins can be functional fragments of the naturally occurring heavy and light chains (a functional fragment retains the ability to bind to its counterpart antibody chain and the ability to bind the cognate antigen is also retained, as well known in the art. Thus the invention provides constructs and methods wherein the coding region ratio of light chain component to heavy chain component is either 1:1 or greater than 1:1. For example, in an embodiment the L:H ratio is 2:1 or greater than 2:1; in other embodiments the ratio is 3:1, 3:2, 4:1, or greater than 4:1.

In a preferred aspect of the invention, the light chain immunoglobulin coding sequence, or component fragment thereof, is duplicated within the polyprotein coding sequence, and heavy and light chain immunoglobulin coding sequences are present at a molar ratio of about 2 light chains to about one heavy chains, and expressed at a ratio of greater than 1:1 light chain:heavy chain. The light and heavy chain sequences are linked in the polyprotein by protease cleavage sites, signal (or leader) peptides, inteins or self-processing sites.

Proteases (endoproteases) and signal peptidases and the amino acid sequences of their recognition sites useful for separating components of the biologically active protein within the polyprotein translation product and their recognition sequences include, without limitation, furin, RXR/K-R (SEQ ID NO:1); VP4 of IPNV, S/TXA-S/AG (SEQ ID NO:2); Tobacco etch virus (TEV) protease, EXXYXQ-G (SEQ ID NO:3); 3C protease of rhinovirus, LEVLFQ-GP (SEQ ID NO:4); PC5/6 protease; PACE protease, LPC/PC7 protease; enterokinase, DDDDK-X (SEQ ID NO:5); Factor Xa protease, IE/DGR-X (SEQ ID NO:6); thrombin, LVPR-GS (SEQ ID NO:7); genenase I, PGAAH-Y(SEQ ID NO:8); and MMP protease; Nuclear inclusion protein a(N1a) of turnip mosaic potyvirus; NS2B/NS3 of Dengue type 4 (DEN4) flaviviruses, NS3 protease of yellow fever virus (YFV); ORF V of cauliflower mosaic virus; and KEX2 protease, MYKR-EAD (SEQ ID). Another internal cleavage site option is CB2. The position within the recognition sequence at which cleavage occurs is shown with a hyphen.

In an embodiment, signal sequences employed are wild-type, mutated, or randomly mutated and selected via screening using techniques understood in the art.

Also within the scope of the invention as set forth above is an expression cassette, wherein the particular polyprotein or pre-protein (proprotein, polyprotein) coding sequence is operably linked to transcription regulatory sequences, expression vectors and recombinant host cells containing the expression vector or expression cassette.

The present invention provides a system for expression of a full length immunoglobulin or fragment thereof based on expression of heavy and light chain coding sequences under the transcriptional control of a single promoter, wherein separation of the heavy and light chains is mediated by inteins or modified inteins (which cleave but not do ligate the released protein molecules, or the antibody or other flanking protein sequences can be modified so as to prevent ligation of the proteins), or by C-terminal auto-processing domain of hedgehog from drosophila, mouse, human, and other species, or by C-terminal auto-processing domains of warthog, groundhog, and other hog-containing gene from nematodes such as Caenorhabditis elegans. Hoglet-C autoprocessing domain from choanoflagellate, or by an A-type bacterial intein-like (BIL) domains such as those from bacteria such as Clostridium thermocellum, or by a B-type BIL domains from bacteria such as Rhodobacter sphaeroides. Inteins useful in the present invention include, without limitation the Saccharomyces cerevisiae VMA, Pyrococcus, Synechocystis, and other inteins known to the art. The separation of heavy and light chains can also be mediated by self-processing cleavage site, e.g., a 2A or 2A-like sequence.

In one aspect, the invention provides a vector for expression of a recombinant immunoglobulin, which includes a promoter operably linked to the coding sequence for a first chain of an immunoglobulin molecule or a fragment thereof, a sequence encoding a self-processing cleavage site and the coding sequence for a second chain of an immunoglobulin molecule or fragment thereof, wherein the sequence encoding the self-processing cleavage site is inserted between the coding sequence for the first chain of the immunoglobulin molecule and the coding sequence for the second chain of the immunoglobulin molecule. Either the first or second chain of the immunoglobulin molecule may be a heavy chain or a light chain, and the sequence encoding the recombinant immunoglobulin may be a full length coding sequence or a fragment thereof. A second region corresponding to light chain is separated from an adjacent region by a protease recognition site, signal peptide or a self-processing site, such as a 2A site. There may be two copies of the L chain sequence and one of the H chain sequence (or multiple copies of each), with the proviso that each antibody chain component has the appropriate processing site or sequence associated with it so that correctly processed antibody chains are produced.

The vector may be any recombinant vector capable of expression of a full length polypeptide, e.g. an immunoglobulin molecule or fragment thereof, for example, a plasmid vector, especially one suitable for gene expression in mammalian cells, a baculovirus vector for expression in insect cells, an adeno-associated virus (AAV) vector, a lentivirus vector, a retrovirus vector, a replication competent adenovirus vector, a replication deficient adenovirus vector and a gutless adenovirus vector, a herpes virus vector or a nonviral vector (plasmid), among others.

Self-processing cleavage sites include a 2A peptide sequence, e.g., a 2A sequence derived from Foot and Mouth Disease Virus (FMDV). In a further preferred aspect, the vector comprises a sequence which encodes an additional proteolytic cleavage site located between the coding sequence for the first chain of the immunoglobulin molecule or fragment thereof and the coding sequence for the second chain of the immunoglobulin molecule or fragment thereof (i.e., adjacent the sequence for a self-processing cleavage site, such as a 2A cleavage site) and also adjacent to the second light chain sequence. In one exemplary approach, the additional proteolytic cleavage site is a furin cleavage site with the consensus sequence RXK/R-R (SEQ ID NO:1). A vector for recombinant immunoglobulin expression using a self-processing peptide may include any of a number of promoters, wherein the promoter is constitutive, regulatable or inducible, cell type specific, tissue-specific, or species specific. The vector may further comprise a sequence encoding a signal sequence for one or more of the coding sequences of immunoglobulin chains, pre-proteins or the like.

The invention further provides host cells or stable clones of host cells infected with a vector that comprises a sequence encoding heavy and light chains of an immunoglobulin (i.e., an antibody); a sequence encoding a self-processing cleavage site; and may further comprise a sequence encoding an additional proteolytic cleavage site, and optionally a protease coding region similarly separated from the remainder of the coding sequence(s) by a self-processing site or a protease recognition sequence. Use of such cells or clones in generating full length recombinant immunoglobulins or fragments thereof is also included within the scope of the invention. Suitable host cells include, without limitation, insect cultured cells such as Spodoptera frugiperda cells, microbes including bacteria, yeast cells such as Saccharomyces cerevisiae or Pichia pastoris, fungi such as Trichoderma reesei, Aspergillus, Aureobasidum and Penicillium species, as well as mammalian cells such as Chinese hamster ovary (e.g., CHO-K1, ATCC CCL 61; CHO DG44, Chasin et al. 1986, Som. Cell. Molec. Genet. 12:555), baby hamster kidney (BHK-21, BHK-570, ATCC CRL 8544, ATCC CRL 10314), COS, mouse embryonic (NIH-3T3, ATCC CRL 1658), Vero cells (African green monkey kidney, available as ATCC CRL 1587), canine kidney cells (e.g., MDCK, ATCC CCL 34), rat pituitary cells (GH1, ATCC CCL 34), certain human cell lines including human embryonic kidney cells (e.g. HEK293, ATCC CRL 1573), and various transgenic animal systems, including without limitation, pigs, mice, rats, sheep, goat, cows, can be used as well. Chicken systems for expression in egg white and transgenic sheep, goat and cow systems are known for expression in milk, among others. Plant cells are also suitable as host cells.

In a related aspect, the invention provides a recombinant immunoglobulin molecule or fragment thereof produced by such a cell or clones, wherein the immunoglobulin comprises amino acids derived from a self processing cleavage site, signal peptide, intein, C-terminal auto-processing hog-containing genes, bacterial intein-like (BIL) domains, or protease recognition sequence, and methods for producing the same. Where an intein is use, it is preferably a modified intein so that the two antibody chains are not spliced together to form a single polypeptide chain or the termini of the antibody polypeptides are such that they cannot be spliced together by the intein. The intein is placed as an in frame fusion between an N-extein and a C-extein, for example, between an immunoglobulin heavy chain and an immunoglobulin light chain, with the proviso that the intein and/or junction proximal amino acid sequence of the polyprotein primary translation product results in cleavage to release the exteins, but no ligation of those extein proteins occurs.

The present invention further provides a post-translational protein processing strategy using a hedgehog protein processing domain positioned between a first expressed protein portion and a second protein portion. Optionally the hedgehog protein processing domain (Hh-C) can be truncated to delete the cholesterol transfer portion so that only protein cleavage occurs. In case complete excision of the Hh-C does not occur, inclusion of a signal peptide domain at the N-terminus of the second protein portion may allow for proteolytic separation of a mature second protein from the Hh-C/first protein portion. Also within the scope of this aspect of the present invention are non-naturally occurring recombinant DNA molecules comprising a sequence encoding a polyprotein which includes a hedgehog protein processing domain positioned between a first expressed protein portion coding sequence and a second protein portion coding sequence so that a polyprotein is produced by translation from a single message.

In an additional aspect of the present invention is a modified furin, characterized by the addition of a peptide region which targets the newly synthesized furin protein to the lumen of the endoplasmic reticulum. Also encompassed is the intein or modified intein strategy, as set forth herein.

Another aspect of the present invention is the application to the polyprotein/self processing, intein processing, signal peptide cleavage or proteolytic cleavage approach to the two-hybrid and three-hybrid (and variants) technology. The first and second or first, second and third proteins are expressed as a polyprotein from a single transcript in a suitable host cell, and the coding sequences for these proteins are separated by a self processing site (e.g., 2A), intein, signal peptide or by protease recognition sites. This strategy eliminates the need for co-transfecting with more than one vector or by expressing each protein off a single transcript, as is done conventionally, with the result using the present invention that there is improved economy, efficiency and protein expression, and the potential binding pairs are within close proximity of one another which is believed to improve the likelihood of binding partners associating with one another. In a particular embodiment, the polyprotein comprises a bait protein, and self processing, intein, signal peptide or protease recognition sequence and inserted cDNA sequences, which represent one or more potential prey proteins that interact with the bait protein of interest. This cloning and expression strategy is shown schematically in FIGS. 8 and 9.

In an embodiment, the invention provides DNA constructs for expression of multiple gene products in a cell comprising a single promoter at the 5′ end of the construct, an intein-containing unit comprising two or more extein sequences encoding separate proteins, and one or more intein sequences fused to the carboxy-terminus encoding portion of each extein sequence, except the last extein sequence to be expressed; and a 3′ termination sequence comprising a polyadenylation signal following the last extein protein coding sequence; wherein the intein-containing unit is expressed as a precursor protein containing at least one intein flanked by extein encoded proteins; wherein at least one of the inteins can catalyze excision of the exteins; and, preferably, wherein at least one amino acid residue is substituted in, or added to, the intein-containing unit so that the excised exteins are not ligated by the intein. In a particular embodiment, the constructs are configured wherein at least two of the extein sequences, upon expression as proteins, are capable of associating in multimeric assembly. In an embodiment, at least two extein sequences are capable of encoding an immunoglobulin or other antigen recognition molecule. In an embodiment, at least one extein sequence, upon expression as a protein, is capable of extracellular secretion. In an embodiment, at least one extein sequence is a mammalian gene.

In embodiments, the invention provides constructs and methods for immunoglobulin expression using a modified or non-modified intein where expressed immunoglobulin segments are not re-ligated/fused, thereby allowing production of a assembled antibody from multiple subunits. In a particular embodiment, the modified intein includes a change in an amino acid residue located in the first position of the C-extein. In a particular embodiment, there is a change at the second to last amino acid within the intein segment.

In embodiments, the invention provides constructs and methods for expression of any gene or combination of genes. In a particular embodiment, the C-extein is modified. In a further particular embodiment, the C-extein is modified using a signal sequence. In another particular embodiment, there is an absence of a terminal C-extein component.

In embodiments, the invention provides constructs and methods for expression of antibody genes using a modified signal peptide for the second chain of immunoglobulin (either heavy chain or light chain), and third if used, which are placed after an intein or a hedgehog auto-processing domain. In an embodiment, an order of segments is as follows: first chain-first intein or hedgehog-first modified signal peptide-second chain-second modified signal peptide-third chain (in a two-chain situation, e.g., the third chain or the ‘second modified signal peptide-third chain’ segment is omitted). In another embodiment, a second intein or hedgehog segment is included after the second chain. In a particular embodiment, the use of such a modified signal peptide gives rise to increased antibody secretion. In an embodiment, the signal peptide used is modified to reduce hydrophobicity. In an embodiment, a signal peptide is unmodified.

In embodiments, sORF vectors are provided for transient expression. In other embodiment, sORF vectors are provided in stable expression systems. In an embodiment, stable host cells are generated as understood in the art, e.g., by transfection and other techniques.

While many exemplary constructs are specifically disclosed herein for the expression of antibody specific for tumor necrosis factor α (alpha), it is understood that constructs can be readily prepared using the same strategies with the substitution of sequences encoding other proteins. Particular examples include other immunoglobulins and biotherapeutic molecules. Further particular examples include antibodies specific for E/L selectin, interleukin-12, interleukin-18 or erythropoietin receptor, or any other antibody of desired specificity for which the amino acid sequence and/or the coding sequence is available to the art.

In an embodiment, the invention provides an expression vector for generating one or more recombinant protein products comprising a sORF insert; said sORF insert comprising a first nucleic acid sequence encoding a first polypeptide, a first intervening nucleic acid sequence encoding a first protein cleavage site, and a second nucleic acid sequence encoding a second polypeptide; wherein said intervening nucleic acid sequence encoding said first protein cleavage site is operably positioned between said first nucleic acid sequence and said second nucleic acid sequence; and wherein said expression vector is capable of expressing a sORF polypeptide cleavable at said first protein cleavage site. In an embodiment, said first protein cleavage site comprises a self-processing cleavage site.

In an embodiment, the self-processing cleavage site comprises an intein segment or modified intein segment, wherein the modified intein segment permits cleavage but not complete ligation of said first polypeptide to said second polypeptide. In an embodiment, the self-processing cleavage site comprises a hedgehog segment or modified hedgehog segment, wherein the modified hedgehog segment permits cleavage of said first polypeptide from said second polypeptide. In an embodiment, the first polypeptide and second polypeptide are capable of multimeric assembly. In an embodiment, at least one of said first polypeptide and second polypeptide are capable of extracellular secretion. In an embodiment, at least one of said first polypeptide and second polypeptide are of mammalian origin.

In an embodiment, at least one of said first polypeptide and second polypeptide comprises an immunoglobulin heavy chain or functional fragment thereof. In an embodiment, at least one of said first polypeptide and second polypeptide comprises an immunoglobulin light chain or functional fragment thereof. In an embodiment, said first polypeptide comprises an immunoglobulin heavy chain or functional fragment thereof and said second polypeptide comprises an immunoglobulin light chain or functional fragment thereof; and wherein said first and second polypeptides are in any order. In an embodiment, said first polypeptide and second polypeptide taken together are capable of associating in multimeric assembly to form a functional antibody or other antigen recognition molecule.

In an embodiment, said first polypeptide is upstream of said second polypeptide. In an embodiment, said second polypeptide is upstream of said first polypeptide.

In an embodiment, an expression vector further comprises a third nucleic acid sequence encoding a third polypeptide, wherein said third nucleic acid sequence is operably positioned after said second nucleic acid sequence; and wherein said third sequence may independently be the same or different from either of said first or second nucleic acid sequence. In an embodiment, at least two of said first, second, and third polypeptides taken together are capable of associating in multimeric assembly.

In an embodiment, the expression vector further comprises a second intervening nucleic acid sequence encoding a second protein cleavage site, wherein said second intervening nucleic acid sequence is operably positioned after said first and said second nucleic acid sequence; and wherein said second intervening sequence may be the same or different from said first intervening nucleic acid sequence. In an embodiment, an expression vector further comprises a third nucleic acid sequence encoding a third polypeptide, and a second intervening nucleic acid sequence encoding a second protein cleavage site; wherein the second intervening nucleic acid sequence and third nucleic acid sequence, in that order, are operably positioned after said second nucleic acid sequence. In an embodiment, said third nucleic acid sequence encodes an immunoglobulin heavy chain, light chain, or respectively a functional fragment thereof. In an embodiment, said third nucleic acid sequence encodes an immunoglobulin light chain or functional fragment thereof. In an embodiment, said third nucleic acid sequence encodes an immunoglobulin heavy chain or functional fragment thereof.

In an embodiment of an expression vector, said first intervening nucleic acid sequence encoding a first protein cleavage site comprises a signal peptide nucleic acid encoding a signal peptide cleavage site or modified signal peptide cleavage site sequence. In an embodiment, the expression vector further comprises a signal peptide nucleic acid sequence encoding a signal peptide cleavage site, operably positioned before said first nucleic acid sequence or said second nucleic acid sequence.

In an embodiment, an expression vector further comprises two signal peptide nucleic acid sequences, each independently encoding a signal peptide cleavage site, wherein one signal peptide nucleic acid sequence is operably positioned before said first nucleic acid encoding said first polypeptide and the other signal peptide nucleic acid sequence is operably positioned before said second nucleic acid encoding said second polypeptide. In embodiments, the two signal peptide sequences are the same or different.

In an embodiment, a signal peptide nucleic acid sequence encodes an immunoglobulin light chain signal peptide cleavage site or modified immunoglobulin light chain signal peptide cleavage site. In an embodiment, a signal peptide nucleic acid sequence encodes a modified or unmodified immunoglobulin light chain signal peptide cleavage site, and wherein said modified site is capable of effecting cleavage and increasing secretion of at least one of said first polypeptide, said second polypeptide, and an assembled molecule of said first and second polypeptides; and wherein a secretion level in the presence of said signal peptide site is about 10% greater to about 100-fold greater than a secretion level in the absence of said signal peptide site.

In an embodiment, an intervening nucleic acid sequence encoding a first protein cleavage site comprises an intein or modified intein sequence selected from the group consisting of: a Pyrococcus horikoshii Pho Pol I sequence, a Saccharomyces cerevisiae VMA sequence, Synechocystis spp. Strain PCC6803 DnaE sequence, Mycobacterium xenopi GyrA sequence, Pyrococcus species GB-D DNA polymerase, A-type bacterial intein-like (BIL) domain, and B-type BIL.

In an embodiment, an intervening nucleic acid sequence encoding a first protein cleavage site comprises a C-terminal auto-processing domain of a hedgehog family member, wherein the hedgehog family member is from Drosophila, mouse, human, or other insect or animal species. In an embodiment, an intervening nucleic acid sequence encoding a first protein cleavage site comprises a C-terminal auto-processing domain from a warthog, groundhog, or other hog-containing gene from a nematode, or Hoglet domain from a choanoflagellate.

In an embodiment, the first and said second polypeptide comprise a functional antibody or other antigen recognition molecule; with an antigen specificity directed to binding an antigen selected from the group consisting of: tumor necrosis factor-a, erythropoietin receptor, RSV, EL/selectin, interleukin-1, interleukin-12, interleukin-13, interleukin-18, interleukin-23, CXCL-13, GLP-1R, and amyloid beta. In an embodiment, the first and second polypeptides comprise a pair of immunoglobulin chains from an antibody of D2E7, ABT-007, ABT-325, EL246, or ABT-874. In an embodiment, the first and second polypeptide are each independently selected from an immunoglobulin heavy chain or an immunoglobulin light chain segment from an analogous segment of D2E7, ABT-007, ABT-325, EL246, ABT-874, or other antibody.

In an embodiment, a vector further comprises a promoter regulatory element for said sORF insert. In an embodiment, said promoter regulatory element is inducible or constitutive. In an embodiment, said promoter regulatory element is tissue specific. In an embodiment, said promoter comprises an adenovirus major late promoter.

In an embodiment, a vector further comprises a nucleic acid encoding a protease capable of cleaving said first protein cleavage site. In an embodiment, said nucleic acid encoding a protease is operably positioned within said sORF insert; said expression vector further comprising an additional nucleic acid encoding a second cleavage site located between said nucleic acid encoding a protease and at least one of said first nucleic acid and said second nucleic acid.

In an embodiment, the invention provides a host cell comprising a vector described herein. In an embodiment, the host cell is a prokaryotic cell. In an embodiment, said host cell is Escherichia coli. In an embodiment, said host cell is a eukaryotic cell. In an embodiment, said eukaryotic cell is selected from the group consisting of a protist cell, animal cell, plant cell and fungal cell. In an embodiment, said eukaryotic cell is an animal cell selected from the group consisting of a mammalian cell, an avian cell, and an insect cell. In a preferred embodiment, said host cell is a CHO cell or a dihydrofolate reductase-deficient CHO cell. In an embodiment, said host cell is a COS cell. In an embodiment, said host cell is a yeast cell. In an embodiment, said yeast cell is Saccharomyces cerevisiae. In an embodiment, said host cell is an insect Spodoptera frugiperda Sf9 cell. In an embodiment, said host cell is a human embryonic kidney cell.

In an embodiment, the invention provides a method for producing a recombinant polyprotein or a plurality of proteins, comprising culturing a host cell in a culture medium under conditions sufficient to allow expression of a vector protein. In an embodiment, the method further comprises recovering and/or purifying said vector protein. In an embodiment, said plurality of proteins are capable of multimeric assembly. In an embodiment, the recombinant polyprotein or plurality of proteins are biologically functional and/or therapeutic.

In an embodiment, the invention provides a method for producing an immunoglobulin protein or functional fragment thereof, assembled antibody, or other antigen recognition molecule, comprising culturing a host cell according to claim 38 in a culture medium under conditions sufficient to produce an immunoglobulin protein or functional fragment thereof, assembled antibody, or other antigen recognition molecule.

In an embodiment, the invention provides a protein or polyprotein produced according to a method herein. In an embodiment, the invention provides an assembled immunoglobulin; assembled other antigen recognition molecule; or individual immunoglobulin chain or functional fragment thereof produced according to the methods herein. In an embodiment, the immunoglobulin; other antigen recognition molecule; or individual immunoglobulin chain or functional fragment thereof has a capability to effect or contribute to specific antigen binding to tumor necrosis factor-a, erythropoietin receptor, interleukin-18, EL/selectin or interleukin-12. In an embodiment, the immunoglobulin is D2E7 or wherein the functional fragment is a fragment of D2E7.

In an embodiment, the invention provides a pharmaceutical composition or medicament comprising a protein and a pharmaceutically acceptable carrier. Excipients and carriers for pharmaceutical formulations are selected as would be understood in the art.

In an embodiment, the invention provides an expression vector wherein the first protein cleavage site comprises a cellular protease cleavage site or a viral protease cleavage site. In an embodiment, said first protein cleavage site comprises a site recognized by furin; VP4 of IPNV; tobacco etch virus (TEV) protease; 3C protease of rhinovirus; PC5/6 protease; PACE protease, LPC/PC7 protease; enterokinase; Factor Xa protease; thrombin; genenase I; MMP protease; Nuclear inclusion protein a(N1a) of turnip mosaic potyvirus; NS2B/NS3 of Dengue type 4 flaviviruses, NS3 protease of yellow fever virus; ORF V of cauliflower mosaic virus; KEX2 protease; CB2; or 2A. In an embodiment, said first protein cleavage site is a viral internally cleavable signal peptide cleavage site. In an embodiment, said viral internally cleavable signal peptide cleavage site comprises a site from influenza C virus, hepatitis C virus, hantavirus, flavivirus, or rubella virus.

In an embodiment, the invention provides a method for expression of proteins of a two hybrid system, wherein said two hybrid system comprises a bait protein and a candidate prey protein, said method comprising the steps of: providing a host cell into which has been introduced an expression vector encoding a polyprotein comprising a bait protein portion and a candidate prey protein portion, said portions separated by a self-processing cleavage sequence, a signal peptide sequence or a protease cleavage site; and culturing the host cell under conditions which allow expression of the polyprotein and self processing or protease cleavage of the polyprotein. In an embodiment, the polyprotein further comprises a cleavable component of a three hybrid system.

In an embodiment, an expression vector does not contain a 2A sequence. In an embodiment, an expression vector is provided wherein said first protein cleavage site comprises a FMDV 2A sequence; a 2A-like domain from other Picornaviridae, an insect virus, Type C rotavirus, trypanosome, or Thermatoga maritima.

In an embodiment, the invention provides an expression vector for expressing a recombinant protein, comprising a coding sequence for a polyprotein, wherein the polyprotein comprises at least a first and a second protein segment, wherein said protein segments are separated by a protein cleavage site therebetween, wherein the protein cleavage site comprises a self processing peptide cleavage sequence, a signal peptide cleavage sequence or a protease cleavage sequence; and wherein said coding sequence is expressible in a host cell and is cleaved within the host cell.

In an embodiment, the invention provides an expression vector where an intervening nucleic acid sequence additionally encodes a tag.

Other aspects, features and advantages of the invention are apparent from the following description of the invention, provided for the purpose of disclosure when taken in conjunction with the accompanying drawings.

In general the terms and phrases used herein have their art-recognized meaning, which can be found by reference to standard texts, journal references and contexts known to those skilled in the art. Definitions provided herein are intended to clarify their specific use in the context of the invention.

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE DRAWINGS

FIG. 1 illustrates a preferred stable sORF expression vector construct.

FIG. 2 illustrates a preferred stable sORF expression vector construct, further comprising additional (second) intervening nucleic acid encoding a second protein cleavage site (which can be an autoprocessing site) and third nucleic acid sequence encoding a third polypeptide. Such a vector is capable of expression of more than two polypeptides.

FIG. 3 illustrates a preferred transient sORF expression vector construct, (e.g., pTT3-HC-Ssp-GA-int-LC-0aa).

FIG. 4 illustrates an expression vector with a 2A segment for a two-hybrid system. The vector expression cassette is structured to translate the bait protein first as a GAL4::bait::2A peptide fusion, which is self processed after the translation of the 2A peptide. The second open reading frame (ORF) is an NFkappaB::library fusion protein.

FIG. 5 is an expanded linear view of the expression region of the plasmid of FIG. 4 (2-hybrid system with 2A cleavage).

FIG. 6 illustrates intein-based sORF vectors for immunoglobulin expression.

FIG. 7 illustrates several sORF constructs with selected point mutations for expression of assembling multimeric molecules such as antibodies.

FIG. 8 illustrates sORF constructs with altered signal peptides, e.g., modified immunoglobulin light chain signal peptides.

FIG. 9 illustrates sORF constructs using hedgehog auto-processing domains.

### DETAILED DESCRIPTION OF THE INVENTION

The invention may be further understood by the following description and non-limiting examples.

The present invention provides systems, e.g., constructs and methods, for expression of a structural or a biologically active protein such as an enzyme, hormone (e.g., insulin), cytokine, chemokine, receptor, antibody, or other molecule. Preferably, the protein is an immunomodulatory protein such as an interleukin, a full length immunoglobulin, fragment thereof, other antigen recognition molecule as understood in the art, or other biotherapeutic molecule. An overview of such systems is in the specific context of an immunoglobulin molecule where recombinant production is based on expression of heavy and light chain coding sequences under the transcriptional control of a single promoter, wherein conversion of a single translation product (polyprotein) to the separate heavy and light chains is mediated by inteins, hog-containing auto-processing domains, 2A or 2A-like sequence that separate the flanking peptides at ribosome during translation or is the result of proteolytic processing at one or more protease recognition sequences located between the two chains of the mature biologically active protein.

The intervening site (whether related to an intein segment, hog domain, 2A or 2A-like, or protease recognition site; and variations thereof for each) may be referred to as a cleavage site. In the case where a plurality of three or more protein segments is expressed, such a cleavage site can be located between at least any two of the multiple segments, or a cleavage site can be located after each segment, optionally and preferably not after the last segment. If multiple cleavage sites are used, each may be the same as or independent from another.

In one aspect, the invention provides a vector for expression of a recombinant immunoglobulin, which includes a promoter operably linked to the coding sequence for a first chain of an immunoglobulin molecule or a fragment thereof, a sequence encoding a self-processing or other proteolytic cleavage site and the coding sequence for a second chain of an immunoglobulin molecule or fragment thereof, wherein the sequence encoding the self-processing or other proteolytic cleavage site is inserted between the coding sequence for the first chain of the immunoglobulin molecule and the coding sequence for the second chain of the immunoglobulin molecule, and a third region, encoding an immunoglobulin light chain, also separated from the remainder of the polyprotein by a self-processing or other proteolytic cleavage site.

In an embodiment, either the first or second chain of the immunoglobulin polyprotein molecule may be a heavy chain or a light chain. A sequence encoding a recombinant immunoglobulin segment may be a full length coding sequence or a fragment thereof. In a specific embodiment, a second light chain coding sequence must be part of the sequence encoding the polyprotein to be processed in the practice of the present invention; i.e., taken together there are three segments comprising two light chains and one heavy chain, in any order. In particular embodiments, constructs are configured with these components and in this order: a) IgH-IgL; b) IgL-IgH; c) IgH-IgL-IgL; d) IgL-IgH-IgL; e) IgL-IgL-IgH; f) IgH-IgH-IgL; g) IgH-IgL-IgH; and/or h) IgL-IgH-IgH. In an embodiment, the hyphen can indicate the location where a cleavage site sequence is located.

Alternatively, the immunoglobulin heavy and light chain coding sequences are fused in frame to an intein coding sequence there between, with the intein either modified so as to lack splicing activity or the termini of the heavy and light chains designed so that splicing preferably does not occur or such that splicing occurs with poor efficiency such that unspliced antibody molecules predominate. In addition, a modified intein can further be modified still further so that there is no endonuclease region (where an endonuclease region had previously existed), with the proviso that site specific proteolytic cleavage activity remains so that the light and heavy antibody polypeptides are freed from the intervening intein portion of the primary translation product. Either the light or the heavy antibody polypeptide can be the N-extein, and either can be the C-extein.

The vector may be any recombinant vector capable of expression of a full length polyprotein, for example, an adeno-associated virus (AAV) vector, a lentivirus vector, a retrovirus vector, a replication competent adenovirus vector, a replication deficient adenovirus vector and a gutless adenovirus vector, a herpes virus vector or a nonviral vector (plasmid) or any other vector known to the art, with the choice of vector appropriate for the host cell in which the immunoglobulin or other protein(s) are expressed. Baculovirus vectors are available for expression of genes in insect cells. Numerous vectors are known to the art, and many are commercially available or otherwise readily accessible to the art.

Cleavage Sites

Preferred self-processing cleavage sites include an intein sequence; modified intein; hedgehog sequence; other hog-family sequence; a 2A sequence, e.g., a 2A sequence derived from Foot and Mouth Disease Virus (FMDV); and variations thereof for each.

Proteases whose recognition sequences can substitute for the 2A sequence include, without limitation, furin, a modified furin targeted to the endoplasmic reticulum rather than the trans Golgi network, VP4 of IPNV, TEV protease, a nuclear localization signal-deficient TEV protease (TEV NIs-), 3C protease of rhinovirus, PC5/6 protease, PACE protease, LPC/PC7 protease, enterokinase, Xa protease, thrombin, genenase I and MMP protease, as discussed above. Other endoproteases useful in the practice of the present invention are proteases including, but not limited to, nuclear inclusion protein a(N1a) of turnip mosaic potyvirus (Kim et al. 1996. Virology 221:245-249); NS2B/NS3 of Dengue type 4 (DEN4) flaviviruses (Falgout et al. 1993. J. Virol. 67:2034-2042; Lai et al. 1994. Arch. Virol. Suppl. 9:359-368), NS3 protease of yellow fever virus (YFV) (Chambers et al. 1991. J. Virol. 65:6042-6050); ORF V of cauliflower mosaic virus (Torruella et al. 1989. EMBO Journal 8:2819-2825); inteins, an example of which is the Psp-GBD Pol intein (Xu, M. Q. 1996. EMBO 15: 5146-5153); an internally cleavable signal peptide, an example of which is the internally cleavable signal peptide of influenza C virus (Pekosz A. 1992. Proc. Natl. Acad. Sci. USA 95: 3233-13238); and KEX2 protease, MYKR-EAD (SEQ ID NO:9); KEX2 and a modified KEX2 which is targeted to the ER (see Chaudhuri et al. 1992. Eur. J. Biochem. 210:811-822). The modified KEX2 which is uniquely directed to the ER has coding and amino acid sequences as given in Table 7A and 7B, respectively; it is called KEX2-sol-KDEL. The primary amino acid sequence of KEX2 from Saccharomyces cerevisiae has been modified to remove the membrane association domain and to add the ER targeting sequence KDEL at the C terminus of the protein. Other human proteases useful for cleaving polyproteins containing the appropriate cleavage recognition sites include those set forth in US Patent Publication 2005/0112565. The sonic hedgehog protein from Drosophila melanogaster, especially the processing domain therefrom, can also serve to free proteins from a polyprotein primary translation product.

Within the scope of the present invention is a modified furin protease, which is targeted to the endoplasmic reticulum (ER) rather than to the trans Golgi network (TGN), as is the naturally occurring furin protease. Vorhees et al. 1995. EMBO Journal 14:4961-4975 described the EEDE (SEQ ID NO:10) portion of furin (amino acids 775-778) as involved in the targeting of the protease to the TGN (Nakayama et al. 1997. Biochem. Journal 327:625-635). Zerangue et al. 2001. Proc. Natl. Acad. Sci. USA 98:2431-2436 reported ER trafficking signals, including KKXX at the C terminus of a protein. Thus a modified furin is developed and used to target furin cleavage activity to the ER compartment instead of or in addition to the TGN and later compartments.

In a further aspect, the vector comprises a sequence which encodes an additional cleavage site located between the coding sequence for the first chain of the immunoglobulin molecule or fragment thereof and the coding sequence for the second and/or third chain (e.g., a duplicate of the first or second chain) of the immunoglobulin molecule or fragment thereof (i.e., adjacent the sequence for a cleavage site, which could be a 2A cleavage site). In one exemplary approach, the additional proteolytic cleavage site is a furin cleavage site with the consensus sequence RXK(R)R (SEQ ID NO:1).

Regulatory Sequences Including Promoters; Host Cells

A vector for recombinant immunoglobulin or other protein expression may include any of a number of promoters known to the art, wherein the promoter is constitutive, regulatable or inducible, cell type specific, tissue-specific, or species specific. Further specific examples include, e.g., tetracycline-responsive promoters (Gossen M, Bujard H, Proc Natl Acad Sci USA. 1992, 15; 89(12):5547-51). The vector is a replicon adapted to the host cell in which the chimeric gene is to be expressed, and it desirably also comprises a replicon functional in a bacterial cell as well, advantageously, Escherichia coli, a convenient cell for molecular biological manipulations.

The host cell for gene expression can be, without limitation, an animal cell, especially a mammalian cell, or it can be a microbial cell (bacteria, yeast, fungus, but preferably eukaryotic) or a plant cell. Particularly suitable host cells include insect cultured cells such as Spodoptera frugiperda cells, yeast cells such as Saccharomyces cerevisiae or Pichia pastoris, fungi such as Trichoderma reesei, Aspergillus, Aureobasidum and Penicillium species as well as mammalian cells such as CHO (Chinese hamster ovary), BHK (baby hamster kidney), COS, 293, 3T3 (mouse), Vero (African green monkey) cells and various transgenic animal systems, including without limitation, pigs, mice, rats, sheep, goat, cows, can be used as well. Chicken systems for expression in egg white and transgenic sheep, goat and cow systems are known for expression in milk, among others. Baculovirus, especially AcNPV, vectors can be used for the single ORF antibody expression and cleavage of the present invention, for example with expression of the sORF under the regulatory control of a polyhedrin promoter or other strong promote in an insect cell line; such vectors and cell lines are well known to the art and commercially available. Promoters used in mammalian cells can be constitutive (Herpes virus TK promoter, McKnight, Cell 31:355, 1982; SV40 early promoter, Benoist et al. Nature 290:304, 1981 Rous sarcoma virus promoter, Gorman et al. Proc. Natl. Acad. Sci. USA 79:6777, 1982; cytomegalovirus promoter, Foecking et al. Gene 45:101, 1980; mouse mammary tumor virus promoter, generally see Etcheverry in Protein Engineering: Principles and Practice, Cleland et al., eds, pp. 162-181, Wiley & Sons, 1996) or regulated (metallothionein promoter, Hamer et al. J. Molec. Appl. Genet. 1:273, 1982, for example). Vectors can be based on viruses that infect particular mammalian cells, especially retroviruses, vaccinia and adenoviruses and their derivatives are known to the art and commercially available. Promoters include, without limitation, cytomegalovirus, adenovirus late, and the vaccinia 7.5K promoters. Yeast and fungal vectors (see, e.g., Van den Handel, C. et al. (1991) In: Bennett, J. W. and Lasure, L. L. (eds.), More Gene Manipulations in Fungi, Academy Press, Inc., New York, 397-428) and promoters are also well known and widely available. Enolase is a well known constitutive yeast promoter, and alcohol dehydrogenase is a well known regulated promoter.

The selection of the specific promoters, transcription termination sequences and other optional sequences, such as sequences encoding tissue specific sequences, will be determined in large part by the type of cell in which expression is desired. The may be bacterial, yeast, fungal, mammalian, insect, chicken or other animal cells.

Signal Sequences

The coding sequence of the protein to be cleaved, proteolytically processed or self processed, which is incorporated in the vector, may further comprise one or more sequences encoding one or more signal sequences. These encoded signal sequences can be associated with one or more of the mature segments within the polyprotein. For example, the sequence encoding the immunoglobulin heavy chain leader sequence can precede the coding sequence for the heavy chain, operably linked and in frame with the remainder of the polyprotein coding sequence. Similarly, a light chain leader peptide coding sequence or other leader peptide coding sequence can be associated in frame with one or both of the immunoglobulin light chain coding sequences, with the leader sequence-chain being separated by the adjacent chain from either a self-processing site (such as 2A) or by a sequence encoding a protease recognition sequence, with the appropriate reading frame being maintained.

Stoichiometry of Immunoglobulin Heavy and Light Chains

In many embodiments herein, immunoglobulin/antibody light chains chains (IgL) and heavy chains (IgH) are present at a vector level or at an expressed intracellular level within a host cell at about a 1:1 ratio (IgL:IgH). Whereas recombinant approaches herein and elsewhere have relied on equimolar expression of heavy and light chains (see, e.g., US Patent Publication 2005/0003482A1 or International Publication WO2004/113493), in other embodiments the present invention provides methods and expression cassettes and vectors with light and heavy chain coding sequences in a ratio of 2:1 and co-expressed with self-processing or proteolytic processing of the chains when the primary translation product is a polyprotein. In embodiments, the ratio is greater than 1:1, such as about 2:1 or greater than 2:1. In a particular embodiment, a light chain coding sequence is used at a ratio of greater than 1:1 (IgL:IgH). In a specific embodiment, the ratio of IgL:IgH is 2:1.

The invention further provides host cells or stable clones of host cells transformed or infected with a vector that comprises a sequence encoding a heavy and either one or at least two light chains of an immunoglobulin (i.e., an antibody); sequences encoding cleavage sites, such as self-processing, protease recognition sites or signal peptides there between; and may further comprise a sequence or sequences encoding an additional proteolytic cleavage site. Also included in the scope of the invention is the use of such cells or clones in generating full length recombinant immunoglobulins or fragments thereof or other biologically active proteins which are comprised of multiple subunits (e.g., two-chain or multi-chain molecules or those which are in nature produced as a pro-protein and cleaved or processed to release a precursor-derived protein and the active portion). Non-limiting examples include insulin, interleukin-18, interleukin-1, bone morphogenic protein 4, bone morphogenic protein 2, any other two chain bone morphogenic proteins, nerve growth factor, renin, chymotrypsin, transforming growth factor 1i, and interleukin 113.

In a related aspect, the invention provides a recombinant immunoglobulin molecule or fragment thereof or other protein produced by such a cell or clones, wherein the immunoglobulin comprises amino acids derived from a self processing cleavage site (such as an intein or hedgehog domain), cleavage site or signal peptide cleavage and methods, vectors and host cells for producing the same. In embodiments, the invention provides host cells containing one or more constructs as described herein.

The present invention provides single vector constructs for expression of an immunoglobulin molecule or fragment thereof and methods for in vitro or in vivo use of the same. The vectors have self-processing or other protease recognition sequences between a first and second and between a second and third immunoglobulin coding sequence, allowing for expression of a functional antibody molecule using a single promoter and transcript. Exemplary vector constructs comprise a sequence encoding a self-processing cleavage site between open reading frames and may further comprise an additional proteolytic cleavage site adjacent to the self-processing cleavage site for removal of amino acids that comprise the self-processing cleavage site following cleavage. The vector constructs find utility in methods relating to enhanced production of full length biologically active immunoglobulins or fragments thereof in vitro and in vivo. Other biologically active proteins with at least two different chains can be made using the same strategy, although it is understood that it may not be required that either chain's coding sequence be present in a ratio greater than 1 relative to the other chain's coding sequence.

Although particular compositions and methods are exemplified herein, it is understood that any of a number of alternative compositions and methods are applicable and suitable for use in practicing the invention. It will also be understood that an evaluation of the polyprotein expression cassette and vectors, host cells and methods of the invention may be carried out using procedures standard in the art. The practice of the present invention will employ, unless otherwise indicated, conventional techniques of cell biology, molecular biology (including recombinant techniques), microbiology, biochemistry and immunology, which are within the scope of those of skill in the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1993); PCR: The Polymerase Chain Reaction, (Mullis et al., eds., 1994); and Current Protocols in Immunology (J. E. Coligan et al., eds., 1991), each of which is expressly incorporated by reference herein.

Unless otherwise indicated, all terms used herein have the same meaning as they would to one skilled in the art and the practice of the present invention will employ, conventional techniques of microbiology and recombinant DNA technology, which are within the knowledge of those of skill of the art.

The term “modified” as generally used herein in the context of a protein refers to a segment wherein at least one amino acid residue is substituted in, deleted from, or added to, the referenced molecule. Similarly, in the context of a nucleic acid the term refers to a segment wherein at least one nucleic acid subunit is substituted in, deleted from, or added to, the referenced molecule.

The term “intein” as used herein typically refers to an internal segment of a protein that facilitates its own removal and effects the joining of flanking segments known as exteins. Many examples of inteins are recognized in a variety of types of organisms, in some cases with shared structural and/or functional features. The invention is broadly able to employ inteins, and variants thereof, as appreciated to exist and further be recognized or discovered. See, e.g., Gogarten J P et al., 2002, Annu Rev Microbiol. 2002; 56:263-87; Perler, F. B. (2002), InBase, the Intein Database. Nucleic Acids Res. 30, 383-384 (also via internet at website of New England Biolabs, Inc., Ipswich, Mass.; http://www.neb.com/neb/inteins.html; Amitai G, et al., Mol. Microbiol. 2003, 47(1):61-73; Gorbalenya A E, Nucleic Acids Res. 1998; 26(7): 1741-1748. Non-canonical inteins). In a protein an intein-containing unit or intein splicing unit can be understood as encompassing portions of the flanking exteins where structural aspects can contribute to reactions of cleavage, ligation, etc. The term can also be understood as a category in referring to an intein-based system with a “modified intein” component.

The term “modified intein” as used herein can refer to a synthetic intein or a natural intein wherein at least one at least one amino acid residue is substituted in, deleted from, or added to, the intein splicing unit so that the cleaved or excised exteins are not completely ligated by the intein.

The term “hedgehog” as used herein refers to a gene family (and corresponding protein segments) with members that have structure effecting autoproteolytic function. Family members include, for example, analogs from Drosophila, mouse, human, and other species. Furthermore, the term “hedgehog segment” is intended to encompass not only such family members but also broadly relates to auto-processing domains of warthog, groundhog, and other hog-containing gene from nematodes such as Caenorhabditis elegans, and Hoglet-C autoprocessing domain from choanoflagellates. See, e.g., Perler F B. Protein splicing of inteins and hedgehog autoproteolysis: structure, function, and evolution, Cell. 1998, 92(1):1-4; Koonin, E V et al., (1995) A protein splice-junction motif in hedgehog family proteins. Trends Biochem Sci. 20(4): 141-2; Hall T M et al., (1997) Crystal structure of a Hedgehog autoprocessing domain: homology between Hedgehog and self-splicing proteins. Cell 91(1): 85-97; Snell E A et al, Proc. R. Soc. B (2006) 273, 401-407; Aspock et al, Genome Research, 1999, 9:909-923. A particular example of a hedgehog segment is the sonic hedgehog protein from Drosophila melanogaster. The term can also be understood as a category in referring to a hedgehog-based system with a “modified hedgehog” component.

The term “modified hedgehog” segment can refer to a synthetic hedgehog segment or a natural hedgehog segment wherein at least one at least one amino acid residue is substituted in, deleted from, or added to, the hedgehog splicing unit so that cleaved segments are not completely ligated.

The term “vector”, as used herein, refers to a DNA or RNA molecule such as a plasmid, virus or other vehicle, which contains one or more heterologous or recombinant DNA sequences and is designed for transfer between different host cells. The terms “expression vector” and “gene therapy vector” refer to any vector that is effective to incorporate and express heterologous DNA fragments in a cell. A cloning or expression vector may comprise additional elements, for example, the expression vector may have two replication systems, thus allowing it to be maintained in two organisms, for example in human cells for expression and in a prokaryotic host for cloning and amplification. Any suitable vector can be employed that is effective for introduction of nucleic acids into cells such that protein or polypeptide expression results, e.g. a viral vector or non-viral plasmid vector. Any cells effective for expression, e.g., insect cells and eukaryotic cells such as yeast or mammalian cells are useful in practicing the invention.

The terms “heterologous DNA” and “heterologous RNA” refer to nucleotides that are not endogenous (native) to the cell or part of the genome or vector in which they are present. Generally heterologous DNA or RNA is added to a cell by transduction, infection, transfection, transformation, electroporation, biolistic transformation or the like. Such nucleotides generally include at least one coding sequence, but the coding sequence need not be expressed. The term “heterologous DNA” may refer to a “heterologous coding sequence” or a “transgene”.

As used herein, the terms “protein” and “polypeptide” may be used interchangeably and typically refer to “proteins” and “polypeptides” of interest that are expresses using the self processing cleavage site-containing vectors of the present invention. Such “proteins” and “polypeptides” may be any protein or polypeptide useful for research, diagnostic or therapeutic purposes, as further described below. As used herein, a polyprotein is a protein which is destined for processing to produce two or more polypeptide products.

As used herein, the term “multimer” refers to a protein comprised of two or more polypeptide chains (sometimes referred to as “subunits”), which assemble to form a function protein. Multimers may be composed of two (dimers), three, (trimers), four (tetramers), or more (e.g., pentamers, and so on) peptide chains. Multimers may result from self-assembly, or may require a component such as a catalyst to assist in assembly. Multimers may be composed solely of identical peptide chains (homo-multimer), or two or more different peptide chains (hetero-multimers). Such multimers may structurally or chemically functional. Many multimers are known and used in the art, including but not limited to enzymes, hormones, antibodies, cytokines, chemokines, and receptors. As such, multimers can have both biological (e.g., pharmaceutical) and industrial (e.g., bioprocessing/bioproduction) utility.

As used herein, the term “tag” refers to a peptide, which may incorporated into an expression vector that that may function to allow detection and/or purification of one or more expression products of the vector inserts. Such tags are well-known in the art and may include a radiolabeled amino acid or attachment to a polypeptide of biotinyl moieties that can be detected by marked avidin (e.g., streptavidin containing a fluorescent marker or enzymatic activity that can be detected by optical or colorimetric methods). Affinity tags such as FLAG, glutathione-S-transferase, maltose binding protein, cellulose-binding domain, thioredoxin, NusA, mistin, chitin-binding domain, cutinase, AGT, GFP and others are widely used such as in protein expression and purification systems. Further nonlimiting examples of tags for polypeptides include, but are not limited to, the following: Histidine tag, radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent tags (e.g., FITC, rhodamine, lanthanide phosphors), enzymatic tags (e.g., horseradish peroxidase, luciferase, alkaline phosphatase); chemiluminescent tags; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “replication defective” as used herein relative to a viral gene therapy vector of the invention means the viral vector cannot independently further replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate.

As used herein, a “retroviral transfer vector” refers to an expression vector that comprises a nucleotide sequence that encodes a transgene and further comprises nucleotide sequences necessary for packaging of the vector. Preferably, the retroviral transfer vector also comprises the necessary sequences for expressing the transgene in cells.

As used herein, “packaging system” refers to a set of viral constructs comprising genes that encode viral proteins involved in packaging a recombinant virus. Typically, the constructs of the packaging system are ultimately incorporated into a packaging cell.

As used herein, a “second generation” lentiviral vector system refers to a lentiviral packaging system that lacks functional accessory genes, such as one from which the accessory genes, vif, vpr, vpu and nef, have been deleted or inactivated. See, e.g., Zufferey et al. 1997. Nat. Biotechnol. 15:871-875.

As used herein, a “third generation” lentiviral vector system refers to a lentiviral packaging system that has the characteristics of a second generation vector system, and further lacks a functional tat gene, such as one from which the tat gene has been deleted or inactivated. Typically, the gene encoding rev is provided on a separate expression construct. See, e.g., Dull et al. 1998. J. Virol. 72:8463-8471.

As used herein with respect to a virus or viral vector, “pseudotyped” refers to the replacement of a native virus envelope protein with a heterologous or functionally modified virus envelope protein.

The term “operably linked” as used herein relative to a recombinant DNA construct or vector means nucleotide components of the recombinant DNA construct or vector are usually covalently joined to one another. Generally, “operably linked” DNA sequences are contiguous, and, in the case of a secretory leader, contiguous and in the same reading frame. However, enhancers do not have to be contiguous with the sequences whose expression is upregulated. The term is consistent with operably positioned.

Enhancer sequences influence promoter-dependent gene expression and may be located in the 5′ or 3′ regions of the native gene. “Enhancers” are cis-acting elements that stimulate or inhibit transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region. In addition, insulator or chromatin opening sequences, such as matrix attachment regions (Chung, Cell, 1993, Aug. 13; 74(3):505-14, Frisch et al, Genome Research, 2001, 12:349-354, Kim et al, J. Biotech 107, 2004, 95-105) may be used to enhance transcription of stably integrated gene cassettes.

As used herein, the term “gene” or “coding sequence” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

A “promoter” is a DNA sequence that directs the binding of RNA polymerase and thereby promotes RNA synthesis, i.e., a minimal sequence sufficient to direct transcription. Promoters and corresponding protein or polypeptide expression may be cell-type specific, tissue-specific, or species specific. Also included in the nucleic acid constructs or vectors of the invention are enhancer sequences which may or may not be contiguous with the promoter sequence.

“Transcription regulatory sequences”, or expression control sequences, as broadly used herein, include a promoter sequence and physically associated sequences which modulate or regulate transcription of an associated coding sequence, often in response to nutritional or environmental signals. Those associated sequences can determine tissue or cell specific expression, response to an environmental signal, binding of a protein which increases or decreases transcription, and the like. A “regulatable promoter” is any promoter whose activity is affected by a cis or trans acting factor (e.g., an inducible promoter, which is activated by an external signal or agent).

A “constitutive promoter” is any promoter that directs RNA production in many or all tissue/cell types at most times, e.g., the human CMV immediate early enhancer/promoter region which promotes constitutive expression of cloned DNA inserts in mammalian cells.

The terms “transcriptional regulatory protein”, “transcriptional regulatory factor” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to a DNA response element.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R. J. et al. 1990. Trends Biochem Sci 15:477-83) and Jackson R. J. and Kaminski, A. 1995. RNA 1:985-1000. The examples described herein are relevant to the use of any IRES element, which is able to promote direct internal ribosome entry to the initiation codon of a cistron. “Under translational control of an IRES” as used herein means that translation is associated with the IRES and proceeds in a cap-independent manner. For example, the heavy and two light chain coding sequences can be translated via IRES separating the individual coding sequences, without the need for proteolytic or self-processing to separate the two chains from one another.

A “self-processing cleavage site” or “self-processing cleavage sequence” is defined herein as a post-translational or co-translational processing cleavage site sequence. Such a “self-processing cleavage” site or sequence refers to a DNA or amino acid sequence, exemplified herein by a 2A site, sequence or domain or a 2A-like site, sequence or domain. As used herein, a “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

As used herein, the term “additional proteolytic cleavage site”, refers to a sequence which is incorporated into an expression construct of the invention adjacent a self-processing cleavage site, such as a 2A or 2A like sequence, and provides a means to remove additional amino acids that remain following cleavage by the self processing cleavage sequence. Exemplary “additional proteolytic cleavage sites” are described herein and include, but are not limited to, furin cleavage sites with the consensus sequence RXK/R-R. Such furin cleavage sites can be cleaved by endogenous subtilisin-like proteases, such as furin and other serine proteases within the protein secretion pathway.

As used herein, the terms “immunoglobulin” and “antibody” refer to intact molecules as well as fragments thereof, such as Fa, F(ab′)2, and Fv, which are capable of binding an antigenic determinant of interest. Such an “immunoglobulin” and “antibody” is composed of two identical light polypeptide chains of molecular weight approximately 23,000 daltons, and two identical heavy chains of molecular weight 53,000-70,000. The four chains are joined by disulfide bonds in a “Y” configuration. Heavy chains are classified as gamma (IgG), mu (IgM), alpha (IgA), delta (IgD) or epsilon (IgE) and are the basis for the class designations of immunoglobulins, which determines the effector function of a given antibody. Light chains are classified-as either kappa or lambda. When reference is made herein to an “immunoglobulin or fragment thereof”, it will be understood that such a “fragment thereof” is an immunologically functional immunoglobulin fragment, especially one which binds its cognate ligand with binding affinity of at least 10% that of the intact immunoglobulin.

An Fab fragment of an antibody is a monovalent antigen-binding fragment of an antibody molecule. An Fv fragment is a genetically engineered fragment containing the variable region of a light chain and the variable regions of a heavy chain expressed as two chains.

The term “humanized antibody” refers to an antibody molecule in which one or more amino acids have been replaced in the non-antigen binding regions in order to more closely resemble a human antibody, while still retaining the original binding activity of the antibody. See, e.g., U.S. Pat. No. 6,602,503.

The term “antigenic determinant”, as used herein, refers to that fragment of a molecule (i.e., an epitope) that makes contact with a particular antibody. Numerous regions of a protein or peptide or glycopeptide of a protein or glycoprotein may induce the production of antibodies which bind specifically to a given region or three-dimensional structure on the protein. These regions or structures are referred to as antigenic determinants or epitopes. An antigenic determinant may compete with the intact antigen (i.e., the immunogen used to elicit the immune response) for binding to an antibody.

The term “fragment,” when referring to a recombinant protein or polypeptide of the invention means a peptide or polypeptide which has an amino acid sequence which is the same as part of, but not all of, the amino acid sequence of the corresponding full length protein or polypeptide, which retains at least one of the functions or activities of the corresponding full length protein or polypeptide. The fragment preferably includes at least 20-100 contiguous amino acid residues of the full length protein or polypeptide.

The terms “administering” or “introducing”, as used herein, mean delivering the protein (include immunoglobulin) to a human or animal in need thereof by any route known to the art. Pharmaceutical carriers and formulations or compositions are also well known to the art. Routes of administration can include intravenous, intramuscular, intradermal, subcutaneous, transdermal, mucosal, intratumoral or mucosal. Alternatively, these terms can refer to delivery of a vector for recombinant protein expression to a cell or to cells in culture and or to cells or organs of a subject. Such administering or introducing may take place in vivo, in vitro or ex vivo. A vector for recombinant protein or polypeptide expression may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

“Transformation” is typically used to refer to bacteria comprising heterologous DNA or cells which express an oncogene and have therefore been converted into a continuous growth mode, for example, tumor cells. A vector used to “transform” a cell may be a plasmid, virus or other vehicle.

Typically, a cell is referred to as “transduced”, “infected”, “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

As used herein, the terms “stably transformed”, “stably transfected” and “transgenic” refer to cells that have a non-native (heterologous) nucleic acid sequence integrated into the genome. Stable transfection is demonstrated by the establishment of cell lines or clones comprised of a population of daughter cells containing the transfected DNA stably replicating by means of integration into their genomes or as an episomal element. In some cases, “transfection” is not stable, i.e., it is transient. In the case of transient transfection, the exogenous or heterologous DNA is expressed, however, the introduced sequence is not integrated into the genome or the host cell is not able to replicate.

As used herein, “ex vivo administration” refers to a process where primary cells are taken from a subject, a vector is administered to the cells to produce transduced, infected or transfected recombinant cells and the recombinant cells are readministered to the same or a different subject.

A “multicistronic transcript” refers to an mRNA molecule that contains more than one protein coding region, or cistron. A mRNA comprising two coding regions is denoted a “bicistronic transcript.” The “5′-proximal” coding region or cistron is the coding region whose translation initiation codon (usually AUG) is closest to the 5′ end of a multicistronic mRNA molecule. A “5′-distal” coding region or cistron is one whose translation initiation codon (usually AUG) is not the closest initiation codon to the 5′ end of the mRNA.

The terms “5′-distal” and “downstream” are used synonymously to refer to coding regions that are not adjacent to the 5′ end of a mRNA molecule.

As used herein, “co-transcribed” means that two (or more) open reading frames or coding regions or polynucleotides are under transcriptional control of a single transcriptional control or regulatory element comprising a promoter.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

As used herein, the terms “biological activity” and “biologically active”, refer to the activity attributed to a particular protein in a cell line in culture or in a cell-free system, such as a ligand-receptor assay in ELISA plates. The “biological activity” of an “immunoglobulin”, “antibody” or fragment thereof refers to the ability to bind an antigenic determinant and thereby facilitate immunological function. The “biological activity” of a hormone or interleukin is as known in the art.

As used herein, the terms “tumor” and “cancer” refer to a cell that exhibits at least a partial loss of control over normal growth and/or development. For example, often tumor or cancer cells generally have lost contact inhibition and may be invasive and/or have the ability to metastasize.

Antibodies are immunoglobulin proteins that are heterodimers of a heavy and light chain. An typical antibody is multimeric with two heavy chains and two light chains (or functional fragments thereof) which associate together. Antibodies can have a further polymeric order of structure in being dimeric, trimeric, tetrameric, pentameric, etc., often dependent on isotype. They have proven extremely difficult to express in a full length form from a single vector or from two vectors in mammalian culture expression systems. Several methods are currently used for production of antibodies: in vivo immunization of animals to produce “polyclonal” antibodies, in vitro cell culture of B-cell hybridomas to produce monoclonal antibodies (Kohler, et al. 1988. Eur. J. Immunol. 6:511; Antibodies: A Laboratory Manual, Cold Spring Harbor Laboratory, 1988; incorporated by reference herein) and recombinant DNA technology (described for example in Cabilly et al., U.S. Pat. No. 6,331,415, incorporated by reference herein).

The basic molecular structure of immunoglobulin polypeptides is well known to include two identical light chains with a molecular weight of approximately 23,000 daltons, and two identical heavy chains with a molecular weight 53,000-70,000, where the four chains are joined by disulfide bonds in a “Y” configuration. The amino acid sequence runs from the N-terminal end at the top of the Y to the C-terminal end at the bottom of each chain. At the N-terminal end is a variable region (of approximately 100 amino acids in length) which provides for the specificity of antigen binding.

The present invention is directed to improved methods for production of immunoglobulins of all types, including, but not limited to, full length antibodies and antibody fragments having a native sequence (i.e. that sequence produced in response to stimulation by an antigen), single chain antibodies which combine the antigen binding variable region of both the heavy and light chains in a single stably-folded polypeptide chain; univalent antibodies (which comprise a heavy chain/light chain dimer bound to the Fc region of a second heavy chain); “Fab fragments” which include the full “Y” region of the immunoglobulin molecule, i.e., the branches of the “Y”, either the light chain or heavy chain alone, or portions, thereof (i.e., aggregates of one heavy and one light chain, commonly known as Fab′); “hybrid immunoglobulins” which have specificity for two or more different antigens (e.g., quadromas or bispecific antibodies as described for example in U.S. Pat. No. 6,623,940); “composite immunoglobulins” wherein the heavy and light chains mimic those from different species or specificities; and “chimeric antibodies” wherein portions of each of the amino acid sequences of the heavy and light chain are derived from more than one species (i.e., the variable region is derived from one source such as a murine antibody, while the constant region is derived from another, such as a human antibody).

The compositions and methods of the invention find utility in production of immunoglobulins or fragments thereof wherein the heavy or light chain is “mammalian”, “chimeric” or modified in a manner to enhance its efficacy. Modified antibodies include both amino acid and nucleic acid sequence variants which retain the same biological activity of the unmodified form and those which are modified such that the activity is altered, i.e., changes in the constant region that improve complement fixation, interaction with membranes, and other effector functions, or changes in the variable region that improve antigen binding characteristics. The compositions and methods of the invention can further include catalytic immunoglobulins or fragments thereof.

A “variant” immunoglobulin-encoding polynucleotide sequence may encode a “variant” immunoglobulin amino acid sequence which is altered by one or more amino acids from the reference polypeptide sequence. This same discussion which follows is applicable to other biologically active protein sequences (and their coding sequences) of interest. The variant polynucleotide sequence may encode a variant amino acid sequence which contains “conservative” substitutions, wherein the substituted amino acid has structural or chemical properties similar to the amino acid which it replaces. It is understood that a variant of a the protein of interest can be made with an amino acid sequence which is substantially identical (at least about 80 to 99% identical, and all integers there between) to the amino acid sequence of the naturally occurring sequence, and it forms a functionally equivalent, three dimensional structure and retains the biological activity of the naturally occurring protein. It is well known in the biological arts that certain amino acid substitutions can be made in protein sequences without affecting the function of the protein. Generally, conservative amino acid substitutions or substitutions of similar amino acids are tolerated without affecting protein function. Similar amino acids can be those that are similar in size and/or charge properties, for example, aspartate and glutamate and isoleucine and valine are both pairs of similar amino acids. Substitutions of one for another are permitted when native secondary and tertiary structure formation are not disrupted except as intended. Similarity between amino acid pairs has been assessed in the art in a number of ways. For example, Dayhoff et al., in Atlas of Protein Sequence and Structure, 1978. Volume 5, Supplement 3, Chapter 22, pages 345-352, which is incorporated by reference herein, provides frequency tables for amino acid substitutions which can be employed as a measure of amino acid similarity. Dayhoff et al.'s frequency tables are based on comparisons of amino acid sequences for proteins having the same function from a variety of evolutionarily different sources.

Substitution mutation, insertional, and deletional variants of the disclosed nucleotide (and amino acid) sequences can be readily prepared by methods which are well known to the art. These variants can be used in the same manner as the specifically exemplified sequences so long as the variants have substantial sequence identity with a specifically exemplified sequence of the present invention and the desired functionality is preserved.

As used herein, substantial sequence identity refers to homology (or identity) which is sufficient to enable the variant polynucleotide or protein to function in the same capacity as the polynucleotide or protein from which the variant is derived. Preferably, this sequence identity is greater than 70% or 80%, more preferably, this identity is greater than 85%, or this identity is greater than 90%, and or alternatively, this is greater than 95%, and all integers between 70 and 100%. It is well within the skill of a person trained in this art to make substitution mutation, insertional, and deletional mutations which are equivalent in function or are designed to improve the function of the sequence or otherwise provide a methodological advantage. No embodiments/variants which may read on any naturally occurring proteins or which read on a qualifying prior art item are intended to be within the scope of the present invention as claimed. It is well known in the art that the polynucleotide sequences of the present invention can be truncated and/or otherwise mutated such that certain of the resulting fragments and/or mutants of the original full-length sequence can retain the desired characteristics of the full-length sequence. A wide variety of restriction enzymes which are suitable for generating fragments from larger nucleic acid molecules are well known. In addition, it is well known that Bal31 exonuclease can be conveniently used for time-controlled limited digestion of DNA. See, for example, Maniatis et al. 1982. Molecular Cloning: A Laboratory Manual, Cold Spring Harbor Laboratory, New York, pages 135-139, incorporated herein by reference. See also Wei et al. 1983. J. Biol. Chem. 258:13006-13512. By use of Bal31 exonuclease (commonly referred to as Aerase-a-base@ procedures), the ordinarily skilled artisan can remove nucleotides from either or both ends of the subject nucleic acids to generate a wide spectrum of fragments which are functionally equivalent to the subject nucleotide sequences. One of ordinary skill in the art can, in this manner, generate hundreds of fragments of controlled, varying lengths from locations all along the original coding sequence. The ordinarily skilled artisan can routinely test or screen the generated fragments for their characteristics and determine the utility of the fragments as taught herein. It is also well known that the mutant sequences of the full length sequence, or fragments thereof, can be easily produced with site directed mutagenesis. See, for example, Larionov, O. A. and Nikiforov, V. G. 1982. Genetika 18:349-59; Shortle et al. (1981) Annu. Rev. Genet. 15:265-94; both incorporated herein by reference. The skilled artisan can routinely produce deletion-, insertion-, or substitution-type mutations and identify those resulting mutants which contain the desired characteristics of the full length wild-type sequence, or fragments thereof, e.g., those which retain hormone, cytokine, antigen-binding or other biological activity.

In addition, or alternatively, the variant polynucleotide sequence may encode a variant amino acid sequence which contains “non-conservative” substitutions, wherein the substituted amino acid has dissimilar structural or chemical properties to the amino acid which it replaces. Variant immunoglobulin-encoding polynucleotides may also encode variant amino acid sequences which contain amino acid insertions or deletions, or both. Furthermore, a variant “immunoglobulin-encoding polynucleotide may encode the same polypeptide as the reference polynucleotide sequence but, due to the degeneracy of the genetic code, has a polynucleotide sequence which is altered by one or more bases from the reference polynucleotide sequence.

The term “fragment,” when referring to a recombinant immunoglobulin of the invention means a polypeptide which has an amino acid sequence which is the same as part of but not all of the amino acid sequence of the corresponding full length immunoglobulin protein, which either retains essentially the same biological function or activity as the corresponding full length protein, or retains at least one of the functions or activities of the corresponding full length protein. The fragment preferably includes at least 20-100 contiguous amino acid residues of the full length immunoglobulin, and preferably, retains the ability to bind the same antigen as the full length antibody.

As used herein, the term “sequence identity” means nucleic acid or amino acid sequence identity in two or more aligned sequences, when aligned using a sequence alignment program. The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence.

Optimal alignment of sequences for comparison can be conducted, e.g., by the local homology algorithm of Smith and Waterman. 1981. Adv. Appl. Math. 2:482, by the homology alignment algorithm of Needleman and Wunsch. 1970. J. Mol. Biol. 48:443, by the search for similarity method of Pearson and Lipman. 1988. Proc. Natl. Acad. Sci. USA 85:2444, by computerized implementations of these algorithms (GAP, BESTFIT, FASTA, and TFASTA in the Wisconsin Genetics software Package, Genetics Computer Group, Madison, Wis.), by the BLAST algorithm, Altschul et al. 1990. J. Mol. Biol. 215:403-410, with software that is publicly available through the National Center for Biotechnology Information website (see nlm.nih.gov/), or by visual inspection (see generally, Ausubel et al., infra). For purposes of the present invention, optimal alignment of sequences for comparison is most preferably conducted by the local homology algorithm of Smith and Waterman. 1981. Adv. Appl. Math. 2:482. See, also, Altschul et al. 1990 and Altschul et al. 1997.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, others known in the art, e.g., BLAST, or by visual inspection.

In accordance with the present invention, also encompassed are sequence variants which encode self-processing cleavage polypeptides and polypeptides themselves that have 80, 85, 88, 89, 90, 91, 92, 93, 94, 95, 96, 97, 98, 99% (and all integers between 80 and 100) or more sequence identity to the native sequence. Also encompassed are amino acid fragments of the polypeptides that represent a continuous stretch of at least 5, at least 10, or at least 15 units; and fragments homologous thereto according to the described identity conditions; and fragments of nucleic acid sequences that represent a continuous stretch of at least 15, at least 30, or at least 45 units.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993. An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. 2A sequence variants that encode a polypeptide with the same biological activity as the naturally occurring protein of interest and hybridize under moderate to high stringency hybridization conditions are considered to be within the scope of the present invention.

As a result of the degeneracy of the genetic code, a number of coding sequences can be produced which encode the same 2A or 2A-like polypeptide sequence or other protease or signal peptidase cleavage sequence. For example, the triplet CGT encodes the amino acid arginine. Arginine is alternatively encoded by CGA, CGC, CGG, AGA, and AGG. Therefore it is appreciated that such substitutions of synonymous codons in the coding region fall within the sequence variants that are covered by the present invention.

It is further appreciated that such sequence variants may or may not hybridize to the parent sequence under conditions of high stringency. This would be possible, for example, when the sequence variant includes a different codon for each of the amino acids encoded by the parent nucleotide. Such variants are, nonetheless, specifically contemplated and encompassed by the present invention.

The potential of antibodies as therapeutic modalities is currently limited by the production capacity and expense of the current technology. An improved viral or non-viral single expression vector for immunoglobulin (or other protein) production facilitates expression and delivery of two or more coding sequences, i.e., immunoglobulins or other proteins with bi- or multiple-specificities from a single vector. The present invention addresses these limitations and is applicable to any immunoglobulin (i.e. an antibody) or fragment thereof or other multipart protein or binding protein pair as further detailed herein, including engineered antibodies such as single chain antibodies, full-length antibodies or antibody fragments, two chain hormones, two chain cytokines, two chain chemokines, two chain receptors, and the like.

IRES

Internal ribosome entry site (IRES) elements were first discovered in picornavirus mRNAs (Jackson et al. 1990. Trends Biochem. Sci. 15:477-83) and Jackson and Kaminski. 1995. RNA 1:985-1000). Examples of IRES generally employed by those of skill in the art include those referenced in Table I, as well as those described in U.S. Pat. No. 6,692,736. Examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18:6178-6190), the fibroblast growth factor 2 (FGF-2), and insulin-like growth factor (IGFII), the translational initiation factor eIF4G and yeast transcription factors TFIID and HAP4, the encephelomyocarditis virus (EMCV) which is commercially available from Novagen (Duke et al. 1992. J. Virol 66:1602-9) and the VEGF IRES (Huez et al. 1998. Mol. Cell. Biol. 18:6178-90). IRES have also been reported in different viruses such as cardiovirus, rhinovirus, aphthovirus, HCV, Friend murine leukemia virus (FrMLV) and Moloney murine leukemia virus (MoMLV). As used herein, “IRES” encompasses functional variations of IRES sequences as long as the variation is able to promote direct internal ribosome entry to the initiation codon of a cistron. An IRES may be mammalian, viral or protozoan.

The IRES promotes direct internal ribosome entry to the initiation codon of a downstream cistron, leading to cap-independent translation. Thus, the product of a downstream cistron can be expressed from a bicistronic (or multicistronic) mRNA, without requiring either cleavage of a polyprotein or generation of a monocistronic mRNA. Internal ribosome entry sites are approximately 450 nucleotides in length and are characterized by moderate conservation of primary sequence and strong conservation of secondary structure. The most significant primary sequence feature of the IRES is a pyrimidine-rich site whose start is located approximately 25 nucleotides upstream of the 3′ end of the IRES. See Jackson et al. (1990).

Three major classes of picornavirus IRES have been identified and characterized: the cardio- and aphthovirus class (for example, the encephelomyocarditis virus, Jang et al. 1990. Gene Dev 4:1560-1572); the entero- and rhinovirus class (for example, polioviruses, Borman et al. 1994. EMBO J. 13:3149-3157); and the hepatitis A virus (HAV) class, Glass et al. 1993. Virol 193:842-852). For the first two classes, two general principles apply. First, most of the 450-nucleotide sequence of the IRES functions to maintain particular secondary and tertiary structures conducive to ribosome binding and translational initiation. Second, the ribosome entry site is an AUG triplet located at the 3′ end of the IRES, approximately 25 nucleotides downstream of a conserved oligopyrimidine tract. Translation initiation can occur either at the ribosome entry site (cardioviruses) or at the next downstream AUG (entero/rhinovirus class). Initiation occurs at both sites in aphthoviruses. HCV and pestiviruses such as bovine viral diarrhea virus (BVDV) or classical swine fever virus (CSFV) have 341 nt and 370 nt long 5′-UTR respectively. These 5′-UTR fragments form similar RNA secondary structures and can have moderately efficient IRES function (Tsukiyama-Kohara et al. 1992. J. Virol. 66:1476-1483; Frolov et al. 1998. RNA 4:1418-1435). Recent studies showed that both Friend-murine leukemia virus (MLV) 5′-UTR and rat retrotransposon virus-like 30S (VL30) sequences contain IRES structure of retroviral origin (Torrent et al. 1996. Hum. Gene Ther 7:603-612).

In eukaryotic cells, translation is normally initiated by the ribosome scanning from the capped mRNA 5′ end, under the control of initiation factors. However, several cellular mRNAs have been found to have IRES structure to mediate the cap-independent translation (van der Velde, et al. 1999. Int J Biochem Cell Biol. 31:87-106). Examples of IRES elements include, without limitation, immunoglobulin heavy-chain binding protein (BiP) (Macejak et al. 1991. Nature 353:90-94), antennapedia mRNA of Drosophila (Oh et al. 1992. Gene and Dev 6:1643-1653), fibroblast growth factor-2 (FGF-2) (Vagner et al. 1995. Mol. Cell. Biol. 15:35-44), platelet-derived growth factor B (PDGF-B) (Bernstein et al. 1997. J. Biol. Chem. 272:9356-9362), insulin-like growth factor II (Teerink et al. (1995) Biochim. Biophys. Acta 1264:403-408), and the translation initiation factor eIF4G (Gan et al. 1996. J. Biol. Chem. 271:623-626). Recently, vascular endothelial growth factor (VEGF) was also found to have IRES element (Stein et al. 1998. Mol. Cell. Biol. 18:3112-3119; Huez et al. 1998. Mol. Cell. Biol. 18:6178-6190). Further examples of IRES sequences include Picornavirus HAV (Glass et al. 1993. Virology 193:842-852); EMCV (Jang and Wimmer. 1990. Gene Dev. 4:1560-1572); Poliovirus (Borman et al. 1994. EMBO J. 13:3149-3157); HCV (Tsukiyama-Kohara et al. 1992. J. Virol. 66:1476-1483); pestivirus BVDV (Frolov et al. 1998. RNA. 4:1418-1435); Leishmania LRV-1 (Maga et al. 1995. Mol. Cell. Biol. 15:4884-4889); Retroviruses: MoMLV (Torrent et al. 1996. Hum. Gene Ther. 7:603-612). VL30, Harvey murine sarcoma virus, REV (Lopez-Lastra et al. 1997. Hum. Gene Ther. 8:1855-1865). IRES may be prepared using standard recombinant and synthetic methods known in the art. For cloning convenience, restriction sites may be engineered into the ends of the IRES fragments to be used.

To express two or more proteins from a single transcript determined by a viral or non-viral vector, an internal ribosome entry site (IRES) sequence is commonly used to drive expression of the second, third, fourth coding sequence, etc. When two coding sequences are linked via an IRES, the translational expression level of the second coding sequence is often significantly reduced (Furler et al. 2001. Gene Therapy 8:864-873). In fact, the use of an IRES to control transcription of two or more coding sequences operably linked to the same promoter can result in lower level expression of the second, third, etc. coding sequence relative to the coding sequence adjacent the promoter. In addition, an IRES sequence may be sufficiently long to impact complete packaging of the vector, e.g., the eCMV IRES has a length of 507 base pairs.

The expression of proteins in the form of polyproteins (as a primary translation product) is a strategy adopted in the replication of many viruses, including but not limited to the picornaviridae. Upon translation, virus-encoded self-processing peptides mediate rapid intramolecular (cis) cleavage of the polyprotein to yield discrete (mature) protein products. The present invention provides advantages over the use of an IRES in that a vector for recombinant protein or polypeptide expression comprising a self-processing peptide sequence (exemplified herein by 2A peptide sequence) or other protease cleavage sites is provided which facilitates expression of two or more protein or polypeptide coding sequences using a single promoter, wherein the two or more proteins or polypeptides are expressed in an advantageous molar ratio. For immunoglobulins the polyprotein is encoded by a coding sequence for one heavy chain and coding sequences for one or two light chains, with a self-processing site or protease recognition site encoded between each.

In an intein-containing construct, there can be just one of each of the heavy and light chain segments, expressed in an in frame fusion polyprotein with an intein between the two immunoglobulin chains, with the appropriate features to enable cleavage at the intein-immunoglobulin chain junctions but not re-ligation of the two immunoglobulin proteins. In another intein-containing construct, one or more additional immunoglobulin segments are present, optionally separated from the first and/or second segment by a cleavage site. For example, the intein approach is used to express one heavy chain segment and one light chain segment or to express one heavy chain and two light chains, and so forth.

A “self-processing cleavage site” or “self-processing cleavage sequence” as defined above refers to a DNA coding or amino acid sequence, wherein upon translation, rapid intramolecular (cis) cleavage of a polypeptide comprising the self-processing cleavage site occurs to yield discrete mature protein products. Such a “self-processing cleavage site”, may also be referred to as a co-translational or post-translational processing cleavage site, exemplified herein by a 2A site, sequence or domain or an intein. A 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site or domain demonstrates “auto-proteolysis” or “cleavage” by cleaving its own C-terminus in cis to produce primary cleavage products (Furler and Palmenberg. 1990. Ann. Rev. Microbiol. 44:603-623). Other protease recognition sequences, including signal peptidase cleavage sites can be substituted for the self-processing site. Inteins are also useful in polyproteins.

INTEINS

As used herein, an intein is a segment within an expressed protein, bounded toward the N-terminus of the primary expression product by an N-extein and bounded toward the C-terminus of the primary expression product by a C-extein. Naturally occurring inteins mediate excision of the inteins and rejoining (protein ligation) of the N- and C-exteins. However, in the context of the present expression products, the primary sequence of the intein or the flanking extein amino acid sequence is such that the cleavage of the protein backbone occurs in the absence of or with reduced or a minimal amount of ligation of the exteins, so that the extein proteins are released from the primary translation product (polyprotein) without their being joined to form a fusion protein. The intein portion of the primary expression product (the protein synthesized by mRNA, prior to any proteolytic cleavage) mediates the proteolytic cleavage at the N-extein/intein and the intein/C-extein junctions. In general, naturally occurring inteins also mediate the splicing together (joining by formation of a peptide bond) of the N-extein and the C-extein. However, in the present invention as applied to the goal of expressing two polypeptides (as specifically exemplified by the heavy and light chains of an antibody molecule), it is preferred that protein ligation does not occur. This can be achieved by incorporating an intein which either naturally or through mutation does not have ligation activity. Alternatively, splicing can be prevented by mutation to change the amino acid(s) at or next to the splice site to prevent ligation of the released proteins. See Xu and Perler, 1996, EMBO J. 15:5146-5153; Ser, Thr or Cys normally occurs at the start of the C-extein.

Inteins are a class of proteins whose genes are found only within the genes of other proteins. Together with the flanking host genes termed exteins, inteins are transcribed as a single mRNA, and translated as a single polypeptide. Post-translationally, inteins initiate an autocatalytic event to remove themselves and joint the flanking host protein segments with a new polypeptide bond. This reaction is catalyzed solely by the intein, require no other cellular proteins, co-factors, or ATP. Inteins are found in a variety of unicellular organisms and they have different sizes. Many inteins contain an endonuclease domain, which accounts for their mobility within genomes.

Intein mediated reactions have been used in biotechnology, especially for in vitro settings such as for purifications and for protein chip construction, and in plant strain improvement (Perler, F. B. 2005. IUBMB Life 57(7):469-76). Mutations have been introduced into native intein nucleotide sequences, and some of these mutants are reported to have altered properties (Xu and Perler, 1996. EMBO J. 15(9), 5146-5153). Besides inteins, bacterial intein-like (BIL) domains and hedgehog (Hog) auto-processing domains, the other 2 members of the Hog/intein (HINT) superfamily, are also know to catalyze post-translational self-processing through similar mechanisms (Dassa et. al. 2004. J. Biol. Chem. 279(31):32001-32007).

Inteins occur as in-frame insertions in specific host proteins. In a self-splicing reaction, inteins excise themselves from a precursor protein, while the flanking regions, the exteins, become joined to restore host gene function. These elements also contain an endonuclease function that accounts for their mobility within genomes. Inteins occur in a range of sizes (134 to 1650 amino acids), and they have been identified in the genomes of eubacteria, eukaryota and archaea. Experiments using model splicing/reporter systems have shown that the endonuclease, protein cleavage, and protein splicing functions can be separated (Xu and Perler. 1996. EMBO J. 15:5146-5153). The example described below uses an intein from Pyrococcus horikoshii Pho Pol I, Saccharomyces cerevisiae VMA, and Synechocystis spp. to create a fusion protein with sequences from an antibody heavy and light chain. Mutation of the intein designed to delete the intein's splicing capability results in a single polypeptide that undergoes a self-cleavage to produce correctly encoded antibody heavy and light chains. This strategy can be similarly employed in the expression of other multichain proteins, hormone or cytokines, and it can also be adapted for processing of precursor proteins (proproteins) to their mature, biologically active forms. While the use of the Pyrococcus horikoshii Pho Pol I, S. cerevisiae VMA, and Synechocystis spp. inteins are specifically exemplified herein, other inteins known to the art can be used in the polyprotein expression vectors and methods of the present invention.

Many other inteins besides the Pyrococcus horikoshii Pho Pol I, S. cerevisiae VMA, and Synechocystis spp. inteins are known to the art (See, e.g., Perler, F. B. 2002, InBase, the Intein Database, Nucl. Acids Res. 30(1):383-384 and the Intein Database and Registry, available via the New England Biolabs website, e.g., at http://tools.neb.com/inbase/). Inteins have been identified in a wide range of organisms such as yeast, mycobacteria and extreme thermophilic archaebacteria. Certain inteins have endonuclease activity as well as the site-specific protein cutting and splicing activities. Endonuclease activity is not necessary for the practice of the present invention; an endonuclease coding region can be deleted, provided that the protein cleavage activity is maintained.

The mechanism of the protein splicing process has been studied in great detail (Chong et al. 1996. J. Biol. Chem. 271: 22159-22168; Xu and Perler. 1996. EMBO J. 15: 5146-5153) and conserved amino acids have been found at the intein and extein splicing points (Xu et al. 1994. EMBO J. 13:5517-5522). The constructs described herein contain an intein sequence fused to the 5′-terminus of the first coding sequence, with a second coding sequence fused in frame a the C-terminus of the intein. Suitable intein sequences can be selected from any of the proteins known to contain protein splicing elements. A database containing all known inteins can be found on the World Wide Web (Perler, F. B. 1999. Nucl. Acids Res. 27: 346-347). The intein coding sequence is fused (in frame) at the 3′ end to the 5′ end of a second coding sequence. For targeting of this protein to a certain organelle, an appropriate peptide signal can be fused to the coding sequence of the protein.

After the second extein coding sequence, the intein coding sequence-extein coding sequence can be repeated as often as desired for expression of multiple proteins in the same cell. For multi-intein containing constructs, it may be useful to use intein elements from different sources. After the sequence of the last gene to be expressed, a transcription termination sequence, and advantageously including a polyadenylation sequence, is desirably inserted. The order of a polyadenylation sequence and a termination sequence can be as understood in the art. In an embodiment, a polyadenylation sequence can precede a termination sequence.

Modified intein splicing units have been designed so that such a modified intein of interest can catalyze excision of the exteins from the inteins but cannot catalyze ligation of the exteins (see, e.g., U.S. Pat. No. 7,026,526 and US Patent Publication 20020129400). Mutagenesis of the C-terminal extein junction in the Pyrococcus species GB-D DNA polymerase produced an altered splicing element that induces cleavage of exteins and inteins but prevents subsequent ligation of the exteins (Xu and Perler. 1996. EMBO J. 15: 5146-5153). Mutation of serine 538 to either an alanine or glycine (Ser to Ala or Gly) induced cleavage but prevented ligation. At such position, Ser to Met or Ser to Thr are also used to achieve expression of a polyprotein that is cleaved into separate segments and at least partially not re-ligated. Mutation of equivalent residues in other intein splicing units can also prevent ligation of extein segments due to the relative conservation of amino acids at the C-terminal extein junction to the intein. In instances of low conservation/homology, for example, the first several, e.g., about five, residues of the C-extein and/or the last several residues of the intein segment are systematically varied and screened for the ability to support cleavage but not splicing of given extein segments, in particular extein segments disclosed herein and as understood in the art. There are inteins that do not contain an endonuclease domain; these include the Synechocystis spp dnaE intein and the Mycobacterium xenopi GyrA protein (Magnasco et al, Biochemistry, 2004, 43, 10265-10276; Telenti et al. 1997. J. Bacteriol. 179: 6378-6382). Others have been found in nature or have been created artificially by removing the endonuclease encoding domains from the sequences encoding endonuclease-containing inteins (Chong et al. 1997. J. Biol. Chem. 272: 15587-15590). Where desired, the intein is selected originally so that it consists of the minimal number of amino acids needed to perform the splicing function, such as the intein from the Mycobacterium xenopi GyrA protein (Telenti et al. 1997.supra). In an alternative embodiment, an intein without endonuclease activity is selected, such as the intein from the Mycobacterium xenopi GyrA protein or the Saccharomyces cerevisiae VMA intein that has been modified to remove endonuclease domains (Chong et al. 1997. supra).

Further modification of the intein splicing unit may allow the reaction rate of the cleavage reaction to be altered, allowing protein dosage to be controlled by simply modifying the gene sequence of the splicing unit.

In an embodiment, the first residue of the C-terminal extein is engineered to contain a glycine or alanine, a modification that was shown to prevent extein ligation with the Pyrococcus species GB-D DNA polymerase (Xu and Perler. 1996. EMBO J. 15: 5146-5153). In this embodiment, preferred C-terminal extein proteins naturally contain a glycine or an alanine residue following the N-terminal methionine in the native amino acid sequence. Fusion of the glycine or alanine of the extein to the C-terminus of the intein provides the native amino acid sequence after processing of the polyprotein. In another embodiment, an artificial glycine or alanine is positioned in the C-terminal extein either by altering the native sequence or by adding an additional amino acid residue onto the N-terminus of the native sequence. In this embodiment, the native amino acid sequence of the protein will be altered by one amino acid after polyprotein processing. In further embodiments, other modifications useful in the present invention are described in U.S. Pat. No. 7,026,526.

The DNA sequence of the Pyrococcus species GB-D DNA Polymerase intein is SEQ ID NO:1 of U.S. Pat. No. 7,026,526. The N-terminal extein junction point is the “aac” sequence (nucleotides 1-3 of SEQ ID NO:1) and encodes an asparagine residue. The splicing sites in the native GB-D DNA Polymerase precursor protein follow nucleotide 3 and nucleotide 1614 in SEQ ID NO:1. The C-terminal extein junction point is the “agc” sequence (nucleotides 1615-1617 of SEQ ID NO:1), which encodes a serine residue. Mutation of the C-terminal extein serine to an alanine or glycine forms a modified intein splicing element that is capable of promoting excision of the polyprotein but not ligation of the extein units.

The DNA sequence of the Mycobacterium xenopi GyrA minimal intein is SEQ ID NO:2 of U.S. Pat. No. 7,026,526. The N-terminal extein junction point is the “tac” sequence (nucleotides 1-3 of SEQ ID NO:2) and encodes a tyrosine residue. The splicing sites in the precursor protein follow nucleotide 3 and nucleotide 597 of SEQ ID NO:2. The C-terminal extein junction point is the “acc” sequence (nucleotides 598-600 of SEQ ID NO:2) and encodes a threonine residue. Mutation of the C-terminal extein threonine to an alanine or glycine forms a modified intein splicing element that promotes excision of the polyprotein but does not ligate the extein units.

2A Systems

Turning now to the 2A protease processing embodiment of the present invention, the activity of 2A may involve ribosomal skipping between codons which prevents formation of peptide bonds (de Felipe et al. 2000. Human Gene Therapy 11:1921-1931; Donnelly et al. 2001. J. Gen. Virol. 82:1013-1025), although it has been considered that the domain acts more like an autolytic enzyme (Ryan et al. 1989. Virology 173:35-45). Studies in which the Foot and Mouth Disease Virus (FMDV) 2A coding region was cloned into expression vectors and transfected into target cells have established that FMDV 2A cleavage of artificial reporter polyproteins is efficient in a broad range of heterologous expression systems (wheat-germ lysate and transgenic tobacco plant (Halpin et al., U.S. Pat. No. 5,846,767 (1998) and Halpin et al. 1999. The Plant Journal 17:453-459); Hs 683 human glioma cell line (de Felipe et al. 1999. Gene Therapy 6:198-208; hereinafter referred to as “de Felipe II”); rabbit reticulocyte lysate and human HTK-143 cells (Ryan et al. 1994. EMBO J. 13:928-933); and insect cells (Roosien et al. 1990. J. Gen. Virol. 71:1703-1711). The FMDV 2A-mediated cleavage of a heterologous polyprotein for a biologically relevant molecule has been shown for IL-12 (p40/p35 heterodimer; Chaplin et al. 1999. J. Interferon Cytokine Res. 19:235-241). In transfected COS-7 cells, FMDV 2A mediated the cleavage of a p40-2A-p35 polyprotein into biologically functional p40 and p35 subunits having activities associated with IL-12.

The FMDV 2A sequence has been incorporated into expression vectors, alone or combined with different IRES sequences to construct bicistronic, tricistronic and tetracistronic vectors. The efficiency of 2A-mediated gene expression in animals was demonstrated by Furler (2001) using recombinant adeno-associated viral (AAV) vectors encoding α-synuclein and EGFP or Cu/Zn superoxide dismutase (SOD-1) and EGFP linked via the FMDV 2A sequence. EGFP and α-synuclein were expressed at substantially higher levels from vectors which included a 2A sequence relative to corresponding IRES-based vectors, while SOD-1 was expressed at comparable or slightly higher levels.

The DNA sequence encoding a self-processing cleavage site is exemplified by viral sequences derived from a picornavirus, including but not limited to an entero-, rhino-, cardio-, aphtho- or Foot-and-Mouth Disease Virus (FMDV). In a preferred embodiment, the self-processing cleavage site coding sequence is derived from a FMDV. Self-processing cleavage sites include but are not limited to 2A and 2A-like domains (Donnelly et al. 2001. J. Gen. Virol. 82:1027-1041, incorporated by reference in its entirety).

Alternatively, a protease recognition site can be substituted for the self-processing site. Suitable protease and cognate recognitions sites include, without limitation, furin, RXR/K-R (SEQ ID NO:1); VP4 of IPNV, S/TXA-S/AG (SEQ ID NO:2); Tobacco etch virus (TEV) protease, EXXYXQ-G (SEQ ID NO:3); 3C protease of rhinovirus, LEVLFQ-GP (SEQ ID NO:4); PC5/6 protease; PACE protease, LPC/PC7 protease; enterokinase, DDDDK-X (SEQ ID NO:5); Factor Xa protease IE/DGR-X (SEQ ID NO:6); thrombin, LVPR-GS (SEQ ID NO:7); genenase I, PGAAH-Y (SEQ ID NO:8); and MMP protease; an internally cleavable signal peptide, an example of which is the internally cleavable signal peptide of influenza C virus (Pekosz A. 1998. Proc. Natl. Acad. Sci. USA 95: 113233-13238) (MGRMAMKWLVVIICFSITSQPASA, SEQ ID NO:11). The protease can be provided in trans or in cis as part of the polyprotein, such that it is encoded within the same transcription and separated from the remainder of the primary translation product, for example, by a self-processing site or protease recognition site.

As more and more antibody therapeutics become approved for clinical applications, there has been steady improvement in the methods for manufacturing these therapeutic proteins over the last 20 years (Wurm, F M, 2004, “Production of recombinant protein therapeutics in cultivated mammalian cells,” Nat. Biotechnol. 22(11): 1393). However, still more efficient and reliable production methods are desired by the industry. Some desirable features include higher levels of antibody secretion into the culture media, improved genetic stability of manufacturing cell lines, and greater speed in the generation of cell lines.

In our search for more efficient methods for producing therapeutic antibodies, we have developed methods for expressing antibody heavy chain and light chain from a single open reading frame. In one such method, an intein coding sequence is used to separate the antibody heavy and light chain genes within a single open reading frame (sORF). Advantages offered by such a sORF antibody expression technology include the ability to manipulate gene dosage ratios for heavy and light chains, the proximity of heavy and light chain polypeptides for multi-subunit assembly in ER, and the potential for high efficiency protein secretion.

Other technology for expressing monoclonal antibodies in mammalian cells involves introducing the heavy and the light chain genes in two separate ORFs, each with its own promoter and regulatory sequences. Promoter interference is a concern associated with this method. An alternative method to introduce the antibody heavy and light chain coding sequences into the expression cell lines is to use internal ribosomal entry site (IRES) to separate the antibody heavy and light chain coding sequences. This method has not been widely used because of the decreased efficiency in translating the coding sequence downstream of the IRES sequence. Recently, a method that uses a sequence encoding the foot-and-mouth virus peptide (2A peptide) to separate the coding sequences for antibody heavy and light chain has been described (Fang et. al. 2005. Nat. Biotechnol. 23(5):584-90). In this method the antibody heavy and light chain and the 2A peptide are transcribed as a single mRNA. However, the antibody heavy and light chain polypeptides are cleaved before they enter the endoplasmic reticulum (ER). In addition, two non-native amino acids are left at the C-terminus of the heavy chain after the cleavage/separation of the heavy and light chains. The intein expression system of the present invention is fundamentally different. It differs from the 2A method in that the heavy and light chain polypeptide are translated and brought into ER as a single polyprotein. Advantageously, it is not necessary for non-native amino acids to be included in the mature antibody molecules.

The following descriptions are all in the context of the antibody-production vectors comprising expression cassettes as follows: Promoter-Secretion signal-heavy chain-wt intein such as p. horikoshii Pol I intein-secretion signal-light chain-polyA; Promoter-Secretion signal-heavy chain-modified intein such as p. horikoshii Pol I intein-light chain-polyA; Promoter-Secretion signal-heavy chain-Pol modified intein such as p. horikoshii Pol I intein-secretion signal-light chain-Pol modified intein such as p. horikoshii Pol I intein-Secretion signal-light chain-polyA; Promoter-Secretion signal-heavy chain-wt or modified intein such as p. horikoshii Pol I intein-modified secretion signal-light chain-polyA; Promoter-Secretion signal-light chain-wt or modified intein such as P. horikoshii Pol I intein-modified secretion signal-heavy chain-polyA; Promoter-Secretion signal-heavy chain-wt or modified intein such as p. horikoshii Pol I intein-modified secretion signal-light chain-wt or modified intein such as P. horikoshii Pol I intein-modified secretion signal-light chain-polyA; Promoter-Secretion signal-heavy chain-Furin cleavage site-modified intein such as P. horikoshii Pol I intein-Furin Cleavage site-secretion signal-Light Chain-polyA; and Promoter-heavy chain-Furin cleavage site-modified intein such as P. horikoshii Pol I intein-Furin Cleavage site-Light Chain-Furin Cleavage site-modified intein such as P. horikoshii Pol I intein-Furin cleavage site-light chain-polyA. In further constructs, a modified Psp-GBD Pol intein is used.

The specifically exemplified polyprotein described here makes use of the P. horikoshii Pol I intein that was fused in frame with the D2E7 heavy chain and light chain before and after it respectively. The amino acid that was in the −1 position was a lysine and the amino acid that was in the +1 position was a Methionine, the first amino acid of the light chain signal peptide. The use of methionine at the +1 position allowed for abolishment of splicing, the joint of the heavy and light chains, as we have demonstrated in the latter sections, with an understanding that a nucleophilic amino acid residue such as serine, cysteine, or threonine is needed at the +1 position to allow for splicing. In addition to wt inteins, mutations that change the last amino acid asparagine and the second to last histidine can be used as these mutations generally abolish splicing and preserve cleavage at the N-terminal splicing junction (Mills, 2004; Xu, 1996, Chong, 1997). Alternatively mutations that change the 1^{st} amino acid of the intein can also be used, as such mutations generally abolishes splicing, preserve the cleavage at the C-terminal splicing junction, and either abolish or preserve attenuated cleavage at the N-terminal splicing junction (Nichols, 2004; Evans, 1999, and Xu, 1996). For example, this has been demonstrated to “completely block splicing and inhibit the formation of the branched intermediate, resulting in the cleavage at both splice junctions” (Xu, M. Q., EMBO vol. 15:5146-5153).

In an alternative version of the polypeptide, inclusion of the furin cleavage site allows alteration of the junction sequence with subsequent excision via furin cleavage during secretion. The wildtype sequence for the intein is given in Table 9. In the DNA polymerase I of Pyrococcus spp. GB-D, the cleavage/splice junctions are RQRAIKILAN/S (SEQ ID NO:138) (N terminal) and HN/SYYGYYGYAK (SEQ ID NO:139) (C terminal). Desirably, the endonuclease coding region is excised by HindIII cleavage. The cleavage, splicing and endonuclease functions are dissociated from one another and this endonuclease region can be substituted with a small linker to create mini-inteins that are still capable of cleavage and splicing (Telenti et al. 1997. J. Bacteriol. 179:6378-6382). It is noted that at least one yeast intein functions in mammalian cells (Mootz et al. 2003. J. Am. Chem. Soc. 125:10561-10569). See Tables 8A and 8B for the coding and amino acid sequences of a D2E7 (immunoglobulin) intein construct; Table 8C provides the complete nucleotide sequence of a D2E7 intein construct expression vector. A fusion construction is described that encodes the heavy chain of D2E7 (Humira—registered trademark for adalimumab) fused to the modified Psp Pol1 intein which is itself fused to the coding region for D2E7 light chain. The light chain sequence can be duplicated, with an intein, signal peptide or protease cleavage site(s) separating it from the remainder of the polyprotein. In this embodiment the mature heavy chain is preceded by the heavy chain secretion signal. The intein has been altered as described above, the serine 1 being changed to a threonine and the internal Hind III fragment excised to remove the endonuclease activity. The intein is fused in-frame to the mature D2E7 light chain region. An alternate embodiment would include the light chain secretion signal 5′ of the mature light chain. See FIGS. 10 and 11 for schematic representation of the D2E7 intein construct and expression vector and Tables 8A-8C for the nucleotide sequences of the expression construct and the complete expression vector and the amino acid sequence of the D2E7 intein construct.

Signal Peptides and Signal Peptidases

The signal hypothesis, wherein proteins contain information within their amino acid sequences for protein targeting to the membrane, has been known for more than thirty years. Milstein and co-workers discovered that the light chain of IgG from myeloma cells was synthesized in a higher molecular weight form and was converted to its mature form when endoplasmic reticulum vesicles (microsomes) were added to the translation system, and proposed a model based on these results in which microsomes contain a protease that converts the precursor protein form to the mature form by removing the amino-terminal extension peptide. The signal hypothesis was soon expanded to include distinct targeting sequences within proteins localized to different intracellular membranes, such as the mitochondria and chloroplast. These distinct targeting sequences were later found to be cleaved from the exported protein by specific signal peptidases (SPases).

There are at least three distinct SPases involved in cleaving signal peptides in bacteria. SPase I can process nonlipoprotein substrates that are exported by the SecYEG pathway or the twin arginine translocation (Tat) pathway. Lipoproteins that are exported by the Sec pathway are cleaved by SPase II. SPase IV cleaves type IV prepilins and prepilin-like proteins that are components of the type II secretion apparatus.

In eukaryotes, proteins that are targeted to the endoplasmic reticulum (ER) membrane are mediated by signal peptides that target the protein either cotranslationally or post-translationally to the Sec61 translocation machinery. The ER signal peptides have features similar to those of their bacterial counterparts. The ER signal peptides are cleaved from the exported protein after export into the ER lumen by the signal peptidase complex (SPC). The signal peptides that sort proteins to different locations within the eukaryotic cell have to be distinct because these cells contain many different membranous and aqueous compartments. Proteins that are targeted to the ER often contain cleavable signal sequences. Amazingly, many artificial peptides can function as translocation signals. The most important key feature is believed to be hydrophobicity above a certain threshold. ER signal peptides have a higher content of leucine residues than do bacterial signal peptides. The signal recognition particle (SRP) binds to cleavable signal peptides after they emerge from the ribosome. The SRP is required for targeting the nascent protein to the ER membrane. After translocation of the protein to the ER lumen, the exported protein is processed by the SPC. Another embodiment takes advantage of signal (leader) peptide processing enzymes which occur naturally in eukaryotic cells. In eukaryotes, proteins that are targeted to the endoplasmic reticulum (ER) membrane are mediated by signal peptides that target the protein either cotranslationally or post-translationally to the Sec61 translocation machinery. The ER signal peptides are cleaved from the exported protein after export into the ER lumen by the signal peptidase complex (SPC). Most of known ER signal peptides are either N-terminal cleavable or internally uncleavable. Recently, a number of viral polyproteins such as those found in the hepatitis C virus, hantavirus, flavivirus, rubella virus, and influenza C virus were found to contain internal signal peptides that are most likely cleaved by the ER SPC. These studies on the maturation of viral polyproteins show that SPC can cleave not only amino-terminally located signal peptides, but also after internal signal peptides.

The presenilin-type aspartic protease signal peptide peptidase (SPP) cleaves signal peptides within their transmembrane region. SPP is essential for generation of signal peptide-derived HLA-E epitopes in humans. Recently, a number of viral polyproteins such as those found in the hepatitis C virus, hantavirus, flavivirus, rubella virus, and influenza C virus were found to contain internal signal peptides that are most likely cleaved by the ER SPC. Mutagenesis of the predicted signal peptidase substrate specificity elements may thus block viral infectivity. These studies on the maturation of polyproteins are also very interesting because they show that SPC can cleave not only amino-terminally located signal peptides, but also after internal signal peptides. Signal peptidases are well known in the art. See, for example, Paetzel M. 2002. Chem. Rev. 102(12): 4549; Pekosz A. 1998. Proc. Natl. Acad. Sci. USA. 95:13233-13238; Marius K. 2002. Molecular Cell 10:735-744; Okamoto K. 2004. J. Virol. 78:6370-6380, Vol. 78; Martoglio B. 2003. Human Molecular Genetics 12: R201-R206; and Xia W. 2003. J. Cell Sci. 116:2839-2844.

Proteins that are targeted to the endoplasmic reticulum (ER) membrane are mediated by signal peptides that target the protein either cotranslationally or post-translationally to the Sec61 translocation machinery. The ER signal peptides are cleaved from the exported protein after export into the ER lumen by the signal peptidase complex (SPC). Most of known ER signal peptides are either N-terminal cleavable or internally uncleavable. Recently, a number of viral polyproteins such as those found in the hepatitis C virus, hantavirus, flavivirus, rubella virus, and influenza C virus were found to contain internal signal peptides that are most likely cleaved by the ER SPC. These studies on the maturation of viral polyproteins show that SPC can cleave not only amino-terminally located signal peptides, but also after internal signal peptides.

This invention utilizes internal cleavable signal peptides for expression of a polypeptide in a single transcript. The single transcribed polypeptide is then cleaved by SPC, leaving individual peptides separately or individual peptides being assembled into a protein. The methods of the present invention are applicable to the expression of immunoglobulin heavy chain and light chain in a single transcribed polypeptide, followed by cleavage, then assembly into a mature immunoglobulin. This technology is applicable to polypeptide cytokines, growth factors, or a variety of other proteins, for example, IL-12p40 and IL-12p35 in a single transcribed polypeptide and then assembly into IL-12, or IL-12p40 and IL-23p19 in a single transcribed polypeptide and then assembly into IL-23.

The signal peptidase approach is applicable to mammalian expression vectors which result in the expression of functional antibody or other processed product from a precursor or polyprotein. In the case of the antibody, it is produced from the vector as a polyprotein containing both heavy and light chains, with an intervening sequence between heavy chain and light chain being an internal cleavable signal peptide. This internal cleavable signal peptide can be cleaved by ER-residing proteases, mainly signal peptidases, presenilin or presenilin-like proteases, leaving heavy and light chains to fold and assemble to give a functional molecule, and desirably it is secreted. In addition to the internal cleavable signal peptide derived from hepatitis C virus, other internal cleavable sequences which can be cleaved by ER-residing proteases can be substituted thereof. Similarly, the practice of the invention need not be limited to host cells in which signal peptidase effects cleavage, but it also includes proteases including, but not limited to, presenilin, presenilin-like protease, and other proteases for processing polypeptides. Those proteases have been reviewed in the cited articles, among others.

In addition, the present invention is not limited to the expression of immunoglobulin heavy and light chains, but it also includes other polypeptides and polyproteins expressed in single transcripts followed by internal signal peptide cleavage to release each individual peptide or protein. These proteins may or may not assemble together in the mature product.

Also within the scope of the present invention are expression constructs in which the individual polypeptides are present in alternate orders, i.e., “Peptide 1-internal cleavable signal peptide-peptide 2” or “Peptide 2-internal cleavable signal peptide-peptide 1”. This invention further includes expression of more than two peptides linked by internal cleavable signal peptides, such as “Peptide 1-internal cleavable signal peptide-peptide 2-internal cleavable signal peptide-peptide 3”, and so on.

In addition, this invention applies to expression of both type I and type II transmembrane proteins and to the addition of other protease cleavage sites surrounding expression constructs. One example is to add a furin or PC5/6 cleavage site after an immunoglobulin heavy chain to facilitate the cleaving off of additional amino acid residues at the carboxyl-terminal of heavy chain peptide, e.g., “Heavy chain-furin cleavage site-internal cleavable signal peptide-Light chain”. The present invention also includes more than one internal cleavable signal peptide separately or in tandem, for example, “Heavy chain-furin cleavage site-internal cleavable signal peptide-internal cleavable signal peptide-Light chain”. Further, this invention includes situations where there is maintenance or removal of self signal peptides of heavy chain and light chain, such as “HC signal peptide-Heavy chain-furin cleavage site-internal cleavable signal peptide-LC signal peptide-Light chain”.

The following descriptions are in the context of antibody-production vectors, some of which are described elsewhere herein. Vector designs include but are not limited to the following.

Table of vector designs.

Promoter - Secretion signal - heavy chain - internal cleavable signal
peptide - secretion signal - light chain - polyA;
Promoter - Secretion signal - heavy chain - internal cleavable signal
peptide - light chain - polyA;
Promoter - Secretion signal - heavy chain - internal cleavable signal
peptide - secretion signal - light chain - internal cleavable signal
peptide - Secretion signal - light chain - polyA;
Promoter - Secretion signal - heavy chain - Furin cleavage site - internal
cleavable signal peptide - Furin Cleavage site - secretion signal - Light
Chain - polyA; and
Promoter - heavy chain - Furin cleavage site - internal cleavable signal
peptide - Furin Cleavage site - Light Chain - Furin Cleavage site -
internal cleavable signal peptide - Furin cleavage site - light chain - polyA.

A specific example of a fusion construct encodes the heavy chain of D2E7 (Humira/adalimumab) fused to internal cleavable signal peptide which is itself fused to the coding region for D2E7 light chain. In this embodiment the mature heavy chain is preceded by the heavy chain secretion signal. The internal cleavable signal peptide sequence is derived from Influenza C virus. A furin cleavage site is included in the carboxyl terminus of heavy chain. To minimize the affect on the mature antibody, the third to last amino residue of heavy chain is mutated from proline to arginine to create a furin cleavage site. An alternate embodiment would include the light chain secretion signal 5′ of the mature light chain. See Tables 9A-9C. The minimal internal cleavable signal peptide sequence from Influenza C virus (MGRMAMKWLVVIICFSITSQPASA, SEQ ID NO:11) is used in the example. A longer sequence may also be used to enhance the cleavage efficiency. See GenBank accession number AB126196. A variety of nucleotide sequence encoding the same amino acid sequence can also be used.

This invention can further utilize internal cleavable signal peptides for maturation of one or more polypeptides within a polyprotein encoded within a single transcript. The single transcribed polypeptide is then cleaved by SPC, leaving individual peptides separately or individual peptides being assembled into a protein. This invention is applicable to express immunoglobulin heavy chain and light chain in a single transcribed polypeptide and then assembly into a mature immunoglobulin. This invention is applicable to express polypeptide cytokines, growth factors, or a variety of other proteins for example to express IL-12p40 and IL-12p35 in a single transcribed polypeptide and then assembly into IL-12, or IL-12p40 and IL-23p19 in a single transcribed polypeptide and then assembly into IL-23.

Positional subcloning of a 2A sequence or other protease or signal peptidase cleavage (recognition) site between two or more heterologous DNA sequences for the inventive vector construct allows the delivery and expression of two or more genes through a single expression vector. Preferably, self processing cleavage sites such as FMDV 2A sequences or protease recognition sequences provide a unique means to express and deliver from a single viral vector, two or multiple proteins, polypeptides or peptides which can be individual parts of, for example, an antibody, heterodimeric receptor or heterodimeric protein.

FMDV 2A is a polyprotein region which functions in the FMDV genome to direct a single cleavage at its own C-terminus, thus functioning in cis. The FMDV 2A domain is typically reported to be about nineteen amino acids in length (LLNFDLLKLAGDVESNPGP, SEQ ID NO:12; TLNFDLLKLAGDVESNPGP, SEQ ID NO:13; Ryan et al. 1991. J. Gen. Virol. 72:2727-2732), however oligopeptides of as few as fourteen amino acid residues (LLKLAGDVESNPGP, SEQ ID NO:14) have been shown to mediate cleavage at the 2A C-terminus in a fashion similar to its role in the native FMDV polyprotein processing.

Variations of the 2A sequence have been studied for their ability to mediate efficient processing of polyproteins (Donnelly et al. 2001). Homologues and variants of a 2A sequence are included within the scope of the invention and include but are not limited to the following sequences: QLLNFDLLKLAGDVESNPGP, SEQ ID NO:15; NFDLLKLAGDVESNPGPFF, SEQ ID NO:16; LLKLAGDVESNPGP, SEQ ID NO:17; NFDLLKLAGDVESNPGP, SEQ ID NO:18; APVKQTLNFDLLKLAGDVESNPGP, SEQ ID NO:19; VTELLYRMKRAETYCPRPLLAIHPTEARHKQKIVAPVKQTLNFDLLKLAGDVESNPGP, SEQ ID NO:20; LLAIHPTEARHKQKIVAPVKQTLNFDLLKLAGDVESNPGP, SEQ ID NO:141; and EARHKQKIVAPVKQTLNFDLLKLAGDVESNPGP, SEQ ID NO:142.

A sequences and variants thereof can be used to make vectors expressing self-processing polyproteins, including any vector (plasmid or virus based) which includes the coding sequences for proteins or polypeptides linked via self-processing cleavage sites or other protease cleavage sites such that the individual proteins are expressed in the appropriate molar ratios and/or amounts following the cleavage of the polyprotein due to the presence of the self-processing or other cleavage site. These proteins may be heterologous to the vector itself, to each other or to the self-processing cleavage site, e.g., FMDV, thus the self-processing cleavage sites for use in practicing the invention do not discriminate between heterologous proteins and coding sequences derived from the same source as the self-processing cleavage site, in the ability to function or mediate cleavage.

In one embodiment, the FMDV 2A sequence included in a vector according to the invention encodes amino acid residues comprising LLNFDLLKLAGDVESNPGP (SEQ ID NO:12). Alternatively, a vector according to the invention may encode amino acid residues for other 2A-like regions as discussed in Donnelly et al. 2001. J. Gen. Virol. 82:1027-1041 and including, but not limited to, a 2A-like domain from picornavirus, insect virus, Type C rotavirus, trypanosome repeated sequences or the bacterium, Thermatoga maritima.

The invention contemplates use of nucleic acid sequence variants that encodes a 2A or 2A-like peptide sequence, such as a nucleic acid coding sequence for a 2A or 2A-like polypeptide which has a different codon for one or more of the amino acids relative to that of the parent nucleotide. Such variants are specifically contemplated and encompassed by the present invention. Sequence variants of 2A peptides and polypeptides are included within the scope of the invention as well. Similarly, proteases supplied in cis or in trans can mediate proteolytic processing via cognate protease recognition (cleavage) sites between the regions of the polyprotein.

In further experiments with intein-antibody expression constructs, we have demonstrated that the Pyrococcus horikoshii Pol I intein-mediated protein splicing reaction can take place in mammalian (293E) cells, in ER, and in the context of an antibody (D2E7) heavy and light chain amino acid sequences. For the purpose of using this type of reaction in antibody expression in a single open reading frame (sORF) format, we demonstrated that this reaction can take place in mammalian cells (293E), in ER, and in the context of antibody heavy and antibody light chain amino acid sequences using two constructs, pTT3-HcintLC1aa-p.hori and pTT3-HcintLC3aa-p.hori. See Tables 11A and 12 A.

These constructs were made on the PTT3 vector backbone. This vector has an Epstein Barr virus (EBV) origin of replication, which allows for its episomal amplification in transfected 293E cells (cells that express Epstein-Barr virus nuclear antigen 1) in suspension culture (Durocher, 2002, “High level and high-throughput recombinant protein production by transient transfection of suspension-growing human 293-EBNA1 cells, Nucleic Acids Research 30(2):E9). Each vector had one ORF, transcriptionally expressed under the regulatory control of a CMV promoter. In the ORF, a P. horikoshii PolI intein was inserted in frame between the D2E7 heavy and light chains, each having a signal peptide (SP). The pTT3-HcintLClaa-p.hori and pTT3-HcintLC3aa-p.hori constructs had 1 native extein amino acid, or 3 native extein amino acids on the either side of the intein, separating the D2E7 antibody heavy and light chain sequences from the intein sequence. These constructs were introduced into 293E cells through transient transfection. Both the culture supernatant and cell pellet samples were analyzed.

Cell pellet samples were lysed under conditions that allow separation of the cytosolic and intracellular membrane fractions. Both of these fractions were analyzed using western blots (WB) with either an anti-heavy chain or an anti-kappa light chain antibody. On these blots we saw the expression of 4 protein species corresponding to a tri-partite form as in the construct's ORF (130 kDa), a fusion of H and L, which was derived from a splicing event (80 kDa), an antibody heavy chain (50 kDa), and an antibody light chain (25 kDa). The first 2 protein species were detected by both the anti-heavy chain and the anti-light chain antibodies, the heavy chain was detected only by the anti-heavy chain antibody, and the light chain was detected by only the anti-light chain antibody. The presence of the 80 kDa protein species, which was detected by both the heavy and the light chain antibodies in both of these constructs, demonstrated that a protein splicing event had taken place. Furthermore, all four protein species were predominantly present in the sub-cellular membrane fraction, which contained endoplasmic reticulum (ER). This indicated that the heavy chain signal peptide (encoded at the beginning of the ORF) had directed the entire polypeptide into ER, where the splicing reaction had taken place. Without wishing to be bound by any particular theory, it is believed that the free heavy and light chain polypeptides were likely to be the result of cleavages at the N-terminal and the C-terminal splicing junctions, resulting from incomplete splicing.

Cell pellet samples were also used for total RNA extraction and Northern blot analysis using both an antibody heavy chain probe and an antibody light chain probe. Northern blot analysis revealed a tripartite mRNA (3.4 kb) in these sORF constructs, which was hybridized with both the heavy chain probe and the light chain probe, but not the mRNA for a separate heavy chain or a light chain. In contrast, in the cell pellet samples that expressed the D2E7 antibody using the conventional approach, that is, introducing the antibody heavy and the light chains from two separate ORFs carried in two pTT3 vectors, mRNAs for the heavy (1.4 kb) and the L chain (0.7 kb) were detected using the heavy chain or light chain probes respectively. No tripartite mRNA was detected in these control cell pellets.

The above described data demonstrate that using constructs containing a single ORF (D2E7 heavy chain-P. horikoshi intein-D2E7 light chain), a single mRNA containing all 3 proteins was transcribed. This tripartite message was translated into a tripartite polypeptide, and co-translationally imported into ER, directed by the heavy chain signal peptide present at the N-terminus of the tripartite polyprotein. With this construct, the intein-mediated protein splicing reaction took place inside the ER. This suggested that intein-mediated reactions could be used in the expression of antibodies, as well as other multi-subunit secreted proteins, i.e., those proteins that need to go through the secretory pathway in order to be folded and properly post-translationally modified.

Culture supernatants were also analyzed. Both Western Blot and ELISA allow detection of antibody secreted from expression of the pTT3-HcintLC1aa-p.hori construct. These studies are discussed in more detail herein below; the amount of secreted antibody expression has been increased through both point mutations and the mutation within the sequence encoding the light chain signal peptide.

Mutations designed to inhibit intein-mediated ligation but preserve the cleavage reactions at either the N-terminal or the C-terminal splicing junctions resulted in increased levels of antibody secretion.

With the goal of enhanced efficiency of antibody secretion, three types of point mutations were designed and tested. The first type of mutation was in the codon of the first serine residue of the C-terminal extein; these constructs had Ser to Met (S>M) changes (construct pTT3-HcintLC-p.hori, construct E, and construct A). The second type of mutation was at the coding for the first serine residue of the intein; such a construct had a Ser to Thr (S>T) change (construct E). The third type of mutation was in the codon for the histidine residue that was the second to last (penultimate) amino acid of the intein; these constructs had a H is to Ala (H>A) substitution mutation (construct A and construct B). These mutations were introduced either alone or in combination. All the mutant constructs were designed to preserve the cleavage at either the N- or the C-terminal splicing junctions and reduce splicing of the released exteins, or both, according to reaction mechanisms described in the literature. As outlined below the secretion of D2E7 antibody is achieved using a number of these constructs.

In one experiment, these constructs were introduced into 293E cells through transient transfection, and after 7 days, the cultured supernatants were analyzed for IgG antibody titers by ELISA analysis. The antibody titers for constructs pTT3-HcintLC3aa-p.hori, pTT3-HcintLClaa-p.hori, pTT3-HcintLC-p.hori, E, A, and B were 17.0+0.6, 113.8+2.6, 225.8+10.0, 9.3+0.5, 161.7+4.4, and 48.2+1.0 ng/ml (average+s.d.), respectively.

These supernatant samples were also analyzed on SDS-PAGE gel under denaturing conditions, and blotted with an antibody against the human IgG heavy chain and an antibody against the human Kappa light chain. On these western blots the antibody heavy chain (˜50 kDa) and the antibody light chain (˜25 kDa) are clearly visible in the supernatants generated from constructs pTT3-HcintLC-p.hori and A, consistent with the rank order of IgG levels measured by ELISA.

Cell pellet samples from these transfections were also characterized using western blot analysis. A tripartite-polypeptide (˜130 kDa) along with the antibody heavy chain (˜50 kDa) and light chain (˜25 kDa) bands are seen in the cell pellets containing all the above-described constructs. Among these the constructs, pTT3-HcintLC-p.hori and construct A gave the strongest heavy chain and the light chain bands; therefore it was concluded that there was a correlation between level of intracellular free heavy and light chains and the assembled and secreted antibodies. The spliced product (˜80 kDa), that is the fusion between the antibody heavy chain and light chain, was present in cell pellets generated using construct pTT3-HcintLC3aa-p.hori and to a lesser extent in cell pellets generated from the construct pTT3-HcintLC1aa-p.hori; it was absent in constructs pTT3-HcintLC-p.hori and constructs A, B, and E. This indicated that the level of protein splicing was inversely correlated with antibody secretion efficiency, consistent with the expectation that the joining of the antibody heavy and light chains would result in misfolding, based on the general knowledge about antibody structure, and this misfolding would consequently prevent secretion due to cellular mechanisms for degradation of misfolded proteins. Another protein species on these blots was intein-light chain fusion (80 kDa, recognized by the light chain antibody but not the heavy chain antibody), which resulted from a cleavage at the N-terminal splicing junction in the absence of any additional cleavages. This band was present in constructs A, B, E, pTT3-HcintLC3aa-p.hori, pTT3-HcintLC1aa-p.hori, and mostly absent in constructs pTT3-HcintLC-p.hori and H, described herein. Therefore the presence of this protein species was also inversely related to the amount of antibody secretion. Finally, an intein band was also detected in these cell lysates using rabbit polyclonal antisera generated against a P. horikoshii peptide, conjugate to KLH.

We demonstrated that the D2E7 antibody secreted using the sORF construct pTT3-HcintLC-p.hori has the correct N-terminal sequences of the heavy and light chains, the correct heavy and light chain molecular weights and intact molecular weights.

The D2E7 antibody secreted using one of sORF construct pTT3-HcintLC-p.hori was purified by Protein A affinity chromatography and analyzed with respect to the N-terminal sequences of both its heavy chain and its light chains. The unambiguous results indicated that the N-terminal peptide sequence of the heavy chain was EVQLVESGGG (SEQ ID NO:21) and the N-terminal sequence of the light chain was DIQMTQSPSS (SEQ ID NO:22). Thus, using this construct, the cleavage sites used by the signal peptidase w DIQMTQSPSS ere the same as those used in the conventional, two ORF/two vector approach to DE27 antibody expression.

These data provided important scientific insights for the design of the next generation of constructs: the mammalian ER peptidase could recognize and accurately cleave a signal peptide in the newly synthesized polyprotein, even though there were some apparent requirements for its presentation (see herein below).

This purified antibody was analyzed by mass spectrometry, along with the D2E7 produced by the conventional manufacturing process. Under denaturing conditions, D2E7 light chain produced from the pTT3-HcintLC-p.hori construct yielded one single peak on the mass spectrum and its molecular weight (MW) was 23408.8, whereas the molecular weight (MW) of the D2E7 light chain produced from standard manufacturing process was 23409.7, in close agreement. Also under denaturing conditions, the D2E7 heavy chain produced from the pTT3-HcintLC-p.hori construct yielded one major peak and 2 minor peaks on the mass spectrum and their molecular weights (MW) were 50640.6, 50768.2, and 50802.4 respectively, where-as the molecular weights (MW) of the D2E7 heavy chain produced from standard manufacturing process were 50641.7, 50768.6, and 50804.1, respectively, again in close agreement. The 3 peaks correspond to the standard variations of the D2E7 heavy chain.

The intact molecular weights (MW) under native conditions for this D2E7 antibody produced from the pTT3-HcintLC-p.hori construct, along with the D2E7 antibody produced from the manufacturing process, were also determined using mass spectrometry. The D2E7 antibody produced from the pTT3-HcintLC-p.hori construct had 3 peaks, with MW of 148097.6, 148246.9, and 148413.1 respectively; the D2E7 antibody produced from the manufacturing process also had 3 peaks, with MW of 148096.0, 148252.3, and 148412.8, respectively.

These data demonstrated clearly that the D2E7 antibody produced from the pTT3-HcintLC-p.hori construct was identical in size to the D2E7 antibody produced from the conventional manufacturing process, under both the denaturing and native conditions. The ability to produce antibodies with completely authentic amino acid sequences as compared to the conventional manufacturing method is one of the advantages of antibody expression system of the present invention. Using the 2A system as described by Fang et. al. in Nature Biotechnology, 2005, for example, the antibody produced had 2 extra non-native amino acids at the C-terminus of its heavy chain, and this could not be avoided due to the nature of the cleavage.

We have also demonstrated that the D2E7 antibody produced using the pTT3-HcintLC-p.hori sORF construct had the same affinity for binding TNF as the D2E7 antibody produced from the manufacturing process. Real-time binding interactions between rhTNFa antagonists captured across a biosensor chip via immobilized goat anti-human IgG, and soluble rhTNFa were measured using a Biacore 3000 instrument (Pharmacia LKB Biotechnology, Uppsala, Sweden) according to the manufacturer's instructions and standard procedures. Briefly, rhTNFa aliquots were diluted into a HBS-EP (Biacore) buffer, and 150-μl aliquots were injected across the immobilized protein matrices at a flow rate of 25 ml/min. Equivalent concentration of analyte was simultaneously injected over an untreated reference surface to serve as blank sensorgrams for subtraction of bulk refractive index background. The sensor chip surface was regenerated between cycles with two 5-min injections of 10 mM Glycine, at 25 ml/min. The resultant experimental binding sensorgrams were then evaluated using the BIA evaluation 4.0.1 software to determine kinetic rate parameters. Datasets for each antagonist were fit to the 1:1 Langmuir model. For these studies, binding and dissociation data were analyzed under global fit analysis protocol while selecting fit locally for maximum analyte binding capacity (RU) or Rmax attribute. In this case, the software calculated a single dissociation constant (kd), association constant (ka), and affinity constant (Kd). The equilibrium dissociation constant is Kd=kd/ka. The kinetic on-rate, the kinetic off rate, and the overall affinities were determined by using different TNFα concentrations in the range of 1-100 nM. The kinetic on-rate, kinetic off rate, and overall affinity for the D2E7 antibody produced from the construct pTT3-HcintLC-p.hori were 1.61 E+6 (M⁻¹s⁻¹), 5.69 E-5(s⁻¹), and 3.54E-11(M) respectively; the kinetic on-rate, kinetic off rate, and overall affinity for the D2E7 antibody produced via the manufacturing process were 1.73E+6(M⁻¹s⁻¹), 6.72E-5(s⁻¹), and 3.89E-11(M) respectively. Biacore analysis indicated that the D2E7 antibody produced using this sORF construct has similar affinity to TNF as the D2E7 antibody produced by the conventional manufacturing process.

Modification of Signal Peptide

We have demonstrated that in the sORF construct design, Heavychain-int-LightChain, the antibody secretion level was increased about 10 fold when the hydrophobicity of the light chain signal peptide sequence was reduced through site-directed mutagenesis.

We designed construct H, in which following the P. horikoshi intein sequence, the light chain signal peptide sequence was changed from “MDMRVPAQLLGLLLLWFPGSRC” (SEQ ID NO:23) to “MDMRVPAQLLG DE WFPGSRC” (SEQ ID NO:24). In the same type of transfection experiment as described above, the supernatant of cells which expressed this construct contained 2047+116 ng/ml antibody as measured by ELISA analysis. This level of antibody secretion is similar to that described using the 2A technology (1.6 μg/ml). Western blot analysis of this supernatant showed strong bands corresponding to the antibody heavy chain and the antibody light chain.

In a control experiment, this same light chain signal peptide mutation was introduced into a vector for expressing this antibody using the conventional approach (expressing the antibody heavy and light chains from two separate open reading frames in two separate vectors). In this construct, the change in SEQ ID NO:23 to provide SEQ ID NO:24 abolished antibody secretion as expected because the hydrophobic region is important for targeting to the signal recognition particle (SRP) complex on the ER and directing the entrance into the translocon, in the conventional construct design. This verified that in the sORF construct design, the targeting function of the light chain signal peptide is dispensable, even though it can be recognized and cleaved by the ER signal peptidase, consistent with the hypothesis that the entire ORF had entered into the ER as directed by the heavy chain signal peptide at the beginning of the ORF.

The D2E7 antibody secreted using sORF construct H was purified by Protein A affinity chromatography and analyzed with respect to the N-terminal sequence of its light chain. The N-terminal peptide sequence of the light chain was MDMRVPAQLL (SEQ ID NO:26) (without ambiguity), which represented the un-cleaved signal peptide. Even though the literature suggests that the H region of a mammalian ER signal peptide functions primarily in targeting to (SRP) complex and directing the translocation through the translocon, our data suggested that the hydrophobic (H) region of the signal peptide also plays a role in recognition and cleavage by signal peptidase.

We have demonstrated that D2E7 antibodies secreted using both the pTT3-HcintLC-p.hori construct and the construct H were biologically active in cell-based assays. The D2E7 antibody produced using construct pTT3-HcintLC-p.hori and construct H were purified and tested in their ability to neutralize TNFa induced cytotoxicity in L929 cells. This assay was carried out essentially as described in U.S. Pat. No. 6,090,382 (see Example 4 therein). Human recombinant TNFa causes cytotoxicity in murine L929 cells and was used in this assay. As D2E7, an anti-TNFa antibody, can neutralize this cytotoxicity, L929 assay is one of the cell based assays that can be used to evaluate the biological activity of a particular D2E7 antibody preparation. When analyzed using this assay D2E7 produced from both the pTT3-HcintLC-p.hori construct and the construct H neutralized TNFa induced cytotoxicity. Their IC50 values were similar to that by D2E7 produced from standard manufacturing process.

We have investigated additional constructs with different designs in the light chain signal peptide area. To identify the optimal sORF construct design that would allow for high antibody secretion efficiency, we have designed several additional constructs that varied the region around the C-terminal splicing site and the following signal peptide. Construct J determined “MDMRVPAQWFPGSRC” (SEQ ID NO:25) following the last N of the intein instead of the “MDMRVPAQLLG DE WFPGSRC” (SEQ ID NO:24) of the H construct, which further removed the hydrophobic region inside this signal peptide while preserving the C-terminal region as well as signal peptidase cleavage site. Construct K directed expression of the mature light chain sequence directly following the last N of the intein. Construct L directed expression of “MDMRVPAQLLGLLLLWFPGSGG” (SEQ ID NO:27) following the last N of the intein instead of “MDMRVPAQLLGLLLLWFPGSRC” (SEQ ID NO:23) as in construct pTT3-HcintLC-p.hori, which changed the −1 and −2 amino acids before the cleavage site by the signal peptidase.

In an experiment, these constructs were introduced into 293E cells through transient transfections, and after 7 days, the cultured supernatants were analyzed for IgG antibody titers by ELISA analysis. The antibody titers for constructs H, J, K, and L were 2328.5+79.9, 1289.7+129.6, 139.3+4.7, and 625.0+20.6 ng/ml (average+s.d.), respectively.

The cell pellet samples from these transfections were also analyzed by western blot analysis. All constructs had the tripartite polypeptide band (˜130 kDa), the heavy chain band (˜50 kDa), and the light chain band (˜25 kDa) described previously, and none had detectable spliced product (80 kDa and recognized by both the heavy chain and the light chain antibody). Among this group of constructs, the construct K produced the most distinctive western blot (WB) pattern in that it produced only a very small amount of the intracellular light chain, and instead it produced the protein species corresponding to intein-light chain fusion, a product of one cleavage event at the N-terminal splice junction. This protein species was absent with the other constructs in this group. The construct K differed from the other constructs in two aspects: it did not have a cleavage site by the signal peptidase, and it had an aspartic acid, instead of a methionine or a serine, as the 1st amino acid residue of the C-terminal extein. Either or both of these features could have prevented the cleavage at the area between the intein and the antibody light chain, resulting in decreased antibody secretion.

The D2E7 antibody secreted using the sORF construct J and L were purified by Protein A affinity chromatography and analyzed for the N-terminal sequences of their light chain. This analysis indicated that the N-terminal peptide sequence of the light chain produced by construct J was MDMRVPAQLL, which represented the un-cleaved signal peptide; whereas the N-terminal peptide sequence of the light chain produced by construct L was DIQMTQSPSS, which represented the mature light chain after correct signal peptide cleavage. Therefore, construct L represent a design that gave increased antibody secretion (0.6-1 ug/ml in different transient transfections) compared to the construct pTT3-HcintLC-p.hori, and its light chain had the correct N-terminal sequence at the same time.

We explored mechanisms of expressing assembled antibody from sORF constructs using inteins and methods for further increasing antibody secretion levels. Intracellular samples of cells transfected with most of the sORF constructs described contained two antibody light chain species corresponding to the un-processed and processed light chains. In cell transfected with either the positive control constructs or the pTT3-HcintLC-p.hori construct only the processed light chain was secreted, indicating that un-processed light chains that have attached wild type light chain signal peptides could not be assembled and secreted. In contrast, the un-processed light chains from the H and the J constructs were able to be assembled and secreted; both had mutated signal peptides. The extent of the light chain signal peptide processing, as seen in the distributions of the intracellular light chain polypeptide between the un-processed and processed forms, varies depending on the construct. Compared to construct pTT3-HcintLC-p.hori, the construct L had an increased amount of processed light chain, and this has translated into increased antibody secretion.

Based on the above experimental data one way to increase antibody secretion from the sORF constructs is to improve processing efficiency of the light chain signal peptide. This is performed by systematically testing mutations in both the hydrophobic region as well as in the area around the cleavage site, and by testing signal peptides of different length. This can also be done by screening in yeast for peptide sequences that can be cleaved efficiently in this presentation, and by doing similar screenings in CHO cells.

Another method that can be used to increase the antibody secretion level from the sORF constructs is to test different 5′ and 3′ untranslated regions (UTRs) to increase the stability of the tripartite mRNA, as these mRNAs are larger than traditional mRNAs coding for the antibody heavy and light chains separately.

Another method for increase the antibody secretion level from the sORF constructs is to generate and select stable CHO or NS0 cell line and amplify using either DHFR or GS to increase the recombinant gene copy numbers. The antibody secretion level is independently increased by changing the location of the recombinant genes from episomal (transient) to genomic (stable). It is also enhanced by increasing copy number, and/or by manipulating 5′ and 3′ UTRs, promoter and enhancer sequences. Vectors expressing dihydrofolate reductase (dhfr) are transfected into dhfr-deficient cell lines. Cell lines with higher vector copy numbers are selected using methotrexate, a competitive inhibitor of dhfr (Kaufman, R. J. and Sharp, P. A. J. Mol. Biol. (1982) 159:601-621). As a further independent alternative, expression vectors carrying the cytomegalovirus promoter enhancer in conjunction with a glutamine synthetase selectable marker are employed to increase expression (Bebbington, C. R. (1991) Methods 2:138-145). In addition to increasing the recombinant gene copy numbers, the cellular lineages that are particularly amenable for the processing from sORF construct designs are also selected in this process.

Using Modified Inteins Containing Insertions

For the purpose of tracking intracellular intein proteins that have been separated from the D2E7 heavy chain and light chain polypeptides, we have made 4 constructs that introduced a Histidine tag at amino acid sequence positions FRKVR ! RGRG (! Represents insertion sites, -HT1), and EGKR ! IPEF (-HT2), in both constructs pTT3-LcintHC-p.hori and construct H. These 2 positions in the P. horikoshi intein was hypothesized to be loops that can tolerate inserts while maintaining its 3-dimentional structure and therefore its function. In one experiment, after 4 days of incubation following transfection of 293E cells, the culture supernatants were analyzed for IgG antibody titers by ELISA analysis. The antibody titers for constructs pTT3-LcintHC-p.hori-HT1, pTT3-LcintHC-p.hori-HT2, construct H-HT1, construct H-HT2, and construct H were 78.3+3.2, 67.3+0.6, 663.0+15.5, 402.7+5.5, 747.0+22.5 ng/ml (average+s.d.), respectively. Use of P. horikoshii intein with insertions at both of the 2 locations have allowed the secretion of assembled antibody. In particular, the use of the intein with an internal inserted tag at the 1st position gave similar antibody secretion level as compared to using intein without any insertion.

The above data demonstrates that sORF construct designs of the present invention include use of modified inteins that contain an internal tag. A variety of tags are known in the art. Tags of the present invention include but are not limited to fluorescent tags and chemiluminescent tags. Using such constructs, the amount of polyprotein expressed can be monitored using fluorescent detection in individual cells. In addition, these cells can be sorted according to the level of protein expression using FACS. The use of such tags are particularly useful in stable cell line generations as this allows the selection of high producing cells or cell lines through FACS analysis. As taught in the present invention, full length inteins have been observed in the cell lysate after their being auto-cleaved from the flanking antibody heavy and light chains. This provides bases for the detections of fluorescent labeled inteins and their use in stable cell line generation. Tags can also be used in purification of proteins.

From the data presented above, we have learned that the P. horikoshii Pol I intein-mediated protein splicing reaction can take place in 293E cells, in ER, and in the context of antibody (as specifically exemplified by D2E7) heavy and light chain amino acid sequences. Point substitution mutations such as S>M at the first amino acid of the C-terminal extein and H>A at the penultimate amino acid of the intein increased the levels of secreted antibody. Reducing the hydrophobicity of the H region of the light chain signal peptide, such as in constructs H and J, produced even higher levels of antibody secretion. The antibody secretion level in a construct that lacks the light chain signal peptide is relatively low, and this appeared to be due to less efficient cleavage at the C-terminal splicing junction. Two approaches are used to increase the efficiency of this cleavage. The first uses an amino acid other than the Aspartic Acid at the +1 position. Also several constructs described here used methionine at the +1 position and gave efficient cleavage at the C-terminal splicing junction. A second approach for increasing the efficiency of this cleavage is to alter the spacing between the C-terminal cleavage site and the light chain globular structure with the use of a linker, optionally followed by a different type of cleavage site such as those described in this disclosure.

While various constructs comprising the P. horikoshii intein and the DE27 antibody have been described and tested, other inteins and intein-like proteins (including hedgehog and related family) are used in sORF designs of the invention, e.g., incorporated between antibody heavy and light chains. Other multiple subunit proteins (including two-subunit proteins and proteins with more than two subunits) are substituted for the heavy and light proteins of antibody as well.

In addition to the P. horikoshii PolI intein constructs described herein above, we have designed analogous constructs using Sce.VMA intein and Ssp. dnaE mini intein: pTT3-Hc-VMAint-LC-0aa, pTT3-Hc-VMAint-LC-1 aa, pTT3-Hc-VMAint-LC-3aa, pTT3-Hc-Ssp-GA-int-LC-0aa, pTT3-Hc-Ssp-GA-int-LC-1aa, and pTT3-Hc-Ssp-GA-int-LC-3aa. These constructs were transfected into 293E cells, and supernatant and cell pellet samples were analyzed.

In one experiment, after 7 days of incubation following transfection of 293E cells, the culture supernatants were analyzed for IgG antibody titers by ELISA analysis. The antibody titers for constructs pTT3-Hc-VMAint-LC-0aa, pTT3-Hc-VMAint-LC-1aa, pTT3-Hc-VMAint-LC-3aa, pTT3-HC-Ssp-GA-int-LC-0aa, pTT3-HC-Ssp-GA-int-LC-1aa, and pTT3-HC-Ssp-GA-int-LC-3aa were 9.0±3.5, 12.0±0.0, 39.7±1.2, 90.0±2.0, 38.7±1.5, and 32±2.6 ng/ml (average+s.d.), respectively.

Cell pellet samples from these transfections were also analyzed by western blot analysis. The tripartite polypeptides were observed in all of these samples. In addition, the heavy chain polypeptide was observed in constructs pTT3-Hc-VMAint-LC-0aa, pTT3-HC-Ssp-GA-int-LC-0aa, pTT3-HC-Ssp-GA-int-LC-1 aa, and pTT3-HC-Ssp-GA-int-LC-3aa; and the light chain polypeptide was observed in pTT3-HC-Ssp-GA-int-LC-0aa, pTT3-HC-Ssp-GA-int-LC-1aa, and pTT3-HC-Ssp-GA-int-LC-3aa.

The results of those experiments indicated that inteins, as a class of proteins, can be used successfully in sORF protein expression strategies as we described. Furthermore, bacterial intein-like (BIL) domains and hedgehog (Hog) auto-processing domains, the other 2 members of the Hog/intein (HINT) superfamily besides intein, are applicable in similar construct designs to those described herein.

Additionally, because endonuclease regions that are present in many inteins, including the P. horikoshii PolI intein and the Sce.VMA intein, are not useful in the present gene expression strategy, the endonuclease domain can be deleted and replace with a small linker to create “mini-inteins”.

These engineered mini-inteins are also useful in the described construct designs, and they present the advantage that the intein coding region is significantly smaller, thus allowing for a larger sequence encoding the polypeptides of interest and/or greater ease of handling the recombinant DNA molecules.

One concern associated with the use of self-processing peptides, such as 2A or 2A-like sequences or protease recognition sequences is that the C or N termini of the one or more of the polypeptide chains contain(s) amino acids derived from the self-processing peptide, i.e. 2A-derived amino acid residues, or protease recognition sequence, depending on the position cleaved and the relative position of the particular chain within the primary translation product. These amino acid residues are “foreign” to the host and may elicit an immune response when the recombinant protein is expressed or delivered in vivo (i.e., expressed from a viral or non-viral vector in the context of gene therapy or administered as an in vitro-produced recombinant protein). In addition, if not removed, 2A-derived or protease site-derived amino acid residues may interfere with protein secretion in producer cells and/or alter protein conformation, resulting in a less than optimal expression level and/or reduced biological activity of the recombinant protein.

Gene expression constructs, engineered such that an additional proteolytic cleavage site is provided between a polypeptide coding sequence and the self processing cleavage site (i.e., a 2A-sequence) or other protease cleavage site as a means for removal of remaining self processing cleavage site derived amino acid residues following cleavage can be used in the practice of the present invention.

Examples of additional proteolytic cleavage sites are furin cleavage sites with the consensus sequence RXK(R)R (SEQ ID NO:1), which can be cleaved by endogenous subtilisin-like proteases, such as furin and other serine proteases within the protein secretion pathway. US Patent Publication 2005/0042721 shows that the 2A residues at the N terminus of the first protein can be efficiently removed by introducing a furin cleavage site RAKR between the first polypeptide and the 2A sequence. In addition, use of a plasmid containing a 2A sequence and a furin cleavage site adjacent to the 2A site was shown to result in a higher level of protein expression than a plasmid containing the 2A sequence alone. This improvement provides a further advantage in that when 2A residues are removed from the N-terminus of the protein, longer 2A- or 2A like sequences or other self-processing sequences can be used. Such longer self-processing sequences such as 2A- or 2A like sequences may facilitate better equimolar expression of two or more polypeptides by way of a single promoter. Still further increased in immunoglobulin expression are achieved when the immunoglobulin light chain coding sequence is present twice and the heavy chain coding sequence is present only once in the polyprotein.

It is advantageous to employ antibodies or analogues thereof with fully human characteristics. These reagents avoid the undesired immune responses induced by antibodies or analogues originating from non-human species. To address possible host immune responses to amino acid residues derived from self-processing peptides, the coding sequence for a proteolytic cleavage site may be inserted (using standard methodology known in the art) between the coding sequence for the first protein and the coding sequence for the self-processing peptide so as to remove the self-processing peptide sequence from the expressed polypeptide, i.e. the antibody. This finds particular utility in therapeutic or diagnostic antibodies for use in vivo.

Any additional proteolytic cleavage site known in the art which can be expressed using recombinant DNA technology vectors may be employed in practicing the invention. Exemplary additional proteolytic cleavage sites which can be inserted between a polypeptide or protein coding sequence and a self processing cleavage sequence (such as a 2A sequence) include, but are not limited to a Furin cleavage site, RXK(R)R (SEQ ID NO:1); a Factor Xa cleavage site, IE(D)GR (SEQ ID NO:6); Signal peptidase I cleavage site, e.g. LAGFATVAQA (SEQ ID NO:28); and thrombin cleavage site, LVPRGS (SEQ ID NO:7).

As an alternative to the IRES, furin, 2A and intein approaches to the expression of more than one mature protein from a single open reading frame, the present invention also provides for protein processing using a hedgehog protein domain positioned within a polyprotein between first and second protein portions. we designed a single open reading frame for expressing antibody heavy chain and light chain with a hedgehog autoprocessing domain to separate the antibody heavy and light chain genes. In cells that carry such an ORF, a single mRNA that consists of at least one antibody heavy chain, one antibody light chain, and one hedgehog autoprocessing domain is transcribed and used to generate a corresponding polyprotein. Post-translationally, the hedgehog autoprocessing domain mediates the separation of the antibody heavy and light chains.

The hedgehog family of proteins contains conserved signaling molecules that act as morphogens in different developmental systems, and are involved in a wide range of human diseases (Kalderon, D. 2005. Biochem Soc Trans. December; 33(Pt 6):1509-12). Hedgehog proteins have 2 structural domains, a N-terminal domain (Hh-N) that functions in cell signaling, and a C-terminal domain (Hh-C) that catalyzes a post-translational autoprocessing event that cleaves between these 2 domains, adds a cholesterol moiety to the C-terminus of the N-terminal domain, and thereby activates the signaling molecule. (Traci et al. 1997. Cell, 91, 85-97).

Advantages offered by such a sORF antibody expression technology include the ability to manipulate gene dosage ratios for heavy and light chains, the proximity of heavy and light chain polypeptides for multi-subunit assembly in ER, and the potential for high efficiency protein secretion.

The Hh-C protein domains can be used to catalyze an autoprocessing reaction in ER that result in a post-translational cleavage between the antibody heavy chain polypeptide and the Hh-C polypeptide in the single open reading frame construct design described below.

Hedgehog family of proteins has a N-terminal signaling domain and a C-terminal autoprocessing domain. Their C-terminal autoprocessing domains cleave themselves from the N-terminal domains, and add to their C-termini a cholesterol moiety through a 2-step reaction mechanism (Porter et al. 1996. Science. 274(5285):255-9). In addition to cholesterol, other nucleophiles such as DTT or glutathione also stimulate the autoprocessing (Lee et al. 1994. Science, 266, 1528-1537). As the cleavage reaction is catalyzed by the C-terminal autoprocessing domain, a similar cleavage reaction takes place when the N-terminal signaling domain of the hedgehog protein is replaced by an antibody heavy chain or light chain polypeptide. This reaction can be used to separate the antibody heavy and light chains contained within a polyprotein encoded by single open reading frame.

First the antibody expression is tested in a transient expression system and for this purpose, constructs are made on a PTT3 vector backbone. This vector has EBV origin of replication, which allows for its episomal amplification in transfected 293E cells (cells that express Epstein-Barr virus nuclear antigen 1) in suspension culture (Durocher et al. 2002). Each vector has a single open reading frame, driven by a CMV promoter. In one construct design, pTT3-HC-Hh-C25-LC, the entire C-terminal domain of the sonic hedgehog protein from Drosophila melanogaster was inserted in frame between the D2E7 heavy and light chains, each of which had a signal peptide (SP). These constructs are introduced into 293E cells through transient transfection. Both the cultured supernatants and cell pellet sample are analyzed.

Cell pellet samples are lysed under conditions that allow separation of the cytosolic and intracellular membrane fractions. Both of these fractions are analyzed using immunoblots techniques with either an anti-heavy chain or an anti-kappa light chain antibody. On these blots protein species are observed include the poly protein (HC-Hh-C25-LC), Hh-C25-LC, and the separate heavy (HC) and light chains (LC). The presence of the latter 3 protein species confirm that the autoprocessing reaction has taken place. The free heavy chain is generated from the cleavage catalyzed by the Hh-C protein domain; the free light chain polypeptides are the results of a cleavage by the signal peptidase. The segregation of protein species in the sub-cellular membrane fraction that contained endoplasmic reticulum (ER) suggest that the heavy chain signal peptide at the beginning of our ORF had directed the entire ORF into ER, where the cleavage reaction takes place.

These cell pellet samples are also subjected to total RNA extraction and Northern blot analysis using both an antibody heavy chain-specific probe and an antibody light chain-specific probe. On these northern blots observations of a tripartite mRNA that hybridizes to both the heavy chain probe and the light chain probe confirms the sORF nature of the construct design. In contrast, in the cell pellet samples that expressed the D2E7 antibody using the conventional approach, that is, introducing the antibody heavy and the light chains from two separate ORFs carried in two pTT3 vectors, mRNAs for the heavy (1.4 kb) and the L chain (0.7 kb) have been detected using the heavy chain or light chain probes respectively.

These experiments demonstrate that using constructs containing a single ORF (D2E7 heavy chain-Hh-C25-D2E7 light chain), a single mRNA containing all 3 proteins is transcribed. This tripartite message is translated into a tripartite polypeptide, and co-translationally imported into ER, directed by the heavy chain signal peptide present at the beginning of the ORF. This indicates that Hh-C protein domain is useful for the expression of antibodies, as well as of other multi-subunit secreted proteins and/or other proteins that need to go through the secretory pathways in order to be folded and properly post-translationally modified.

In addition to the cell pellets the cultured supernatants are analyzed, using both western blots and ELISA, for secreted antibodies, as discussed herein. Constructs using deleted hh-C25 can be tested to compare efficiencies of polyprotein processing and antibody secretion level.

It has been shown that deletion of the C-terminal 63 amino acid from the Hh-C25 protein domain yielded a protein domain, Hh-C17, which can catalyze protein processing but not the cholesterol addition. Hh-C17 expressed well as a recombinant protein and its crystal structure has been determined (Traci et al. 1997. supra). Therefore, in another construct design, pTT3-HC-C17-LC, this truncated protein domain was inserted between the D2E7 antibody heavy and light chains.

In the homology alignment of hedgehog proteins and inteins, which we have tested in similar construct designs as described in detail herein, the last 8 amino acids are extensions beyond the last predicted β-sheet secondary structure, and they may or may not contribute to the efficiency of the auto-processing. Therefore, an additional construct, pTT3-HC-C17sc-LC, is also tested.

These constructs are introduced into 293E cells through transient transfection, and after 7 days, the cultured supernatants can be analyzed for IgG antibody titers by ELISA analysis. The antibody titers for pTT3-HC-C25-LC, pTT3-HC-C17-LC, pTT3-HC-C17sc-LC, and pTT3-HC-C17hn-LC are 0.038, 0.042, 0.040 and 0.046 ug/ml respectively.

These supernatant samples are also analyzed on SDS-PAGE gels (denaturing conditions), and blotted with antibody specific for the human IgG heavy chain and an antibody specific for the human Kappa light chain. On these western blots the antibody heavy chain (˜50 kDa) and the antibody light chain (˜25 kDa) proteins can be observed and correlated with IgG levels measured by ELISA.

The cell pellet samples from these transfections are also analyzed by western blot analysis. The presence and relative density of the four protein species described can be compared among different constructs to determine the protein processing efficiencies afforded by each of the construct designs.

In another class of self-processing proteins, inteins, the last two amino acids tend to be HisAsn. In the process of protein-splicing catalyzed by inteins the Asn undergoes a cyclization, assisted by the His, which results in a cleavage of a peptide bond between the intein and its C-terminal flanking polypeptide. In contrast to inteins, hedgehog auto-processing proteins do not in nature have a C-terminal flanking polypeptide and they do not have a conserved Asn at this position of the polypeptide. In one construct design, pTT3-HC-C17hn-LC, we have introduced His-Asn at this position, replacing Ser-Cys. Without wishing to be bound by theory, the engineered cleavage site at this position makes the separation between the hedgehog auto-processing protein and the antibody light chain in this particular construct design more efficient. The efficiency of antibody secretion is tested as described above.

Antibodies produced through sORF constructs containing hedgehog auto-processing protein are characterized. The D2E7 antibody secreted using the above sORF construct are purified by Protein A affinity chromatography and analyzed for the N-terminal sequences of both its heavy chain and its light chain. These purified antibodies are analyzed by mass spectrometry as previously described, along with the D2E7 produced from the standard manufacturing process, under the denaturing conditions. Using mass spectrometry the intact molecular weights (MW) under native conditions are determined for the D2E7 antibody produced from these constructs, along with the D2E7 antibody produced from the manufacturing process.

The binding between D2E7 antibody and human TNFα is analyzed using Biacore as described before. The kinetic on-rate, kinetic off rate, and overall affinities are determined by using different TNFα concentrations in the range of 1-100 nM.

The present invention contemplates the use of any of a variety of vectors for introduction of constructs comprising the coding sequence for two or more polypeptides or proteins and a self processing cleavage sequence into cells. Numerous examples of gene expression vectors are known in the art and may be of viral or non-viral origin. Non-viral gene delivery methods which may be employed in the practice of the invention include but are not limited to plasmids, liposomes, nucleic acid/liposome complexes, cationic lipids and the like.

Viral Vectors

Viral and other vectors can efficiently transduce cells and introduce their own DNA into a host cell. In generating recombinant viral vectors, non-essential genes are replaced with expressible sequences encoding proteins or polypeptides of interest. Exemplary vectors include but are not limited to viral and non-viral vectors, such a retroviral vector (including lentiviral vectors), adenoviral (Ad) vectors including replication competent, replication deficient and gutless forms thereof, adeno-associated virus (AAV) vectors, simian virus 40 (SV-40) vectors, bovine papilloma vectors, Epstein-Barr vectors, herpes vectors, vaccinia vectors, Moloney murine leukemia vectors, Harvey murine sarcoma virus vectors, murine mammary tumor virus vectors, Rous sarcoma virus vectors and nonviral plasmids. Baculovirus vectors are well known and are suitable for expression in insect cells. A plethora of vectors suitable for expression in mammalian or other eukaryotic cells are well known to the art, and many are commercially available. Commercial sources include, without limitation, Stratagene, La Jolla, Calif.; Invitrogen, Carlsbad, Calif.; Promega, Madison, Wis. and Sigma-Aldrich, St. Louis, Mo. Many vector sequences are available through GenBank, and additional information concerning vectors is available on the internet via the Riken BioSource Center.

The vector typically comprises an origin of replication and the vector may or may not in addition comprise a “marker” or “selectable marker” function by which the vector can be identified and selected. While any selectable marker can be used, selectable markers for use in recombinant vectors are generally known in the art and the choice of the proper selectable marker will depend on the host cell. Examples of selectable marker genes which encode proteins that confer resistance to antibiotics or other toxins include, but are not limited to ampicillin, methotrexate, tetracycline, neomycin (Southern et al. 1982. J Mol Appl Genet. 1:327-41), mycophenolic acid (Mulligan et al. 1980. Science 209:1422-7), puromycin, zeomycin, hygromycin (Sugden et al. 1985. Mol Cell Biol. 5:410-3), dihydrofolate reductase, glutamine synthetase, and G418. As will be understood by those of skill in the art, expression vectors typically include an origin of replication, a promoter operably linked to the coding sequence or sequences to be expressed, as well as ribosome binding sites, RNA splice sites, a polyadenylation site, and transcriptional terminator sequences, as appropriate to the coding sequence(s) being expressed.

Reference to a vector or other DNA sequences as “recombinant” merely acknowledges the operable linkage of DNA sequences which are not typically operably linked as isolated from or found in nature. Regulatory (expression and/or control) sequences are operatively linked to a nucleic acid coding sequence when the expression and/or control sequences regulate the transcription and, as appropriate, translation of the nucleic acid sequence. Thus expression and/or control sequences can include promoters, enhancers, transcription terminators, a start codon (i.e., ATG) 5′ to the coding sequence, splicing signals for introns and stop codons.

Adenovirus gene therapy vectors are known to exhibit strong transient expression, excellent titer, and the ability to transduce dividing and non-dividing cells in vivo (Hitt et al. 2000. Adv in Virus Res 55:479-505). The recombinant Ad vectors of the instant invention comprise a packaging site enabling the vector to be incorporated into replication-defective Ad virions; the coding sequence for two or more polypeptides or proteins of interest, e.g., heavy and light chains of an immunoglobulin of interest; and a sequence encoding a self-processing cleavage site alone or in combination with an additional proteolytic cleavage site. Other elements necessary or helpful for incorporation into infectious virions, include the 5′ and 3′ Ad ITRs, the E2 genes, portions of the E4 gene and optionally the E3 gene.

Replication-defective Ad virions encapsulating the recombinant Ad vectors are made by standard techniques known in the art using Ad packaging cells and packaging technology. Examples of these methods may be found, for example, in U.S. Pat. No. 5,872,005. The coding sequence for two or more polypeptides or proteins of interest is commonly inserted into adenovirus in the deleted E3 region of the virus genome. Preferred adenoviral vectors for use in practicing the invention do not express one or more wild-type Ad gene products, e.g., E1a, E1b, E2, E3, and E4. Preferred embodiments are virions that are typically used together with packaging cell lines that complement the functions of E1, E2A, E4 and optionally the E3 gene regions. See, e.g. U.S. Pat. Nos. 5,872,005, 5,994,106, 6,133,028 and 6,127,175.

Thus, as used herein, “adenovirus” and “adenovirus particle” refer to the virus itself or derivatives thereof and cover all serotypes and subtypes and both naturally occurring and recombinant forms, except where indicated otherwise. Such adenoviruses may be wild type or may be modified in various ways known in the art or as disclosed herein. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. Exemplary packaging and producer cells are derived from 293, A549 or HeLa cells. Adenovirus vectors are purified and formulated using standard techniques known in the art.

Adeno-associated virus (AAV) is a helper-dependent human parvovirus which is able to infect cells latently by chromosomal integration. Because of its ability to integrate chromosomally and its nonpathogenic nature, AAV has significant potential as a human gene therapy vector. For use in practicing the present invention rAAV virions may be produced using standard methodology, known to those of skill in the art and are constructed such that they include, as operatively linked components in the direction of transcription, control sequences including transcriptional initiation and termination sequences, and the coding sequence(s) of interest. More specifically, the recombinant AAV vectors of the instant invention comprise a packaging site enabling the vector to be incorporated into replication-defective AAV virions; the coding sequence for two or more polypeptides or proteins of interest, e.g., heavy and light chains of an immunoglobulin of interest; a sequence encoding a self-processing cleavage site alone or in combination with one or more additional proteolytic cleavage sites. AAV vectors for use in practicing the invention are constructed such that they also include, as operatively linked components in the direction of transcription, control sequences including transcriptional initiation and termination sequences. These components are flanked on the 5′ and 3′ end by functional AAV ITR sequences. By “functional AAV ITR sequences” is meant that the ITR sequences function as intended for the rescue, replication and packaging of the AAV virion.

Recombinant AAV vectors are also characterized in that they are capable of directing the expression and production of selected recombinant polypeptide or protein products in target cells. Thus, the recombinant vectors comprise at least all of the sequences of AAV essential for encapsidation and the physical structures for infection of the recombinant AAV (rAAV) virions. Hence, AAV ITRs for use in expression vectors need not have a wild-type nucleotide sequence (e.g., as described in Kotin. 1994. Hum. Gene Ther. 5:793-801), and may be altered by the insertion, deletion or substitution of nucleotides or the AAV ITRs may be derived from any of several AAV serotypes. Generally, an AAV vector can be any vector derived from an adeno-associated virus serotype known to the art.

Typically, an AAV expression vector is introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large Rep proteins (Rep78 and Rep68), typically by mutating the start codon following p5 from ATG to ACG, as described in U.S. Pat. No. 6,548,286, incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, incorporated by reference herein in their entireties. Further compositions and methods for packaging are described in Wang et al. (US Patent Publication 2002/0168342), also incorporated by reference herein in its entirety, and include those techniques within the knowledge of those of skill in the art.

In practicing the invention, host cells for producing rAAV or other vector expression vector virions include mammalian cells, insect cells, microorganisms and yeast. Host cells can also be packaging cells in which the AAV (or other) rep and cap genes are stably maintained in the host cell or producer cells in which the AAV vector genome is stably maintained and packaged. Exemplary packaging and producer cells are derived from 293, A549 or HeLa cells. AAV vectors are purified and formulated using standard techniques known in the art. Additional suitable host cells (depending on the vector) include Chinese Hamster Ovary (CHO) cells, CHO dihydrofolate reductase deficient variants such as CHO DX B11 or CHO DG44 cells (see, e.g., Urlaub and Chasin. 1980. Proc. Natl. Acad. Sci. 77:4216-4220), PerC.6 cells (Jones et al. 2003. Biotechnol. Prog. 19:163-168) or Sp/20 mouse myeloma cells (Coney et al. 1994. Cancer Res. 54:2448-2455).

Retroviral Vectors

Retroviral vectors are also a common tool for gene delivery (Miller. 1992. Nature 357: 455-460). Retroviral vectors and more particularly lentiviral vectors may be used in practicing the present invention. Accordingly, the term “retrovirus” or “retroviral vector”, as used herein is meant to include “lentivirus” and “lentiviral vectors” respectively. Retroviral vectors have been tested and found to be suitable delivery vehicles for the stable introduction of genes of interest into the genome of a broad range of target cells. The ability of retroviral vectors to deliver unrearranged, single copy transgenes into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) may also find utility in practicing the present invention. The ability to direct the delivery of retroviral vectors encoding one or more target protein coding sequences to specific target cells is desirable in practice of the present invention.

The present invention provides retroviral vectors which include e.g., retroviral transfer vectors comprising one or more transgene sequences and retroviral packaging vectors comprising one or more packaging elements. In particular, the present invention provides pseudotyped retroviral vectors encoding a heterologous or functionally modified envelope protein for producing pseudotyped retrovirus.

The core sequence of the retroviral vectors of the present invention may be readily derived from a wide variety of retroviruses, including for example, B, C, and D type retroviruses as well as spumaviruses and lentiviruses (see RNA Tumor Viruses, Second Edition, Cold Spring Harbor Laboratory, 1985). An example of a retrovirus suitable for use in the compositions and methods of the present invention includes, but is not limited to, lentivirus. Other retroviruses suitable for use in the compositions and methods of the present invention include, but are not limited to, Avian Leukosis Virus, Bovine Leukemia Virus, Murine Leukemia Virus, Mink-Cell Focus-Inducing Virus, Murine Sarcoma Virus, Reticuloendotheliosis virus and Rous Sarcoma Virus. Particularly preferred Murine Leukemia Viruses include 4070A and 1504A (Hartley and Rowe. 1976. J. Virol. 19:19-25), Abelson (ATCC No. VR-999), Friend (ATCC No. VR-245), Graffi, Gross (ATCC No. VR-590), Kirsteni Harvey Sarcoma Virus and Rauscher (ATCC No. VR-998), and Moloney Murine Leukemia Virus (ATCC No. VR-190). Such retroviruses may be readily obtained from depositories or collections such as the American Type Culture Collection (ATCC; Manassas, Va.), or isolated from known sources using commonly available techniques. Others are available commercially.

A retroviral vector sequence of the present invention can be derived from a lentivirus. A preferred lentivirus is a human immunodeficiency virus, e.g., type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. Other lentivirus include, a sheep Visna/maedi virus, a feline immunodeficiency virus (FIV), a bovine lentivirus, simian immunodeficiency virus (SIV), an equine infectious anemia virus (EIAV), and a caprine arthritis-encephalitis virus (CAEV).

Suitable genera and strains of retroviruses are well known in the art (see, e.g., Fields Virology, Third Edition, edited by B. N. Fields et al. 1996. Lippincott-Raven Publishers, see e.g., Chapter 58, Retroviridae: The Viruses and Their Replication, Classification, pages 1768-1771, including Table 1, incorporated herein by reference). Retroviral packaging systems for generating producer cells and producer cell lines that produce retroviruses, and methods of making such packaging systems are also known in the art.

Typical packaging systems comprise at least two packaging vectors: a first packaging vector which comprises a first nucleotide sequence comprising a gag, a pol, or gag and pol genes; and a second packaging vector which comprises a second nucleotide sequence comprising a heterologous or functionally modified envelope gene. The retroviral elements can be derived from a lentivirus, such as HIV. The vectors can lack a functional tat gene and/or functional accessory genes (vif, vpr, vpu, vpx, nef). The system can further comprise a third packaging vector with a nucleotide sequence comprising a rev gene. The packaging system can be provided in the form of a packaging cell that contains the first, second, and, optionally, third nucleotide sequences.

The invention is applicable to a variety of expression systems, especially those with eukaryotic cells, and advantageously mammalian cells. Where native proteins are glycosylated, it is preferred that the expression system be one which will provide native-like glycosylation to the expressed proteins.

Lentiviruses share several structural virion proteins in common, including the envelope glycoproteins SU (gp120) and TM (gp41), which are encoded by the env gene; CA (p24), MA (p17) and NC (p7-11), which are encoded by the gag gene; and RT, PR and IN encoded by the pol gene. HIV-1 and HIV-2 contain accessory and other proteins involved in regulation of synthesis and processing virus RNA and other replicative functions. The accessory proteins, encoded by the vif, vpr, vpu/vpx, and nef genes, can be omitted (or inactivated) from the recombinant system. In addition, tat and rev can be omitted or inactivated, e.g., by mutation or deletion.

First generation lentiviral vector packaging systems provide separate packaging constructs for gag/pol and env, and typically employ a heterologous or functionally modified envelope protein for safety reasons. In second generation lentiviral vector systems, the accessory genes, vif, vpr, vpu and nef, are deleted or inactivated. Third generation lentiviral vector systems are those from which the tat gene has been deleted or otherwise inactivated (e.g., via mutation).

Compensation for the regulation of transcription normally provided by tat can be provided by the use of a strong constitutive promoter, such as the human cytomegalovirus immediate early (HCAAV-IE) enhancer/promoter. Other promoters/enhancers can be selected based on strength of constitutive promoter activity, specificity for target tissue (e.g., a liver-specific promoter), or other factors relating to desired control over expression, as is understood in the art. For example, in some embodiments, it is desirable to employ an inducible promoter such as tet to achieve controlled expression. The gene encoding rev can be provided on a separate expression construct, such that a typical third generation lentiviral vector system will involve four plasmids: one each for gagpol, rev, envelope and the transfer vector. Regardless of the generation of packaging system employed, gag and pol can be provided on a single construct or on separate constructs.

Typically, the packaging vectors are included in a packaging cell, and are introduced into the cell via transfection, transduction or infection. Methods for transfection, transduction or infection are well known by those of skill in the art. A retroviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some embodiments, the packaging vectors are introduced into the cells together with a dominant selectable marker, such as neo, dihydrofolate reductase (DHFR), glutamine synthetase or ADA, followed by selection in the presence of the appropriate drug and isolation of clones. A selectable marker gene can be linked physically to genes encoded by the packaging vector.

Stable cell lines, wherein the packaging functions are configured to be expressed by a suitable packaging cell, are known. For example, see U.S. Pat. No. 5,686,279; and Ory et al. 1996. Proc. Natl. Acad. Sci. 93:11400-11406, which describe packaging cells. Further description of stable cell line production can be found in Dull et al. 1998. J. Virol. 72(11):8463-8471; and in Zufferey et al. 1998. J. Virol. 72:9873-9880.

Zufferey et al. 1997. Nat. Biotechnol. 15:871-75, teach a lentiviral packaging plasmid wherein sequences 3′ of pol including the HIV-1 envelope gene are deleted. The construct contains tat and rev sequences and the 3′ LTR is replaced with poly A sequences. The 5′ LTR and psi sequences are replaced by another promoter, such as one which is inducible. For example, a CMV promoter or derivative thereof can be used.

The packaging vectors may contain additional changes to the packaging functions to enhance lentiviral protein expression and to enhance safety. For example, all of the HIV sequences upstream of gag can be removed. Also, sequences downstream of the envelope can be removed. Moreover, steps can be taken to modify the vector to enhance the splicing and translation of the RNA.

Optionally, a conditional packaging system is used, such as that described by Dull et al. 1998. supra. Also preferred is the use of a self-inactivating vector (SIN), which improves the biosafety of the vector by deletion of the HIV-1 long terminal repeat (LTR) as described, for example, by Zufferey et al. 1998. J. Virol. 72:9873-9880. Inducible vectors can also be used, such as through a tetracycline-inducible LTR.

Promoters

The vectors of the invention typically include heterologous control sequences, which include, but are not limited to, constitutive promoters, such as the cytomegalovirus (CMV) immediate early promoter, the RSV LTR, the MOMLV LTR, and the PGK promoter; tissue or cell type specific promoters including mTTR, TK, HBV, hAAT, regulatable or inducible promoters, enhancers, etc.

Useful promoters include the LSP promoter (III et al. 1997. Blood Coagul. Fibrinolysis 8S2:23-30), the EF1-alpha promoter (Kim et al. 1990. Gene 91(2):217-23) and Guo et al. 1996. Gene Ther. 3(9):802-10). Most preferred promoters include the elongation factor 1-alpha (EF1a) promoter, a phosphoglycerate kinase-1 (PGK) promoter, a cytomegalovirus immediate early gene (CMV) promoter, chimeric liver-specific promoters (LSPs), a cytomegalovirus enhancer/chicken beta-actin (CAG) promoter, a tetracycline responsive promoter (TRE), a transthyretin promoter (TTR), an simian virus 40 (SV40) promoter and a CK6 promoter. An advantageous promoter useful in the practice of the present invention is the adenovirus major late promoter (Berkner and Sharp. 1985. Nucl. Acids Res. 13:841-857). The sequence of a specifically exemplified expression vector employing the adenovirus major late promoter is provided herein below. The sequences of these and numerous additional promoters are known in the art. The relevant sequences may be readily obtained from public databases and incorporated into vectors for use in practicing the present invention.

A particular preferred promoter in the practice of the present invention is the Adenovirus major late promoter. An expression cassette can comprise, in the 5′ to 3′ direction, an adenovirus major late promoter, a tripartite leader sequence operably to a first coding sequence for a protein of interest or protein chain of interest, a sequence encoding a self processing sequence or protease cleavage sequence, a second coding sequence for a protein or protein chain of interest, and optionally a sequence encoding a self processing sequence or protease cleavage sequence, followed by a third coding sequence for a protein or protein chain of interest. All of these coding sequences are covalently joined and in the same reading frame such that translation is not terminated within the polyprotein coding sequence. During protein synthesis or after completion of the synthesis of the polypeptide self processing or proteolytic processing cleaves the polyprotein into the appropriate protein chains or proteins. In the case of immunoglobulin synthesis, the coding sequence for light chain is present twice within the polyprotein coding sequence. Advantageously, leader sequence coding regions can be associated with the protein or protein chain sequences; processing by signal peptidases can have the added benefit of removing certain residual amino acid residues at the N-termini of proteins downstream of processing sites. Components for immunoglobulin heavy chain are Met, protein initiation methionine; HC, heavy chain; LC, light chain, SPPC, self-processing or protease cleavage site. Expression constructs for immunoglobulin synthesis can include the following: Met-protease-SPPC-HC leader sequence-HC-SPPC-LC leader sequence-LC-SPPC-LC leader sequence-LC; Met-protease-SPPC-LC leader sequence-LC-SPPC-LC leader sequence-LC-SPPC-HC leader sequence-HC; Met-protease-SPPC-LC leader sequence-LC-SPPC-HC leader sequence-HC-SPPC-LC leader sequence-LC; HC leader sequence-HC-SPPC-LC leader sequence-LC-SPPC-LC leader sequence-LC; LC leader sequence-LC-SPPC-HC leader sequence-HC-SPPC-LC leader sequence-LC; LC leader sequence-LC-SPPC-LC leader sequence-LC-SPPC-HC leader sequence-HC; Met-protease-SPPC-HC leader-HC-SPPC-LC leader-LC.

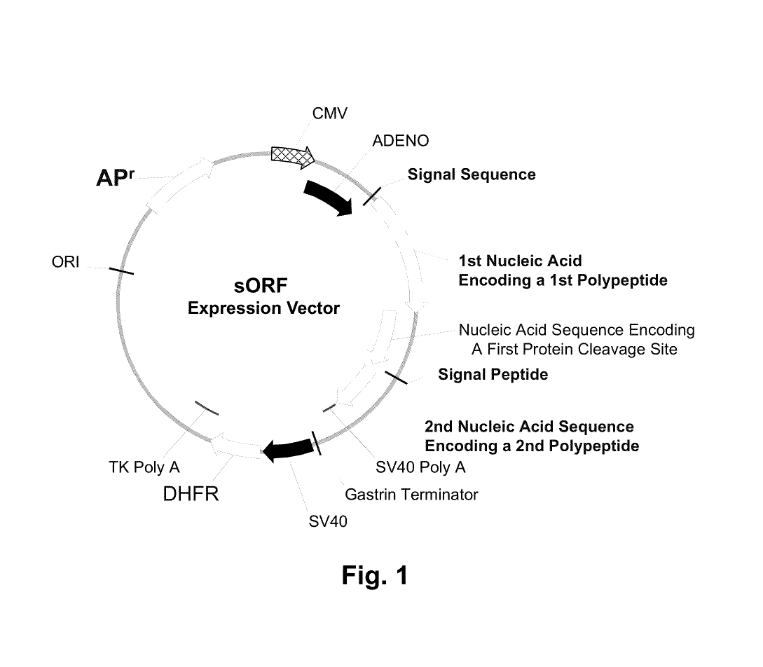

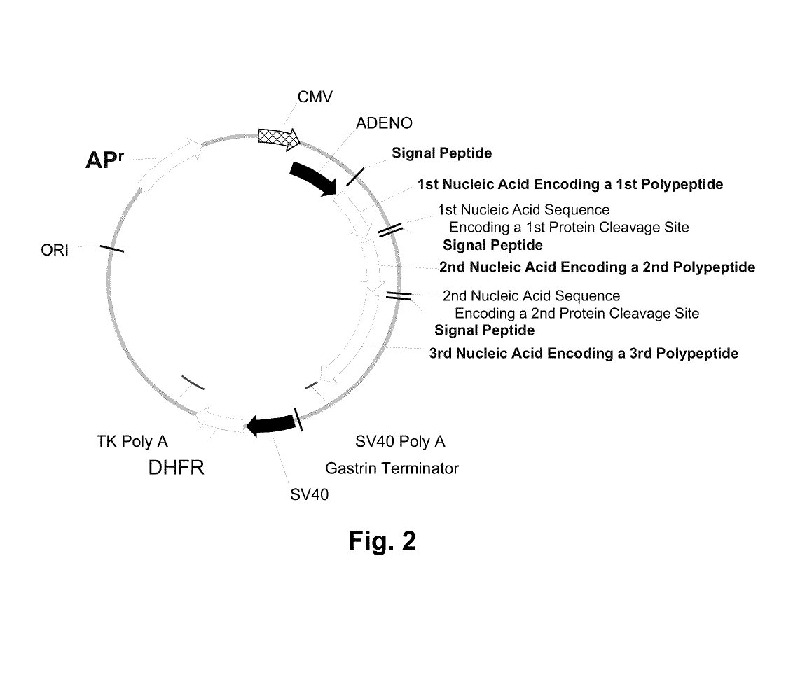

A specifically exemplified polyprotein coding sequence (product Met-HC leader-HC-engineered furin site-TEV cleavage site-TEV Nia protease-TEV cleavage site-LC leader-LC is schematically shown in FIG. 1, and schematic of the expression vector for the expression of this construct is shown in FIG. 2. Anti-TNFα (D2E7) is an exemplary antibody with respect to its HC and LC sequences. The LC leader sequence may not be required for the production of a therapeutic antibody. The SPPS is a TEV protease recognition site, and there is a furin site encoded 5′ to the TEV site. Furin cleavage after TEV cleavage restores the “correct” C terminal lysine residue to the heavy chain. The complete DNA sequence of the D2E7-TEV expression vector is shown in Table 1.

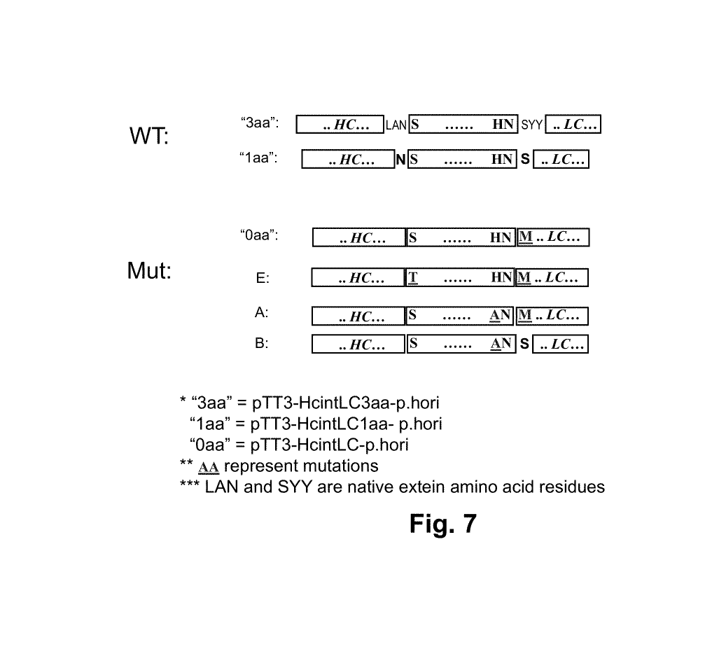

A specifically exemplified D2E7 polyprotein expression construct (D2E7-Lc-LC-HC) encoding a tandem repeat of the LC and cleaved using the 2A protease sequence as cleavage sites has been designed. The D2E7 light chain C termini have been modified to add the Furin cleavage sites. This results in a Glu to Arg change in the (normally) penultimate amino acid and the addition of a lysine to the C-terminus. By placing the two LC sequences 5′ to the HC, the two LC copies maintain the same amino acid sequence. The complete nucleotide sequence of the expression vector is shown in Table 6C, and the amino acid sequence and coding sequence of the polyprotein are shown in Tables 6B and 6A, respectively. See also SEQ ID NOs:29-31. A schematic expression vector map is shown in FIG. 7.

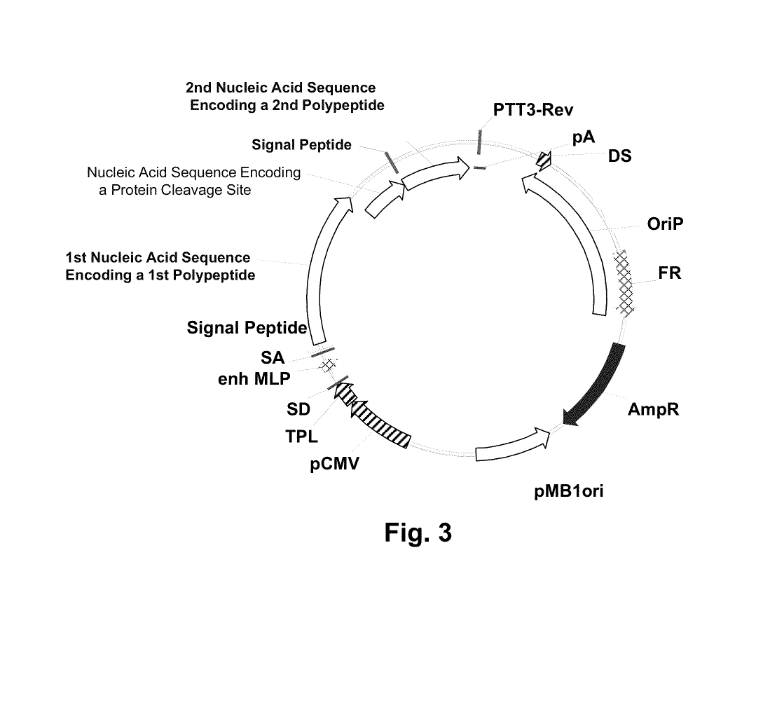

Another specifically exemplified polyprotein (and its coding sequence) is that of ABT-007-TEV; see Tables 2B and 2A, respectively. See SEQ ID NOs:33 and 32. This recombinant antibody specifically binds to erythropoietin receptor (EpoR). The complete sequence of the expression vector encoding the engineered ABT-007-TEV polyprotein is shown in Table 2C (SEQ ID NO:35. See also SEQ ID NO:34. The schematic representation of the vector is shown in FIG. 3.

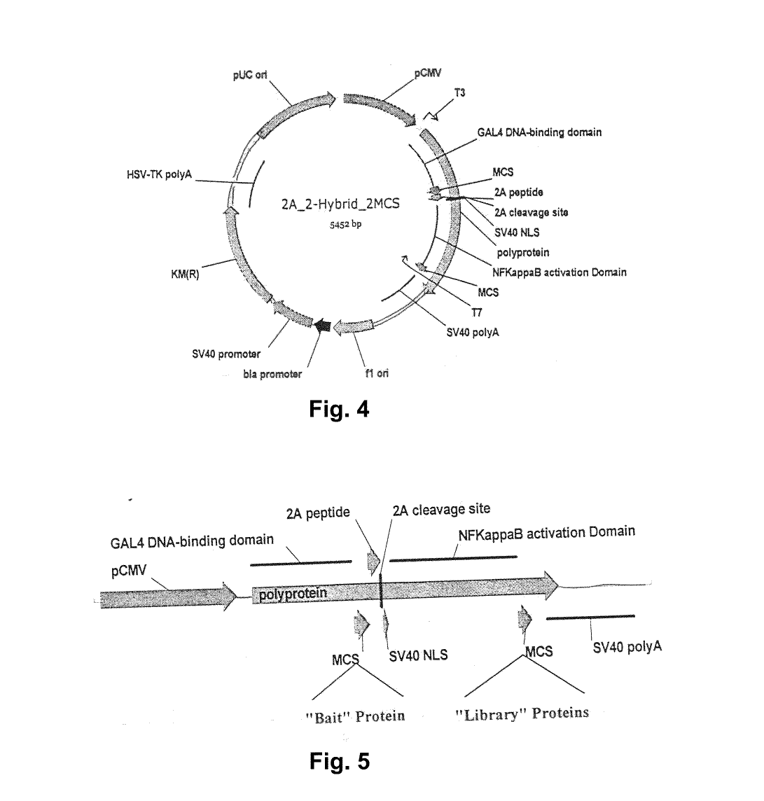

An additional specifically exemplified polyprotein and its coding sequence is that of ABT-874-TEV; see Tables 3B and 3A, respectively. This antibody specifically binds to interleukin-12. The schematic representation of the expression vector is shown in FIG. 4. See also SEQ ID NOs:35-37.

Yet another specifically exemplified polyprotein (and its coding sequence) is that of EL246-GG-TEV; see Tables 4B and 4A. The antibody encoded therein specifically binds to E/L selectin. The expression vector is provided in schematic form in FIG. 5. See also SEQ ID NOs:38-40.

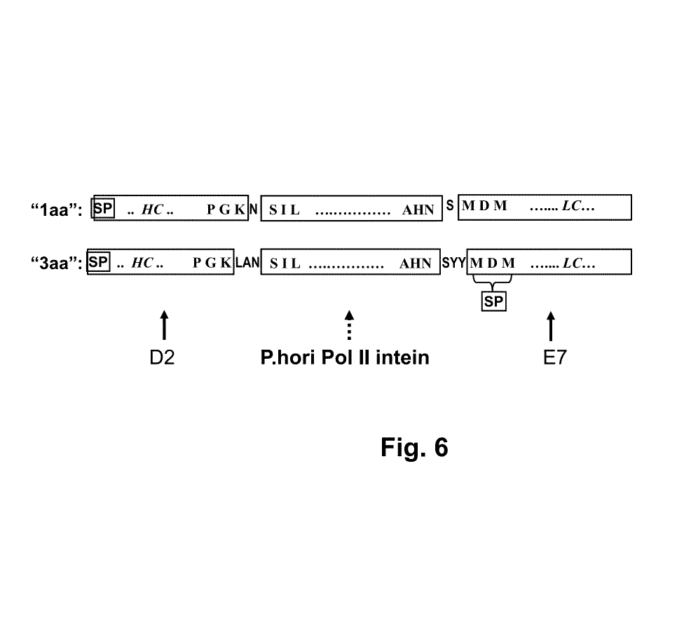

ABT-325-TEV is an engineered antibody with binding specificity for interleukin-18. The coding and amino acid sequences of the polyprotein are given in Tables 5A and 5B, respectively, and the complete expression vector sequence is provided in Table 5C. The expression vector for its synthesis is shown in FIG. 6. See also SEQ ID NOs:41-43.

Also provided is a TEV protease with its nuclear localization signal (NLS) removed (TEV NLS-). The TEV or TEV(NLS-) protease can also be expressed in cells transiently or stably as part of a separate vector or separate transcript. The TEV(NLS-) protein may be anchored to the ER or to the ribosome by including an ER anchor sequence or by fusing to a small ribosome binding protein, respectively at the previous NLS portion.

While the present application contains discussion of proteolytic cleavage of precursor proteins and polyproteins during synthesis or in the cell after synthesis, it is understood that the polyproteins and precursor proteins (proproteins) can be achieved after collection of those proteins with the use of appropriate protease(s) in vitro.

Within the scope of the present invention, particular expressed antibodies (immunoglobulins) can include, inter alia, those which specifically bind tumor necrosis factor (engineered antibody corresponding to and/or derived from HUMIRA/D2E7; trademark for adalimumab of Abbott Biotechnology Ltd., Hamilton, Bermuda); interleukin-12 (engineered antibody derived from ABT-874); interleukin-18 (engineered antibody derived from ABT-325); recombinant erythropoietin receptor (engineered antibody derived from ABT-007); interleukin-18 (engineered antibody derived from ABT-325); or E/L selectin (engineered antibody derived from EL246-GG). Coding and amino acid sequences of the engineered polyproteins are shown in Tables 1-5. Further antibodies which are suitable to the present invention include, e.g., Remicade (infliximab); Rituxan/Mabthera (rituximab); Herceptin (trastuzumab); Avastin (bevacizumab); Synagis (palivizumab); Erbitux (cetuximab); Reopro (abciximab); Orthoclone OKT3 (muromonab-CD3); Zenapax (daclizumab); Simulect (basiliximab); Mylotarg (gemtuzumab); Campath (alemtuzumab); Zevalin (ibritumomab); Xolair (omalizumab); Bexxar (tositumomab); and Raptiva (efalizumab); wherein generally a trademark-brand name is followed by a respective generic name in parentheses. Additional suitable proteins include, e.g., one or more of epoetin alfa, epoetin beta, etanercept, darbepoetin alfa, filgrastim, interferon beta 1a, interferon beta 1b, interferon alfa-2b, insulin glargine, somatropin, teriparatide, follitropin alfa, dornase, Factor VIII, Factor VII, Factor IX, imiglucerase, nesiritide, lenograstim, and Von Willebrand factor; wherein one or more generic designations may each correspond to one or more trademark-brand names of products. Other antibodies and proteins are suitable to the present invention as would be understood in the art.

The present invention also contemplates the controlled expression of the coding sequence for two or more polypeptides or proteins or proproteins of interest. Gene regulation systems are useful in the modulated expression of a particular gene or genes. In one exemplary approach, a gene regulation system or switch includes a chimeric transcription factor that has a ligand binding domain, a transcriptional activation domain and a DNA binding domain. The domains may be obtained from virtually any source and may be combined in any of a number of ways to obtain a novel protein. A regulatable gene system also includes a DNA response element which interacts with the chimeric transcription factor. This transcription regulatory element is located adjacent to the gene to be regulated.

Exemplary transcription regulation systems that may be employed in practicing the present invention include, for example, the Drosophila ecdysone system (Yao et al. 1996. Proc. Natl. Acad. Sci. 93:3346), the Bombyx ecdysone system (Suhr et al. 1998. Proc. Natl. Acad. Sci. 95:7999), the GeneSwitch (trademark of Valentis, The Woodlands, Tex.) synthetic progesterone receptor system which employs RU486 as the inducer (Osterwalder et al. 2001. Proc. Natl. Acad. Sci. USA 98(22):12596-601); the Tet and RevTet Systems (tetracycline regulated expression systems, trademarks of BD Biosciences Clontech, Mountain View, Calif.), which employ small molecules, such as tetracycline (Tc) or analogues, e.g. doxycycline, to regulate (turn on or off) transcription of the target (Knott et al. 2002. Biotechniques 32(4):796, 798, 800); ARIAD Regulation Technology (Ariad, Cambridge, Mass.) which is based on the use of a small molecule to bring together two intracellular molecules, each of which is linked to either a transcriptional activator or a DNA binding protein. When these components come together, transcription of the gene of interest is activated. Ariad has a system based on homodimerization and a system based on heterodimerization (Rivera et al. 1996. Nature Med. 2(9):1028-1032; Ye et al. 2000. Science 283:88-91).

The expression vector constructs of the invention comprising nucleic acid sequences encoding antibodies or fragments thereof or other heterologous proteins or pro-proteins in the form of self-processing or protease-cleaved recombinant polypeptides may be introduced into cells in vitro, ex vivo or in vivo for delivery of foreign, therapeutic or transgenes to cells, e.g., somatic cells, or in the production of recombinant polypeptides by vector-transduced cells.

Host Cells and Delivery of Vectors

The vector constructs of the present invention may be introduced into suitable cells in vitro or ex vivo using standard methodology known in the art. Such techniques include, e.g., transfection using calcium phosphate, microinjection into cultured cells (Capecchi. 1980. Cell 22:479-488), electroporation (Shigekawa et al. 1988. BioTechnology 6:742-751), liposome-mediated gene transfer (Mannino et al. 1988. BioTechnology 6:682-690), lipid-mediated transduction (Feigner et al. 1987. Proc. Natl. Acad. Sci. USA 84:7413-7417), and nucleic acid delivery using high-velocity microprojectiles (Klein et al. 1987. Nature 327:70-73).

For in vitro or ex vivo expression, any cell effective to express a functional protein product may be employed. Numerous examples of cells and cell lines used for protein expression are known in the art. For example, prokaryotic cells and insect cells may be used for expression. In addition, eukaryotic microorganisms, such as yeast may be used. The expression of recombinant proteins in prokaryotic, insect and yeast systems are generally known in the art and may be adapted for antibody or other protein expression using the compositions and methods of the present invention.

Examples of cells useful for expression further include mammalian cells, such as fibroblast cells, cells from non-human mammals such as ovine, porcine, murine and bovine cells, insect cells and the like. Specific examples of mammalian cells include, without limitation, COS cells, VERO cells, HeLa cells, Chinese hamster ovary (CHO) cells, CHO DX B11 cells, CHO DG44 cells, PerC.6 cells, Sp2/0 cells, 293 cells, NSO cells, 3T3 fibroblast cells, W138 cells, BHK cells, HEPG2 cells, and MDCK cells.

Host cells are cultured in conventional nutrient media, modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium (MEM), Sigma), RPMI 1640 (Sigma), and Dulbecco's Modified Eagle's Medium (DMEM), Sigma) are typically suitable for culturing host cells. A given medium is generally supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations as well known to those skilled in the art. The appropriate culture conditions for a particular cell line, such as temperature, pH and the like, are generally known in the art, with suggested culture conditions for culture of numerous cell lines, for example, in the ATCC Catalogue (available on the internet at “atcc.org/SearchCatalogs/AllCollections.cfm” or as instructed by commercial suppliers.

The expression vectors may be administered in vivo via various routes (e.g., intradermally, intravenously, intratumorally, into the brain, intraportally, intraperitoneally, intramuscularly, into the bladder etc.), to deliver multiple genes connected via a self processing cleavage sequence to express two or more proteins or polypeptides in animal models or human subjects. Dependent upon the route of administration, the therapeutic proteins elicit their effect locally (in brain or bladder) or systemically (other routes of administration). The use of tissue specific promoters 5′ to the open reading frame(s) results in tissue specific expression of the proteins or polypeptides encoded by the entire open reading frame.

Various methods that introduce a recombinant expression vector carrying a transgene into target cells in vitro, ex vivo or in vivo have been previously described and are well known in the art. The present invention provides for therapeutic methods, vaccines, and cancer therapies by infecting targeted cells with the recombinant vectors containing the coding sequence for two or more proteins or polypeptides of interest, and expressing the proteins or polypeptides in the targeted cell.

For example, in vivo delivery of the recombinant vectors of the invention may be targeted to a wide variety of organ types including, but not limited to brain, liver, blood vessels, muscle, heart, lung and skin.

In the case of ex vivo gene transfer, the target cells are removed from the host and genetically modified in the laboratory using recombinant vectors of the present invention and methods well known in the art.

The recombinant vectors of the invention can be administered using conventional modes of administration including but not limited to the modes described above. The recombinant vectors of the invention may be in a variety of formulations which include but are not limited to liquid solutions and suspensions, microvesicles, liposomes and injectable or infusible solutions. The preferred form depends upon the mode of administration and the therapeutic application.

Advantages of the present inventive recombinant expression vector constructs of the invention in immunoglobulin or other biologically active protein production in vivo include administration of a single vector for long-term and sustained antibody expression in patients; in vivo expression of an antibody or fragment thereof (or other biologically active protein) having full biological activities; and the natural posttranslational modifications of the antibody generated in human cells. Desirably, the expressed protein is identical to or sufficiently identical to a naturally occurring protein so that immunological responses are not triggered where the expressed protein is administered to on multiple occasions or expressed continually in a patient in need of said protein.

The recombinant vector constructs of the present invention find further utility in the in vitro production of recombinant antibodies and other biologically active proteins for use in therapy or in research. Methods for recombinant protein production are well known in the art and may be utilized for expression of recombinant antibodies using the self processing cleavage site or other protease cleavage site-containing vector constructs described herein.

In one aspect, the invention provides methods for producing a recombinant immunoglobulin or fragment thereof, by introducing an expression vector such as described above into a cell to obtain a transfected cell, wherein the vector comprises in the 5′ to 3′ direction: a promoter operably linked to the coding sequences for immunoglobulin heavy and two light chains or fragment thereof, a self processing sequence such as a 2A or 2A-like sequence or protease cleavage site between each of said chains. It is appreciated that the coding sequence for either the immunoglobulin heavy chain or the coding sequence for the immunoglobulin light chain may be 5′ to the 2A sequence (i.e. first) in a given vector construct. Alternatively, the protease cognate to the protease cleavage site can be expressed as part of the polyprotein so that it is either self-processed from the remainder of the polyprotein or proteolytically cleaved by a separate (or the same) protease. Other multichain proteins or other proteins (such as those from the two- or three-hybrid systems) can be expressed in processed, active form by substituting the relevant coding sequences, interspersed by self-processing sites or protease recognition sites also correctly sized, separate proteins are produced.

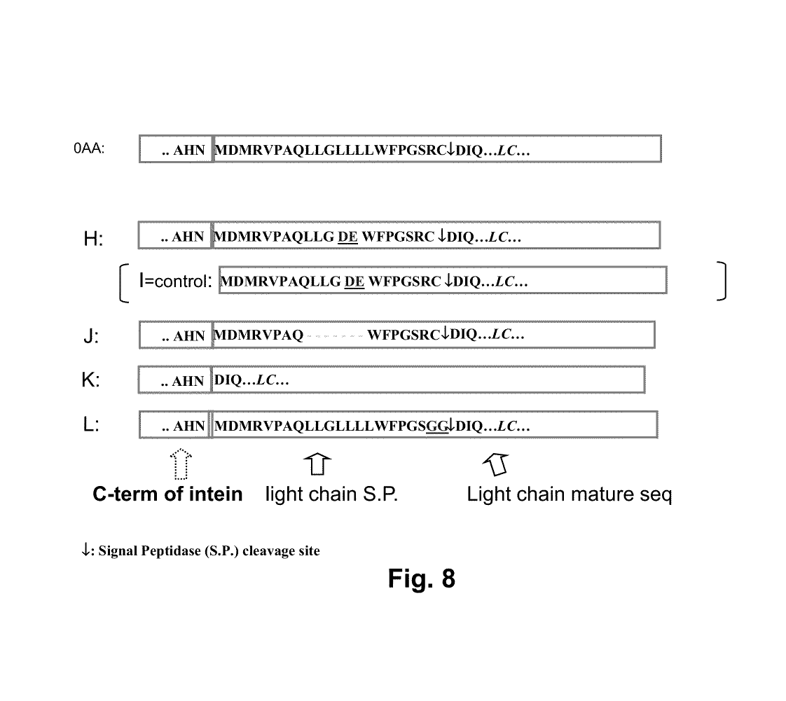

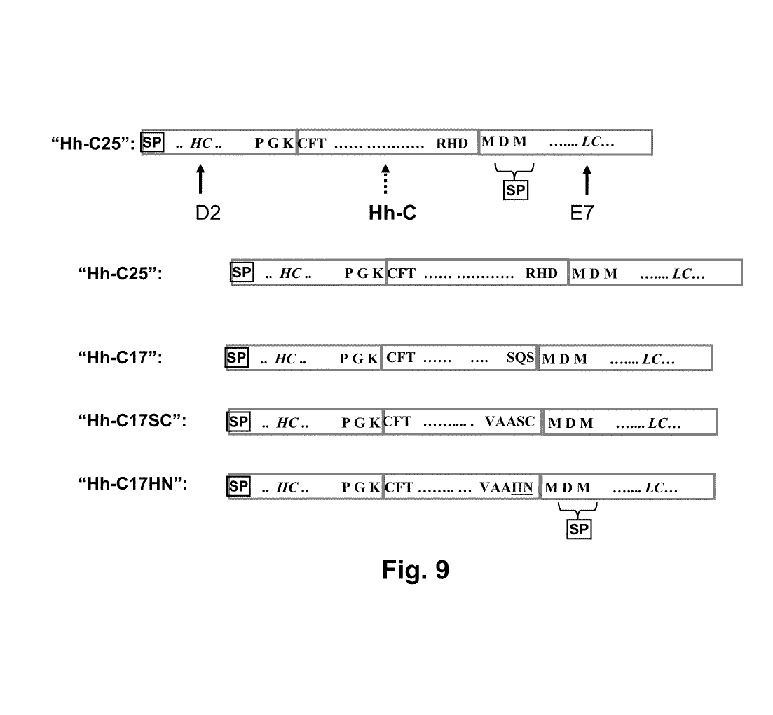

The two (and other) hybrid system approach has been used to screen cDNA libraries for previously unrecognized binding partners to a know ligand or subunit of a protein complex. With appropriate variations to this system, proteins or subunits which inhibit, compete or disrupt binding in a known complex can also be identified. Although the two (and other) hybrid systems have been applied to a variety of scientific inquiries, these systems can be inefficient because of the significance frequency of false positive or false negative results. Those false signals have been at least in some instances, attributed to an imbalance in the relative expression of the “bait” protein relative to candidate binding partner proteins or candidate disrupter proteins. An additional advantage of the strategy of the present invention is that only one plasmid is transfected or transformed into the host cell, and only a single selection is needed for that plasmid, instead of two selections in the binary vector two hybrid schemes. The approach can also be adapted for use in three hybrid systems. For discussions of the two hybrid systems, see Toby and Golemis. 2001. Methods 24:201-217; Vidal and Legrain. 1999. Nucl. Acids Res. 27:919-929; Drees, B. 1999. Curr. Op. Chem. Biol. 3:64-70; and Fields and Song. 1989. Nature 340:245-246. FIG. 9 shows a schematic representation of a polyprotein/self-processing or protease cleavage expression strategy for bait and prey proteins (or candidate prey proteins), and FIG. 8 shows a vector containing an expression cassette for bait and prey protein production using this approach. The vector expression cassette is structured to translate the bait protein first as a GAL4::bait::2A peptide fusion, which is self processed after the translation of the 2A peptide. The second open reading frame (ORF) is an NFkappaB::library fusion protein. Engineering of the bait protein into MCS1 requires an in-frame translation into the 2A self-processing peptide sequence. Engineering of an expression library in the downstream MCS2 is less critical.

The strategy provided herein can be similarly adapted to the expression of proteins that are expressed as pro-forms that are processed to the mature, active form by proteolytic cleavage, thus providing compositions and methods for recombinant expression. Examples of such proteins include, but are not limited to interleukins 1 and 18 (IL-1 and IL-18) insulin, among others. IL-1 and IL-18 are produced in the cytoplasm of inflammatory cells. These molecules lack a traditional secretion signal and must be cleaved by a protease in order to be secreted as the biologically active form. IL-1 is processed to the mature form by interleukin converting enzyme (ICE). Pro-IL-18 is converted to mature IL-18 by caspases. Production of these molecules in recombinant form is difficult because the cells frequently used as hosts do not express the proteases needed to produce biologically active mature forms of these proteins. Expression of these cytokines without the pro domains leads to inactive molecules and/or low levels of production. The present invention provides primary translation products which contain an engineered self processing site (e.g., 2A sequence) or an inserted protease cleavage site between the pro domain and the amino acid of the mature polypeptide, without the need to express a potentially toxic protease in parallel with the protein of interest.

In a related aspect, the invention provides a method for producing a recombinant immunoglobulin or fragment thereof, by introducing an expression vector such as described above into a cell, wherein the vector further comprises an additional proteolytic cleavage site between the first and second immunoglobulin coding sequences. A preferred additional proteolytic cleavage site is a furin cleavage site with the consensus sequence RXK/R-R (SEQ ID NO:1). For a discussion, see US Patent Publication 200510003482A1.

In one exemplary aspect of the invention, vector introduction or administration to a cell is followed by one or more of the following steps: culturing the transfected cell under conditions for selecting a cell and expressing the polyprotein or proprotein; measuring expression of the immunoglobulin or the fragment thereof or other protein(s); and collecting the immunoglobulin or the fragment thereof or other protein(s).

Another aspect of the invention provides a cell for expressing a recombinant immunoglobulin or a fragment thereof or other protein(s) or protein of interest, wherein the cell comprises an expression vector for the expression of two or more immunoglobulin chains or fragments thereof or other proprotein or proteins, a promoter operably linked to a first coding sequence for an immunoglobulin or other chain or fragment thereof, a self processing or other cleavage coding sequence, such as a 2A or 2A-like sequence or a protease recognition site, and a second coding sequence for an immunoglobulin or other chain or a fragment thereof, wherein the self processing cleavage sequence or protease recognition site coding sequence is inserted between the first and the second coding sequences. In a related aspect, the cell comprises an expression vector as described above wherein the expression vector further comprises an additional proteolytic cleavage site between the first and second immunoglobulin or other coding sequences of interest. A preferred additional proteolytic cleavage site is a furin cleavage site with the consensus sequence RXR/K-R (SEQ ID NO:1).

As used herein, “the coding sequence for a first chain of an immunoglobulin molecule or a fragment thereof” refers to a nucleic acid sequence encoding a protein molecule including, but not limited to a light chain or heavy chain for an antibody or immunoglobulin, or a fragment thereof.

As used herein, a “the coding sequence for a second chain of an immunoglobulin molecule or a fragment thereof” refers to a nucleic acid sequence encoding a protein molecule including, but not limited to a light chain or heavy chain for an antibody or immunoglobulin, or a fragment thereof. It is understood, in one aspect of the present invention, that improved expression results when there are two copies of the immunoglobulin light chain coding sequence per copy of the heavy chain coding sequence.

The sequence encoding the first or second chain for an antibody or immunoglobulin or a fragment thereof includes a heavy chain or a fragment thereof derived from an IgG, IgM, IgD, IgE or IgA. As broadly stated, the sequence encoding the chain for an antibody or immunoglobulin or a fragment thereof also includes the light chain or a fragment thereof from an IgG, IgM, IgD, IgE or IgA. Genes for whole antibody molecules as well as modified or derived forms thereof, include, e.g., other antigen recognition molecules fragments like Fab, single chain Fv (scFv) and F(ab′)₂. The antibodies and fragments can be animal-derived, human-mouse chimeric, humanized, altered by Deimmunisation™ (Biovation Ltd), altered to change affinity for Fc receptors, or fully human. Desirably, the antibody or other recombinant protein does not elicit an immune response in a human or animal to which it is administered.

The antibodies can be bispecific and include, but are not limited to, diantibodies, quadroma, mini-antibodies, ScBs antibodies and knobs-into-holes antibodies.

The production and recovery of the antibodies themselves can be achieved in various ways well known in the art (Harlow et al. 1988. Antibodies, A Laboratory Manual, Cold Spring Harbor Laboratory. Other proteins of interest are collected and/or purified and/or used according to methods well known to the art.

In practicing the invention, the production of an antibody or variant (analogue) thereof using recombinant DNA technology can be achieved by culturing a modified recombinant host cell under culture conditions appropriate for the growth of the host cell and the expression of the coding sequences. In order to monitor the success of expression, the antibody levels with respect to the antigen may be monitored using standard techniques such as ELISA, RIA and the like. The antibodies are recovered from the culture supernatant using standard techniques known in the art. Purified forms of these antibodies can, of course, be readily prepared by standard purification techniques including but not limited to, affinity chromatography via protein A, protein G or protein L columns, or with respect to the particular antigen, or even with respect to the particular epitope of the antigen for which specificity is desired. Antibodies can also be purified with conventional chromatography, such as an ion exchange or size exclusion column, in conjunction with other technologies, such as ammonia sulfate precipitation and size-limited membrane filtration. Where expression systems are designed to include signal peptides, the resulting antibodies are secreted into the culture medium or supernatant; however, intracellular production is also possible.

The production and selection of antigen-specific, fully human monoclonal antibodies from mice engineered with human Ig loci, has previously been described (Jakobovits et al. 1998. Advanced Drug Delivery Reviews 31:33-42; Mendez et al. 1997. Nature Genetics 15: 146-156; Jakobovits et al. 1995. Curr Opin Biotechnol 6: 561-566; Green et al. 1994. Nature Genetics Vol. 7:13-21).

High level expression of therapeutic monoclonal antibodies has been achieved in the milk of transgenic goats, and it has been shown that antigen binding levels are equivalent to that of monoclonal antibodies produced using conventional cell culture technology. This method is based on development of human therapeutic proteins in the milk of transgenic animals, which carry genetic information allowing them to express human therapeutic proteins in their milk. Once they are produced, these recombinant proteins can be efficiently purified from milk using standard technology. See e.g., Pollock et al. 1999. J. Immunol. Meth. 231:147-157 and Young et al. 1998. Res Immunol. 149(6): 609-610. Animal milk, egg white, blood, urine, seminal plasma and silk worm cocoons from transgenic animals have demonstrated potential as sources for production of recombinant proteins at an industrial scale (Houdebine L M. 2002. Curr Opin Biotechnol 13:625-629; Little et al. 2000. Immunol Today, 21(8):364-70; and Gura T. 2002. Nature, 417:584-5860. The invention contemplates use of transgenic animal expression systems for expression of a recombinant an antibody or variant (analogue) or other protein(s) of interest thereof using the self-processing cleavage site-encoding and/or protease recognition site vectors of the invention.

Production of recombinant proteins in plants has also been successfully demonstrated including, but not limited to, potatoes, tomatoes, tobacco, rice, and other plants transformed by Agrobacterium infection, biolistic transformation, protoplast transformation, and the like. Recombinant human GM-CSF expression in the seeds of transgenic tobacco plants and expression of antibodies including single-chain antibodies in plants has been demonstrated. See, e.g., Streaffield and Howard. 2003. Int. J. Parasitol. 33:479-93; Schillberg et al. 2003. Cell Mol Life Sci. 60:433 A5; Pogue et al. 2002. Annu. Rev. Phytopathol. 40:45-74; and McCormick et al. 2003. J Immunological Methods, 278:95-104. The invention contemplates use of transgenic plant expression systems for expression of a recombinant immunoglobulin or fragment thereof or other protein(s) of interest using the protease cleavage site or self-processing cleavage site-encoding vectors of the invention.

Baculovirus vector expression systems in conjunction with insect cells are also gaining ground as a viable platform for recombinant protein production. Baculovirus vector expression systems have been reported to provide advantages relative to mammalian cell culture such as ease of culture and higher expression levels. See, e.g., Ghosh et al. 2002. Mol. Ther. 6:5-11, and Ikonomou et al. 2003. Appl Microbiol Biotechnol. 62:1-20. The invention further contemplates use of baculovirus vector expression systems for expression of a recombinant immunoglobulin or fragment thereof using the self-processing cleavage site-encoding vectors of the invention. Baculovirus vectors and suitable host cells are well known to the art and commercially available.

Yeast-based systems may also be employed for expression of a recombinant immunoglobulin or fragment thereof or other protein(s) of interest, including two- or three-hybrid systems, using the self-processing cleavage site-encoding vectors of the invention. See, e.g., U.S. Pat. No. 5,643,745, incorporated by reference herein.

It is understood that the expression cassettes and vectors and recombinant host cells of the present invention which comprise the coding sequences for a self-processing peptide alone or in combination with additional coding sequences for a proteolytic cleavage site find utility in the expression of recombinant immunoglobulins or fragments thereof, proproteins, biologically active proteins and protein components of two- and three-hybrid systems, in any protein expression system, a number of which are known in the art and examples of which are described herein. One of skill in the art may easily adapt the vectors of the invention for use in any protein expression system.

When a compound, construct or composition is claimed, it should be understood that compounds, constructs and compositions known in the art including those taught in the references disclosed herein are not intended to be included. When a Markush group or other grouping is used herein, all individual members of the group and all combinations and subcombinations possible from within the group the group are intended to be individually included in the disclosure.

### Example 1

### Expression of Immunoglobulins with Intein-Mediated Processing

A strategy for the efficient expression of antibody molecules is via polyprotein expression, wherein an intein is located between the heavy and light chains, with modification of the intein sequence and/or junction sequences such that there is release of the component proteins without ligation of the N-terminal and C-terminal proteins. Within such constructs, there can be one copy of each of the relevant heavy and light chains, or the light chain can be duplicated, or there can be multiple copies of both heavy and light chains, provided that functional cleavage sequence is provided to promote separation of each immunoglobulin-derived protein within the polyprotein. The intein strategy can be employed more than once or a different proteolytic processing sequence or enzyme can be positioned at at least one terminus of an immunoglobulin derived protein.

The intein from Pyrococcus horikoshii has been incorporated into a construct as briefly described above and has been shown to successfully produce correctly processed and fully functional D2E7 antibody. Additional inteins tested are from Saccharomyces cerevisiae and Synechocystis spp. Strain PCC6803 and have been shown to produce secreted antibody via ELISA.

PCR Amplification and Subcloning of the Pyrococcus horikoshii Pho Pol I Intein:

The following oligonucleotides were used for the amplification of the p. horikoshii Pho Pol I intein (NCBI/protein accession #059610, the GenBank accession # for the entire DNA Polymerase I DNA sequence is BA000001.2:1686361.1690068 as taken from the entire genomic sequence for P. horikoshii) using genomic DNA as template and Platinum Taq Hi Fidelity DNA Polymerase Supermix (Invitrogen, Carlsbad, Calif.). Genomic DNA was purchased from ATCC.

P. horikoshii int-5′

(SEQ ID NO: 52)

AGCATTTTACCAGATGAATGGCTCCC
P. horikoshii int-3′

(SEQ ID NO: 53)

AACGAGGAAGTTCTCATTATCCTCAAC

PCR was run according to the following program:

| | Step | | | | | | | |
|---|---|---|---|---|---|---|---|---|
| | 1 | 2 | 3 | 4 | 5 | 6 | 7 | 8 |
| Temp | 94° C. | 94° C. | 55° C. | 72° C. | Go to step 2 (34 times) | 72° C. | 4° C. | End |
| Time | 2 min | 1 min | 1 min | 2 min | | 5 min | hold | |

The PCR product was subcloned into pCR2.1-TOPO (Invitrogen) and the insert was sequenced and proven correct. At this time it was realized that there was sequence missing from the 3′ end of the intein due to a printout error. The missing sequence was then filled in during subsequent PCR reactions to link the intein to heavy and light chain of D2E7.

Oligonucleotide primers were designed in order to generate the fusion of D2E7 Heavy Chain-Intein-D2E7 Light Chain. Primers were designed so that PCR product could be used as primers in subsequent PCR reactions.

| | | SEQ ID |
|---|---|---|
| Item | Sequence | NO: |
| HC- intein- 5′ | 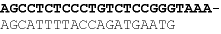 | 54 |
| Revised LC- | GGGCGGGCACGCGCATGTCCAT- | 55 |
| intein-3′ | GTTGTGTGCGTAAAGTAGTC | |
| HC- intein(1aa)- 5′ | 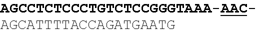 | 56 |
| Revised LC- intein(1aa)- 3′ | 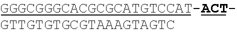 | 57 |
| HC- intein(3aa)- 5′ | 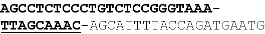 | 58 |
| Revised LC- intein(3aa)- 3′ | 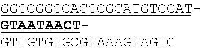 | 59 |
| HC-Srgl-5′ | 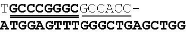 | 60 |
| LC-BamHI-3′ | 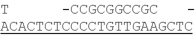 | 61 |

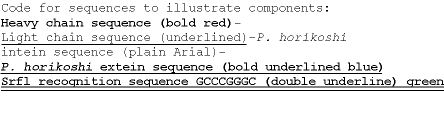

PCR Amplification and assembly of D2E7 Heavy Chain-Intein-D2E7 Light Chain fusion: Using the pCR2.1-TOPO-p. horikoshii intein clone generated above as template, PCR was performed using the primers P. horikoshii int-5′ and revised P.hori-3′ to restore the proper 3′ end to the intein. The polymerase used was Pful DNA Polymerase to avoid the A-tailing that occurs with Platinum Taq.

PCR was run according to the following program:

| | Step | | | | | | | |
|---|---|---|---|---|---|---|---|---|
| | 1 | 2 | 3 | 4 | 5 | 6 | 7 | 8 |
| Temp | 94° C. | 94° C. | 55° C. | 72° C. | Go to step 2 (34 times) | 72° C. | 4° C. | End |
| Time | 2 min | 1 min | 1 min | 2 min | | 5 min | hold | |

The PCR amplification product was gel purified using the Qiaquick Gel Extraction kit (Qiagen, Valencia, Calif.). This product was used as template in the next set of reactions.

Three sets of PCR reactions were performed to generate intein coding sequences with varied numbers of extein residues 5′ and 3′ of the intein coding sequence. The extein codons come from the native DNA polymerase gene in P. horikoshii which this intein is naturally part of. Primers were used as follows: Set 1 introduces zero extein sequence (HC-intein-5′ and Revised LC-intein-3′), Set 2 introduces one amino acid (3 base pairs) at both ends of the intein (HC-intein(1aa)-5′ and Revised LC-intein(1aa)-3′) and Set 3 introduces three amino acids (9 base pairs) at both ends of the intein (HC-intein(3aa)-5′ and Revised LC-intein(3aa)-3′).

The PCR program was the same as given above. PCR products were gel purified using the Qiaquick Gel Extraction kit (Qiagen). These products were used as primers in the next set of reactions.

Three sets of PCR reactions were performed to generate the fusion of D2E7 Heavy Chain to intein, with 0, 1 or 3 extein amino acids in between. The template for the reactions is the D2E7 Heavy Chain DNA. The PCR products described above were used as the 3′ primers, respectively, and HC-SrfI-5′ was used as the 5′ primer in all reactions. Pful DNA Polymerase was used.

PCR was run according to the following program:

| | Step | | | | | | | |
|---|---|---|---|---|---|---|---|---|
| | 1 | 2 | 3 | 4 | 5 | 6 | 7 | 8 |
| Temp | 94° C. | 94° C. | 50° C. | 72° C. | Go to step 2 (39 times) | 72° C. | 4° C. | End |
| Time | 2 min | 1 min | 1 min | 3 min | | 5 min | hold | |

PCR product was gel purified using the Qiaquick Gel Extraction kit (Qiagen). This product was used as primers in the next set of reactions.

Three sets of PCR reactions were performed to generate the fusion of D2E7 Heavy Chain-intein to D2E7 Light Chain, with 0, 1 or 3 extein amino acids in between. The template for the reactions is the D2E7 Light Chain DNA. The PCR products described directly above were used as the 5′ primers, respectively, and LC-BamHI-3′ was used as the 3′ primer in all reactions. Pful DNA Polymerase was used.

PCR was run according to the following program:

| | Step | | | | | | | |
|---|---|---|---|---|---|---|---|---|
| | 1 | 2 | 3 | 4 | 5 | 6 | 7 | 8 |
| Temp | 94° C. | 94° C. | 55° C. | 72° C. | Go to step 2 (39 times) | 72° C. | 4° C. | End |
| Time | 2 min | 1 min | 1 min | 5 min | | 5 min | hold | |

The PCR product produced was diffuse and sparse when run on a gel. These reactions were directly used as template in the final round of PCR, using HC-SrfI-5′ and LC-BamHI-3′ as primers. Pful DNA Polymerase was used. The same PCR program was used as set forth above. PCR products were gel purified using the Qiaquick Gel Extraction kit (Qiagen).

The purified PCR products described above were subcloned into pCR-BluntII-TOPO (Invitrogen) using the Zero Blunt TOPO PCR Cloning Kit (Invitrogen). Clones were sequenced to verify that the constructs exhibited the expected nucleic acid sequences. Correct clones were found for each type of product. The D2E7 Heavy Chain-intein-D2E7 Light Chain cassette was excised from pCR-BluntII-TOPO using SrfI and NotI and subcloned into pTT3 restricted with the same enzymes and gel purified.

Three Expression Constructs for D2E7 Heavy Chain-intein-D2E7 Light Chain, utilizing the P. horikoshii intein were designed: pTT3-HcintLC-p.hori (See FIG. 14 for plasmid map); pTT3-HcintLC1aa-p.hori; and pTT3-HcintLC3aa-p.hori.

TABLE 10A
Nucleotide sequence of pTT3-HcintLC-p.hori (SEQ ID NO: 62)

5′-
gcggccgctcgaggccggcaaggccggatcccccgacctcgacctctggctaataaaggaaatttattttcattgcaatag
tgtgttggaattttttgtgtctctcactcggaaggacatatgggagggcaaatcatttggtcgagatccctcggagatctctagc
tagaggatcgatccccgccccggacgaactaaacctgactacgacatctctgccccttcttcgcggggcagtgcatgtaat
cccttcagttggttggtacaacttgccaactgggccctgttccacatgtgacacggggggggaccaaacacaaaggggtt
ctctgactgtagttgacatccttataaatggatgtgcacatttgccaacactgagtggctttcatcctggagcagactttgcagt
ctgtggactgcaacacaacattgcctttatgtgtaactcttggctgaagctcttacaccaatgctgggggacatgtacctccc
aggggcccaggaagactacgggaggctacaccaacgtcaatcagaggggcctgtgtagctaccgataagcggaccct
caagagggcattagcaatagtgtttataaggcccccttgttaaccctaaacgggtagcatatgcttcccgggtagtagtatat
actatccagactaaccctaattcaatagcatatgttacccaacgggaagcatatgctatcgaattagggttagtaaaagggt
cctaaggaacagcgatatctcccaccccatgagctgtcacggttttatttacatggggtcaggattccacgagggtagtgaa
ccattttagtcacaagggcagtggctgaagatcaaggagcgggcagtgaactctcctgaatcttcgcctgcttcttcattctc
cttcgtttagctaatagaataactgctgagttgtgaacagtaaggtgtatgtgaggtgctcgaaaacaaggtttcaggtgacg
cccccagaataaaatttggacggggggttcagtggtggcattgtgctatgacaccaatataaccctcacaaaccccttggg
caataaatactagtgtaggaatgaaacattctgaatatctttaacaatagaaatccatggggtggggacaagccgtaaag
actggatgtccatctcacacgaatttatggctatgggcaacacataatcctagtgcaatatgatactggggttattaagatgtg
tcccaggcagggaccaagacaggtgaaccatgttgttacactctatttgtaacaaggggaaagagagtggacgccgac
agcagcggactccactggttgtctctaacacccccgaaaattaaacggggctccacgccaatggggcccataaacaaa
gacaagtggccactcttttttttgaaattgtggagtgggggcacgcgtcagcccccacacgccgccctgcggttttggactgt
aaaataagggtgtaataacttggctgattgtaaccccgctaaccactgcggtcaaaccacttgcccacaaaaccactaat
ggcaccccggggaatacctgcataagtaggtgggcgggccaagataggggcgcgattgctgcgatctggaggacaaa
ttacacacacttgcgcctgagcgccaagcacagggttgttggtcctcatattcacgaggtcgctgagagcacggtgggcta
atgttgccatgggtagcatatactacccaaatatctggatagcatatgctatcctaatctatatctgggtagcataggctatcct
aatctatatctgggtagcatatgctatcctaatctatatctgggtagtatatgctatcctaatttatatctgggtagcataggctatc
ctaatctatatctgggtagcatatgctatcctaatctatatctgggtagtatatgctatcctaatctgtatccgggtagcatatgct
atcctaatagagattagggtagtatatgctatcctaatttatatctgggtagcatatactacccaaatatctggatagcatatgct
atcctaatctatatctgggtagcatatgctatcctaatctatatctgggtagcataggctatcctaatctatatctgggtagcatat
gctatcctaatctatatctgggtagtatatgctatcctaatttatatctgggtagcataggctatcctaatctatatctgggtagcat
atgctatcctaatctatatctgggtagtatatgctatcctaatctgtatccgggtagcatatgctatcctcatgataagctgtcaa
acatgagaattttcttgaagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatga
gacaataaccctgataaatgcttcaataatattgaaaaaggaagagtatgagtattcaacatttccgtgtcgcccttattccct
tttttgcggcattttgccttcctgtttttgctcacccagaaacgctggtgaaagtaaaagatgctgaagatcagttgggtgcacg
agtgggttacatcgaactggatctcaacagcggtaagatccttgagagttttcgccccgaagaacgttttccaatgatgagc
acttttaaagttctgctatgtggcgcggtattatcccgtgttgacgccgggcaagagcaactcggtcgccgcatacactattct
cagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttg
cacaacatgggggatcatgtaactcgccttgatcgttgggaaccggagctgaatgaagccataccaaacgacgagcgtg
acaccacgatgcctgcagcaatggcaacaacgttgcgcaaactattaactggcgaactacttactctagcttcccggcaa
caattaatagactggatggaggcggataaagttgcaggaccacttctgcgctcggcccttccggctggctggtttattgctga
taaatctggagccggtgagcgtgggtctcgcggtatcattgcagcactggggccagatggtaagccctcccgtatcgtagtt
atctacacgacggggagtcaggcaactatggatgaacgaaatagacagatcgctgagataggtgcctcactgattaagc
attggtaactgtcagaccaagtttactcatatatactttagattgatttaaaacttcatttttaatttaaaaggatctaggtgaagat
cctttttgataatctcatgaccaaaatcccttaacgtgagttttcgttccactgagcgtcagaccccgtagaaaagatcaaag
gatcttcttgagatcctttttttctgcgcgtaatctgctgcttgcaaacaaaaaaaccaccgctaccagcggtggtttgtttgccg
gatcaagagctaccaactctttttccgaaggtaactggcttcagcagagcgcagataccaaatactgttcttctagtgtagcc
gtagttaggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacggg
gggttcgtgcacacagcccagcttggagcgaacgacctacaccgaactgagatacctacagcgtgagctatgagaaag
cgccacgcttcccgaagggagaaaggcggacaggtatccggtaagcggcagggtcggaacaggagagcgcacgag
ggagcttccagggggaaacgcctggtatctttatagtcctgtcgggtttcgccacctctgacttgagcgtcgatttttgtgatgct
cgtcaggggggcggagcctatggaaaaacgccagcaacgcggcctttttacggttcctggccttttgctggccttttgctcac
atgttctttcctgcgttatcccctgattctgtggataaccgtattaccgcctttgagtgagctgataccgctcgccgcagccgaa
cgaccgagcgcagcgagtcagtgagcgaggaagcggaagagcgcccaatacgcaaaccgcctctccccgcgcgttg
gccgattcattaatgcagctggcacgacaggtttcccgactggaaagcgggcagtgagcgcaacgcaattaatgtgagtt
agctcactcattaggcaccccaggctttacactttatgcttccggctcgtatgttgtgtggaattgtgagcggataacaatttca
cacaggaaacagctatgaccatgattacgccaagctctagctagaggtcgaccaattctcatgtttgacagcttatcatcgc
agatccgggcaacgttgttgccattgctgcaggcgcagaactggtaggtatggaagatctatacattgaatcaatattggca
attagccatattagtcattggttatatagcataaatcaatattggctattggccattgcatacgttgtatctatatcataatatgtac
atttatattggctcatgtccaatatgaccgccatgttgacattgattattgactagttattaatagtaatcaattacggggtcatta
gttcatagcccatatatggagttccgcgttacataacttacggtaaatggcccgcctggctgaccgcccaacgacccccgc
ccattgacgtcaataatgacgtatgttcccatagtaacgccaatagggactttccattgacgtcaatgggtggagtatttacg
gtaaactgcccacttggcagtacatcaagtgtatcatatgccaagtccgccccctattgacgtcaatgacggtaaatggccc
gcctggcattatgcccagtacatgaccttacgggactttcctacttggcagtacatctacgtattagtcatcgctattaccatgg
tgatgcggttttggcagtacaccaatgggcgtggatagcggtttgactcacggggatttccaagtctccaccccattgacgtc
aatgggagtttgttttggcaccaaaatcaacgggactttccaaaatgtcgtaataaccccgccccgttgacgcaaatgggc
ggtaggcgtgtacggtgggaggtctatataagcagagctcgtttagtgaaccgtcagatcctcactctcttccgcatcgctgt
ctgcgagggccagctgttgggctcgcggttgaggacaaactcttcgcggtctttccagtactcttggatcggaaacccgtcg
gcctccgaacggtactccgccaccgagggacctgagcgagtccgcatcgaccggatcggaaaacctctcgagaaagg
cgtctaaccagtcacagtcgcaaggtaggctgagcaccgtggcgggcggcagcgggtggcggtcggggttgtttctggc
ggaggtgctgctgatgatgtaattaaagtaggcggtcttgagacggcggatggtcgaggtgaggtgtggcaggcttgagat
ccagctgttggggtgagtactccctctcaaaagcgggcattacttctgcgctaagattgtcagtttccaaaaacgaggagga
tttgatattcacctggcccgatctggccatacacttgagtgacaatgacatccactttgcctttctctccacaggtgtccactccc
aggtccaagtttgggcgccaccatggagtttgggctgagctggctttttcttgtcgcgattttaaaaggtgtccagtgt-
gaggtgcagctggtggagtctgggggaggcttggtacagcccggcaggtccctgagactctcctgtgcggcctctggattc
acctttgatgattatgccatgcactgggtccggcaagctccagggaagggcctggaatgggtctcagctatcacttggaata
gtggtcacatagactatgcggactctgtggagggccgattcaccatctccagagacaacgccaagaactccctgtatctgc
aaatgaacagtctgagagctgaggatacggccgtatattactgtgcgaaagtctcgtaccttagcaccgcgtcctcccttga
ctattggggccaaggtaccctggtcaccgtctcgagtgcgtcgaccaagggcccatcggtcttccccctggcaccctcctc
caagagcacctctgggggcacagcggccctgggctgcctggtcaaggactacttccccgaaccggtgacggtgtcgtgg
aactcaggcgccctgaccagcggcgtgcacaccttcccggctgtcctacagtcctcaggactctactccctcagcagcgt
ggtgaccgtgccctccagcagcttgggcacccagacctacatctgcaacgtgaatcacaagcccagcaacaccaaggt
ggacaagaaagttgagcccaaatcttgtgacaaaactcacacatgcccaccgtgcccagcacctgaactcctgggggg
accgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatg
gcaaggagtacaagtgcaaggtctccaacaaagccctcccagcccccatcgagaaaaccatctccaaagccaaagg
gcagccccgagaaccacaggtgtacaccctgcccccatcccgggatgagctgaccaagaaccaggtcagcctgacct
gcctggtcaaaggcttctatcccagcgacatcgccgtggagtgggagagcaatgggcagccggagaacaactacaag
accacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtctccggg
taaa-
agcattttaccagatgaatggctcccaattgttgaaaatgaaaaagttcgattcgtaaaaattggagacttcatagataggg
agattgaggaaaacgctgagagagtgaagagggatggtgaaactgaaattctagaggttaaagatcttaaagccctttcc
ttcaatagagaaacaaaaaagagcgagctcaagaaggtaaaggccctaattagacaccgctattcagggaaggtttac
agcattaaactaaagtcagggagaaggatcaaaataacctcaggtcatagtctgttctcagtaaaaaatggaaagctagt
taaggtcaggggagatgaactcaagcctggtgatctcgttgtcgttccaggaaggttaaaacttccagaaagcaagcaag
tgctaaatctcgttgaactactcctgaaattacccgaagaggagacatcgaacatcgtaatgatgatcccagttaaaggta
gaaagaatttcttcaaagggatgctcaaaacattatactggatcttcggggagggagaaaggccaagaaccgcagggc
gctatctcaagcatcttgaaagattaggatacgttaagctcaagagaagaggctgtgaagttctcgactgggagtcacttaa
gaggtacaggaagctttacgagaccctcattaagaacctgaaatataacggtaatagcagggcatacatggttgaattta
actctctcagggatgtagtgagcttaatgccaatagaagaacttaaggagtggataattggagaacctaggggtcctaag
ataggtaccttcattgatgtagatgattcatttgcaaagctcctaggttactacataagtagcggagatgtagagaaagatag
ggtgaagttccacagtaaagatcaaaacgttctcgaggatatagcgaaacttgccgagaagttatttggaaaggtgagga
gaggaagaggatatattgaggtatcagggaaaattagccatgccatatttagagttttagcggaaggtaagagaattcca
gagttcatcttcacatccccaatggatattaaggtagccttccttaagggactcaacggtaatgctgaagaattaacgttctcc
actaagagtgagctattagttaaccagcttatccttctcctgaactccattggagtttcggatataaagattgaacatgagaaa
ggggtttacagagtttacataaataagaaggaatcctccaatggggatatagtacttgatagcgtcgaatctatcgaagttg
aaaaatacgagggctacgtttatgatctaagtgttgaggataatgagaacttcctcgttggcttcggactactttacgcacac
aac-
atggacatgcgcgtgcccgcccagctgctgggcctgctgctgctgtggttccccggctcgcgatgcgacatccagatgacc
cagtctccatcctccctgtctgcatctgtaggggacagagtcaccatcacttgtcgggcaagtcagggcatcagaaattactt
agcctggtatcagcaaaaaccagggaaagcccctaagctcctgatctatgctgcatccactttgcaatcaggggtcccatc
tcggttcagtggcagtggatctgggacagatttcactctcaccatcagcagcctacagcctgaagatgttgcaacttattact
gtcaaaggtataaccgtgcaccgtatacttttggccaggggaccaaggtggaaatcaaacgtacggtggctgcaccatct
gtcttcatcttcccgccatctgatgagcagttgaaatctggaactgcctctgttgtgtgcctgctgaataacttctatcccagag
aggccaaagtacagtggaaggtggataacgccctccaatcgggtaactcccaggagagtgtcacagagcaggacagc
aaggacagcacctacagcctcagcagcaccctgacgctgagcaaagcagactacgagaaacacaaagtctacgcct
gcgaagtcacccatcagggcctgagctcgcccgtcacaaagagcttcaacaggggagagtgt-3′

TABLE 10B
Amino Acid Sequence of the open reading frame in pTT3-HcintLC-p.hori (SEQ ID NO: 63)

Mefglswlflvailkgvqcevqlvesggglvqpgrslrlscaasgftfddyamhwvrqapgkglewvsaitwnsghidyad
svegrftisrdnaknslylqmnslraedtavyycakvsylstassidywgqgtlvtvssastkgpsvfplapsskstsggtaal
gclvkdyfpepvtvswnsgaltsgvhtfpavlqssglyslssvvtvpssslgtqtyicnvnhkpsntkvdkkvepkscdktht
cppcpapellggpsvflfppkpkdtlmisrtpevtcvvvdvshedpevkfnwyvdgvevhnaktkpreeqynstyrvvsvl
tvlhqdwlngkeykckvsnkalpapiektiskakgqprepqvytlppsrdeltknqvsltclvkgfypsdiavewesngqp
ennykttppvldsdgsfflyskltvdksrwqqgnvfscsvmhealhnhytqkslslspgk-
silpdewlpivenekvrfvkigdfidreieenaervkrdgeteilevkdlkalsfnretkkselkkvkalirhrysgkvysiklksg
rrikitsghslfsvkngklvkvrgdelkpgdlvvvpgrlklpeskqvlnlvelllklpeeetsnivmmipvkgrknffkgmlktly
wifgegerprtagrylkhlerlgyvklkrrgcevldweslkryrklyetliknlkyngnsraymvefnslrdvvslmpieelkewi
igeprgpkigtfidvddsfakllgyyissgdvekdrvkfhskdqnvlediaklaeklfgkvrrgrgyievsgkishaifrvlaegk
ripefiftspmdikvaflkglngnaeeltfstksellvnqlilllnsigvsdikiehekgvyrvyinkkessngdivldsvesievek
yegyvydlsvednenflvgfgllyahn-
mdmrvpaqllgllllwfpgsrcdiqmtqspsslsasvgdrvtitcrasqgirnylawyqqkpgkapklliyaastlqsgvpsrf
sgsgsgtdftltisslqpedvatyycqrynrapytfgqgtkveikrtvaapsvfifppsdeqlksgtasvvcllnnfypreakvq
wkvdnalqsgnsqesvteqdskdstyslsstltlskadyekhkvyacevthqglsspvtksfnrgec
Text/font symbol code for sequences:
pTT3 Vector-Heavy Chain-Intein-Light Chain

In the following 2 constructs, the only difference from the construct above is the inclusion of extein sequences native to P. horikoshii (underlined). The sequences shown are from the end of the D2E7 heavy chain coding region (last 9 base pairs as shown in red) to the 5′ end of the D2E7 light chain coding region (first 9 base pairs as shown in pink, on a separate line)

TABLE 11A
pTT3-HcintLC1aa-p.hori partial coding sequence (SEQ ID NO: 64)

5′-ccgggtaaa-
aacagcattttaccagatgaatggctcccaattgttgaaaatgaaaaagttcgattcgtaaaaattggagacttcatagata
gggagattgaggaaaacgctgagagagtgaagagggatggtgaaactgaaattctagaggttaaagatcttaaagccc
tttccttcaatagagaaacaaaaaagagcgagctcaagaaggtaaaggccctaattagacaccgctattcagggaaggt
ttacagcattaaactaaagtcagggagaaggatcaaaataacctcaggtcatagtctgttctcagtaaaaaatggaaagc
tagttaaggtcaggggagatgaactcaagcctggtgatctcgttgtcgttccaggaaggttaaaacttccagaaagcaagc
aagtgctaaatctcgttgaactactcctgaaattacccgaagaggagacatcgaacatcgtaatgatgatcccagttaaag
gtagaaagaatttcttcaaagggatgctcaaaacattatactggatcttcggggagggagaaaggccaagaaccgcag
ggcgctatctcaagcatcttgaaagattaggatacgttaagctcaagagaagaggctgtgaagttctcgactgggagtcac
ttaagaggtacaggaagctttacgagaccctcattaagaacctgaaatataacggtaatagcagggcatacatggttgaa
tttaactctctcagggatgtagtgagcttaatgccaatagaagaacttaaggagtggataattggagaacctaggggtccta
agataggtaccttcattgatgtagatgattcatttgcaaagctcctaggttactacataagtagcggagatgtagagaaagat
agggtgaagttccacagtaaagatcaaaacgttctcgaggatatagcgaaacttgccgagaagttatttggaaaggtgag
gagaggaagaggatatattgaggtatcagggaaaattagccatgccatatttagagttttagcggaaggtaagagaattc
cagagttcatcttcacatccccaatggatattaaggtagccttccttaagggactcaacggtaatgctgaagaattaacgttct
ccactaagagtgagctattagttaaccagcttatccttctcctgaactccattggagtttcggatataaagattgaacatgaga
aaggggtttacagagtttacataaataagaaggaatcctccaatggggatatagtacttgatagcgtcgaatctatcgaagt
tgaaaaatacgagggctacgtttatgatctaagtgttgaggataatgagaacttcctcgttggcttcggactactttacgcaca
caacagt-
atggacatg-3′

TABLE 11B
pTT3-HcintLC1aa-p.hori partial amino acid sequence showing 4
amino acids upstream of the heavy chain and four amino acids downstream
of the intein (SEQ ID NO: 65)

Pgknsilpdewlpivenekvrfvkigdfidreieenaervkrdgeteilevkdlkalsfnretkkselkkvkalirhrysgkvysi
klksgrrikitsghslfsvkngklvkvrgdelkpgdlvvvpgrlklpeskqvlnlvelllklpeeetsnivmmipvkgrknffkgm
lktlywifgegerprtagrylkhlerlgyvklkrrgcevldweslkryrklyetliknlkyngnsraymvefnslrdvvslmpieel
kewiigeprgpkigtfidvddsfakllgyyissgdvekdrvkfhskdqnvlediaklaeklfgkvrrgrgyievsgkishaifrvl
aegkripefiftspmdikvaflkglngnaeeltfstksellvnqlilllnsigvsdikiehekgvyrvyinkkessngdivldsvesi
evekyegyvydlsvednenflvgfgllyahn-s-mdm
Heavy Chain 3′ sequence-Intein-Extein-Light Chain 5′ sequence

TABLE 12A
pTT3-HcintLC3aa-p.hori partial coding sequence (SEQ ID NO: 66)

5′-ccgggtaaa-ttagcaaac-
agcattttaccagatgaatggctcccaattgttgaaaatgaaaaagttcgattcgtaaaaattggagacttcatagataggg
agattgaggaaaacgctgagagagtgaagagggatggtgaaactgaaattctagaggttaaagatcttaaagccctttcc
ttcaatagagaaacaaaaaagagcgagctcaagaaggtaaaggccctaattagacaccgctattcagggaaggtttac
agcattaaactaaagtcagggagaaggatcaaaataacctcaggtcatagtctgttctcagtaaaaaatggaaagctagt
taaggtcaggggagatgaactcaagcctggtgatctcgttgtcgttccaggaaggttaaaacttccagaaagcaagcaag
tgctaaatctcgttgaactactcctgaaattacccgaagaggagacatcgaacatcgtaatgatgatcccagttaaaggta
gaaagaatttcttcaaagggatgctcaaaacattatactggatcttcggggagggagaaaggccaagaaccgcagggc
gctatctcaagcatcttgaaagattaggatacgttaagctcaagagaagaggctgtgaagttctcgactgggagtcacttaa
gaggtacaggaagctttacgagaccctcattaagaacctgaaatataacggtaatagcagggcatacatggttgaattta
actctctcagggatgtagtgagcttaatgccaatagaagaacttaaggagtggataattggagaacctaggggtcctaag
ataggtaccttcattgatgtagatgattcatttgcaaagctcctaggttactacataagtagcggagatgtagagaaagatag
ggtgaagttccacagtaaagatcaaaacgttctcgaggatatagcgaaacttgccgagaagttatttggaaaggtgagga
gaggaagaggatatattgaggtatcagggaaaattagccatgccatatttagagttttagcggaaggtaagagaattcca
gagttcatcttcacatccccaatggatattaaggtagccttccttaagggactcaacggtaatgctgaagaattaacgttctcc
actaagagtgagctattagttaaccagcttatccttctcctgaactccattggagtttcggatataaagattgaacatgagaaa
ggggtttacagagtttacataaataagaaggaatcctccaatggggatatagtacttgatagcgtcgaatctatcgaagttg
aaaaatacgagggctacgtttatgatctaagtgttgaggataatgagaacttcctcgttggcttcggactactttacgcacac
aac-agttattac-atggacatg-3′

TABLE 12B
pTT3-HcintLC3aa-p.hori partial amino acid sequence showing
intein and flanking sequences (SEQ ID NO: 67)

Pgk-lan-
silpdewlpivenekvrfvkigdfidreieenaervkrdgeteilevkdlkalsfnretkkselkkvkalirhrysgkvysiklksg
rrikitsghslfsvkngklvkvrgdelkpgdlvvvpgrlklpeskqvlnlyelllklpeeetsnivmmipvkgrknffkgmlktly
wifgegerprtagrylkhlerlgyvklkrrgcevldweslkryrklyetliknlkyngnsraymvefnslrdvvslmpieelkewi
igeprgpkigtfidvddsfakllgyyissgdvekdrvkfhskdqnvlediaklaeklfgkvrrgrgyievsgkishaifrvlaegk
ripefiftspmdikvaflkglngnaeeltfstksellvnqlilllnsigvsdikiehekgvyrvyinkkessngdivldsvesievek
yegyvydlsvednenflvgfgllyahn-syy-mdm
Heavy Chain 3′ sequence-Intein-Extein-Light Chain 5′ sequence

Primers used for constructs A, B, E, H, I, J, K, and L were:

YKF1:

(SEQ ID NO: 68)

GGACTACTTTACGCAGCCAACATGGACATGC
YKR1:

(SEQ ID NO: 69)

GCATGTCCATGTTGGCTGCGTAAAGTAGTCC
YKF2:

(SEQ ID NO: 70)

GGACTACTTTACGCAGCCAACAGTATGGACATGC
YKR2:

(SEQ ID NO: 71)

GCATGTCCATACTGTTGGCTGCGTAAAGTAGTCC
YKF3:

(SEQ ID NO: 72)

GGTGAGGAGAGGAAGAGG
YKR3:

(SEQ ID NO: 73)

CCAGAGGTCGAGGTCG
YKF4:

(SEQ ID NO: 74)

CGGCGTGGAGGTGC
YKR4:

(SEQ ID NO: 75)

CAACAATTGGGAGCCATTCATCTGGTAAAATGGTTTTACCCGGAG
YKF5:

(SEQ ID NO: 76)

CCGCCCAGCTGCTGGGCGACGAGTGGTTCCCCGGCTCGCG
YKR5:

(SEQ ID NO: 77)

Cgcgagccggggaaccactcgtcgcccagcagctgggcgg
YKF6:

(SEQ ID NO: 78)

tgagcggccgctcga
YKR6:

(SEQ ID NO: 79)

gttgtgtgcgtaaag
YKF7:

(SEQ ID NO: 80)

agcattttaccagat
YKR7:

(SEQ ID NO: 81)

ggtggcgcccaaact
YKF8:

(SEQ ID NO: 82)

ctttacgcacacaacatggacatgcgcgtg
YKR8:

(SEQ ID NO: 83)

tcgagcggccgctcaacactctcccct
YKF9:

(SEQ ID NO: 84)

agtttgggcgccaccatggagtttgggctg
YKR9:

(SEQ ID NO: 85)

atctggtaaaatgcttttacccggagacag
YKF10:

(SEQ ID NO: 86)

agtttgggcgccaccatggacatgcgcgtg
YKR10:

(SEQ ID NO: 87)

atctggtaaaatgctacactctcccctgttg
YKF11:

(SEQ ID NO: 88)

ctttacgcacacaacatggagtttgggctg
YKR11:

(SEQ ID NO: 89)

tcgagcggccgctcatttacccggagacag
YKF12:

(SEQ ID NO: 90)

cgccaagctctagc
YKR12:

(SEQ ID NO: 91)

ggtcgaggtcgggg
YKF13:

(SEQ ID NO: 92)

acatgcgcgtgcccgcccagtggttccccggctcgcgatg
YKR13:

(SEQ ID NO: 93)

catcgcgagccggggaaccactgggcgggcacgcgcatgt
YKF14:

(SEQ ID NO: 94)

ctttacgcacacaacgacatccagatgacc
YKR14:

(SEQ ID NO: 95)

ggtcatctggatgtcgttgtgtgcgtaaag
YKF15:

(SEQ ID NO: 96)

tggttccccggctcgGgaGgcgacatccagatgacc
YKR15:

(SEQ ID NO: 97)

ggtcatctggatgtcgcctcccgagccggggaacca

To prepare Construct A, plasmid pTT3 HC-int-LC P.hori was used as template 2 and overlapping DNA fragments were amplified using mutagenesis primer YKF1 and primer YKR3, and mutagenesis primer YKR1 with primer YKF3, respectively. A DNA fragment linking the above 2 fragments was generated by PCR amplification using the mixture of the above 2 PCR fragments as template, and primers YKF3 and YKR3. This PCR fragment is then cut with restriction enzymes EcoR I and Not I, and cloned into pTT3 HC-int-LC P.hori cut with the same restriction enzymes.

Construct B was generated in a similar manner as for construct A, except that mutagenesis primers YKF2 and YKR2 were used in place of YKF1 and YKR1, and plasmid pTT3 HC-int-LC-1 as P.hori was used as the PCR template in the place of plasmid pTT3 HC-int-LC P.hori, and pTT3 HC-int-LC P.hori vector was used as the backbone for cloning.

To prepare Construct E, a DNA fragment was amplified using plasmid pTT3 HC-int-LC-1 aa P.hori as template, and primer YKF4 and mutagenesis primer YKR4. This PCR fragment was cut with Sac II and Mfe I, and cloned into pTT3 HC-int-LC P.hori cut with the same restriction enzymes.

For Construct H, pTT3 HC-int-LC P.hori was used as template 2, and overlapping fragments were amplified using mutagenesis primer YKF5 and primer YKR3 for one fragment and primer F3 and mutagenesis primer R5 for the other. A second round of PCR amplification was carried out using the above 2 fragments as templates and primers YKF3 and YKR3. This fragment was digested with restriction enzymes EcoR I and Not I, and cloned into pTT3 HC-int-LC P.hori cut with the same enzymes.

To prepare Construct J, pTT3 HC-int-LC P.hori was used as template 2, and overlapping fragments were amplified using mutagenesis primer YKF13 and primer YKR3 for one fragment and primer F3 and mutagenesis primer R13 for the other. A second round of PCR amplification was carried out using the above 2 fragments as templates and primers YKF3 and YKR3. This fragment was cut with restriction enzymes EcoR I and Not I and cloned into pTT3 HC-int-LC P.hori cut with the same enzymes.

For Construct K, pTT3 HC-int-LC P.hori served as template 2. Overlapping fragments were amplified using mutagenesis primer YKF14 and primer YKR3 for one fragment and primer F3 and mutagenesis primer R14 for the other. A second round of PCR amplification was carried out using the above 2 fragments as templates and primers YKF3 and YKR3. This fragment was digested with restriction enzymes EcoR I and Not I, and cloned into pTT3 HC-int-LC P.hori cut with the same enzymes.

To make Constructs L, Using pTT3 HC-int-LC P.hori was used as template 2, and overlapping fragments were amplified using mutagenesis primer YKF15 and primer YKR3 for one fragment and primer F3 and mutagenesis primer R15 for the other. A second round of PCR amplification was carried out using the above 2 fragments as templates and primers YKF3 and YKR3. This fragment was digested with restriction enzymes EcoR I and Not I, and cloned into pTT3 HC-int-LC P.hori cut with the same enzymes.

The nucleotide sequences of all constructs were verified. All constructs have the same sequence as pTT3 HC-int-LC P.hori except for the sequences between the last codons of the D2E7 heavy chain (encoding PGK) and the first codons of the D2E7 light chain mature sequence (encoding DIQ). Sequences in this region, which include wt or mutant intein in conjunction with wt or mutant light chain signal sequence, are provided for all the constructs as below.

TABLE 13A
Partial coding sequence of construct A (SEQ ID NO: 98)

Ccgggtaaa-
agcattttaccagatgaatggctcccaattgttgaaaatgaaaaagttcgattcgtaaaaattggagacttcatagataggg
agattgaggaaaacgctgagagagtgaagagggatggtgaaactgaaattctagaggttaaagatcttaaagccctttcc
ttcaatagagaaacaaaaaagagcgagctcaagaaggtaaaggccctaattagacaccgctattcagggaaggtttac
agcattaaactaaagtcagggagaaggatcaaaataacctcaggtcatagtctgttctcagtaaaaaatggaaagctagt
taaggtcaggggagatgaactcaagcctggtgatctcgttgtcgttccaggaaggttaaaacttccagaaagcaagcaag
tgctaaatctcgttgaactactcctgaaattacccgaagaggagacatcgaacatcgtaatgatgatcccagttaaaggta
gaaagaatttcttcaaagggatgctcaaaacattatactggatcttcggggagggagaaaggccaagaaccgcagggc
gctatctcaagcatcttgaaagattaggatacgttaagctcaagagaagaggctgtgaagttctcgactgggagtcacttaa
gaggtacaggaagctttacgagaccctcattaagaacctgaaatataacggtaatagcagggcatacatggttgaattta
actctctcagggatgtagtgagcttaatgccaatagaagaacttaaggagtggataattggagaacctaggggtcctaag
ataggtaccttcattgatgtagatgattcatttgcaaagctcctaggttactacataagtagcggagatgtagagaaagatag
ggtgaagttccacagtaaagatcaaaacgttctcgaggatatagcgaaacttgccgagaagttatttggaaaggtgagga
gaggaagaggatatattgaggtatcagggaaaattagccatgccatatttagagttttagcggaaggtaagagaattcca
gagttcatcttcacatccccaatggatattaaggtagccttccttaagggactcaacggtaatgctgaagaattaacgttctcc
actaagagtgagctattagttaaccagcttatccttctcctgaactccattggagtttcggatataaagattgaacatgagaaa
ggggtttacagagtttacataaataagaaggaatcctccaatggggatatagtacttgatagcgtcgaatctatcgaagttg
aaaaatacgagggctacgtttatgatctaagtgttgaggataatgagaacttcctcgttggcttcggactactttacgcagcc
aacatggacatgcgcgtgcccgcccagctgctgggcctgctgctgctgtggttccccggctcgcgatgc-gacatccag

TABLE 13B
Partial amino acid sequence showing intein and flanking
sequences in construct A (SEQ ID NO: 99)

Pgk-
silpdewlpivenekvrfvkigdfidreieenaervkrdgeteilevkdlkalsfnretkkselkkvkalirhrysgkvysiklksg
rrikitsghslfsvkngklvkvrgdelkpgdlvvvpgrlklpeskqvlnlvelllklpeeetsnivmmipvkgrknffkgmlktly
wifgegerprtagrylkhlerlgyvklkrrgcevldweslkryrklyetliknlkyngnsraymvefnslrdvvslmpieelkewi
igeprgpkigtfidvddsfakllgyyissgdvekdrvkfhskdqnvlediaklaeklfgkvrrgrgyievsgkishaifrvlaegk
ripefiftspmdikvaflkglngnaeeltfstksellvnqlilllnsigvsdikiehekgvyrvyinkkessngdivldsvesievek
yegyvydlsvednenflvgfgllyaanmdmrvpaqllgllllwfpgsrc-diq

TABLE 14A
Partial coding sequence in construct B (SEQ ID NO: 100)

Ccgggtaaa-
agcattttaccagatgaatggctcccaattgttgaaaatgaaaaagttcgattcgtaaaaattggagacttcatagataggg
agattgaggaaaacgctgagagagtgaagagggatggtgaaactgaaattctagaggttaaagatcttaaagccctttcc
ttcaatagagaaacaaaaaagagcgagctcaagaaggtaaaggccctaattagacaccgctattcagggaaggtttac
agcattaaactaaagtcagggagaaggatcaaaataacctcaggtcatagtctgttctcagtaaaaaatggaaagctagt
taaggtcaggggagatgaactcaagcctggtgatctcgttgtcgttccaggaaggttaaaacttccagaaagcaagcaag
tgctaaatctcgttgaactactcctgaaattacccgaagaggagacatcgaacatcgtaatgatgatcccagttaaaggta
gaaagaatttcttcaaagggatgctcaaaacattatactggatcttcggggagggagaaaggccaagaaccgcagggc
gctatctcaagcatcttgaaagattaggatacgttaagctcaagagaagaggctgtgaagttctcgactgggagtcacttaa
gaggtacaggaagctttacgagaccctcattaagaacctgaaatataacggtaatagcagggcatacatggttgaattta
actctctcagggatgtagtgagcttaatgccaatagaagaacttaaggagtggataattggagaacctaggggtcctaag
ataggtaccttcattgatgtagatgattcatttgcaaagctcctaggttactacataagtagcggagatgtagagaaagatag
ggtgaagttccacagtaaagatcaaaacgttctcgaggatatagcgaaacttgccgagaagttatttggaaaggtgagga
gaggaagaggatatattgaggtatcagggaaaattagccatgccatatttagagttttagcggaaggtaagagaattcca
gagttcatcttcacatccccaatggatattaaggtagccttccttaagggactcaacggtaatgctgaagaattaacgttctcc
actaagagtgagctattagttaaccagcttatccttctcctgaactccattggagtttcggatataaagattgaacatgagaaa
ggggtttacagagtttacataaataagaaggaatcctccaatggggatatagtacttgatagcgtcgaatctatcgaagttg
aaaaatacgagggctacgtttatgatctaagtgttgaggataatgagaacttcctcgttggcttcggactactttacgcagcc
aacagtatggacatgcgcgtgcccgcccagctgctgggcctgctgctgctgtggttccccggctcgcgatgc-
gacatccag

TABLE 14B
Partial amino acid sequence in construct B (SEQ ID NO: 101)

Pgk-
silpdewlpivenekvrfvkigdfidreieenaervkrdgeteilevkdlkalsfnretkkselkkvkalirhrysgkvysiklksg
rrikitsghslfsvkngklvkvrgdelkpgdlvvvpgrlklpeskqvlnlvelllklpeeetsnivmmipvkgrknffkgmlktly
wifgegerprtagrylkhlerlgyvklkrrgcevldweslkryrklyetliknlkyngnsraymvefnslrdvvslmpieelkewi
igeprgpkigtfidvddsfakllgyyissgdvekdrvkfhskdqnvlediaklaeklfgkvrrgrgyievsgkishaifrvlaegk
ripefiftspmdikvaflkglngnaeeltfstksellvnqlilllnsigvsdikiehekgvyrvyinkkessngdivldsvesievek
yegyvydlsvednenflvgfgllyaansmdmrvpaqllgllllwfpgsrc-diq

TABLE 15A
Partial coding sequence in construct E (SEQ ID NO: 102)

Ccgggtaaa-
accattttaccagatgaatggctcccaattgttgaaaatgaaaaagttcgattcgtaaaaattggagacttcatagataggg
agattgaggaaaacgctgagagagtgaagagggatggtgaaactgaaattctagaggttaaagatcttaaagccctttcc
ttcaatagagaaacaaaaaagagcgagctcaagaaggtaaaggccctaattagacaccgctattcagggaaggtttac
agcattaaactaaagtcagggagaaggatcaaaataacctcaggtcatagtctgttctcagtaaaaaatggaaagctagt
taaggtcaggggagatgaactcaagcctggtgatctcgttgtcgttccaggaaggttaaaacttccagaaagcaagcaag
tgctaaatctcgttgaactactcctgaaattacccgaagaggagacatcgaacatcgtaatgatgatcccagttaaaggta
gaaagaatttcttcaaagggatgctcaaaacattatactggatcttcggggagggagaaaggccaagaaccgcagggc
gctatctcaagcatcttgaaagattaggatacgttaagctcaagagaagaggctgtgaagttctcgactgggagtcacttaa
gaggtacaggaagctttacgagaccctcattaagaacctgaaatataacggtaatagcagggcatacatggttgaattta
actctctcagggatgtagtgagcttaatgccaatagaagaacttaaggagtggataattggagaacctaggggtcctaag
ataggtaccttcattgatgtagatgattcatttgcaaagctcctaggttactacataagtagcggagatgtagagaaagatag
ggtgaagttccacagtaaagatcaaaacgttctcgaggatatagcgaaacttgccgagaagttatttggaaaggtgagga
gaggaagaggatatattgaggtatcagggaaaattagccatgccatatttagagttttagcggaaggtaagagaattcca
gagttcatcttcacatccccaatggatattaaggtagccttccttaagggactcaacggtaatgctgaagaattaacgttctcc
actaagagtgagctattagttaaccagcttatccttctcctgaactccattggagtttcggatataaagattgaacatgagaaa
ggggtttacagagtttacataaataagaaggaatcctccaatggggatatagtacttgatagcgtcgaatctatcgaagttg
aaaaatacgagggctacgtttatgatctaagtgttgaggataatgagaacttcctcgttggcttcggactactttacgcacac
aacagtatggacatgcgcgtgcccgcccagctgctgggcctgctgctgctgtggttccccggctcgcgatgc-
gacatccag

TABLE 15B
Partial amino acid sequence in construct E (SEQ ID NO: 103)

Pgk-
tilpdewlpivenekvrfvkigdfidreieenaervkrdgeteilevkdlkalsfnretkkselkkvkalirhrysgkvysiklksgr
rikitsghslfsvkngklvkvrgdelkpgdlvvvpgrlklpeskqvlnlvelllklpeeetsnivmmipvkgrknffkgmlktlywi
fgegerprtagrylkhlerlgyvklkrrgcevldweslkryrklyetliknlkyngnsraymvefnslrdvvslmpieelkewiig
eprgpkigtfidvddsfakllgyyissgdvekdrvkfhskdqnvlediaklaeklfgkvrrgrgyievsgkishaifrvlaegkri
pefiftspmdikvaflkglngnaeeltfstksellvnqlilllnsigvsdikiehekgvyrvyinkkessngdivldsvesieveky
egyvydlsvednenflvgfgllyahnsmdmrvpaqllgllllwfpgsrc-diq

TABLE 16A
Partial coding sequence in construct H (SEQ ID NO: 104)

Ccgggtaaa-
agcattttaccagatgaatggctcccaattgttgaaaatgaaaaagttcgattcgtaaaaattggagacttcatagataggg
agattgaggaaaacgctgagagagtgaagagggatggtgaaactgaaattctagaggttaaagatcttaaagccctttcc
ttcaatagagaaacaaaaaagagcgagctcaagaaggtaaaggccctaattagacaccgctattcagggaaggtttac
agcattaaactaaagtcagggagaaggatcaaaataacctcaggtcatagtctgttctcagtaaaaaatggaaagctagt
taaggtcaggggagatgaactcaagcctggtgatctcgttgtcgttccaggaaggttaaaacttccagaaagcaagcaag
tgctaaatctcgttgaactactcctgaaattacccgaagaggagacatcgaacatcgtaatgatgatcccagttaaaggta
gaaagaatttcttcaaagggatgctcaaaacattatactggatcttcggggagggagaaaggccaagaaccgcagggc
gctatctcaagcatcttgaaagattaggatacgttaagctcaagagaagaggctgtgaagttctcgactgggagtcacttaa
gaggtacaggaagctttacgagaccctcattaagaacctgaaatataacggtaatagcagggcatacatggttgaattta
actctctcagggatgtagtgagcttaatgccaatagaagaacttaaggagtggataattggagaacctaggggtcctaag
ataggtaccttcattgatgtagatgattcatttgcaaagctcctaggttactacataagtagcggagatgtagagaaagatag
ggtgaagttccacagtaaagatcaaaacgttctcgaggatatagcgaaacttgccgagaagttatttggaaaggtgagga
gaggaagaggatatattgaggtatcagggaaaattagccatgccatatttagagttttagcggaaggtaagagaattcca
gagttcatcttcacatccccaatggatattaaggtagccttccttaagggactcaacggtaatgctgaagaattaacgttctcc
actaagagtgagctattagttaaccagcttatccttctcctgaactccattggagtttcggatataaagattgaacatgagaaa
ggggtttacagagtttacataaataagaaggaatcctccaatggggatatagtacttgatagcgtcgaatctatcgaagttg
aaaaatacgagggctacgtttatgatctaagtgttgaggataatgagaacttcctcgttggcttcggactactttacgcacac
aacatggacatgcgcgtgcccgcccagctgctgggcgacgagtggttccccggctcgcgatgc-gacatccag

TABLE 16B
Partial amino acid sequence in construct H (SEQ ID NO: 105)

Pgk-
silpdewlpivenekvrfvkigdfidreieenaervkrdgeteilevkdlkalsfnretkkselkkvkalirhrysgkvysiklksg
rrikitsghslfsvkngklvkvrgdelkpgdlvvvpgrlklpeskqvlnlvelllklpeeetsnivmmipvkgrknffkgmlktly
wifgegerprtagrylkhlerlgyvklkrrgcevldweslkryrklyetliknlkyngnsraymvefnslrdvvslmpieelkewi
igeprgpkigtfidvddsfakllgyyissgdvekdrvkfhskdqnvlediaklaeklfgkvrrgrgyievsgkishaifrvlaegk
ripefiftspmdikvaflkglngnaeeltfstksellvnqlilllnsigvsdikiehekgvyrvyinkkessngdivldsvesievek
yegyvydlsvednenflvgfgllyahnmdmrvpaqllgdewfpgsrc-diq

TABLE 17A
Partial coding sequence in construct J (SEQ ID NO: 106)

Ccgggtaaa-
agcattttaccagatgaatggctcccaattgttgaaaatgaaaaagttcgattcgtaaaaattggagacttcatagataggg
agattgaggaaaacgctgagagagtgaagagggatggtgaaactgaaattctagaggttaaagatcttaaagccctttcc
ttcaatagagaaacaaaaaagagcgagctcaagaaggtaaaggccctaattagacaccgctattcagggaaggtttac
agcattaaactaaagtcagggagaaggatcaaaataacctcaggtcatagtctgttctcagtaaaaaatggaaagctagt
taaggtcaggggagatgaactcaagcctggtgatctcgttgtcgttccaggaaggttaaaacttccagaaagcaagcaag
tgctaaatctcgttgaactactcctgaaattacccgaagaggagacatcgaacatcgtaatgatgatcccagttaaaggta
gaaagaatttcttcaaagggatgctcaaaacattatactggatcttcggggagggagaaaggccaagaaccgcagggc
gctatctcaagcatcttgaaagattaggatacgttaagctcaagagaagaggctgtgaagttctcgactgggagtcacttaa
gaggtacaggaagctttacgagaccctcattaagaacctgaaatataacggtaatagcagggcatacatggttgaattta
actctctcagggatgtagtgagcttaatgccaatagaagaacttaaggagtggataattggagaacctaggggtcctaag
ataggtaccttcattgatgtagatgattcatttgcaaagctcctaggttactacataagtagcggagatgtagagaaagatag
ggtgaagttccacagtaaagatcaaaacgttctcgaggatatagcgaaacttgccgagaagttatttggaaaggtgagga
gaggaagaggatatattgaggtatcagggaaaattagccatgccatatttagagttttagcggaaggtaagagaattcca
gagttcatcttcacatccccaatggatattaaggtagccttccttaagggactcaacggtaatgctgaagaattaacgttctcc
actaagagtgagctattagttaaccagcttatccttctcctgaactccattggagtttcggatataaagattgaacatgagaaa
ggggtttacagagtttacataaataagaaggaatcctccaatggggatatagtacttgatagcgtcgaatctatcgaagttg
aaaaatacgagggctacgtttatgatctaagtgttgaggataatgagaacttcctcgttggcttcggactactttacgcacac
aacatggacatgcgcgtgcccgcccagtggttccccggctcgcgatgc-gacatccag

TABLE 17B
Partial amino acid sequence in construct J (SEQ ID NO: 107)

Pgk-
silpdewlpivenekvrfvkigdfidreieenaervkrdgeteilevkdlkalsfnretkkselkkvkalirhrysgkvysiklksg
rrikitsghslfsvkngklvkvrgdelkpgdlvvvpgrlklpeskqvlnlvelllklpeeetsnivmmipvkgrknffkgmlktly
wifgegerprtagrylkhlerlgyvklkrrgcevldweslkryrklyetliknlkyngnsraymvefnslrdvvslmpieelkewi
igeprgpkigtfidvddsfakllgyyissgdvekdrvkfhskdqnvlediaklaeklfgkvrrgrgyievsgkishaifrvlaegk
ripefiftspmdikvaflkglngnaeeltfstksellvnqlilllnsigvsdikiehekgvyrvyinkkessngdivldsvesievek
yegyvydlsvednenflvgfgllyahnmdmrvpaqwfpgsrc-diq

TABLE 18A
Partial coding sequence in construct K
(SEQ ID NO: 108)

Ccgggtaaa-
agcattttaccagatgaatggctcccaattgttgaaaatgaaaaagttc
gattcgtaaaaattggagacttcatagatagggagattgaggaaaacgc
tgagagagtgaagagggatggtgaaactgaaattctagaggttaaagat
cttaaagccctttccttcaatagagaaacaaaaaagagcgagctcaaga
aggtaaaggccctaattagacaccgctattcagggaaggtttacagcat
taaactaaagtcagggagaaggatcaaaataacctcaggtcatagtctg
ttctcagtaaaaaatggaaagctagttaaggtcaggggagatgaactca
agcctggtgatctcgttgtcgttccaggaaggttaaaacttccagaaagc
aagcaagtgctaaatctcgttgaactactcctgaaattacccgaagagg
agacatcgaacatcgtaatgatgatcccagttaaaggtagaaagaattt
cttcaaagggatgctcaaaacattatactggatcttcggggagggagaa
aggccaagaaccgcagggcgctatctcaagcatcttgaaagattaggat
acgttaagctcaagagaagaggctgtgaagttctcgactgggagtcact
taagaggtacaggaagctttacgagaccctcattaagaacctgaaatat
aacggtaatagcagggcatacatggttgaatttaactctctcagggatg
tagtgagcttaatgccaatagaagaacttaaggagtggataattggaga
acctaggggtcctaagataggtaccttcattgatgtagatgattcattt
gcaaagctcctaggttactacataagtagcggagatgtagagaaagata
gggtgaagttccacagtaaagatcaaaacgttctcgaggatatagcgaa
acttgccgagaagttatttggaaaggtgaggagaggaagaggatatatt
gaggtatcagggaaaattagccatgccatatttagagttttagcggaag
gtaagagaattccagagttcatcttcacatccccaatggatattaaggt
agccttccttaagggactcaacggtaatgctgaagaattaacgttctcc
actaagagtgagctattagttaaccagcttatccttctcctgaactcca
ttggagtttcggatataaagattgaacatgagaaaggggtttacagagt
ttacataaataagaaggaatcctccaatggggatatagtacttgatagc
gtcgaatctatcgaagttgaaaaatacgagggctacgtttatgatctaa
gtgttgaggataatgagaacttcctcgttggcttcggactactttacgc
acacaac-gacatccag

TABLE 18B
Partial amino acid sequence in construct K
(SEQ ID NO: 109)

Pgk-
silpdewlpivenekvrfvkigdfidreieenaervkrdgeteilevkd
lkalsfnretkkselkkvkalirhrysgkvysiklksgrrikitsghsl
fsvkngklvkvrgdelkpgdlvvvpgrlklpeskqvlnlvelllklpee
etsnivmmipvkgrknffkgmlktlywifgegerprtagrylkhlerlg
yvklkrrgcevldweslkryrklyetliknlkyngnsraymvefnslrd
vvslmpieelkewiigeprgpkigtfidvddsfakllgyyissgdvekd
rvkfhskdqnvlediaklaeklfgkvrrgrgyievsgkishaifrvlae
gkripefiftspmdikvaflkglngnaeeltfstksellvnqlilllns
igvsdikiehekgvyrvyinkkessngdivldsvesievekyegyvydl
svednenflvgfgllyahn-diq

TABLE 19A
Partial coding sequence in construct L
(SEQ ID NO: 110)

Ccgggtaaa-
agcattttaccagatgaatggctcccaattgttgaaaatgaaaaagttc
gattcgtaaaaattggagacttcatagatagggagattgaggaaaacgc
tgagagagtgaagagggatggtgaaactgaaattctagaggttaaagat
cttaaagccctttccttcaatagagaaacaaaaaagagcgagctcaaga
aggtaaaggccctaattagacaccgctattcagggaaggtttacagcat
taaactaaagtcagggagaaggatcaaaataacctcaggtcatagtctg
ttctcagtaaaaaatggaaagctagttaaggtcaggggagatgaactca
agcctggtgatctcgttgtcgttccaggaaggttaaaacttccagaaag
caagcaagtgctaaatctcgttgaactactcctgaaattacccgaagag
gagacatcgaacatcgtaatgatgatcccagttaaaggtagaaagaatt
tcttcaaagggatgctcaaaacattatactggatcttcggggagggaga
aaggccaagaaccgcagggcgctatctcaagcatcttgaaagattagga
tacgttaagctcaagagaagaggctgtgaagttctcgactgggagtcac
ttaagaggtacaggaagctttacgagaccctcattaagaacctgaaata
taacggtaatagcagggcatacatggttgaatttaactctctcagggat
gtagtgagcttaatgccaatagaagaacttaaggagtggataattggag
aacctaggggtcctaagataggtaccttcattgatgtagatgattcatt
tgcaaagctcctaggttactacataagtagcggagatgtagagaaagat
agggtgaagttccacagtaaagatcaaaacgttctcgaggatatagcga
aacttgccgagaagttatttggaaaggtgaggagaggaagaggatatat
tgaggtatcagggaaaattagccatgccatatttagagttttagcggaa
ggtaagagaattccagagttcatcttcacatccccaatggatattaagg
tagccttccttaagggactcaacggtaatgctgaagaattaacgttctc
cactaagagtgagctattagttaaccagcttatccttctcctgaactcc
attggagtttcggatataaagattgaacatgagaaaggggtttacagag
tttacataaataagaaggaatcctccaatggggatatagtacttgatag
cgtcgaatctatcgaagttgaaaaatacgagggctacgtttatgatcta
agtgttgaggataatgagaacttcctcgttggcttcggactactttacg
cacacaacatggacatgcgcgtgcccgcccagctgctgggcctgctgct
gctgtggttccccggctcgggaggc-gacatccag

TABLE 19B
Partial amino acid sequence in construct L
(SEQ ID NO: 111)

Pgk-
silpdewlpivenekvrfvkigdfidreieenaervkrdgeteilevkd
lkalsfnretkkselkkvkalirhrysgkvysiklksgrrikitsghsl
fsvkngklvkvrgdelkpgdlvvvpgrlklpeskqvlnlvelllklpee
etsnivmmipvkgrknffkgmlktlywifgegerprtagrylkhlerlg
yvklkrrgcevldweslkryrklyetliknlkyngnsraymvefnslrd
vvslmpieelkewiigeprgpkigtfidvddsfakllgyyissgdvekd
rvkfhskdqnvlediaklaeklfgkvrrgrgyievsgkishaifrvlae
gkripefiftspmdikvaflkglngnaeeltfstksellvnqlilllns
igvsdikiehekgvyrvyinkkessngdivldsvesievekyegyvydl
svednenflvgfgllyahnmdmrvpaqllgllllwfpgsgg-diq
Heavy Chain 3′ sequence-Intein + light chain
signal peptide sequence-Light Chain mature
sequence

The following oligonucleotides were used for the amplification of the Saccharomyces cerevisiae VMA intein (GenBank accession #AB093499) using genomic DNA as template and Pfu-1Hi Fidelity DNA Polymerase (Stratagene).

Genomic DNA was prepared from a culture of Saccharomyces cerevisiae using the Yeast-Geno-DNA-Template kit (G Biosciences, cat. #786-134).

Sce VMA intein 5′:

(SEQ ID NO: 112)

TGCTTTGCCAAGGGTACCAATGTTTT
Sce VMA intein 3′

(SEQ ID NO: 113)

ATTATGGACGACAACCTGGTTGGCAA

PCR run according to the following program:

| | Step | | | | | | | |
|---|---|---|---|---|---|---|---|---|
| | 1 | 2 | 3 | 4 | 5 | 6 | 7 | 8 |
| Temp | 94° C. | 94° C. | 55° C. | 72° C. | Go to step 2 (39 times) | 72° C. | 4° C. | End |
| Time | 2 min | 1 min | 1 min | 2 min | | 5 min | hold | |

The PCR product was used as template using the following pairs of primers to produce 0aa, 1aa or 3aa versions of the intein as for the P. horikoshii intein constructs. Pfu-1Hi Fidelity DNA Polymerase (Stratagene) used.

Sce-5′-Sap
(SEQ ID NO: 114)
CCGCAGAAGAGCCTCTCCCTGTCTCCGGGTAAATGCTTTGCCAAGGGTAC
CAATGTTTT
Sce-5′-1aa-Sap
(SEQ ID NO: 115)
CCGCAGAAGAGCCTCTCCCTGTCTCCGGGTAAAGGGTGCTTTGCCAAGGG
TACCAATGTTTT
Sce-5′-3aa-Sap
(SEQ ID NO: 116)
CCGCAGAAGAGCCTCTCCCTGTCTCCGGGTAAATATGTCGGGTGCTTTGC
CAAGGGTACCAATGTTTT
Sce-3′-Van911
(SEQ ID NO: 117)
CAGCAGGCCCAGCAGCTGGGCGGGCACGCGCATGTCCATATTATGGACGA
CAACCTGGTTGGCAA
Sce-3′-1aa-Van911
(SEQ ID NO: 118)
CAGCAGGCCCAGCAGCTGGGCGGGCACGCGCATGTCCATGCAATTATGGA
CGACAACCTGGTTGGCAA
Sce-3′-3aa-Van911
(SEQ ID NO: 119)
CAGCAGGCCCAGCAGCTGGGCGGGCACGCGCATGTCCATTTCTCCGCAAT
TATGGACGACAACCTGGTTGGCAA

PCR was run using the same program provided above. The PCR product from each reaction type was subcloned into pCR-BluntII-TOPO (Invitrogen) and the insert of each type was sequenced and proven correct.

Oligonucleotide primers were designed in order to generate the fusion of D2E7 Heavy Chain-Intein-D2E7 Light Chain by way of homologous recombination into the pTT3-HcintLC p. horikoshii construct in E. coli. By engineering a 40 base pair overhang between PCR generated vector (containing pTT3 vector, heavy chain and light chain regions but not the P. horikoshii intein) and the VMA intein insert, the two DNAs can be mixed and transformed into E. coli without the benefit of ligation, resulting in E. coli homologous recombination of the two fragments into pTT3-HC-VMAint-LC in the 0aa, 1aa and 3aa versions.

### VMA Homologous Recombination Primers:

VMA-HR5′:
(SEQ ID NO: 120)
CCACTACACGCAGAAGAGCCTCTCCCTGTCTCCGGGTAAA
VMA-HR3′:
(SEQ ID NO: 121)
GCAGCAGGCCCAGCAGCTGGGCGGGCACGCGCATGTCCAT
pTT3-HcintLC homologous recombination primers:
pTT3int-HR5′:
(SEQ ID NO: 122)
ATGGACATGCGCGTGCCCGCCCAGCTGCTGGGCCTGCTGC
pTT3int-HR3′:
(SEQ ID NO: 123)
TTTACCCGGAGACAGGGAGAGGCTCTTCTGCGTGTAGTGGT

PCR for intein was run on the following program: Pfu-1Hi Fidelity DNA Polymerase (Stratagene) used.

| | Step | | | | | | | |
|---|---|---|---|---|---|---|---|---|
| | 1 | 2 | 3 | 4 | 5 | 6 | 7 | 8 |
| Temp | 94° C. | 94° C. | 60° C. | 72° C. | Go to step 2 (34 times) | 72° C. | 4° C. | End |
| Time | 2 min | 1 min | 1 min | 1.5 min | | | 5 min | hold |

PCR for the vector was run per the following program: Platinum Taq Hi Fidelity Supermix (Invitrogen) used.

| | Step | | | | | | | |
|---|---|---|---|---|---|---|---|---|
| | 1 | 2 | 3 | 4 | 5 | 6 | 7 | 8 |
| Temp | 94° C. | 94° C. | 60° C. | 68° C. | Go to step 2 (24 times) | 68° C. | 4° C. | End |
| Time | 2 min | 30 sec | 30 sec | 10 min | | 5 min | hold | |

To effect homologous recombination of the VMA intein into pTT3-HcintLC the following strategy was employed. PCR products were gel purified, and each was eluted into 50 μl elution buffer using a Qiaquick Gel Extraction kit (Qiagen). 3 μl of the vector PCR product was mixed in an eppendorf tube, and 3 μl of the desired VMA intein PCR product was added (either 0aa, 1aa or 3aa in separate tubes). Each mixture was transformed into E. coli, and the cells were then plated onto LB+Ampicillin plates and incubated at 37C overnight. Colonies were grown to 2 ml cultures, plasmid DNA was prepared using Wizard Prep Kits (Promega) and analyzed by restriction endonuclease digestion and agarose gel electrophoresis. Clones that produced the correct restriction pattern were analyzed with respect to DNA sequence.

Three Expression Constructs for D2E7 Heavy Chain-intein-D2E7 Light Chain, utilizing the S. cerevisiae VMA intein, were created: pTT3-Hc-VMAint-LC-0aa; pTT3-Hc-VMAint-LC-1aa; and pTT3-Hc-VMAint-LC-3aa. See also FIG. 15 for a plasmid map.

TABLE 20
Sequence of entire plasmid pTT3-D2E7 Heavy Chain-intein-D2E7 Light Chain
(SEQ ID NO: 124)

5′-
gcggccgctcgaggccggcaaggccggatcccccgacctcgacctctggctaataaaggaaatttattttcattgcaatag
tgtgttggaattttttgtgtctctcactcggaaggacatatgggagggcaaatcatttggtcgagatccctcggagatctctagc
tagaggatcgatccccgccccggacgaactaaacctgactacgacatctctgccccttcttcgcggggcagtgcatgtaat
cccttcagttggttggtacaacttgccaactgggccctgttccacatgtgacacggggggggaccaaacacaaaggggtt
ctctgactgtagttgacatccttataaatggatgtgcacatttgccaacactgagtggctttcatcctggagcagactttgcagt
ctgtggactgcaacacaacattgcctttatgtgtaactcttggctgaagctcttacaccaatgctgggggacatgtacctccc
aggggcccaggaagactacgggaggctacaccaacgtcaatcagaggggcctgtgtagctaccgataagcggaccct
caagagggcattagcaatagtgtttataaggcccccttgttaaccctaaacgggtagcatatgcttcccgggtagtagtatat
actatccagactaaccctaattcaatagcatatgttacccaacgggaagcatatgctatcgaattagggttagtaaaagggt
cctaaggaacagcgatatctcccaccccatgagctgtcacggttttatttacatggggtcaggattccacgagggtagtgaa
ccattttagtcacaagggcagtggctgaagatcaaggagcgggcagtgaactctcctgaatcttcgcctgcttcttcattctc
cttcgtttagctaatagaataactgctgagttgtgaacagtaaggtgtatgtgaggtgctcgaaaacaaggtttcaggtgacg
cccccagaataaaatttggacggggggttcagtggtggcattgtgctatgacaccaatataaccctcacaaaccccttggg
caataaatactagtgtaggaatgaaacattctgaatatctttaacaatagaaatccatggggtggggacaagccgtaaag
actggatgtccatctcacacgaatttatggctatgggcaacacataatcctagtgcaatatgatactggggttattaagatgtg
tcccaggcagggaccaagacaggtgaaccatgttgttacactctatttgtaacaaggggaaagagagtggacgccgac
agcagcggactccactggttgtctctaacacccccgaaaattaaacggggctccacgccaatggggcccataaacaaa
gacaagtggccactcttttttttgaaattgtggagtgggggcacgcgtcagcccccacacgccgccctgcggttttggactgt
aaaataagggtgtaataacttggctgattgtaaccccgctaaccactgcggtcaaaccacttgcccacaaaaccactaat
ggcaccccggggaatacctgcataagtaggtgggcgggccaagataggggcgcgattgctgcgatctggaggacaaa
ttacacacacttgcgcctgagcgccaagcacagggttgttggtcctcatattcacgaggtcgctgagagcacggtgggcta
atgttgccatgggtagcatatactacccaaatatctggatagcatatgctatcctaatctatatctgggtagcataggctatcct
aatctatatctgggtagcatatgctatcctaatctatatctgggtagtatatgctatcctaatttatatctgggtagcataggctatc
ctaatctatatctgggtagcatatgctatcctaatctatatctgggtagtatatgctatcctaatctgtatccgggtagcatatgct
atcctaatagagattagggtagtatatgctatcctaatttatatctgggtagcatatactacccaaatatctggatagcatatgct
atcctaatctatatctgggtagcatatgctatcctaatctatatctgggtagcataggctatcctaatctatatctgggtagcatat
gctatcctaatctatatctgggtagtatatgctatcctaatttatatctgggtagcataggctatcctaatctatatctgggtagcat
atgctatcctaatctatatctgggtagtatatgctatcctaatctgtatccgggtagcatatgctatcctcatgataagctgtcaa
acatgagaattttcttgaagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatga
gacaataaccctgataaatgcttcaataatattgaaaaaggaagagtatgagtattcaacatttccgtgtcgcccttattccct
tttttgcggcattttgccttcctgtttttgctcacccagaaacgctggtgaaagtaaaagatgctgaagatcagttgggtgcacg
agtgggttacatcgaactggatctcaacagcggtaagatccttgagagttttcgccccgaagaacgttttccaatgatgagc
acttttaaagttctgctatgtggcgcggtattatcccgtgttgacgccgggcaagagcaactcggtcgccgcatacactattct
cagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttg
cacaacatgggggatcatgtaactcgccttgatcgttgggaaccggagctgaatgaagccataccaaacgacgagcgtg
acaccacgatgcctgcagcaatggcaacaacgttgcgcaaactattaactggcgaactacttactctagcttcccggcaa
caattaatagactggatggaggcggataaagttgcaggaccacttctgcgctcggcccttccggctggctggtttattgctga
taaatctggagccggtgagcgtgggtctcgcggtatcattgcagcactggggccagatggtaagccctcccgtatcgtagtt
atctacacgacggggagtcaggcaactatggatgaacgaaatagacagatcgctgagataggtgcctcactgattaagc
attggtaactgtcagaccaagtttactcatatatactttagattgatttaaaacttcatttttaatttaaaaggatctaggtgaagat
cctttttgataatctcatgaccaaaatcccttaacgtgagttttcgttccactgagcgtcagaccccgtagaaaagatcaaag
gatcttcttgagatcctttttttctgcgcgtaatctgctgcttgcaaacaaaaaaaccaccgctaccagcggtggtttgtttgccg
gatcaagagctaccaactctttttccgaaggtaactggcttcagcagagcgcagataccaaatactgttcttctagtgtagcc
gtagttaggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacggg
gggttcgtgcacacagcccagcttggagcgaacgacctacaccgaactgagatacctacagcgtgagctatgagaaag
cgccacgcttcccgaagggagaaaggcggacaggtatccggtaagcggcagggtcggaacaggagagcgcacgag
ggagcttccagggggaaacgcctggtatctttatagtcctgtcgggtttcgccacctctgacttgagcgtcgatttttgtgatgct
cgtcaggggggcggagcctatggaaaaacgccagcaacgcggcctttttacggttcctggccttttgctggccttttgctcac
atgttctttcctgcgttatcccctgattctgtggataaccgtattaccgcctttgagtgagctgataccgctcgccgcagccgaa
cgaccgagcgcagcgagtcagtgagcgaggaagcggaagagcgcccaatacgcaaaccgcctctccccgcgcgttg
gccgattcattaatgcagctggcacgacaggtttcccgactggaaagcgggcagtgagcgcaacgcaattaatgtgagtt
agctcactcattaggcaccccaggctttacactttatgcttccggctcgtatgttgtgtggaattgtgagcggataacaatttca
cacaggaaacagctatgaccatgattacgccaagctctagctagaggtcgaccaattctcatgtttgacagcttatcatcgc
agatccgggcaacgttgttgccattgctgcaggcgcagaactggtaggtatggaagatctatacattgaatcaatattggca
attagccatattagtcattggttatatagcataaatcaatattggctattggccattgcatacgttgtatctatatcataatatgtac
atttatattggctcatgtccaatatgaccgccatgttgacattgattattgactagttattaatagtaatcaattacggggtcatta
gttcatagcccatatatggagttccgcgttacataacttacggtaaatggcccgcctggctgaccgcccaacgacccccgc
ccattgacgtcaataatgacgtatgttcccatagtaacgccaatagggactttccattgacgtcaatgggtggagtatttacg
gtaaactgcccacttggcagtacatcaagtgtatcatatgccaagtccgccccctattgacgtcaatgacggtaaatggccc
gcctggcattatgcccagtacatgaccttacgggactttcctacttggcagtacatctacgtattagtcatcgctattaccatgg
tgatgcggttttggcagtacaccaatgggcgtggatagcggtttgactcacggggatttccaagtctccaccccattgacgtc
aatgggagtttgttttggcaccaaaatcaacgggactttccaaaatgtcgtaataaccccgccccgttgacgcaaatgggc
ggtaggcgtgtacggtgggaggtctatataagcagagctcgtttagtgaaccgtcagatcctcactctcttccgcatcgctgt
ctgcgagggccagctgttgggctcgcggttgaggacaaactcttcgcggtctttccagtactcttggatcggaaacccgtcg
gcctccgaacggtactccgccaccgagggacctgagcgagtccgcatcgaccggatcggaaaacctctcgagaaagg
cgtctaaccagtcacagtcgcaaggtaggctgagcaccgtggcgggcggcagcgggtggcggtcggggttgtttctggc
ggaggtgctgctgatgatgtaattaaagtaggcggtcttgagacggcggatggtcgaggtgaggtgtggcaggcttgagat
ccagctgttggggtgagtactccctctcaaaagcgggcattacttctgcgctaagattgtcagtttccaaaaacgaggagga
tttgatattcacctggcccgatctggccatacacttgagtgacaatgacatccactttgcctttctctccacaggtgtccactccc
aggtccaagtttgggcgccaccatggagtttgggctgagctggctttttcttgtcgcgattttaaaaggtgtccagtgt-
gaggtgcagctggtggagtctgggggaggcttggtacagcccggcaggtccctgagactctcctgtgcggcctctggattc
acctttgatgattatgccatgcactgggtccggcaagctccagggaagggcctggaatgggtctcagctatcacttggaata
gtggtcacatagactatgcggactctgtggagggccgattcaccatctccagagacaacgccaagaactccctgtatctgc
aaatgaacagtctgagagctgaggatacggccgtatattactgtgcgaaagtctcgtaccttagcaccgcgtcctcccttga
ctattggggccaaggtaccctggtcaccgtctcgagtgcgtcgaccaagggcccatcggtcttccccctggcaccctcctc
caagagcacctctgggggcacagcggccctgggctgcctggtcaaggactacttccccgaaccggtgacggtgtcgtgg
aactcaggcgccctgaccagcggcgtgcacaccttcccggctgtcctacagtcctcaggactctactccctcagcagcgt
ggtgaccgtgccctccagcagcttgggcacccagacctacatctgcaacgtgaatcacaagcccagcaacaccaaggt
ggacaagaaagttgagcccaaatcttgtgacaaaactcacacatgcccaccgtgcccagcacctgaactcctgggggg
accgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatg
gcaaggagtacaagtgcaaggtctccaacaaagccctcccagcccccatcgagaaaaccatctccaaagccaaagg
gcagccccgagaaccacaggtgtacaccctgcccccatcccgggatgagctgaccaagaaccaggtcagcctgacct
gcctggtcaaaggcttctatcccagcgacatcgccgtggagtgggagagcaatgggcagccggagaacaactacaag
accacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtctccggg
taaa-
tgctttgccaagggtaccaatgttttaatggcggatgggtctattgaatgtattgaaaacattgaggttggtaataaggtcatgg
gtaaagatggcagacctcgtgaggtaattaaattgcccagaggaagagaaactatgtacagcgtcgtgcagaaaagtc
agcacagagcccacaaaagtgactcaagtcgtgaagtgccagaattactcaagtttacgtgtaatgcgacccatgagttg
gttgttagaacacctcgtagtgtccgccgtttgtctcgtaccattaagggtgtcgaatattttgaagttattacttttgagatgggc
caaaagaaagcccccgacggtagaattgttgagcttgtcaaggaagtttcaaagagctacccaatatctgaggggcctg
agagagccaacgaattagtagaatcctatagaaaggcttcaaataaagcttattttgagtggactattgaggccagagatc
tttctctgttgggttcccatgttcgtaaagctacctaccagacttacgctccaattctttatgagaatgaccactttttcgactacat
gcaaaaaagtaagtttcatctcaccattgaaggtccaaaagtacttgcttatttacttggtttatggattggtgatggattgtctg
acagggcaactttttcggttgattccagagatacttctttgatggaacgtgttactgaatatgctgaaaagttgaatttgtgcgcc
gagtataaggacagaaaagaaccacaagttgccaaaactgttaatttgtactctaaagttgtcagaggtaatggtattcgc
aataatcttaatactgagaatccattatgggacgctattgttggcttaggattcttgaaggacggtgtcaaaaatattccttctttc
ttgtctacggacaatatcggtactcgtgaaacatttcttgctggtctaattgattctgatggctatgttactgatgagcatggtatta
aagcaacaataaagacaattcatacttctgtcagagatggtttggtttcccttgctcgttctttaggcttagtagtctcggttaac
gcagaacctgctaaggttgacatgaatggcaccaaacataaaattagttatgctatttatatgtctggtggagatgttttgctta
acgttctttcgaagtgtgccggctctaaaaaattcaggcctgctcccgccgctgcttttgcacgtgagtgccgcggattttatttc
gagttacaagaattgaaggaagacgattattatgggattactttatctgatgattctgatcatcagtttttgcttgccaaccaggt
tgtcgtccataat-
atggacatgcgcgtgcccgcccagctgctgggcctgctgctgctgtggttccccggctcgcgatgcgacatccagatgacc
cagtctccatcctccctgtctgcatctgtaggggacagagtcaccatcacttgtcgggcaagtcagggcatcagaaattactt
agcctggtatcagcaaaaaccagggaaagcccctaagctcctgatctatgctgcatccactttgcaatcaggggtcccatc
tcggttcagtggcagtggatctgggacagatttcactctcaccatcagcagcctacagcctgaagatgttgcaacttattact
gtcaaaggtataaccgtgcaccgtatacttttggccaggggaccaaggtggaaatcaaacgtacggtggctgcaccatct
gtcttcatcttcccgccatctgatgagcagttgaaatctggaactgcctctgttgtgtgcctgctgaataacttctatcccagag
aggccaaagtacagtggaaggtggataacgccctccaatcgggtaactcccaggagagtgtcacagagcaggacagc
aaggacagcacctacagcctcagcagcaccctgacgctgagcaaagcagactacgagaaacacaaagtctacgcct
gcgaagtcacccatcagggcctgagctcgcccgtcacaaagagcttcaacaggggagagtgt-3′
pTT3 Vector-Heavy Chain-Intein-Light Chain

In the following constructs, the only difference from the construct above is the inclusion of extein sequences native to S. cerevisiae (shown in blue). The sequences shown are from the end of the D2E7 heavy chain coding region (last 9 base pairs as shown in red) to the 5′ end of the D2E7 light chain coding region (first 9 base pairs as shown in pink)

TABLE 21
Partial coding sequence in pTT3-HC-VMAint-LC-1aa
(SEQ ID NO: 125)

5′-ccgggtaaa-ggg-
tgctttgccaagggtaccaatgttttaatggcggatgggtctattgaat
gtattgaaaacattgaggttggtaataaggtcatgggtaaagatggcag
acctcgtgaggtaattaaattgcccagaggaagagaaactatgtacagc
gtcgtgcagaaaagtcagcacagagcccacaaaagtgactcaagtcgtg
aagtgccagaattactcaagtttacgtgtaatgcgacccatgagttggt
tgttagaacacctcgtagtgtccgccgtttgtctcgtaccattaagggt
gtcgaatattttgaagttattacttttgagatgggccaaaagaaagccc
ccgacggtagaattgttgagcttgtcaaggaagtttcaaagagctaccc
aatatctgaggggcctgagagagccaacgaattagtagaatcctataga
aaggcttcaaataaagcttattttgagtggactattgaggccagagatc
tttctctgttgggttcccatgttcgtaaagctacctaccagacttacgc
tccaattctttatgagaatgaccactttttcgactacatgcaaaaaagt
aagtttcatctcaccattgaaggtccaaaagtacttgcttatttacttg
gtttatggattggtgatggattgtctgacagggcaactttttcggttga
ttccagagatacttctttgatggaacgtgttactgaatatgctgaaaag
ttgaatttgtgcgccgagtataaggacagaaaagaaccacaagttgcca
aaactgttaatttgtactctaaagttgtcagaggtaatggtattcgcaa
taatcttaatactgagaatccattatgggacgctattgttggcttagga
ttcttgaaggacggtgtcaaaaatattccttctttcttgtctacggaca
atatcggtactcgtgaaacatttcttgctggtctaattgattctgatgg
ctatgttactgatgagcatggtattaaagcaacaataaagacaattcat
acttctgtcagagatggtttggtttcccttgctcgttctttaggcttag
tagtctcggttaacgcagaacctgctaaggttgacatgaatggcaccaa
acataaaattagttatgctatttatatgtctggtggagatgttttgctt
aacgttctttcgaagtgtgccggctctaaaaaattcaggcctgctcccg
ccgctgcttttgcacgtgagtgccgcggattttatttcgagttacaaga
attgaaggaagacgattattatgggattactttatctgatgattctgat
catcagtttttgcttgccaaccaggttgtcgtccataat-tgc-atgga
catg-3′
Heavy Chain 3′ sequence-Intein-Extein-Light
Chain 5′ sequence

TABLE 22
pTT3-HC-VMAint-LC-3aa (SEQ ID NO: 126)

ccgggtaaatatgtcgggtgctttgccaagggtaccaatgttttaatgg
cggatgggtctattgaatgtattgaaaacattgaggttggtaataaggt
catgggtaaagatggcagacctcgtgaggtaattaaattgcccagagga
agagaaactatgtacagcgtcgtgcagaaaagtcagcacagagcccaca
aaagtgactcaagtcgtgaagtgccagaattactcaagtttacgtgtaa
tgcgacccatgagttggttgttagaacacctcgtagtgtccgccgtttg
tctcgtaccattaagggtgtcgaatattttgaagttattacttttgaga
tgggccaaaagaaagcccccgacggtagaattgttgagcttgtcaagga
agtttcaaagagctacccaatatctgaggggcctgagagagccaacgaa
ttagtagaatcctatagaaaggcttcaaataaagcttattttgagtgga
ctattgaggccagagatctttctctgttgggttcccatgttcgtaaagc
tacctaccagacttacgctccaattctttatgagaatgaccactttttc
gactacatgcaaaaaagtaagtttcatctcaccattgaaggtccaaaag
tacttgcttatttacttggtttatggattggtgatggattgtctgacag
ggcaactttttcggttgattccagagatacttctttgatggaacgtgtt
actgaatatgctgaaaagttgaatttgtgcgccgagtataaggacagaa
aagaaccacaagttgccaaaactgttaatttgtactctaaagttgtcag
aggtaatggtattcgcaataatcttaatactgagaatccattatgggac
gctattgttggcttaggattcttgaaggacggtgtcaaaaatattcctt
ctttcttgtctacggacaatatcggtactcgtgaaacatttcttgctgg
tctaattgattctgatggctatgttactgatgagcatggtattaaagca
acaataaagacaattcatacttctgtcagagatggtttggtttcccttg
ctcgttctttaggcttagtagtctcggttaacgcagaacctgctaaggt
tgacatgaatggcaccaaacataaaattagttatgctatttatatgtct
ggtggagatgttttgcttaacgttctttcgaagtgtgccggctctaaaa
aattcaggcctgctcccgccgctgcttttgcacgtgagtgccgcggatt
ttatttcgagttacaagaattgaaggaagacgattattatgggattact
ttatctgatgattctgatcatcagtttttgcttgccaaccaggttgtcg
tccataattgcggagaaatggacatg
Heavy Chain 3′ sequence-Intein-Extein-Light
Chain 5′ sequence

Synechocystis spp. Strain PCC6803 DnaE Intein: Synthesis, PCR Amplification and Cloning

The Synechocystis spp. Strain PCC6803 DnaE intein is a naturally split intein (NCBI accession #s S76958 and S75328). We have linked the N′ terminal and C-terminal halves of this intein as one open reading frame by having it synthetically synthesized. The coding sequence for the desired protein sequence was codon-optimized for expression in CHO cells (www.geneart.com). The resulting nucleotide sequence is given in Table 23.

Table 23. Ssp-Di (Coding Sequence Optimized for Expression in Cricetulus griseus) (See Also SEQ ID NOs:127 and 128)

KpnI EcoRI
GGGCGAATTGGGTACCGAATTCTGCCTGTCCTTCGGCACCGAGATCCTGACCGTGGAGTA
1 ---------+---------+---------+---------+---------+---------+
CCCGCTTAACCCATGGCTTAAGACGGACAGGAAGCCGTGGCTCTAGGACTGGCACCTCAT
C_{——}L_{——}S_{——}F_{——}G_{——}T_{——}E_{——}I_{——}L_{——}T_{——}V_{——}E_{——}Y_{—}
CGGCCCTCTGCCTATCGGCAAGATCGTGTCCGAAGAGATCAACTGCTCCGTGTACTCCGT
61 ---------+---------+---------+---------+---------+---------+
GCCGGGAGACGGATAGCCGTTCTAGCACAGGCTTCTCTAGTTGACGAGGCACATGAGGCA
_G_{——}P_{——}L_{——}P_{——}I_{——}G_{——}K_{——}I_{——}V_{——}S_{——}E_{——}E_{——}I_{——}N_{——}C_{——}S_{——}V_{——}Y_{——}S_{——}V_{—}
AccI
GGACCCTGAGGGCCGGGTGTATACTCAGGCCATCGCCCAGTGGCACGACCGGGGCGAGCA
121 ---------+---------+---------+---------+---------+---------+
CCTGGGACTCCCGGCCCACATATGAGTCCGGTAGCGGGTCACCGTGCTGGCCCCGCTCGT
_D_{——}P_{——}E_{——}G_{——}R_{——}V_{——}Y_{——}T_{——}Q_{——}A_{——}I_{——}A_{——}Q_{——}W_{——}H_{——}D_{——}R_{——}G_{——}E_{——}Q_{—}
AgeI
GGAGGTGCTGGAGTACGAGCTGGAGGACGGCTCCGTGATCCGGGCCACCTCCGACCACCG
181 ---------+---------+---------+---------+---------+---------+
CCTCCACGACCTCATGCTCGACCTCCTGCCGAGGCACTAGGCCCGGTGGAGGCTGGTGGC
_E_{——}V_{——}L_{——}E_{——}Y_{——}E_{——}L_{——}E_{——}D_{——}G_{——}S_{——}V_{——}I_{——}R_{——}A_{——}T_{——}S_{——}D_{——}H_{——}R_{—}
PvuII BglII PvuII BspMI
GTTTCTGACCACCGACTATCAGCTGCTGGCCATCGAGGAGATCTTCGCCCGGCAGCTGGA
241 ---------+---------+---------+---------+---------+---------+
CAAAGACTGGTGGCTGATAGTCGACGACCGGTAGCTCCTCTAGAAGCGGGCCGTCGACCT
_F_{——}L_{——}T_{——}T_{——}D_{——}Y_{——}Q_{——}L_{——}L_{——}A_{——}I_{——}E_{——}E_{——}I_{——}F_{——}A_{——}R_{——}Q_{——}L_{——}D_{—}
BstNI BstNI
CCTGCTGACCCTGGAGAACATCAAGCAGACCGAGGAGGCCCTGGACAACCACCGGCTGCC
301 ---------+---------+---------+---------+---------+---------+
GGACGACTGGGACCTCTTGTAGTTCGTCTGGCTCCTCCGGGACCTGTTGGTGGCCGACGG
_L_{——}L_{——}T_{——}L_{——}E_{——}N_{——}I_{——}K_{——}Q_{——}T_{——}E_{——}E_{——}A_{——}L_{——}D_{——}N_{——}H_{——}R_{——}L_{——}P_{—}
BstXI BstNI
TTTCCCTCTGCTGGACGCCGGCACCATCAAGATGGTGAAGGTGATCGGCAGGCGGTCCCT
361 ---------+---------+---------+---------+---------+---------+
AAAGGGAGACGACCTGCGGCCGTGGTAGTTCTACCACTTCCACTAGCCGTCCGCCAGGGA
_F_{——}P_{——}L_{——}L_{——}D_{——}A_{——}G_{——}T_{——}I_{——}K_{——}M_{——}V_{——}K_{——}V_{——}I_{——}G_{——}R_{——}R_{——}S_{——}L_{—}
GGGCGTGCAGCGGATCTTCGACATCGGCCTGCCTCAGGACCACAACTTTCTGCTGGCCAA
421 ---------+---------+---------+---------+---------+---------+
CCCGCACGTCGCCTAGAAGCTGTAGCCGGACGGAGTCCTGGTGTTGAAAGACGACCGGTT
_G_{——}V_{——}Q_{——}R_{——}I_{——}F_{——}D_{——}I_{——}G_{——}L_{——}P_{——}Q_{——}D_{——}H_{——}N_{——}F_{——}L_{——}L_{——}A_{——}N_{—}
NarI
KasI SacI
HaeII HindIII
CGGCGCCATCGCCGCCAACAAGCTTGAGCTCCAGCTTTTGTTCCC
481 ---------+---------+---------+---------+-----
GCCGCGGTAGCGGCGGTTGTTCGAACTCGAGGTCGAAAACAAGGG
_G_{——}A_{——}I_{——}A_{——}A_{——}N_{——}
1

The following oligonucleotides were used for the amplification of the Synechocystis spp. Strain PCC6803 DnaE intein using the synthetic DNA above as template and Platinum Taq Hi Fidelity Supermix (Invitrogen). These primers also introduce extein sequences to generate the 0aa, 1aa and 3aa versions, as well as sequences for the homologous recombination of the PCR product into the pTT3-HcintLC vector as done with the S. cerevisiae VMA intein:

Ssp-geneart-5′ HR:
(SEQ ID NO: 129)
CCACTACACGCAGAAGAGCCTCTCCCTGTCTCCGGGTAAATGCCTGTC
CTTCGGCACCGAG
Ssp-geneart-3′-HR:
(SEQ ID NO: 130)
GCAGCAGGCCCAGCAGCTGGGCGGGCACGCGCATGTCCATGTTGGCGGC
GATGGCGCCGTTGGCC
Ssp-GA-1aa-5′-HR:
(SEQ ID NO: 131)
CCACTACACGCAGAAGAGCCTCTCCCTGTCTCCGGGTAAATATTGCCT
GTCCTTCGGCACCGAG
Ssp-GA-1aa-3′-HR:
(SEQ ID NO: 132)
GCAGCAGGCCCAGCAGCTGGGCGGGCACGCGCATGTCCATACAGTTGGC
GGCGATGGCGCCGT
Ssp-GA-3aa-5′-HR:
(SEQ ID NO: 133)
CCACTACACGCAGAAGAGCCTCTCCCTGTCTCCGGGTAAAGCCGAGTA
TTGCCTGTCCTTCGGCACCGAG
Ssp-GA-3aa-3′-HR:
(SEQ ID NO: 134)
CCACTACACGCAGAAGAGCCTCTCCCTGTCTCCGGGTAAAGCCGAGTA
TTGCCTGTCCTTCGGCACCGAG

PCR run on the following program:

| | Step | | | | | | | |
|---|---|---|---|---|---|---|---|---|
| | 1 | 2 | 3 | 4 | 5 | 6 | 7 | 8 |
| Temp | 94° C. | 94° C. | 60° C. | 68° C. | Go to step 2 (34 times) | 68° C. | 4° C. | End |
| Time | 2 min | 30 sec | 30 sec | 1 min | | 5 min | hold | |

To obtain homologous recombination of the codon-optimized Synechocystis spp. Strain PCC6803 DnaE intein into pTT3-HcintLC, the following strategy was used. PCR products were gel purified and each eluted into 50 ul elution buffer (Qiaquick Gel Extraction kit (Qiagen). 2 μl of the vector PCR product (same as used in the homologous recombination with the VMA intein) was mixed in an Eppendorf tube 2 μl of the desired Synechocystis spp. Strain PCC6803 DnaE intein PCR product (either 0aa, 1aa or 3aa in separate tubes). The nucleic acids are then transformed into E. coli and plated onto LB+Ampicillin plates and then incubated at 37° C. overnight. Colonies were grown to 2 ml cultures, prepped for DNA using the Wizard prep kit (Promega) and assayed by restriction endonuclease digestion and agarose gel electrophoresis. Clones that produce the correct restriction pattern are analyzed with respect to DNA sequence to confirm that the desired sequences are present.

Three Expression Constructs for D2E7 Heavy Chain-intein-D2E7 Light Chain, utilizing the Synechocystis spp. Strain PCC6803 DnaE intein were designed: pTT3-Hc-Ssp-GA-int-LC-0aa (See FIG. 16 for plasmid map); pTT3-Hc-Ssp-GA-int-LC-1aa; and pTT3-Hc-Ssp-GA-int-LC-3aa.

TABLE 24
Sequence of entire plasmid pTT3-D2E7 Heavy Chain-Ssp-GA-
intein-D2E7 Light Chain (SEQ ID NO: 135)

5′-
gcggccgctcgaggccggcaaggccggatcccccgacctcgacctctggctaataaaggaaatttattttcattgcaatag
tgtgttggaattttttgtgtctctcactcggaaggacatatgggagggcaaatcatttggtcgagatccctcggagatctctagc
tagaggatcgatccccgccccggacgaactaaacctgactacgacatctctgccccttcttcgcggggcagtgcatgtaat
cccttcagttggttggtacaacttgccaactgggccctgttccacatgtgacacggggggggaccaaacacaaaggggtt
ctctgactgtagttgacatccttataaatggatgtgcacatttgccaacactgagtggctttcatcctggagcagactttgcagt
ctgtggactgcaacacaacattgcctttatgtgtaactcttggctgaagctcttacaccaatgctgggggacatgtacctccc
aggggcccaggaagactacgggaggctacaccaacgtcaatcagaggggcctgtgtagctaccgataagcggaccct
caagagggcattagcaatagtgtttataaggcccccttgttaaccctaaacgggtagcatatgcttcccgggtagtagtatat
actatccagactaaccctaattcaatagcatatgttacccaacgggaagcatatgctatcgaattagggttagtaaaagggt
cctaaggaacagcgatatctcccaccccatgagctgtcacggttttatttacatggggtcaggattccacgagggtagtgaa
ccattttagtcacaagggcagtggctgaagatcaaggagcgggcagtgaactctcctgaatcttcgcctgcttcttcattctc
cttcgtttagctaatagaataactgctgagttgtgaacagtaaggtgtatgtgaggtgctcgaaaacaaggtttcaggtgacg
cccccagaataaaatttggacggggggttcagtggtggcattgtgctatgacaccaatataaccctcacaaaccccttggg
caataaatactagtgtaggaatgaaacattctgaatatctttaacaatagaaatccatggggtggggacaagccgtaaag
actggatgtccatctcacacgaatttatggctatgggcaacacataatcctagtgcaatatgatactggggttattaagatgtg
tcccaggcagggaccaagacaggtgaaccatgttgttacactctatttgtaacaaggggaaagagagtggacgccgac
agcagcggactccactggttgtctctaacacccccgaaaattaaacggggctccacgccaatggggcccataaacaaa
gacaagtggccactcttttttttgaaattgtggagtgggggcacgcgtcagcccccacacgccgccctgcggttttggactgt
aaaataagggtgtaataacttggctgattgtaaccccgctaaccactgcggtcaaaccacttgcccacaaaaccactaat
ggcaccccggggaatacctgcataagtaggtgggcgggccaagataggggcgcgattgctgcgatctggaggacaaa
ttacacacacttgcgcctgagcgccaagcacagggttgttggtcctcatattcacgaggtcgctgagagcacggtgggcta
atgttgccatgggtagcatatactacccaaatatctggatagcatatgctatcctaatctatatctgggtagcataggctatcct
aatctatatctgggtagcatatgctatcctaatctatatctgggtagtatatgctatcctaatttatatctgggtagcataggctatc
ctaatctatatctgggtagcatatgctatcctaatctatatctgggtagtatatgctatcctaatctgtatccgggtagcatatgct
atcctaatagagattagggtagtatatgctatcctaatttatatctgggtagcatatactacccaaatatctggatagcatatgct
atcctaatctatatctgggtagcatatgctatcctaatctatatctgggtagcataggctatcctaatctatatctgggtagcatat
gctatcctaatctatatctgggtagtatatgctatcctaatttatatctgggtagcataggctatcctaatctatatctgggtagcat
atgctatcctaatctatatctgggtagtatatgctatcctaatctgtatccgggtagcatatgctatcctcatgataagctgtcaa
acatgagaattttcttgaagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatga
gacaataaccctgataaatgcttcaataatattgaaaaaggaagagtatgagtattcaacatttccgtgtcgcccttattccct
tttttgcggcattttgccttcctgtttttgctcacccagaaacgctggtgaaagtaaaagatgctgaagatcagttgggtgcacg
agtgggttacatcgaactggatctcaacagcggtaagatccttgagagttttcgccccgaagaacgttttccaatgatgagc
acttttaaagttctgctatgtggcgcggtattatcccgtgttgacgccgggcaagagcaactcggtcgccgcatacactattct
cagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttg
cacaacatgggggatcatgtaactcgccttgatcgttgggaaccggagctgaatgaagccataccaaacgacgagcgtg
acaccacgatgcctgcagcaatggcaacaacgttgcgcaaactattaactggcgaactacttactctagcttcccggcaa
caattaatagactggatggaggcggataaagttgcaggaccacttctgcgctcggcccttccggctggctggtttattgctga
taaatctggagccggtgagcgtgggtctcgcggtatcattgcagcactggggccagatggtaagccctcccgtatcgtagtt
atctacacgacggggagtcaggcaactatggatgaacgaaatagacagatcgctgagataggtgcctcactgattaagc
attggtaactgtcagaccaagtttactcatatatactttagattgatttaaaacttcatttttaatttaaaaggatctaggtgaagat
cctttttgataatctcatgaccaaaatcccttaacgtgagttttcgttccactgagcgtcagaccccgtagaaaagatcaaag
gatcttcttgagatcctttttttctgcgcgtaatctgctgcttgcaaacaaaaaaaccaccgctaccagcggtggtttgtttgccg
gatcaagagctaccaactctttttccgaaggtaactggcttcagcagagcgcagataccaaatactgttcttctagtgtagcc
gtagttaggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacggg
gggttcgtgcacacagcccagcttggagcgaacgacctacaccgaactgagatacctacagcgtgagctatgagaaag
cgccacgcttcccgaagggagaaaggcggacaggtatccggtaagcggcagggtcggaacaggagagcgcacgag
ggagcttccagggggaaacgcctggtatctttatagtcctgtcgggtttcgccacctctgacttgagcgtcgatttttgtgatgct
cgtcaggggggcggagcctatggaaaaacgccagcaacgcggcctttttacggttcctggccttttgctggccttttgctcac
atgttctttcctgcgttatcccctgattctgtggataaccgtattaccgcctttgagtgagctgataccgctcgccgcagccgaa
cgaccgagcgcagcgagtcagtgagcgaggaagcggaagagcgcccaatacgcaaaccgcctctccccgcgcgttg
gccgattcattaatgcagctggcacgacaggtttcccgactggaaagcgggcagtgagcgcaacgcaattaatgtgagtt
agctcactcattaggcaccccaggctttacactttatgcttccggctcgtatgttgtgtggaattgtgagcggataacaatttca
cacaggaaacagctatgaccatgattacgccaagctctagctagaggtcgaccaattctcatgtttgacagcttatcatcgc
agatccgggcaacgttgttgccattgctgcaggcgcagaactggtaggtatggaagatctatacattgaatcaatattggca
attagccatattagtcattggttatatagcataaatcaatattggctattggccattgcatacgttgtatctatatcataatatgtac
atttatattggctcatgtccaatatgaccgccatgttgacattgattattgactagttattaatagtaatcaattacggggtcatta
gttcatagcccatatatggagttccgcgttacataacttacggtaaatggcccgcctggctgaccgcccaacgacccccgc
ccattgacgtcaataatgacgtatgttcccatagtaacgccaatagggactttccattgacgtcaatgggtggagtatttacg
gtaaactgcccacttggcagtacatcaagtgtatcatatgccaagtccgccccctattgacgtcaatgacggtaaatggccc
gcctggcattatgcccagtacatgaccttacgggactttcctacttggcagtacatctacgtattagtcatcgctattaccatgg
tgatgcggttttggcagtacaccaatgggcgtggatagcggtttgactcacggggatttccaagtctccaccccattgacgtc
aatgggagtttgttttggcaccaaaatcaacgggactttccaaaatgtcgtaataaccccgccccgttgacgcaaatgggc
ggtaggcgtgtacggtgggaggtctatataagcagagctcgtttagtgaaccgtcagatcctcactctcttccgcatcgctgt
ctgcgagggccagctgttgggctcgcggttgaggacaaactcttcgcggtctttccagtactcttggatcggaaacccgtcg
gcctccgaacggtactccgccaccgagggacctgagcgagtccgcatcgaccggatcggaaaacctctcgagaaagg
cgtctaaccagtcacagtcgcaaggtaggctgagcaccgtggcgggcggcagcgggtggcggtcggggttgtttctggc
ggaggtgctgctgatgatgtaattaaagtaggcggtcttgagacggcggatggtcgaggtgaggtgtggcaggcttgagat
ccagctgttggggtgagtactccctctcaaaagcgggcattacttctgcgctaagattgtcagtttccaaaaacgaggagga
tttgatattcacctggcccgatctggccatacacttgagtgacaatgacatccactttgcctttctctccacaggtgtccactccc
aggtccaagtttgggcgccaccatggagtttgggctgagctggctttttcttgtcgcgattttaaaaggtgtccagtgt-
gaggtgcagctggtggagtctgggggaggcttggtacagcccggcaggtccctgagactctcctgtgcggcctctggattc
acctttgatgattatgccatgcactgggtccggcaagctccagggaagggcctggaatgggtctcagctatcacttggaata
gtggtcacatagactatgcggactctgtggagggccgattcaccatctccagagacaacgccaagaactccctgtatctgc
aaatgaacagtctgagagctgaggatacggccgtatattactgtgcgaaagtctcgtaccttagcaccgcgtcctcccttga
ctattggggccaaggtaccctggtcaccgtctcgagtgcgtcgaccaagggcccatcggtcttccccctggcaccctcctc
caagagcacctctgggggcacagcggccctgggctgcctggtcaaggactacttccccgaaccggtgacggtgtcgtgg
aactcaggcgccctgaccagcggcgtgcacaccttcccggctgtcctacagtcctcaggactctactccctcagcagcgt
ggtgaccgtgccctccagcagcttgggcacccagacctacatctgcaacgtgaatcacaagcccagcaacaccaaggt
ggacaagaaagttgagcccaaatcttgtgacaaaactcacacatgcccaccgtgcccagcacctgaactcctgggggg
accgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatg
gcaaggagtacaagtgcaaggtctccaacaaagccctcccagcccccatcgagaaaaccatctccaaagccaaagg
gcagccccgagaaccacaggtgtacaccctgcccccatcccgggatgagctgaccaagaaccaggtcagcctgacct
gcctggtcaaaggcttctatcccagcgacatcgccgtggagtgggagagcaatgggcagccggagaacaactacaag
accacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtctccggg
taaa-
tgcctgtccttcggcaccgagatcctgaccgtggagtacggccctctgcctatcggcaagatcgtgtccgaagagatcaac
tgctccgtgtactccgtggaccctgagggccgggtgtatactcaggccatcgcccagtggcacgaccggggcgagcagg
aggtgctggagtacgagctggaggacggctccgtgatccgggccacctccgaccaccggtttctgaccaccgactatca
gctgctggccatcgaggagatcttcgcccggcagctggacctgctgaccctggagaacatcaagcagaccgaggaggc
cctggacaaccaccggctgcctttccctctgctggacgccggcaccatcaagatggtgaaggtgatcggcaggcggtcc
ctgggcgtgcagcggatcttcgacatcggcctgcctcaggaccacaactttctgctggccaacggcgccatcgccgccaa
c-
atggacatgcgcgtgcccgcccagctgctgggcctgctgctgctgtggttccccggctcgcgatgcgacatccagatgacc
cagtctccatcctccctgtctgcatctgtaggggacagagtcaccatcacttgtcgggcaagtcagggcatcagaaattactt
agcctggtatcagcaaaaaccagggaaagcccctaagctcctgatctatgctgcatccactttgcaatcaggggtcccatc
tcggttcagtggcagtggatctgggacagatttcactctcaccatcagcagcctacagcctgaagatgttgcaacttattact
gtcaaaggtataaccgtgcaccgtatacttttggccaggggaccaaggtggaaatcaaacgtacggtggctgcaccatct
gtcttcatcttcccgccatctgatgagcagttgaaatctggaactgcctctgttgtgtgcctgctgaataacttctatcccagag
aggccaaagtacagtggaaggtggataacgccctccaatcgggtaactcccaggagagtgtcacagagcaggacagc
aaggacagcacctacagcctcagcagcaccctgacgctgagcaaagcagactacgagaaacacaaagtctacgcct
gcgaagtcacccatcagggcctgagctcgcccgtcacaaagagcttcaacaggggagagtgt-3′
pTT3 Vector-Heavy Chain-Intein-Light Chain

In the following constructs, the only difference from the construct above is the inclusion of extein sequences native to Synechocystis spp. Strain PCC6803 (shown in blue). The sequences shown are from the end of the D2E7 heavy chain coding region (last 9 base pairs as shown in red) to the 5′ end of the D2E7 light chain coding region (first 9 base pairs as shown in pink).

TABLE 25
pTT3-HC-Ssp-GA-int-LC-1aa, relevant portion of
coding sequence (SEQ ID NO: 136)

Ccgggtaaa-tatt-
gcctgtccttcggcaccgagatcctgaccgtggagtacggccctctgcc
tatcggcaagatcgtgtccgaagagatcaactgctccgtgtactccgtg
gaccctgagggccgggtgtatactcaggccatcgcccagtggcacgacc
ggggcgagcaggaggtgctggagtacgagctggaggacggctccgtgat
ccgggccacctccgaccaccggtttctgaccaccgactatcagctgctg
gccatcgaggagatcttcgcccggcagctggacctgctgaccctggaga
acatcaagcagaccgaggaggccctggacaaccaccggctgcctttccc
tctgctggacgccggcaccatcaagatggtgaaggtgatcggcaggcgg
tccctgggcgtgcagcggatcttcgacatcggcctgcctcaggaccaca
actttctgctggccaacggcgccatcgccgccaac-tgt-atggacatg
pTT3 Vector-Heavy Chain-Intein-Light Chain

TABLE 26
pTT3-HC-Ssp-GA-int-LC-3aa-relevant portion of
coding sequence (SEQ ID NO: 137)

Ccgggtaaa-gccgagtatt-
gcctgtccttcggcaccgagatcctgaccgtggagtacggccctctgcc
tatcggcaagatcgtgtccgaagagatcaactgctccgtgtactccgtg
gaccctgagggccgggtgtatactcaggccatcgcccagtggcacgacc
ggggcgagcaggaggtgctggagtacgagctggaggacggctccgtgat
ccgggccacctccgaccaccggtttctgaccaccgactatcagctgctg
gccatcgaggagatcttcgcccggcagctggacctgctgaccctggaga
acatcaagcagaccgaggaggccctggacaaccaccggctgcctttccc
tctgctggacgccggcaccatcaagatggtgaaggtgatcggcaggcgg
tccctgggcgtgcagcggatcttcgacatcggcctgcctcaggaccaca
actttctgctggccaacggcgccatcgccgccaac-tgtttcaac-atg
gacatg
pTT3 Vector-Heavy Chain-Intein-Light Chain

In addition, tables 8A-8C provide relevant sequences for a D2E7 intein fusion protein, expression vector and coding sequence using the mutated (Serine to Threonine) Pyrococcus Ssp. GBD Pol intein.

TABLE 8A
Coding Sequence of D2E7 Intein Fusion Protein (SEQ ID NO: 48)

ATGGAGTTTGGGCTGAGCTGGCTTTTTCTTGTCGCGATTTTAAAAGGTGTCCAGTGTGAGGTGCAGCTGGTGGAGTCTGGGGG
AGGCTTGGTACAGCCCGGCAGGTCCCTGAGACTCTCCTGTGCGGCCTCTGGATTCACCTTTGATGATTATGCCATGCACTGGG
TCCGGCAAGCTCCAGGGAAGGGCCTGGAATGGGTCTCAGCTATCACTTGGAATAGTGGTCACATAGACTATGCGGACTCTGTG
GAGGGCCGATTCACCATCTCCAGAGACAACGCCAAGAACTCCCTGTATCTGCAAATGAACAGTCTGAGAGCTGAGGATACGGC
CGTATATTACTGTGCGAAAGTCTCGTACCTTAGCACCGCGTCCTCCCTTGACTATTGGGGCCAAGGTACCCTGGTCACCGTCTC
GAGTGCGTCGACCAAGGGCCCATCGGTCTTCCCCCTGGCACCCTCCTCCAAGAGCACCTCTGGGGGCACAGCGGCCCTGGG
CTGCCTGGTCAAGGACTACTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCAGCTTGGGCACCCAGACCTA
CATCTGCAACGTGAATCACAAGCCCAGCAACACCAAGGTGGACAAGAAAGTTGAGCCCAAATCTTGTGACAAAACTCACACATG
CCCACCGTGCCCAGCACCTGAACTCCTGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCCTCCCAGCCCCCATCGAGAAAACCAT
CTCCAAAGCCAAAGGGCAGCCCCGAGAACCACAGGTGTACACCCTGCCCCCATCCCGGGATGAGCTGACCAAGAACCAGGTC
AGCCTGACCTGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAGCAGGTGGCA
GCAGGGGAACGTCTTCTCATGCTCCGTGATGCATGAGGCTCTGCACAACCACTACACGCAGAAGAGCCTCTCCCTGTCTCCGG
GTAAAACCATTTTACCGGAAGAATGGGTTCCACTAATTAAAAACGGTAAAGTTAAGATATTCCGCATTGGGGACTTCGTTGATGG
ACTTATGAAGGCGAACCAAGGAAAAGTGAAGAAAACGGGGGATACAGAAGTTTTAGAAGTTGCAGGAATTCATGCGTTTTCCTT
TGACAGGAAGTCCAAGAAGGCCCGTGTAATGGCAGTGAAAGCCGTGATAAGACACCGTTATTCCGGAAATGTTTATAGAATAGT
CTTAAACTCTGGTAGAAAAATAACAATAACAGAAGGGCATAGCCTATTTGTCTATAGGAACGGGGATCTCGTTGAGGCAACTGG
GGAGGATGTCAAAATTGGGGATCTTCTTGCAGTTCCAAGATCAGTAAACCTACCAGAGAAAAGGGAACGCTTGAATATTGTTGA
ACTTCTTCTGAATCTCTCACCGGAAGAGACAGAAGATATAATACTTACGATTCCAGTTAAAGGCAGAAAGAACTTCTTCAAGGGA
ATGTTGAGAACATTACGTTGGATTTTTGGTGAGGAAAAGAGAGTAAGGACAGCGAGCCGCTATCTAAGACACCTTGAAAATCTC
GGATACATAAGGTTGAGGAAAATTGGATACGACATCATTGATAAGGAGGGGCTTGAGAAATATAGAACGTTGTACGAGAAACTT
GTTGATGTTGTCCGCTATAATGGCAACAAGAGAGAGTATTTAGTTGAATTTAATGCTGTCCGGGACGTTATCTCACTAATGCCAG
AGGAAGAACTGAAGGAATGGCGTATTGGAACTAGAAATGGATTCAGAATGGGTACGTTCGTAGATATTGATGAAGATTTTGCCA
AGCTTGGATACGATAGCGGAGTCTACAGGGTTTATGTAAACGAGGAACTTAAGTTTACGGAATACAGAAAGAAAAAGAATGTAT
ATCACTCTCACATTGTTCCAAAGGATATTCTCAAAGAAACTTTTGGTAAGGTCTTCCAGAAAAATATAAGTTACAAGAAATTTAGA
GAGCTTGTAGAAAATGGAAAACTTGACAGGGAGAAAGCCAAACGCATTGAGTGGTTACTTAACGGAGATATAGTCCTAGATAGA
GTCGTAGAGATTAAGAGAGAGTACTATGATGGTTACGTTTACGATCTAAGTGTCGATGAAGATGAGAATTTCCTTGCTGGCTTTG
GATTCCTCTATGCACATAATGACATCCAGATGACCCAGTCTCCATCCTCCCTGTCTGCATCTGTAGGGGACAGAGTCACCATCA
CTTGTCGGGCAAGTCAGGGCATCAGAAATTACTTAGCCTGGTATCAGCAAAAACCAGGGAAAGCCCCTAAGCTCCTGATCTATG
CTGCATCCACTTTGCAATCAGGGGTCCCATCTCGGTTCAGTGGCAGTGGATCTGGGACAGATTTCACTCTCACCATCAGCAGCC
TACAGCCTGAAGATGTTGCAACTTATTACTGTCAAAGGTATAACCGTGCACCGTATACTTTTGGCCAGGGGACCAAGGTGGAAA
TCAAACGTACGGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCCTGACGCTGAGCAAAGCAGACTACGAGAAAC
ACAAAGTCTACGCCTGCGAAGTCACCCATCAGGGCCTGAGCTCGCCCGTCACAAAGAGCTTCAACAGGGGAGAGTGTTGA

TABLE 8B
Amino Acid Sequence of D2E7 Intein Fusion Construct (SEQ ID NO: 49)

MEFGLSWLFLVAILKGVQCEVQLVESGGGLVQPGRSLRLSCAASGFTFDDYAMHWVRQAPGKGLEWVSAITWNSGHIDYADSVEG
RFTISRDNAKNSLYLQMNSLRAEDTAVYYCAKVSYLSTASSLDYWGQGTLVTVSSASTKGPSVFPLAPSSKSTSGGTAALGCLVKDY
FPEPVTVSWNSGALTSGVHTFPAVLQSSGLYSLSSWTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGKTILPEEWVPLIKNGKVKIFRIGDFVDGLMKANQGKVKKTGDTEVLEVAGI
HAFSFDRKSKKARVMAVKAVIRHRYSGNVYRIVLNSGRKITITEGHSLFVYRNGDLVEATGEDVKIGDLLAVPRSVNLPEKRERLNIVE
LLLNLSPEETEDIILTIPVKGRKNFFKGMLRTLRWIFGEEKRVRTASRYLRHLENLGYIRLRKIGYDIIDKEGLEKYRTLYEKLVDWRYN
GNKREYLVEFNAVRDVISLMPEEELKEWRIGTRNGFRMGTFVDIDEDFAKLGYDSGVYRVYVNEELKFTEYRKKKNVYHSHIVPKDIL
KETFGKVFQKNISYKKFRELVENGKLDREKAKRIEWLLNGDIVLDRVVEIKREYYDGYVYDLSVDEDENFLAGFGFLYAHNDIQMTQS
PSSLSASVGDRVTITCRASQGIRNYLAWYQQKPGKAPKLLIYAASTLQSGVPSRFSGSGSGTDFTLTISSLQPEDVATYYCQRYNRAP
YTFGQGTKVEIKRTVAAPSVFIFPPSDEQLKSGTASVVCLLNNFYPREAKVQWKVDNALQSGNSQESVTEQDSKDSTYSLSSTLTLSK
ADYEKHKVYACEVTHQGLSSPVTKSFNRGEC*

TABLE 8C
Complete Nucleotide Sequence of Expression Vector for the D2E7
Intein Fusion Construct (SEQ ID NO: 50)

GAAGTTCCTATTCCGAAGTTCCTATTCTCTAGACGTTACATAACTTACGGTAAATGGCCCGCCTGGCT
GACCGCCCAACGACCCCCGCCCATTGACGTCAATAATGACGTATGTTCCCATAGTAACGCCAATAGG
GACTTTCCATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCATTATGCCC
AGTACATGACCTTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGGATTTCCAAGT
CTCCACCCCATTGACGTCAATGGGAGTTTGTTTTGGCACCAAAATCAACGGGACTTTCCAAAATGTC
GTAACAACTCCGCCCCAATGACGCAAATGGGCAGGGAATTCGAGCTCGGTACTCGAGCGGTGTTCC
GCGGTCCTCCTCGTATAGAAACTCGGACCACTCTGAGACGAAGGCTCGCGTCCAGGCCAGCACGAA
GGAGGCTAAGTGGGAGGGGTAGCGGTCGTTGTCCACTAGGGGGTCCACTCGCTCCAGGGTGTGAA
GACACATGTCGCCCTCTTCGGCATCAAGGAAGGTGATTGGTTTATAGGTGTAGGCCACGTGACCGG
GTGTTCCTGAAGGGGGGCTATAAAAGGGGGTGGGGGCGCGTTCGTCCTCACTCTCTTCCGCATCGC
TGTCTGCGAGGGCCAGCTGTTGGGCTCGCGGTTGAGGACAAACTCTTCGCGGTCTTTCCAGTACTC
TTGGATCGGAAACCCGTCGGCCTCCGAACGGTACTCCGCCACCGAGGGACCTGAGCGAGTCCGCA
TCGACCGGATCGGAAAACCTCTCGACTGTTGGGGTGAGTACTCCCTCTCAAAAGCGGGCATGACTT
CTGCGCTAAGATTGTCAGTTTCCAAAAACGAGGAGGATTTGATATTCACCTGGCCCGCGGTGATGCC
TTTGAGGGTGGCCGCGTCCATCTGGTCAGAAAAGACAATCTTTTTGTTGTCAAGCTTGAGGTGTGGC
AGGCTTGAGATCTGGCCATACACTTGAGTGACAATGACATCCACTTTGCCTTTCTCTCCACAGGTGT
CCACTCCCAGGTCCAACCGGAATTGTACCCGCGGCCAGAGCTTGCCCGGGCGCCACCATGGAGTTT
GGGCTGAGCTGGCTTTTTCTTGTCGCGATTTTAAAAGGTGTCCAGTGTGAGGTGCAGCTGGTGGAGT
CTGGGGGAGGCTTGGTACAGCCCGGCAGGTCCCTGAGACTCTCCTGTGCGGCCTCTGGATTCACCT
TTGATGATTATGCCATGCACTGGGTCCGGCAAGCTCCAGGGAAGGGCCTGGAATGGGTCTCAGCTA
TCACTTGGAATAGTGGTCACATAGACTATGCGGACTCTGTGGAGGGCCGATTCACCATCTCCAGAGA
CAACGCCAAGAACTCCCTGTATCTGCAAATGAACAGTCTGAGAGCTGAGGATACGGCCGTATATTAC
TGTGCGAAAGTCTCGTACCTTAGCACCGCGTCCTCCCTTGACTATTGGGGCCAAGGTACCCTGGTCA
CCGTCTCGAGTGCGTCGACCAAGGGCCCATCGGTCTTCCCCCTGGCACCCTCCTCCAAGAGCACCT
CTGGGGGCACAGCGGCCCTGGGCTGCCTGGTCAAGGACTACTTCCCCGAACCGGTGACGGTGTCG
TGGAACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAAAGTTGAGCCCAAATCTTGTGACAAAACTC
ACACATGCCCACCGTGCCCAGCACCTGAACTCCTGGGGGGACCGTCAGTCTTCCTCTTCCCCCCAA
AACCCAAGGACACCCTCATGATCTCCCGGACCCCTGAGGTCACATGCGTGGTGGTGGACGTGAGCC
ACGAAGACCCTGAGGTCAAGTTCAACTGGTACGTGGACGGCGTGGAGGTGCATAATGCCAAGACAA
AGCCGCGGGAGGAGCAGTACAACAGCACGTACCGTGTGGTCAGCGTCCTCACCGTCCTGCACCAG
GACTGGCTGAATGGCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGA
CTCCGACGGCTCCTTCTTCCTCTACAGCAAGCTCACCGTGGACAAGAGCAGGTGGCAGCAGGGGAA
CGTCTTCTCATGCTCCGTGATGCATGAGGCTCTGCACAACCACTACACGCAGAAGAGCCTCTCCCTG
TCTCCGGGTAAAACCATTTTACCGGAAGAATGGGTTCCACTAATTAAAAACGGTAAAGTTAAGATATT
CCGCATTGGGGACTTCGTTGATGGACTTATGAAGGCGAACCAAGGAAAAGTGAAGAAAACGGGGGA
TACAGAAGTTTTAGAAGTTGCAGGAATTCATGCGTTTTCCTTTGACAGGAAGTCCAAGAAGGCCCGT
GTAATGGCAGTGAAAGCCGTGATAAGACACCGTTATTCCGGAAATGTTTATAGAATAGTCTTAAACTC
TGGTAGAAAAATAACAATAACAGAAGGGCATAGCCTATTTGTCTATAGGAACGGGGATCTCGTTGAG
GCAACTGGGGAGGATGTCAAAATTGGGGATCTTCTTGCAGTTCCAAGATCAGTAAACCTACCAGAGA
AAAGGGAACGCTTGAATATTGTTGAACTTCTTCTGAATCTCTCACCGGAAGAGACAGAAGATATAATA
CTTACGATTCCAGTTAAAGGCAGAAAGAACTTCTTCAAGGGAATGTTGAGAACATTACGTTGGATTTT
TGGTGAGGAAAAGAGAGTAAGGACAGCGAGCCGCTATCTAAGACACCTTGAAAATCTCGGATACATA
AGGTTGAGGAAAATTGGATACGACATCATTGATAAGGAGGGGCTTGAGAAATATAGAACGTTGTACG
AGAAACTTGTTGATGTTGTCCGCTATAATGGCAACAAGAGAGAGTATTTAGTTGAATTTAATGCTGTC
CGGGACGTTATCTCACTAATGCCAGAGGAAGAACTGAAGGAATGGCGTATTGGAACTAGAAATGGAT
TCAGAATGGGTACGTTCGTAGATATTGATGAAGATTTTGCCAAGCTTGGATACGATAGCGGAGTCTA
CAGGGTTTATGTAAACGAGGAACTTAAGTTTACGGAATACAGAAAGAAAAAGAATGTATATCACTCTC
ACATTGTTCCAAAGGATATTCTCAAAGAAACTTTTGGTAAGGTCTTCCAGAAAAATATAAGTTACAAGA
AATTTAGAGAGCTTGTAGAAAATGGAAAACTTGACAGGGAGAAAGCCAAACGCATTGAGTGGTTACT
TAACGGAGATATAGTCCTAGATAGAGTCGTAGAGATTAAGAGAGAGTACTATGATGGTTACGTTTACG
ATCTAAGTGTCGATGAAGATGAGAATTTCCTTGCTGGCTTTGGATTCCTCTATGCACATAATGACATC
CAGATGACCCAGTCTCCATCCTCCCTGTCTGCATCTGTAGGGGACAGAGTCACCATCACTTGTCGGG
CAAGTCAGGGCATCAGAAATTACTTAGCCTGGTATCAGCAAAAACCAGGGAAAGCCCCTAAGCTCCT
GATCTATGCTGCATCCACTTTGCAATCAGGGGTCCCATCTCGGTTCAGTGGCAGTGGATCTGGGACA
GATTTCACTCTCACCATCAGCAGCCTACAGCCTGAAGATGTTGCAACTTATTACTGTCAAAGGTATAA
CCGTGCACCGTATACTTTTGGCCAGGGGACCAAGGTGGAAATCAAACGTACGGTGGCTGCACCATC
TGTCTTCATCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAGCACCCTGACG
CTGAGCAAAGCAGACTACGAGAAACACAAAGTCTACGCCTGCGAAGTCACCCATCAGGGCCTGAGC
TCGCCCGTCACAAAGAGCTTCAACAGGGGAGAGTGTTGAGCGGCCGCGTTTAAACTGAATGAGCGC
GTCCATCCAGACATGATAAGATACATTGATGAGTTTGGACAAACCACAACTAGAATGCAGTGAAAAAA
ATGCTTTATTTGTGAAATTTGTGATGCTATTGCTTTATTTGTAACCATTATAAGCTGCAATAAACAAGTT
AACAACAACAATTGCATTCATTTTATGTTTCAGGTTCAGGGGGAGGTGTGGGAGGTTTTTTAAAGCAA
GTAAAACCTCTACAAATGTGGTATGGCTGATTATGATCCGGCTGCCTCGCGCGTTTCGGTGATGACG
GTGAAAACCTCTGACACATGCAGCTCCCGGAGACGGTCACAGCTTGTCTGTAAGCGGATGCCGGGA
GCAGACAAGCCCGTCAGGGCGCGTCAGCGGGTGTTGGCGGGTGTCGGGGCGCAGCCATGACCGG
TCGACGGCGCGCCTTTTTTTTTAATTTTTATTTTATTTTATTTTTGACGCGCCGAAGGCGCGATCTGAG
CTCGGTACAGCTTGGCTGTGGAATGTGTGTCAGTTAGGGTGTGGAAAGTCCCCAGGCTCCCCAGCA
GGCAGAAGTATGCAAAGCATGCATCTCAATTAGTCAGCAACCAGGTGTGGAAAGTCCCCAGGCTCC
CCAGCAGGCAGAAGTATGCAAAGCATGCATCTCAATTAGTCAGCAACCATAGTCCCGCCCCTAACTC
CGCCCATCCCGCCCCTAACTCCGCCCAGTTCCGCCCATTCTCCGCCCCATGGCTGACTAATTTTTTT
TATTTATGCAGAGGCCGAGGCCGCCTCGGCCTCTGAGCTATTCCAGAAGTAGTGAGGAGGCTTTTTT
GGAGGCCTAGGCTTTTGCAAAAAGCTCCTCGAGGAACTGAAAAACCAGAAAGTTAACTGGTAAGTTT
AGTCTTTTTGTCTTTTATTTCAGGTCCCGGATCCGGTGGTGGTGCAAATCAAAGAACTGCTCCTCAGT
GGATGTTGCCTTTACTTCTAGGCCTGTACGGAAGTGTTACTTCTGCTCTAAAAGCTGCGGAATTGTAC
CCGCGGCCTAATACGACTCACTATAGGGACTAGTATGGTTCGACCATTGAACTGCATCGTCGCCGTG
TCCCAAAATATGGGGATTGGCAAGAACGGAGACCTACCCTGGCCTCCGCTCAGGAACGAGTTCAAG
TACTTCCAAAGAATGACCACAACCTCTTCAGTGGAAGGTAAACAGAATCTGGTGATTATGGGTAGGA
AAACCTGGTTCTCCATTCCTGAGAAGAATCGACCTTTAAAGGACAGAATTAATATAGTTCTCAGTAGA
GAACTCAAAGAACCACCACGAGGAGCTCATTTTCTTGCCAAAAGTTTAGATGATGCCTTAAGACTTAT
TGAACAACCGGAATTGGCAAGTAAAGTAGACATGGTTTGGATAGTCGGAGGCAGTTCTGTTTACCAG
GAAGCCATGAATCAACCAGGCCACCTCAGACTCTTTGTGACAAGGATCATGCAGGAATTTGAAAGTG
ACACGTTTTTCCCAGAAATTGATTTGGGGAAATATAAACTTCTCCCAGAATACCCAGGCGTCCTCTCT
GAGGTCCAGGAGGAAAAAGGCATCAAGTATAAGTTTGAAGTCTACGAGAAGAAAGACTAAGCGGCC
GAGCGCGCGGATCTGGAAACGGGAGATGGGGGAGGCTAACTGAAGCACGGAAGGAGACAATACCG
GAAGGAACCCGCGCTATGACGGCAATAAAAAGACAGAATAAAACGCACGGGTGTTGGGTCGTTTGT
TCATAAACGCGGGGTTCGGTCCCAGGGCTGGCACTCTGTCGATACCCCACCGAGACCCCATTGGGG
CCAATACGCCCGCGTTTCTTCCTTTTCCCCACCCCACCCCCCAAGTTCGGGTGAAGGCCCAGGGCT
CGCAGCCAACGTCGGGGCGGCAGGCCCTGCCATAGCCACTGGCCCCGTGGGTTAGGGACGGGGT
CCCCCATGGGGAATGGTTTATGGTTCGTGGGGGTTATTATTTTGGGCGTTGCGTGGGGTCTGGAGA
TCCCCCGGGCTGCAGGAATTCCGTTACATTACTTACGGTAAATGGCCCGCCTGGCTGACCGCCCAA
CGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGC
CAAGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTGATGCGGT
TTTGGCAGTACATCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTCC
GCCCCATTGACGCAAAAGGGCGGGAATTCGAGCTCGGTACTCGAGCGGTGTTCCGCGGTCCTCCTC
GTATAGAAACTCGGACCACTCTGAGACGAAGGCTCGCGTCCAGGCCAGCACGAAGGAGGCTAAGTG
GGAGGGGTAGCGGTCGTTGTCCACTAGGGGGTCCACTCGCTCCAGGGTGTGAAGACACATGTCGC
CCTCTTCGGCATCAAGGAAGGTGATTGGTTTATAGGTGTAGGCCACGTGACCGGGTGTTCCTGAAG
GGGGGCTATAAAAGGGGGTGGGGGCGCGTTCGTCCTCACTCTCTTCCGCATCGCTGTCTGCGAGG
GCCAGCTGTTGGGCTCGCGGTTGAGGACAAACTCTTCGCGGTCTTTCCAGTACTCTTGGATCGGAA
ACCCGTCGGCCTCCGAACGGTACTCCGCCACCGAGGGACCTGAGCGAGTCCGCATCGACCGGATC
GGAAAACCTCTCGACTGTTGGGGTGAGTACTCCCTCTCAAAAGCGGGCATGACTTCTGCGCTAAGAT
TGTCAGTTTCCAAAAACGAGGAGGATTTGATATTCACCTGGCCCGCGGTGATGCCTTTGAGGGTGGC
CGCGTCCATCTGGTCAGAAAAGACAATCTTTTTGTTGTCAAGCTTGAGGTGTGGCAGGCTTGAGATC
TGGCCATACACTTGAGTGACAATGACATCCACTTTGCCTTTCTCTCCACAGGTGTCCACTCCCAGGT
CCAACCGGAATTGTACCCGCGGCCAGAGCTTGCGGGCGCCACCGCGGCCGCGGGGATCCAGACAT
GATAAGATACATTGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTCGGATCCTCTTGGCGTAAT
CATGGTCATAGCTGTTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGGGA
AAGGCGGTTTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAAAAGGCCGCGTTGC
TGGCGTTCTTCCATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTTAAGGGATTTTG
GTCATGAGATTATCAAAAAGGATCTTCACCTAGATCCCTTTTAATTAAAAATGAAGTTTTAAATCAATC
TAAAGTATATATGAGTAAACTTGGTCTGACAGTTACCAATGCTTAATCAGTGAGGCACCTATCTCAGC
GATCTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATTTA
TCAGCAATAAACCAGCCAGCCGGAAGGGCCGAGCGCAGAAGTGGTCCTGCAACTTTATCCGCCTCC
ATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGGGAATAAGGGCGAC
ACGGAAATGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACATTTCCCC
GAAAAGTGCCACCTGACGTCTAAGAAACCATTATTATCATGACATTAACCTATAAAAATAGGCGTATC
ACGAGGCCCTTTCGTCTCGCGCGTTTCGGTGATGACGGTGAAAACCTCTGACACATGCAGCTCCCG
GAGACGGTCACAGCTTGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGATTGTACTGAGAGTGCA
CCATATGCGGTGTGAAATACCGCACAGATGCGTAAGGAGAAAATACCGCATCAGGCGCCATTCGCC
ATTCAGGCTGCGCAACTGTTGGGAAGGGCGATCGGTGCGGGCCTCTTCGCTATTACGCCAGCTGGC
GAAAGGGGGATGTGCTGCAAGGCGATTAAGTTGGGTAACGCCAGGGTTTTCCCAGTTACGACGTTG
TAAAACGACGGCCAGTGAATT

TABLE 9
Amino acid sequence of the native Psp-GBD Pol intein sequence
with limited flanking sequence information
(NCBI Accession No. AAA67132.1) (SEQ ID NO: 51)

N/SILPEEWVPLIKNGKVKIFRIGDFVDGLMKANQGKVKKTGDTEVLEVAGIHAFSFDRK
SKKARVMAVKAVIRHRYSGNVYRIVLNSGRKITITEGHSLFVYRNGDLVEATGEDVKIG
DLLAVPRSVNLPEKRERLNIVELLLNLSPEETEDIILTIPVKGRKNFFKGMLRTLRWIFGE
EKRVRTASRYLRHLENLGYIRLRKIGYDIIDKEGLEKYRTLYEKLVDVVRYNGNKREYLV
EFNAVRDVISLMPEEELKEWRIGTRNGFRMGTFVDIDEDFAKLLGYYVSEGSARKWKN
QTGGWSYTVRLYNENDEVLDDMEHLAKKFFGKVKRGKNYVEIPKKMAYIIFESLCGTL
AENKRVPEVIFTSSKGVRWAFLEGYFIGDGDVHPSKRVRLSTKSELLVNGLVLLLNSLG
VSAIKLGYDSGVYRVYVNEELKFTEYRKKKNVYHSHIVPKDILKETFGKVFQKNISYKKF
RELVENGKLDREKAKRIEWLLNGDIVLDRVVEIKREYYDGYVYDLSVDEDENFLAGFGF
LYAHN/SYYGYYGYA

/ represents splice junction, and underlined amino acids represent intein sequences, the remainder represents extein sequence information.

### Example 2

### Construction of Immunoglobulin Polyprotein Sequences and Vectors with Drosophila melanogaster Hedgehog Auto Processing Domain, C17 and C25 Sequences

A further strategy for the efficient expression of antibody molecules is polyprotein expression, wherein an Hedgehog domain is located between the heavy and light chains, with modification of the Hedgehog domain sequence and/or junction sequences such that there is release of the component proteins without cholesterol addition to the N-terminal protein. Within such constructs, there can be one copy of each of the relevant heavy and light chains, or the light chain can be duplicated to provide at least two light chains, or there can be multiple copies of both heavy and light chains, provided that a functional cleavage sequence is provided to promote separation of each immunoglobulin-derived protein within the polyprotein. A particular cleavage site strategy (e.g., the Hedgehog domain) can be employed more than once, or for multiple cleavage sites each can be independent. Thus a different proteolytic processing sequence or enzyme can be positioned relative to at least one terminus of an immunoglobulin or immunoglobulin-derived protein.

The following oligonucleotides were used for the amplification of the Drosophila melanogaster Hedgehog C-terminal auto processing domain (Hh-C), sequences Hh-C17, Hh-C17 truncations (and one with mutation) and Hh-C25 (GenBank accession #L02793.1) using genomic DNA as template and Platinum Taq Hi Fidelity PCR Supermix (Invitrogen). Genomic DNA was prepared from a frozen vial of Drosophila D.MeI-2 cells (Invitrogen, cat. #10831-014).

C17-5′:

(SEQ ID NO: 141)

TGCTTCACGCCGGAGAGCAC
C17-full-3′:

(SEQ ID NO: 142)

ATTATGGACGACAACCTGGTTGGCAA
C25-actual-3′:

(SEQ ID NO: 143)

ATCGTGGCGCCAGCTCTGCG
C17-3′:

(SEQ ID NO: 144)

GCAACTGGCGGCCACCGAGT
C17-scya-3′:

(SEQ ID NO: 145)

CGCATAGCAACTGGCGGCCA
C17-sc/hn-3′:

(SEQ ID NO: 146)

GTTGTGGGCGGCCACCGAGT

PCR run on the following program:

| | Step | | | | | | | |
|---|---|---|---|---|---|---|---|---|
| | 1 | 2 | 3 | 4 | 5 | 6 | 7 | 8 |
| Temp | 94° C. | 94° C. | 55° C. | 68° C. | Go to step 2 (34 times) | 68° C. | 4° C. | End |
| Time | 2 min | 1 min | 1 min | 2.5 min | | | 5 min | hold |

Oligonucleotide primers were designed to generate the fusion of D2E7 Heavy Chain-Hh-C-D2E7 Light Chain by way of homologous recombination into the pTT3-HcintLC p. horikoshii construct in E. coli. By engineering a 40 base pair overhang between PCR generated vector (containing pTT3 vector, heavy chain and light chain regions but not the P. horikoshii intein) and the Hh-C domain inserts, the two DNA fragments are mixed and transformed into E. coli without the benefit of ligation, resulting in E. coli homologous recombination of the two fragments into pTT3-HC-Hh-C-LC (in various versions as the initial PCR products dictate).

### Hh-C Domain Homologous Recombination Primers:

C17-HR5′:

(SEQ ID NO: 147)

CCACTACACGCAGAAGAGCCTCTCCCTGTCTCCGGGTAAATGCTTCACGC
CGGAGAGCAC
C17-full-HR-3′:

(SEQ ID NO: 148)

GCAGCAGGCCCAGCAGCTGGGCGGGCACGCGCATGTCCATGCACTGGCT
GTTGATCACCG
C25-actual-HR-3′:

(SEQ ID NO: 149)

GCAGCAGGCCCAGCAGCTGGGCGGGCACGCGCATGTCCATATCGTGGCG
CCAGCTCTGCG
C17-HR3′:

(SEQ ID NO: 150)

GCAGCAGGCCCAGCAGCTGGGCGGGCACGCGCATGTCCATGCAACTGGC
GGCCACCGAGT
C17-scya-HR-3′:

(SEQ ID NO: 151)

GCAGCAGGCCCAGCAGCTGGGCGGGCACGCGCATGTCCATCGCATAGCAA
CTGGCGGCCA
C17-sc/hn-HR-3′:

(SEQ ID NO: 152)

GCAGCAGGCCCAGCAGCTGGGCGGGCACGCGCATGTCCATGTTGTGGGC
GGCCACCGAGT

pTT3-HcintLC Homologous Recombination Primers:

pTT3int-HR5′:

(SEQ ID NO: 153)

ATGGACATGCGCGTGCCCGCCCAGCTGCTGGGCCTGCTGC
pTT3int-HR3′:

(SEQ ID NO: 154)

TTTACCCGGAGACAGGGAGAGGCTCTTCTGCGTGTAGTGGT

PCR for Hh-C domain run on the following program: Pfu-I Hi Fidelity DNA Polymerase (Stratagene) used.

| | Step | | | | | | | |
|---|---|---|---|---|---|---|---|---|
| | 1 | 2 | 3 | 4 | 5 | 6 | 7 | 8 |
| Temp | 94° C. | 94° C. | 60° C. | 72° C. | Go to step 2 (34 times) | 72° C. | 4° C. | End |
| Time | 2 min | 1 min | 1 min | 1.5 min | | | 5 min | hold |

PCR for the vector run on the following program: Platinum Taq Hi Fidelity Supermix (Invitrogen) used.

| | Step | | | | | | | |
|---|---|---|---|---|---|---|---|---|
| | 1 | 2 | 3 | 4 | 5 | 6 | 7 | 8 |
| Temp | 94° C. | 94° C. | 60° C. | 68° C. | Go to step 2 (24 times) | 68° C. | 4° C. | End |
| Time | 2 min | 30 sec | 30 sec | 10 min | | 5 min | hold | |

To achieve homologous recombination of Hh-C domains into pTT3-HcintLC, the following strategy was employed. PCR products were gel purified and each eluted into 50 μl elution buffer (Qiaquick Gel Extraction kit, Qiagen). 3 μl of the vector PCR product was mixed in an eppendorf tube 3 μl of the desired Hint domain PCR product (various versions). The PCR amplification products were transformed into E. coli and plated onto LB+Ampicillin plates, incubated at 37° C. overnight, and colonies were grown to 2 ml cultures, plasmid DNA was extracted using the Wizard prep kit (Promega) and the DNA samples were assayed by restriction endonuclease digestion and agarose gel electrophoresis. Clones that produced the correct restriction pattern were analyzed with respect to DNA sequence to confirm that the desired sequence had been produced.

Five expression constructs for D2E7 Heavy Chain-Hh-C-D2E7 Light Chain expression, utilizing the Drosophila melanogaster Hedgehog C-terminal auto-processing domain, were designed: pTT3-HC-Hh-C17-LC; pTT3-HC-Hh-C17-SC-LC; pTT3-HC-Hh-C17-HN-LC; and pTT3-HC-Hh-C25-LC.

TABLE 27
Sequence of entire plasmid pTT3-D2E7 Heavy Chain-Hh-C17-D2E7
Light Chain (SEQ ID NO: 155)

5′-
gcggccgctcgaggccggcaaggccggatcccccgacctcgacctctggctaataaaggaaatttattttcattgcaatag
tgtgttggaattttttgtgtctctcactcggaaggacatatgggagggcaaatcatttggtcgagatccctcggagatctctagc
tagaggatcgatccccgccccggacgaactaaacctgactacgacatctctgccccttcttcgcggggcagtgcatgtaat
cccttcagttggttggtacaacttgccaactgggccctgttccacatgtgacacggggggggaccaaacacaaaggggtt
ctctgactgtagttgacatccttataaatggatgtgcacatttgccaacactgagtggctttcatcctggagcagactttgcagt
ctgtggactgcaacacaacattgcctttatgtgtaactcttggctgaagctcttacaccaatgctgggggacatgtacctccc
aggggcccaggaagactacgggaggctacaccaacgtcaatcagaggggcctgtgtagctaccgataagcggaccct
caagagggcattagcaatagtgtttataaggcccccttgttaaccctaaacgggtagcatatgcttcccgggtagtagtatat
actatccagactaaccctaattcaatagcatatgttacccaacgggaagcatatgctatcgaattagggttagtaaaagggt
cctaaggaacagcgatatctcccaccccatgagctgtcacggttttatttacatggggtcaggattccacgagggtagtgaa
ccattttagtcacaagggcagtggctgaagatcaaggagcgggcagtgaactctcctgaatcttcgcctgcttcttcattctc
cttcgtttagctaatagaataactgctgagttgtgaacagtaaggtgtatgtgaggtgctcgaaaacaaggtttcaggtgacg
cccccagaataaaatttggacggggggttcagtggtggcattgtgctatgacaccaatataaccctcacaaaccccttggg
caataaatactagtgtaggaatgaaacattctgaatatctttaacaatagaaatccatggggtggggacaagccgtaaag
actggatgtccatctcacacgaatttatggctatgggcaacacataatcctagtgcaatatgatactggggttattaagatgtg
tcccaggcagggaccaagacaggtgaaccatgttgttacactctatttgtaacaaggggaaagagagtggacgccgac
agcagcggactccactggttgtctctaacacccccgaaaattaaacggggctccacgccaatggggcccataaacaaa
gacaagtggccactcttttttttgaaattgtggagtgggggcacgcgtcagcccccacacgccgccctgcggttttggactgt
aaaataagggtgtaataacttggctgattgtaaccccgctaaccactgcggtcaaaccacttgcccacaaaaccactaat
ggcaccccggggaatacctgcataagtaggtgggcgggccaagataggggcgcgattgctgcgatctggaggacaaa
ttacacacacttgcgcctgagcgccaagcacagggttgttggtcctcatattcacgaggtcgctgagagcacggtgggcta
atgttgccatgggtagcatatactacccaaatatctggatagcatatgctatcctaatctatatctgggtagcataggctatcct
aatctatatctgggtagcatatgctatcctaatctatatctgggtagtatatgctatcctaatttatatctgggtagcataggctatc
ctaatctatatctgggtagcatatgctatcctaatctatatctgggtagtatatgctatcctaatctgtatccgggtagcatatgct
atcctaatagagattagggtagtatatgctatcctaatttatatctgggtagcatatactacccaaatatctggatagcatatgct
atcctaatctatatctgggtagcatatgctatcctaatctatatctgggtagcataggctatcctaatctatatctgggtagcatat
gctatcctaatctatatctgggtagtatatgctatcctaatttatatctgggtagcataggctatcctaatctatatctgggtagcat
atgctatcctaatctatatctgggtagtatatgctatcctaatctgtatccgggtagcatatgctatcctcatgataagctgtcaa
acatgagaattttcttgaagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatga
gacaataaccctgataaatgcttcaataatattgaaaaaggaagagtatgagtattcaacatttccgtgtcgcccttattccct
tttttgcggcattttgccttcctgtttttgctcacccagaaacgctggtgaaagtaaaagatgctgaagatcagttgggtgcacg
agtgggttacatcgaactggatctcaacagcggtaagatccttgagagttttcgccccgaagaacgttttccaatgatgagc
acttttaaagttctgctatgtggcgcggtattatcccgtgttgacgccgggcaagagcaactcggtcgccgcatacactattct
cagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttg
cacaacatgggggatcatgtaactcgccttgatcgttgggaaccggagctgaatgaagccataccaaacgacgagcgtg
acaccacgatgcctgcagcaatggcaacaacgttgcgcaaactattaactggcgaactacttactctagcttcccggcaa
caattaatagactggatggaggcggataaagttgcaggaccacttctgcgctcggcccttccggctggctggtttattgctga
taaatctggagccggtgagcgtgggtctcgcggtatcattgcagcactggggccagatggtaagccctcccgtatcgtagtt
atctacacgacggggagtcaggcaactatggatgaacgaaatagacagatcgctgagataggtgcctcactgattaagc
attggtaactgtcagaccaagtttactcatatatactttagattgatttaaaacttcatttttaatttaaaaggatctaggtgaagat
cctttttgataatctcatgaccaaaatcccttaacgtgagttttcgttccactgagcgtcagaccccgtagaaaagatcaaag
gatcttcttgagatcctttttttctgcgcgtaatctgctgcttgcaaacaaaaaaaccaccgctaccagcggtggtttgtttgccg
gatcaagagctaccaactctttttccgaaggtaactggcttcagcagagcgcagataccaaatactgttcttctagtgtagcc
gtagttaggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacggg
gggttcgtgcacacagcccagcttggagcgaacgacctacaccgaactgagatacctacagcgtgagctatgagaaag
cgccacgcttcccgaagggagaaaggcggacaggtatccggtaagcggcagggtcggaacaggagagcgcacgag
ggagcttccagggggaaacgcctggtatctttatagtcctgtcgggtttcgccacctctgacttgagcgtcgatttttgtgatgct
cgtcaggggggcggagcctatggaaaaacgccagcaacgcggcctttttacggttcctggccttttgctggccttttgctcac
atgttctttcctgcgttatcccctgattctgtggataaccgtattaccgcctttgagtgagctgataccgctcgccgcagccgaa
cgaccgagcgcagcgagtcagtgagcgaggaagcggaagagcgcccaatacgcaaaccgcctctccccgcgcgttg
gccgattcattaatgcagctggcacgacaggtttcccgactggaaagcgggcagtgagcgcaacgcaattaatgtgagtt
agctcactcattaggcaccccaggctttacactttatgcttccggctcgtatgttgtgtggaattgtgagcggataacaatttca
cacaggaaacagctatgaccatgattacgccaagctctagctagaggtcgaccaattctcatgtttgacagcttatcatcgc
agatccgggcaacgttgttgccattgctgcaggcgcagaactggtaggtatggaagatctatacattgaatcaatattggca
attagccatattagtcattggttatatagcataaatcaatattggctattggccattgcatacgttgtatctatatcataatatgtac
atttatattggctcatgtccaatatgaccgccatgttgacattgattattgactagttattaatagtaatcaattacggggtcatta
gttcatagcccatatatggagttccgcgttacataacttacggtaaatggcccgcctggctgaccgcccaacgacccccgc
ccattgacgtcaataatgacgtatgttcccatagtaacgccaatagggactttccattgacgtcaatgggtggagtatttacg
gtaaactgcccacttggcagtacatcaagtgtatcatatgccaagtccgccccctattgacgtcaatgacggtaaatggccc
gcctggcattatgcccagtacatgaccttacgggactttcctacttggcagtacatctacgtattagtcatcgctattaccatgg
tgatgcggttttggcagtacaccaatgggcgtggatagcggtttgactcacggggatttccaagtctccaccccattgacgtc
aatgggagtttgttttggcaccaaaatcaacgggactttccaaaatgtcgtaataaccccgccccgttgacgcaaatgggc
ggtaggcgtgtacggtgggaggtctatataagcagagctcgtttagtgaaccgtcagatcctcactctcttccgcatcgctgt
ctgcgagggccagctgttgggctcgcggttgaggacaaactcttcgcggtctttccagtactcttggatcggaaacccgtcg
gcctccgaacggtactccgccaccgagggacctgagcgagtccgcatcgaccggatcggaaaacctctcgagaaagg
cgtctaaccagtcacagtcgcaaggtaggctgagcaccgtggcgggcggcagcgggtggcggtcggggttgtttctggc
ggaggtgctgctgatgatgtaattaaagtaggcggtcttgagacggcggatggtcgaggtgaggtgtggcaggcttgagat
ccagctgttggggtgagtactccctctcaaaagcgggcattacttctgcgctaagattgtcagtttccaaaaacgaggagga
tttgatattcacctggcccgatctggccatacacttgagtgacaatgacatccactttgcctttctctccacaggtgtccactccc
aggtccaagtttgggcgccaccatggagtttgggctgagctggctttttcttgtcgcgattttaaaaggtgtccagtgt-
gaggtgcagctggtggagtctgggggaggcttggtacagcccggcaggtccctgagactctcctgtgcggcctctggattc
acctttgatgattatgccatgcactgggtccggcaagctccagggaagggcctggaatgggtctcagctatcacttggaata
gtggtcacatagactatgcggactctgtggagggccgattcaccatctccagagacaacgccaagaactccctgtatctgc
aaatgaacagtctgagagclgaggatacggccgtatattactgtgcgaaagtctcgtaccttagcaccgcgtcctcccttga
ctattggggccaaggtaccctggtcaccgtctcgagtgcgtcgaccaagggcccatcggtcttccccctggcaccctcctc
caagagcacctctgggggcacagcggccctgggctgcctggtcaaggactacttccccgaaccggtgacggtgtcgtgg
aactcaggcgccctgaccagcggcgtgcacaccttcccggctgtcctacagtcctcaggactctactccctcagcagcgt
ggtgaccgtgccctccagcagcttgggcacccagacctacatctgcaacgtgaatcacaagcccagcaacaccaaggt
ggacaagaaagttgagcccaaatcttgtgacaaaactcacacatgcccaccgtgcccagcacctgaactcctgggggg
accgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatg
gcaaggagtacaagtgcaaggtctccaacaaagccctcccagcccccatcgagaaaaccatctccaaagccaaagg
gcagccccgagaaccacaggtgtacaccctgcccccatcccgggatgagctgaccaagaaccaggtcagcctgacct
gcctggtcaaaggcttctatcccagcgacatcgccgtggagtgggagagcaatgggcagccggagaacaactacaag
accacgcctoccgtgctggactccgacggctccttcttcctctacagcaagctcaccgtggacaagagcaggtggcagca
ggggaacgtcttctcalgctccgtgatgcatgaggctctgcacaaccactacacgcagaagagcctctccctgtctccggg
taaa-
tgcttcacgccggagagcacagcgctgctggagagtggagtccggaagccgctcggcgagctctctatcggagatcgtg
ttttgagcatgaccgccaacggacaggccgtctacagcgaagtgatcctcttcatggaccgcaacctcgagcagatgcaa
aactttgtgcagctgcacacggacggtggagcagtgctcacggtgacgccggctcacctggttagcgtttggcagccgga
gagccagaagctcacgtttgtgtttgcggatcgcatcgaggagaagaaccaggtgctcgtacgggatgtggagacgggc
gagctgaggccccagcgagtcgtcaaggtgggcagtgtgcgcagtaagggcgtggtcgcgccgctgacccgcgaggg
caccattgtggtcaactcggtggccgccagttgctatgcggtgatcaacagccagtcg-
atggacatgcgcgtgcccgcccagctgctgggcctgctgctgctgtggttccccggctcgcgatgcgacatccagatgacc
cagtctccatcctccctgtctgcatctgtaggggacagagtcaccatcacttgtcgggcaagtcagggcatcagaaattactt
agcctggtatcagcaaaaaccagggaaagcccctaagctcctgstctatgctgcatccactttgcaatcaggggtcccatc
tcggttcagtggcagtggatctgggacagatttcactctcaccatcagcagcctacagcctgaagatgttgcaacttattact
gtcaaaggtataaccgtgcaccgtatacttttggccaggggaccaaggtggaaatcaaacgtacggtggctgcaccatct
gtcttcatcttcccgccatctgatgagcagttgaaatctggaactgcctctgttgtgtcctgctgaataacttctatcccagag
aggccaaagtacagtggaaggtggataacgccctccaatcgggtaactcccaggagagtgtcacagagcaggacagc
aaggacagcacctacagcctcagcagcaccctgacgctgagcaaagcagactacgagaaacacaaagtctacgcct
gcgaagtcacccatcagggcctgagctcgcccgtcacaaagagcttcaacaggggagagtgt-3′
pTT3 Vector-Heavy Chain-Hh-C17-Light Chain

In the following constructs, the only difference from the construct above is the truncation of the C17 region, with the result that cholesterol transferred activity is ablated. The sequences shown are from the end of the D2E7 heavy chain coding region (last 9 base pairs of the HC coding sequence, first line of table) to the 5′ end of the D2E7 light chain coding region (first 9 base pairs of LC coding sequence, last line of table).

TABLE 28
Partial coding sequence of plasmid
pTT3-HC-C17-sc-LC (SEQ ID NO: 156)

Ccgggtaaa-
tgcttcacgccggagagcacagcgctgctggagagtggagtccggaagcc
gctcggcgagctctctatcggagatcgtgttttgagcatgaccgccaacg
gacaggccgtctacagcgaagtgatcctcttcatggaccgcaacctcgag
cagatgcaaaactttgtgcagctgcacacggacggtggagcagtgctca
cggtgacgccggctcacctggttagcgtttggcagccggagagccagaa
gctcacgtttgtgtttgcggatcgcatcgaggagaagaaccaggtgctc
gtacgggatgtggagacgggcgagctgaggccccagcgagtcgtcaagg
tgggcagtgtgcgcagtaagggcgtggtcgcgccgctgacccgcgagg
gcaccattgtggtcaactcggtggccgccagttgc-atggacatg
Heavy Chain 3′ sequence-Hh-C17-Light Chain
5′ sequence

In the following construct, the only difference from construct pTT3-HC-C17-sc-LC above is the mutation of the last two amino acids in the hedgehog C17 region from SC to HN (underlined). The sequences shown are from the end of the D2E7 heavy chain coding region (last 9 base pairs of HC coding sequence, first line of table) to the 5′ end of the D2E7 light chain coding region (last line of table).

TABLE 29
Partial coding sequence from plasmid
pTT3-HC-C17-hn-LC (SEQ ID NO: 157)

ccgggtaaa-
tgcttcacgccggagagcacagcgctgctggagagtggagtccggaagc
cgctcggcgagctctctatcggagatcgtgttttgagcatgaccgccaac
ggacaggccgtctacagcgaagtgatcctcttcatggaccgcaacctcga
gcagatgcaaaactttgtgcagctgcacacggacggtggagcagtgctca
cggtgacgccggctcacctggttagcgtttggcagccggagagccagaag
ctcacgtttgtgtttgcggatcgcatcgaggagaagaaccaggtgctcgt
acgggatgtggagacgggcgagctgaggccccagcgagtcgtcaaggtgg
gcagtgtgcgcagtaagggcgtggtcgcgccgctgacccgcgagggcacc
attgtggtcaactcggtggccgcccacaac-atggacatg
Heavy Chain 3′ sequence-Hh-C17-Mutation-Light
Chain 5′ sequence

In the following construct, the full C25 region of the Hint domain is used, rather than the C17. The sequences shown are from the end of the D2E7 heavy chain coding region (last 9 base pairs of HC coding sequence, first line of table) to the 5′ end of the D2E7 light chain coding region (first 9 base pairs of LC coding sequence, last line of table)

TABLE 29B
Partial coding sequence from pTT3-HC-C25-Hint-LC (SEQ ID NO: 158)

ccgggtaaa-
tgcttcacgccggagagcacagcgctgctggagagtggagtccggaagccgctcggcgagctctctatcggagatcgtg
ttttgagcatgaccgccaacggacaggccgtctacagcgaagtgatcctcttcatggaccgcaacctcgagcagatgcaa
aactttgtgcagctgcacacggacggtggagcagtgctcacggtgacgccggctcacctggttagcgtttggcagccgga
gagccagaagctcacgtttgtgtttgcggatcgcatcgaggagaagaaccaggtgctcgtacgggatgtggagacgggc
gagctgaggccccagcgagtcgtcaaggtgggcagtgtgcgcagtaagggcgtggtcgcgccgctgacccgcgaggg
caccattgtggtcaactcggtggccgccagttgctatgcggtgatcaacagccagtcgctggcccactggggactggctcc
catgcgcctgctgtccacgctggaggcgtggctgcccgccaaggagcagttgcacagttcgccgaaggtggtgagctcg
gcgcagcagcagaatggcatccattggtatgccaatgcgctctacaaggtcaaggactacgttctgccgcagagctggc
gccacgat-
atggacatg
[Heavy Chain 3′ sequence-Hh-C25 domain-Light Chain 5′ sequence;
(SEQ ID NO: 140)
Amino acid sequence of Hh-C25 and related constructs (down arrow indicates cleavage
site;↓:Hh-C17 ↓: Hh-C17sc):
cftpestallesgvrkplgelsigdrvlsmtangqavysevilfmdrnleqmqnfvqlhtdggavltvtpahlvsvwqpesqk
ltfvfadrieeknqvlvrdvetgelrpqrvykygsvrskgvvapltregtivvnsvaasc ↓ ya
vinsqs↓lahwglapmrllstleawlpakeqlhsspkvvssaqqqngihwyanalykvkdyvlpqswrhd

### Example 3

### Antibody Expression with TEV Recognition Sequence for Proteolytic Processing

Constructs and expression vectors are generated to direct the expression of antibodies specific for tumor necrosis factor-α, interleukin-12, interleukin-18 and erythropoietin receptor, with a TEV recognition sequence between the immunoglobulin heavy and light chain sequence segments that comprise the antibody of interest. Preferably, constructs include expression vectors comprising an adenovirus major late promoter and cytomegalovirus enhancer directing transcription of the antibody heavy chain of interest which is preceeded by an in-frame leader sequence. The heavy chain coding sequence is linked to an in-frame furin cleavage site and a TEV recognition sequence (E-P-V-Y-F-Q-G) followed by the coding region for the nuclear-localization-region-deleted TEV protease (Ceriani et al. (1998) Plant Molec Biol. 36:239), followed by a second TEV recognition sequence. The second TEV recognition sequence is linked in-frame to the leader sequence for the antibody light chain linked to the coding region for the antibody light chain of interest and stop codon. The coding region is followed by a polyadenylation signal. Relevant sequences are provided herein below.

TABLE 1
D2E7 (Humira/adalimumab) TEV Expression Vector Complete DNA
Sequence (SEQ ID NO: 44)

GAAGTTCCTATTCCGAAGTTCCTATTCTCTAGACGTTACATAACTTACGGTAAATGGCCCGCCTGGCTG
ACCGCCCAACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGTAC
ATGACCTTATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCTCCACCC
CATTGACGTCAATGGGAGTTTGTTTTGGCACCAAAATCAACGGGACTTTCCAAAATGTCGTAACAACT
CCGCCCCAATGACGCAAATGGGCAGGGAATTCGAGCTCGGTACTCGAGCGGTGTTCCGCGGTCCTCCT
CGTATAGAAACTCGGACCACTCTGAGACGAAGGCTCGCGTCCAGGCCAGCACGAAGGAGGCTAAGTG
GGAGGGGTAGCGGTCGTTGTCCACTAGGGGGTCCACTCGCTCCAGGGTGTGAAGACACATGTCGCCCT
CTTCGGCATCAAGGAAGGTGATTGGTTTATAGGTGTAGGCCACGTGACCGGGTGTTCCTGAAGGGGGG
CTATAAAAGGGGGTGGGGGCGCGTTCGTCCTCACTCTCTTCCGCATCGCTGTCTGCGAGGGCCAGCTG
TTGGGCTCGCGGTTGAGGACAAACTCTTCGCGGTCTTTCCAGTACTCTTGGATCGGAAACCCGTCGGCC
TCCGAACGGTACTCCGCCACCGAGGGACCTGAGCGAGTCCGCATCGACCGGATCGGAAAACCTCTCGA
CTGTTGGGGTGAGTACTCCCTCTCAAAAGCGGGCATGACTTCTGCGCTAAGATTGTCAGTTTCCAAAA
ACGAGGAGGATTTGATATTCACCTGGCCCGCGGTGATGCCTTTGAGGGTGGCCGCGTCCATCTGGTCA
GAAAAGACAATCTTTTTGTTGTCAAGCTTGAGGTGTGGCAGGCTTGAGATCTGGCCATACACTTGAGT
GACAATGACATCCACTTTGCCTTTCTCTCCACAGGTGTCCACTCCCAGGTCCAACCGGAATTGTACCCG
CGGCCAGAGCTTGCCCGGGCGCCACCATGGAGTTTGGGCTGAGCTGGCTTTTTCTTGTCGCGATTTTAA
AAGGTGTCCAGTGTGAGGTGCAGCTGGTGGAGTCTGGGGGAGGCTTGGTACAGCCCGGCAGGTCCCTG
AGACTCTCCTGTGCGGCCTCTGGATTCACCTTTGATGATTATGCCATGCACTGGGTCCGGCAAGCTCCA
GGGAAGGGCCTGGAATGGGTCTCAGCTATCACTTGGAATAGTGGTCACATAGACTATGCGGACTCTGT
GGAGGGCCGATTCACCATCTCCAGAGACAACGCCAAGAACTCCCTGTATCTGCAAATGAACAGTCTGA
GAGCTGAGGATACGGCCGTATATTACTGTGCGAAAGTCTCGTACCTTAGCACCGCGTCCTCCCTTGACT
ATTGGGGCCAAGGTACCCTGGTCACCGTCTCGAGTGCGTCGACCAAGGGCCCATCGGTCTTCCCCCTG
GCACCCTCCTCCAAGAGCACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGCACACCTTCCCGGCTGTCC
TACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAAAGTTGAGCCCAAAT
CTTGTGACAAAACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACATGCGTGGTGGT
GGACGTGAGCCACGAAGACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGTCAGCGTCCTCACCGTCC
TGCACCAGGACTGGCTGAATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTG
GACTCCGACGGCTCCTTCTTCCTCTACAGCAAGCTCACCGTGGACAAGAGCAGGTGGCAGCAGGGGAA
CGTCTTCTCATGCTCCGTGATGCATGAGGCTCTGCACAACCACTACACGCAGAAGAGCCTCTCCCTGTC
TAGGGGTAAACGCGAACCAGTTTATTTCCAGGGGAGCTTGTTTAAGGGGCCGCGTGATTATAACCCAA
TATCGAGTGCCATTTGTCATCTAACGAATGAATCTGATGGGCACACAACATCGTTGTATGGTATTGGTT
TTGGCCCTTTCATCATCACAAACAAGCATTTGTTTAGAAGAAATAATGGTACACTGTTAGTTCAATCAC
TACATGGTGTGTTCAAGGTAAAGAATACCACAACTTTGCAACAACACCTCATTGATGGGAGGGACATG
ATGCTCATTCGCATGCCTAAGGATTTCCCACCATTTCCTCAAAAGCTGAAATTCAGAGAGCCACAAAG
GGAAGAGCGCATATGTCTTGTGACAACCAACTTCCAAACTAAGAGCATGTCTAGCATGGTTTCAGATA
CTAGTTGCACATTCCCTTCATCTGATGGTATATTCTGGAAACATTGGATTCAGACCAAGGATGGGCACT
GTGGTAGCCCGTTGGTGTCAACTAGAGATGGGTTTATTGTTGGTATACACTCAGCATCAAATTTCACCA
ACACAAACAATTATTTTACAAGTGTGCCGAAAGACTTCATGGATTTATTGACAAATCAAGAGGCGCAG
CAATGGGTTAGTGGTTGGCGATTGAATGCTGACTCAGTGTTATGGGGAGGCCACAAAGTTTTCATGAG
CAAACCTGAAGAACCCTTTCAGCCAGTCAAAGAAGCAACTCAACTCATGAGTGAATTAGTCTACTCGC
AAGGGATGGACATGCGCGTGCCCGCCCAGCTGCTGGGCCTGCTGCTGCTGTGGTTCCCCGGCTCGCGA
TGCGACATCCAGATGACCCAGTCTCCATCCTCCCTGTCTGCATCTGTAGGGGACAGAGTCACCATCACT
TGTCGGGCAAGTCAGGGCATCAGAAATTACTTAGCCTGGTATCAGCAAAAACCAGGGAAAGCCCCTA
AGCTCCTGATCTATGCTGCATCCACTTTGCAATCAGGGGTCCCATCTCGGTTCAGTGGCAGTGGATCTG
GGACAGATTTCACTCTCACCATCAGCAGCCTACAGCCTGAAGATGTTGCAACTTATTACTGTCAAAGG
TATAACCGTGCACCGTATACTTTTGGCCAGGGGACCAAGGTGGAAATCAAACGTACGGTGGCTGCACC
ATCTGTCTTCATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCG
CCCGTCACAAAGAGCTTCAACAGGGGAGAGTGTTGAGCGGCCGCGTTTAAACTGAATGAGCGCGTCC
ATCCAGACATGATAAGATACATTGATGAGTTTGGACAAACCACAACTAGAATGCAGTGAAAAAAATG
CTTTATTTGTGAAATTTGTGATGCTATTGCTTTATTTGTAACCATTATAAGCTGCAATAAACAAGTTAA
CAACAACAATTGCATTCATTTTATGTTTCAGGTTCAGGGGGAGGTGTGGGAGGTTTTTTAAAGCAAGT
AAAACCTCTACAAATGTGGTATGGCTGATTATGATCCGGCTGCCTCGCGCGTTTCGGTGATGACGGTG
AAAACCTCTGACACATGCAGCTCCCGGAGACGGTCACAGCTTGTCTGTAAGCGGATGCCGGGAGCAG
ACAAGCCCGTCAGGGCGCGTCAGCGGGTGTTGGCGGGTGTCGGGGCGCAGCCATGACCGGTCGACGG
CGCGCCTTTTTTTTTAATTTTTATTTTATTTTATTTTTGACGCGCCGAAGGCGCGATCTGAGCTCGGTAC
AGCTTGGCTGTGGAATGTGTGTCAGTTAGGGTGTGGAAAGTCCCCAGGCTCCCCAGCAGGCAGAAGTA
TGCAAAGCATGCATCTCAATTAGTCAGCAACCAGGTGTGGAAAGTCCCCAGGCTCCCCAGCAGGCAGA
AGTATGCAAAGCATGCATCTCAATTAGTCAGCAACCATAGTCCCGCCCCTAACTCCGCCCATCCCGCC
CCTAACTCCGCCCAGTTCCGCCCATTCTCCGCCCCATGGCTGACTAATTTTTTTTATTTATGCAGAGGCC
GAGGCCGCCTCGGCCTCTGAGCTATTCCAGAAGTAGTGAGGAGGCTTTTTTGGAGGCCTAGGCTTTTG
CAAAAAGCTCCTCGAGGAACTGAAAAACCAGAAAGTTAACTGGTAAGTTTAGTCTTTTTGTCTTTTATT
TCAGGTCCCGGATCCGGTGGTGGTGCAAATCAAAGAACTGCTCCTCAGTGGATGTTGCCTTTACTTCTA
GGCCTGTACGGAAGTGTTACTTCTGCTCTAAAAGCTGCGGAATTGTACCCGCGGCCTAATACGACTCA
CTATAGGGACTAGTATGGTTCGACCATTGAACTGCATCGTCGCCGTGTCCCAAAATATGGGGATTGGC
AAGAACGGAGACCTACCCTGGCCTCCGCTCAGGAACGAGTTCAAGTACTTCCAAAGAATGACCACAA
CCTCTTCAGTGGAAGGTAAACAGAATCTGGTGATTATGGGTAGGAAAACCTGGTTCTCCATTCCTGAG
AAGAATCGACCTTTAAAGGACAGAATTAATATAGTTCTCAGTAGAGAACTCAAAGAACCACCACGAG
GAGCTCATTTTCTTGCCAAAAGTTTAGATGATGCCTTAAGACTTATTGAACAACCGGAATTGGCAAGT
AAAGTAGACATGGTTTGGATAGTCGGAGGCAGTTCTGTTTACCAGGAAGCCATGAATCAACCAGGCCA
CCTCAGACTCTTTGTGACAAGGATCATGCAGGAATTTGAAAGTGACACGTTTTTCCCAGAAATTGATTT
GGGGAAATATAAACTTCTCCCAGAATACCCAGGCGTCCTCTCTGAGGTCCAGGAGGAAAAAGGCATC
AAGTATAAGTTTGAAGTCTACGAGAAGAAAGACTAAGCGGCCGAGCGCGCGGATCTGGAAACGGGAG
ATGGGGGAGGCTAACTGAAGCACGGAAGGAGACAATACCGGAAGGAACCCGCGCTATGACGGCAAT
AAAAAGACAGAATAAAACGCACGGGTGTTGGGTCGTTTGTTCATAAACGCGGGGTTCGGTCCCAGGG
CTGGCACTCTGTCGATACCCCACCGAGACCCCATTGGGGCCAATACGCCCGCGTTTCTTCCTTTTCCCC
ACCCCACCCCCCAAGTTCGGGTGAAGGCCCAGGGCTCGCAGCCAACGTCGGGGCGGCAGGCCCTGCC
ATAGCCACTGGCCCCGTGGGTTAGGGACGGGGTCCCCCATGGGGAATGGTTTATGGTTCGTGGGGGTT
ATTATTTTGGGCGTTGCGTGGGGTCTGGAGATCCCCCGGGCTGCAGGAATTCCGTTACATTACTTACGG
TAAATGGCCCGCCTGGCTGACCGCCCAACGACCCCCGCCCATTGACGTCAATAATGACGTATGTTCCC
ATAGTAACGCCAATAGGGACTTTCCATTGACGTCAATGGGTGGAGTATTTACGGTAAACTGCCCACTT
GGCAGTACATCAAGTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACATCTACGTATTAGTCA
TCGCTATTACCATGGTGATGCGGTTTTGGCAGTACATCAATGGGCGTGGATAGCGGTTTGACTCACGG
GGATTTCCAAGTCTCCACCCCATTGACGTCAATGGGAGTTTGTTTTGGCACCAAAATCAACGGGACTTT
CCAAAATGTCGTAACAACTCCGCCCCATTGACGCAAAAGGGCGGGAATTCGAGCTCGGTACTCGAGCG
GTGTTCCGCGGTCCTCCTCGTATAGAAACTCGGACCACTCTGAGACGAAGGCTCGCGTCCAGGCCAGC
ACGAAGGAGGCTAAGTGGGAGGGGTAGCGGTCGTTGTCCACTAGGGGGTCCACTCGCTCCAGGGTGT
GAAGACACATGTCGCCCTCTTCGGCATCAAGGAAGGTGATTGGTTTATAGGTGTAGGCCACGTGACCG
GGTGTTCCTGAAGGGGGGCTATAAAAGGGGGTGGGGGCGCGTTCGTCCTCACTCTCTTCCGCATCGCT
GTCTGCGAGGGCCAGCTGTTGGGCTCGCGGTTGAGGACAAACTCTTCGCGGTCTTTCCAGTACTCTTGG
ATCGGAAACCCGTCGGCCTCCGAACGGTACTCCGCCACCGAGGGACCTGAGCGAGTCCGCATCGACCG
GATCGGAAAACCTCTCGACTGTTGGGGTGAGTACTCCCTCTCAAAAGCGGGCATGACTTCTGCGCTAA
GATTGTCAGTTTCCAAAAACGAGGAGGATTTGATATTCACCTGGCCCGCGGTGATGCCTTTGAGGGTG
GCCGCGTCCATCTGGTCAGAAAAGACAATCTTTTTGTTGTCAAGCTTGAGGTGTGGCAGGCTTGAGAT
CTGGCCATACACTTGAGTGACAATGACATCCACTTTGCCTTTCTCTCCACAGGTGTCCACTCCCAGGTC
CAACCGGAATTGTACCCGCGGCCAGAGCTTGCGGGCGCCACCGCGGCCGCGGGGATCCAGACATGAT
AAGATACATTGATGAGTTTGGACAAACCACAACTAGAATGCAGTGAAAAAAATGCTTTATTTGTGAAA
TTTGTGATGCTATTGCTTTATTTGTAACCATTATAAGCTGCAATAAACAAGTTAACAACAACAATTGCA
TTCATTTTATGTTTCAGGTTCAGGGGGAGGTGTGGGAGGTTTTTTCGGATCCTCTTGGCGTAATCATGG
TCATAGCTGTTTCCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGCTCACTGCCCGC
TTTCCAGTCGGGAAACCTGTCGTGCCAGCTGCATTAATGAATCGGCCAACGCGCGGGGAAAGGCGGTT
TGCGTATTGGGCGCTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTCTTCCAT
AGGCTCCGCCCCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCCGACC
GCTGCGCCTTATCCGGTAACTATCGTCTTGAGTCCAACCCGGTAAGACACGACTTATCGCCACTGGCA
GCAGCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGGATCTTCACC
TAGATCCCTTTTAATTAAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGTTCATCCATAGTTGCC
TGACTCCCCGTCGTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTGGTGTCACGCTCG
TCGTTTGGTATGGCTTCATTCAGCTCCGGTTCCCAACGATCAAGGCGAGTTACATGATCCCCCATGTTG
TGCAAAAAAGCGGTTAGCTCCTTCGGTCCTCCGATCGTTGTCAGAAGTAAGTTGGCCGCAGTGTTATC
ACTCATGGTTATGGCAGCACTGCATAATTCTCTTACTGTCATGCCATCCGTAAGATGCTTTTCTGTGAC
TGGTGAGTACTCAACCAAGTCATTCTGAGAATAGTGTATGCGGCGACCGAGTTGCTCTTGCCCGGCGT
CAATACGGGATAATACCGCGCCACATAGCAGAACTTTAAAAGTGCTCATCATTGGAAAACGTTCTTCG
GGGCGAAAACTCTCAAGGATCTTACCGCTGTTGAGATCCAGTTCGATGTAACCCACTCGTGCACCCAA
CTGATCTTCAGCATCTTTTACTTTCACCAGCGTTTCTGGGTGAGCAAAAACAGGAAGGCAAAATGCCG
CAAAAAAGGGAATAAGGGCGACACGGAAATGTTGAATACTCATACTCTTCCTTTTTCAATATTATTGA
AGCATTTATCAGGGTTATTGTCTCATGAGCGGATACATATTTGAATGTATTTAGAAAAATAAACAAAT
AGGGGTTCCGCGCACATTTCCCCGAAAAGTGCCACCTGACGTCTAAGAAACCATTATTATCATGACAT
TAACCTATAAAAATAGGCGTATCACGAGGCCCTTTCGTCTCGCGCGTTTCGGTGATGACGGTGAAAAC
CTCTGACACATGCAGCTCCCGGAGACGGTCACAGCTTGTCTGTAAGCGGATGCCGGGAGCAGACAAGC
CCGTCAGGGCGCGTCAGCGGGTGTTGGCGGGTGTCGGGGCTGGCTTAACTATGCGGCATCAGAGCAGA
TTGTACTGAGAGTGCACCATATGCGGTGTGAAATACCGCACAGATGCGTAAGGAGAAAATACCGCATC
AGGCGCCATTCGCCATTCAGGCTGCGCAACTGTTGGGAAGGGCGATCGGTGCGGGCCTCTTCGCTATT
ACGCCAGCTGGCGAAAGGGGGATGTGCTGCAAGGCGATTAAGTTGGGTAACGCCAGGGTTTTCCCAGT
TACGACGTTGTAAAACGACGGCCAGTGAATT

TABLE 2A
ABT-007 TEV Construct: Coding Sequence for Polyprotein (SEQ ID NO: 32)

ATGGAGTTTGGGCTGAGCTGGCTTTTTCTTGTCGCGATTTTAAAAGGTGTCCAGTGTCAGGTGCAGCTGCAGGAGTCGGGCCC
AGGACTGGTGAAGCCTTCGGAGACCCTGTCCCTCACCTGCACTGTCTCTGGTGCCTCCATCAGTAGTTACTACTGGAGCTGGA
TCCGGCAGCCCCCAGGGAAGGGACTGGAGTGGATTGGGTATATCGGGGGGGAGGGGAGCACCAACTACAACCCCTCCCTCA
AGAGTCGAGTCACCATATCAGTAGACACGTCCAAGAACCAGTTCTCCCTGAAGCTGAGGTCTGTGACCGCTGCGGACACGGCC
GTGTATTACTGTGCGAGAGAGCGACTGGGGATCGGGGACTACTGGGGCCAGGGAACCCTGGTCACCGTCTCCTCAGCGTCGA
CCAAGGGCCCATCGGTCTTCCCCCTGGCGCCCTGCTCTAGAAGCACCTCCGAGAGCACAGCGGCCCTGGGCTGCCTGGTCAA
GGACTACTTCCCCGAACCGGTGACGGTGTCGTGGAACTCAGGCGCTCTGACCAGCGGCGTGCACACCTTCCCAGCTGTCCTG
CAGTCCTCAGGACTCTACTCCCTCAGCAGCGTGGTGACCGTGCCCTCCAGCAACTTCGGCACCCAGACCTACACATGCAACGT
AGATCACAAGCCCAGCAACACCAAGGTGGACAAGACAGTTGAGCGCAAATGTTGTGTCGAGTGCCCACCGTGCCCAGCACCA
CCTGTGGCAGGACCGTCAGTCTTCCTCTTCCCCCCAAAACCCAAGGACACCCTCATGATCTCCCGGACCCCTGAGGTCACGTG
CGTGGTGGTGGACGTGAGCCACGAAGACCCCGAGGTCCAGTTCAACTGGTACGTGGACGGCGTGGAGGTGCATAATGCCAAG
ACAAAGCCACGGGAGGAGCAGTTCAACAGCACGTTCCGTGTGGTCAGCGTCCTCACCGTTGTGCACCAGGACTGGCTGAACG
GCAAGGAGTACAAGTGCAAGGTCTCCAACAAAGGCCTCCCAGCCCCCATCGAGAAAACCATCTCCAAAACCAAAGGGCAGCCC
CGAGAACCACAGGTGTACACCCTGCCCCCATCCCGGGAGGAGATGACCAAGAACCAGGTCAGCCTGACCTGCCTGGTCAAAG
GCTTCTACCCCAGCGACATCGCCGTGGAGTGGGAGAGCAATGGGCAGCCGGAGAACAACTACAAGACCACACCTCCCATGCT
GGACTCCGACGGCTCCTTCTTCCTCTACAGCAAGCTCACCGTGGACAAGAGCAGGTGGCAGCAGGGGAACGTCTTCTCATGCT
CCGTGATGCATGAGGCTCTGCACAACCACTACACGCAGAAGAGCCTCTCCCTGTCTAGGGGTAAACGCGAACCAGTTTATTTC
CAGGGGAGCTTGTTTAAGGGGCCGCGTGATTATAACCCAATATCGAGTGCCATTTGTCATCTAACGAATGAATCTGATGGGCAC
ACAACATCGTTGTATGGTATTGGTTTTGGCCCTTTCATCATCACAAACAAGCATTTGTTTAGAAGAAATAATGGTACACTGTTAGT
TCAATCACTACATGGTGTGTTCAAGGTAAAGAATACCACAACTTTGCAACAACACCTCATTGATGGGAGGGACATGATGCTCATT
CGCATGCCTAAGGATTTCCCACCATTTCCTCAAAAGCTGAAATTCAGAGAGCCACAAAGGGAAGAGCGCATATGTCTTGTGACA
ACCAACTTCCAAACTAAGAGCATGTCTAGCATGGTTTCAGATACTAGTTGCACATTCCCTTCATCTGATGGTATATTCTGGAAAC
ATTGGATTCAGACCAAGGATGGGCACTGTGGTAGCCCGTTGGTGTCAACTAGAGATGGGTTTATTGTTGGTATACACTCAGCAT
CAAATTTCACCAACACAAACAATTATTTTACAAGTGTGCCGAAAGACTTCATGGATTTATTGACAAATCAAGAGGCGCAGCAATG
GGTTAGTGGTTGGCGATTGAATGCTGACTCAGTGTTATGGGGAGGCCACAAAGTTTTCATGAGCAAACCTGAAGAACCCTTTCA
GCCAGTCAAAGAAGCAACTCAACTCATGAGTGAATTAGTCTACTCGCAAGGGATGCGCGTGCCCGCCCAGCTGCTGGGCCTGC
TGCTGCTGTGGTTCCCCGGCTCGCGATGCGACATCCAGCTGACCCAATCTCCATCCTCCCTGTCTGCATCTGTAGGAGACAGA
GTCACCATCACTTGCCGGGCAAGTCAGGGCATTAGAAATGATTTAGGCTGGTATCAGCAGAAACCAGGGAAAGCCCCTAAGCG
CCTGATCTATGCTGCATCCAGTTTGCAAAGTGGGGTCCCATCAAGGTTCAGCGGCAGTGGATCTGGGACAGAATTCACTCTCA
CAATCAGCAGCCTGCAGCCTGAAGATTTTGCAACTTATTACTGTCTACAGCATAATACTTACCCTCCGACGTTCGGCCAAGGGA
CCAAGGTGGAAATCAAACGTACGGTGGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGCACCCTGACGCTGAGCAAAGCAG
ACTACGAGAAACACAAAGTCTACGCCTGCGAAGTCACCCATCAGGGCCTGAGCTCGCCCGTCACAAAGAGCTTCAACAGGGGA
GAGTGTTGA

TABLE 2B
ABT-007 TEV Polyprotein Amino Acid Sequence (SEQ ID NO: 33)

MEFGLSWLFLVAILKGVQCQVQLQESGPGLVKPSETLSLTCTVSGASISSYYWSWIRQPPGKGLEWIGYIGGE
GSTNYNPSLKSRVTISVDTSKNQFSLKLRSVTAADTAVYYCARERLGIGDYWGQGTLVTVSSASTKGPSVFPLA
PCSRSTSESTAALGCLVKDYFPEPVTVSWNSGALTSGVHTFPAVLQSSGLYSLSSWTVPSSNFGTQTYTCNV
DHKPSNTKVDKTVERKCCVECPPCPAPPVAGPSVFLFPPKPKDTLMISRTPEVTCWVDVSHEDPEVQFNWYV
DGVEVHNAKTKPREEQFNSTFRWSVLTWHQDWLNGKEYKCKVSNKGLPAPIEKTISKTKGQPREPQVYTLP
PSREEMTKNQVSLTCLVKGFYPSDIAVEWESNGQPENNYKTTPPMLDSDGSFFLYSKLTVDKSRWQQGNVFS
CSVMHEALHNHYTQKSLSLSRGKREPVYFQGSLFKGPRDYNPISSAICHLTNESDGHTTSLYGIGFGPFIITNKH
LFRRNNGTLLVQSLHGVFKVKNTTTLQQHLIDGRDMMLIRMPKDFPPFPQKLKFREPQREERICLVTTNFQTKS
MSSMVSDTSCTFPSSDGIFWKHWIQTKDGHCGSPLVSTRDGFIVGIHSASNFTNTNNYFTSVPKDFMDLLTNQ
EAQQWVSGWRLNADSVLWGGHKVFMSKPEEPFQPVKEATQLMSELVYSQGMRVPAQLLGLLLLWFPGSRC
DIQLTQSPSSLSASVGDRVTITCRASQGIRNDLGWYQQKPGKAPKRLIYAASSLQSGVPSRFSGSGSGTEFTLT
ISSLQPEDFATYYCLQHNTYPPTFGQGTKVEIKRTVAAPSVFIFPPSDEQLKSGTASWCLLNNFYPREAKVQW
KVDNALQSGNSQESVTEQDSKDSTYSLSSTLTLSKADYEKHKVYACEVTHQGLSSPVTKSFNRGEC*

TABLE 2C
Complete ABT-007 TEV Construct Expression Vector Sequence
(SEQ ID NO: 34)

GAAGTTCCTATTCCGAAGTTCCTATTCTCTAGACGTTACATAACTTACGGTAAATGGCCCGCCTGGCT
GACCGCCCAACGACCCCCGCCCATTGACGTCAATAATGACGTATGTTCCCATAGTAACGCCAATAGG
GACTTTCCATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCATTATGCCC
AGTACATGACCTTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGGATTTCCAAGT
CTCCACCCCATTGACGTCAATGGGAGTTTGTTTTGGCACCAAAATCAACGGGACTTTCCAAAATGTC
GTAACAACTCCGCCCCAATGACGCAAATGGGCAGGGAATTCGAGCTCGGTACTCGAGCGGTGTTCC
GCGGTCCTCCTCGTATAGAAACTCGGACCACTCTGAGACGAAGGCTCGCGTCCAGGCCAGCACGAA
GGAGGCTAAGTGGGAGGGGTAGCGGTCGTTGTCCACTAGGGGGTCCACTCGCTCCAGGGTGTGAA
GACACATGTCGCCCTCTTCGGCATCAAGGAAGGTGATTGGTTTATAGGTGTAGGCCACGTGACCGG
GTGTTCCTGAAGGGGGGCTATAAAAGGGGGTGGGGGCGCGTTCGTCCTCACTCTCTTCCGCATCGC
TGTCTGCGAGGGCCAGCTGTTGGGCTCGCGGTTGAGGACAAACTCTTCGCGGTCTTTCCAGTACTC
TTGGATCGGAAACCCGTCGGCCTCCGAACGGTACTCCGCCACCGAGGGACCTGAGCGAGTCCGCA
TCGACCGGATCGGAAAACCTCTCGACTGTTGGGGTGAGTACTCCCTCTCAAAAGCGGGCATGACTT
CTGCGCTAAGATTGTCAGTTTCCAAAAACGAGGAGGATTTGATATTCACCTGGCCCGCGGTGATGCC
TTTGAGGGTGGCCGCGTCCATCTGGTCAGAAAAGACAATCTTTTTGTTGTCAAGCTTGAGGTGTGGC
AGGCTTGAGATCTGGCCATACACTTGAGTGACAATGACATCCACTTTGCCTTTCTCTCCACAGGTGT
CCACTCCCAGGTCCAACCGGAATTGTACCCGCGGCCAGAGCTTGCCCGGGCGCCACCATGGAGTTT
GGGCTGAGCTGGCTTTTTCTTGTCGCGATTTTAAAAGGTGTCCAGTGTCAGGTGCAGCTGCAGGAGT
CGGGCCCAGGACTGGTGAAGCCTTCGGAGACCCTGTCCCTCACCTGCACTGTCTCTGGTGCCTCCA
TCAGTAGTTACTACTGGAGCTGGATCCGGCAGCCCCCAGGGAAGGGACTGGAGTGGATTGGGTATA
TCGGGGGGGAGGGGAGCACCAACTACAACCCCTCCCTCAAGAGTCGAGTCACCATATCAGTAGACA
CGTCCAAGAACCAGTTCTCCCTGAAGCTGAGGTCTGTGACCGCTGCGGACACGGCCGTGTATTACT
GTGCGAGAGAGCGACTGGGGATCGGGGACTACTGGGGCCAGGGAACCCTGGTCACCGTCTCCTCA
GCGTCGACCAAGGGCCCATCGGTCTTCCCCCTGGCGCCCTGCTCTAGAAGCACCTCCGAGAGCAC
AGCGGCCCTGGGCTGCCTGGTCAAGGACTACTTCCCCGAACCGGTGACGGTGTCGTGGAACTCAG
GCGCTCTGACCAGCGGCGTGCACACCTTCCCAGCTGTCCTGCAGTCCTCAGGACTCTACTCCCTCA
GCAGCGTGGTGACCGTGCCCTCCAGCAACTTCGGCACCCAGACCTACACATGCAACGTAGATCACA
AGCCCAGCAACACCAAGGTGGACAAGACAGTTGAGCGCAAATGTTGTGTCGAGTGCCCACCGTGCC
CAGCACCACCTGTGGCAGGACCGTCAGTCTTCCTCTTCCCCCCAAAACCCAAGGACACCCTCATGAT
CTCCCGGACCCCTGAGGTCACGTGCGTGGTGGTGGACGTGAGCCACGAAGACCCCGAGGTCCAGT
TCAACTGGTACGTGGACGGCGTGGAGGTGCATAATGCCAAGACAAAGCCACGGGAGGAGCAGTTCA
ACAGCACGTTCCGTGTGGTCAGCGTCCTCACCGTTGTGCACCAGGACTGGCTGAACGGCAAGGAGT
ACAAGTGCAAGGTCTCCAACAAAGGCCTCCCAGCCCCCATCGAGAAAACCATCTCCAAAACCAAAG
GGCAGCCCCGAGAACCACAGGTGTACACCCTGCCCCCATCCCGGGAGGAGATGACCAAGAACCAG
GTCAGCCTGACCTGCCTGGTCAAAGGCTTCTACCCCAGCGACATCGCCGTGGAGTGGGAGAGCAAT
GGGCAGCCGGAGAACAACTACAAGACCACACCTCCCATGCTGGACTCCGACGGCTCCTTCTTCCTC
TACAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTAGGGGTAAACGCGAACCA
GTTTATTTCCAGGGGAGCTTGTTTAAGGGGCCGCGTGATTATAACCCAATATCGAGTGCCATTTGTC
ATCTAACGAATGAATCTGATGGGCACACAACATCGTTGTATGGTATTGGTTTTGGCCCTTTCATCATC
ACAAACAAGCATTTGTTTAGAAGAAATAATGGTACACTGTTAGTTCAATCACTACATGGTGTGTTCAA
GGTAAAGAATACCACAACTTTGCAACAACACCTCATTGATGGGAGGGACATGATGCTCATTCGCATG
CCTAAGGATTTCCCACCATTTCCTCAAAAGCTGAAATTCAGAGAGCCACAAAGGGAAGAGCGCATAT
GTCTTGTGACAACCAACTTCCAAACTAAGAGCATGTCTAGCATGGTTTCAGATACTAGTTGCACATTC
CCTTCATCTGATGGTATATTCTGGAAACATTGGATTCAGACCAAGGATGGGCACTGTGGTAGCCCGT
TGGTGTCAACTAGAGATGGGTTTATTGTTGGTATACACTCAGCATCAAATTTCACCAACACAAACAAT
TATTTTACAAGTGTGCCGAAAGACTTCATGGATTTATTGACAAATCAAGAGGCGCAGCAATGGGTTAG
TGGTTGGCGATTGAATGCTGACTCAGTGTTATGGGGAGGCCACAAAGTTTTCATGAGCAAACCTGAA
GAACCCTTTCAGCCAGTCAAAGAAGCAACTCAACTCATGAGTGAATTAGTCTACTCGCAAGGGATGC
GCGTGCCCGCCCAGCTGCTGGGCCTGCTGCTGCTGTGGTTCCCCGGCTCGCGATGCGACATCCAG
CTGACCCAATCTCCATCCTCCCTGTCTGCATCTGTAGGAGACAGAGTCACCATCACTTGCCGGGCAA
GTCAGGGCATTAGAAATGATTTAGGCTGGTATCAGCAGAAACCAGGGAAAGCCCCTAAGCGCCTGA
TCTATGCTGCATCCAGTTTGCAAAGTGGGGTCCCATCAAGGTTCAGCGGCAGTGGATCTGGGACAG
AATTCACTCTCACAATCAGCAGCCTGCAGCCTGAAGATTTTGCAACTTATTACTGTCTACAGCATAAT
ACTTACCCTCCGACGTTCGGCCAAGGGACCAAGGTGGAAATCAAACGTACGGTGGCTGCACCATCT
GTCTTCATCTTCCCGCCATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCCCTCCAATCGGGTAACTC
CCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCT
CGCCCGTCACAAAGAGCTTCAACAGGGGAGAGTGTTGAGCGGCCGCGTTTAAACTGAATGAGCGCG
TCCATCCAGACATGATAAGATACATTGATGAGTTTGGACAAACCACAACTAGAATGCAGTGAAAAAAA
TGCTTTATTTGTGAAATTTGTGATGCTATTGCTTTATTTGTAACCATTATAAGCTGCAATAAACAAGTTA
ACAACAACAATTGCATTCATTTTATGTTTCAGGTTCAGGGGGAGGTGTGGGAGGTTTTTTAAAGCAAG
TAAAACCTCTACAAATGTGGTATGGCTGATTATGATCCGGCTGCCTCGCGCGTTTCGGTGATGACGG
TGAAAACCTCTGACACATGCAGCTCCCGGAGACGGTCACAGCTTGTCTGTAAGCGGATGCCGGGAG
CAGACAAGCCCGTCAGGGCGCGTCAGCGGGTGTTGGCGGGTGTCGGGGCGCAGCCATGACCGGT
CGACGGCGCGCCTTTTTTTTTAATTTTTATTTTATTTTATTTTTGACGCGCCGAAGGCGCGATCTGAG
CTCGGTACAGCTTGGCTGTGGAATGTGTGTCAGTTAGGGTGTGGAAAGTCCCCAGGCTCCCCAGCA
GGCAGAAGTATGCAAAGCATGCATCTCAATTAGTCAGCAACCAGGTGTGGAAAGTCCCCAGGCTCC
CCAGCAGGCAGAAGTATGCAAAGCATGCATCTCAATTAGTCAGCAACCATAGTCCCGCCCCTAACTC
CGCCCATCCCGCCCCTAACTCCGCCCAGTTCCGCCCATTCTCCGCCCCATGGCTGACTAATTTTTTT
TATTTATGCAGAGGCCGAGGCCGCCTCGGCCTCTGAGCTATTCCAGAAGTAGTGAGGAGGCTTTTTT
GGAGGCCTAGGCTTTTGCAAAAAGCTCCTCGAGGAACTGAAAAACCAGAAAGTTAACTGGTAAGTTT
AGTCTTTTTGTCTTTTATTTCAGGTCCCGGATCCGGTGGTGGTGCAAATCAAAGAACTGCTCCTCAGT
GGATGTTGCCTTTACTTCTAGGCCTGTACGGAAGTGTTACTTCTGCTCTAAAAGCTGCGGAATTGTAC
CCGCGGCCTAATACGACTCACTATAGGGACTAGTATGGTTCGACCATTGAACTGCATCGTCGCCGTG
TCCCAAAATATGGGGATTGGCAAGAACGGAGACCTACCCTGGCCTCCGCTCAGGAACGAGTTCAAG
TACTTCCAAAGAATGACCACAACCTCTTCAGTGGAAGGTAAACAGAATCTGGTGATTATGGGTAGGA
AAACCTGGTTCTCCATTCCTGAGAAGAATCGACCTTTAAAGGACAGAATTAATATAGTTCTCAGTAGA
GAACTCAAAGAACCACCACGAGGAGCTCATTTTCTTGCCAAAAGTTTAGATGATGCCTTAAGACTTAT
TGAACAACCGGAATTGGCAAGTAAAGTAGACATGGTTTGGATAGTCGGAGGCAGTTCTGTTTACCAG
GAAGCCATGAATCAACCAGGCCACCTCAGACTCTTTGTGACAAGGATCATGCAGGAATTTGAAAGTG
ACACGTTTTTCCCAGAAATTGATTTGGGGAAATATAAACTTCTCCCAGAATACCCAGGCGTCCTCTCT
GAGGTCCAGGAGGAAAAAGGCATCAAGTATAAGTTTGAAGTCTACGAGAAGAAAGACTAAGCGGCC
GAGCGCGCGGATCTGGAAACGGGAGATGGGGGAGGCTAACTGAAGCACGGAAGGAGACAATACCG
GAAGGAACCCGCGCTATGACGGCAATAAAAAGACAGAATAAAACGCACGGGTGTTGGGTCGTTTGT
TCATAAACGCGGGGTTCGGTCCCAGGGCTGGCACTCTGTCGATACCCCACCGAGACCCCATTGGGG
CCAATACGCCCGCGTTTCTTCCTTTTCCCCACCCCACCCCCCAAGTTCGGGTGAAGGCCCAGGGCT
CGCAGCCAACGTCGGGGCGGCAGGCCCTGCCATAGCCACTGGCCCCGTGGGTTAGGGACGGGGT
CCCCCATGGGGAATGGTTTATGGTTCGTGGGGGTTATTATTTTGGGCGTTGCGTGGGGTCTGGAGA
TCCCCCGGGCTGCAGGAATTCCGTTACATTACTTACGGTAAATGGCCCGCCTGGCTGACCGCCCAA
CGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATATGC
CAAGTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTGATGCGGT
TTTGGCAGTACATCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTCC
GCCCCATTGACGCAAAAGGGCGGGAATTCGAGCTCGGTACTCGAGCGGTGTTCCGCGGTCCTCCTC
GTATAGAAACTCGGACCACTCTGAGACGAAGGCTCGCGTCCAGGCCAGCACGAAGGAGGCTAAGTG
GGAGGGGTAGCGGTCGTTGTCCACTAGGGGGTCCACTCGCTCCAGGGTGTGAAGACACATGTCGC
CCTCTTCGGCATCAAGGAAGGTGATTGGTTTATAGGTGTAGGCCACGTGACCGGGTGTTCCTGAAG
GGGGGCTATAAAAGGGGGTGGGGGCGCGTTCGTCCTCACTCTCTTCCGCATCGCTGTCTGCGAGG
GCCAGCTGTTGGGCTCGCGGTTGAGGACAAACTCTTCGCGGTCTTTCCAGTACTCTTGGATCGGAA
ACCCGTCGGCCTCCGAACGGTACTCCGCCACCGAGGGACCTGAGCGAGTCCGCATCGACCGGATC
GGAAAACCTCTCGACTGTTGGGGTGAGTACTCCCTCTCAAAAGCGGGCATGACTTCTGCGCTAAGAT
TGTCAGTTTCCAAAAACGAGGAGGATTTGATATTCACCTGGCCCGCGGTGATGCCTTTGAGGGTGGC
CGCGTCCATCTGGTCAGAAAAGACAATCTTTTTGTTGTCAAGCTTGAGGTGTGGCAGGCTTGAGATC
TGGCCATACACTTGAGTGACAATGACATCCACTTTGCCTTTCTCTCCACAGGTGTCCACTCCCAGGT
CCAACCGGAATTGTACCCGCGGCCAGAGCTTGCGGGCGCCACCGCGGCCGCGGGGATCCAGACAT
GATAAGATACATTGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTCGGATCCTCTTGGCGTAAT
CATGGTCATAGCTGTTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGGGA
AAGGCGGTTTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAAAAGGCCGCGTTGC
TGGCGTTCTTCCATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTTAAGGGATTTTG
GTCATGAGATTATCAAAAAGGATCTTCACCTAGATCCCTTTTAATTAAAAATGAAGTTTTAAATCAATC
TAAAGTATATATGAGTAAACTTGGTCTGACAGTTACCAATGCTTAATCAGTGAGGCACCTATCTCAGC
GATCTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATTTA
TCAGCAATAAACCAGCCAGCCGGAAGGGCCGAGCGCAGAAGTGGTCCTGCAACTTTATCCGCCTCC
ATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGGGAATAAGGGCGAC
ACGGAAATGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACATTTCCCC
GAAAAGTGCCACCTGACGTCTAAGAAACCATTATTATCATGACATTAACCTATAAAAATAGGCGTATC
ACGAGGCCCTTTCGTCTCGCGCGTTTCGGTGATGACGGTGAAAACCTCTGACACATGCAGCTCCCG
GAGACGGTCACAGCTTGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGATTGTACTGAGAGTGCA
CCATATGCGGTGTGAAATACCGCACAGATGCGTAAGGAGAAAATACCGCATCAGGCGCCATTCGCC
ATTCAGGCTGCGCAACTGTTGGGAAGGGCGATCGGTGCGGGCCTCTTCGCTATTACGCCAGCTGGC
GAAAGGGGGATGTGCTGCAAGGCGATTAAGTTGGGTAACGCCAGGGTTTTCCCAGTTACGACGTTG
TAAAACGACGGCCAGTGAATT

TABLE 3A
Coding Sequence for ABT-874 (J695) TEV Polyprotein (SEQ ID NO: 35)

ATGGAGTTTGGGCTGAGCTGGCTTTTTCTTGTCGCGATTTTAAAAGGTGTCCAGTGTCAGGTGCAGCTGGTGGAGTCTGGGGG
AGGCGTGGTCCAGCCTGGGAGGTCCCTGAGACTCTCCTGTGCAGCGTCTGGATTCACCTTCAGTAGCTATGGCATGCACTGGG
TCCGCCAGGCTCCAGGCAAGGGGCTGGAGTGGGTGGCATTTATACGGTATGATGGAAGTAATAAATACTATGCAGACTCCGTG
AAGGGCCGATTCACCATCTCCAGAGACAATTCCAAGAACACGCTGTATCTGCAGATGAACAGCCTGAGAGCTGAGGACACGGC
TGTGTATTACTGTAAGACCCATGGTAGCCATGACAACTGGGGCCAAGGGACAATGGTCACCGTCTCTTCAGCGTCGACCAAGG
GCCCATCGGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACCCAGACCTACATCTGCAACGTGAATCA
CAAGCCCAGCAACACCAAGGTGGACAAGAAAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCCCCATCGAGAAAACCATCTCCAAAGCCAAAGGGC
AGCCCCGAGAACCACAGGTGTACACCCTGCCCCCATCCCGCGAGGAGATGACCAAGAACCAGGTCAGCCTGACCTGCCTGGT
CAAAGGCTTCTATCCCAGCGACATCGCCGTGGAGTGGGAGAGCAATGGGCAGCCGGAGAACAACTACAAGACCACGCCTCCC
GTGCTGGACTCCGACGGCTCCTTCTTCCTCTACAGCAAGCTCACCGTGGACAAGAGCAGGTGGCAGCAGGGGAACGTCTTCT
CATGCTCCGTGATGCATGAGGCTCTGCACAACCACTACACGCAGAAGAGCCTCTCCCTGTCTAGGGGTAAACGCGAACCAGTT
TATTTCCAGGGGAGCTTGTTTAAGGGGCCGCGTGATTATAACCCAATATCGAGTGCCATTTGTCATCTAACGAATGAATCTGATG
GGCACACAACATCGTTGTATGGTATTGGTTTTGGCCCTTTCATCATCACAAACAAGCATTTGTTTAGAAGAAATAATGGTACACT
GTTAGTTCAATCACTACATGGTGTGTTCAAGGTAAAGAATACCACAACTTTGCAACAACACCTCATTGATGGGAGGGACATGATG
CTCATTCGCATGCCTAAGGATTTCCCACCATTTCCTCAAAAGCTGAAATTCAGAGAGCCACAAAGGGAAGAGCGCATATGTCTT
GTGACAACCAACTTCCAAACTAAGAGCATGTCTAGCATGGTTTCAGATACTAGTTGCACATTCCCTTCATCTGATGGTATATTCT
GGAAACATTGGATTCAGACCAAGGATGGGCACTGTGGTAGCCCGTTGGTGTCAACTAGAGATGGGTTTATTGTTGGTATACACT
CAGCATCAAATTTCACCAACACAAACAATTATTTTACAAGTGTGCCGAAAGACTTCATGGATTTATTGACAAATCAAGAGGCGCA
GCAATGGGTTAGTGGTTGGCGATTGAATGCTGACTCAGTGTTATGGGGAGGCCACAAAGTTTTCATGAGCAAACCTGAAGAAC
CCTTTCAGCCAGTCAAAGAAGCAACTCAACTCATGAGTGAATTAGTCTACTCGCAAGGGATGACTTGGACCCCACTCCTCTTCC
TCACCCTCCTCCTCCACTGCACAGGAAGCTTATCCCAGTCTGTGCTGACTCAGCCCCCCTCAGTGTCTGGGGCCCCCGGGCAG
AGAGTCACCATCTCTTGTTCTGGAAGCAGATCCAACATCGGCAGTAATACTGTAAAGTGGTATCAGCAGCTCCCAGGAACGGCC
CCCAAACTCCTCATCTATTACAATGATCAGCGGCCCTCAGGGGTCCCTGACCGATTCTCTGGATCCAAGTCTGGCACCTCAGCC
TCCCTCGCCATCACTGGGCTCCAGGCTGAAGACGAGGCTGACTATTACTGCCAGTCATATGACAGATACACCCACCCCGCCCT
GCTCTTCGGAACTGGGACCAAGGTCACAGTACTAGGTCAGCCCAAGGCTGCCCCCTCGGTCACTCTGTTCCCGCCCTCCTCTG
AGGAGCTTCAAGCCAACAAGGCCACACTGGTGTGTCTCATAAGTGACTTCTACCCGGGAGCCGTGACAGTGGCCTGGAAGGC
AGATAGCAGCCCCGTCAAGGCGGGAGTGGAGACCACCACACCCTCCAAACAAAGCAACAACAAGTACGCGGCCAGCAGCTAC
CTGAGCCTGACGCCTGAGCAGTGGAAGTCCCACAGAAGCTACAGCTGCCAGGTCACGCATGAAGGGAGCACCGTGGAGAAGA
CAGTGGCCCCTACAGAATGTTCATGA

TABLE 3B
Amino Acid Sequence of ABT-874 (J695) TEV Polyprotein (SEQ ID NO: 36)

MEFGLSWLFLVAILKGVQCQVQLVESGGGVVQPGRSLRLSCAASGFTFSSYGMHWVRQAPGKGLEWVAF
IRYDGSNKYYADSVKGRFTISRDNSKNTLYLQMNSLRAEDTAVYYCKTHGSHDNWGQGTMVTVSSASTK
GPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSRGKREPVYFQGSLFKGPRDYNPISSAICHLTNE
SDGHTTSLYGIGFGPFIITNKHLFRRNNGTLLVQSLHGVFKVKNTTTLQQHLIDGRDMMLIRMPKDFPPFPQ
KLKFREPQREERICLVTTNFQTKSMSSMVSDTSCTFPSSDGIFWKHWIQTKDGHCGSPLVSTRDGFIVGIHSA
SNFTNTNNYFTSVPKDFMDLLTNQEAQQWVSGWRLNADSVLWGGHKVFMSKPEEPFQPVKEATQLMSEL
VYSQGMTWTPLLFLTLLLHCTGSLSQSVLTQPPSVSGAPGQRVTISCSGSRSNIGSNTVKWYQQLPGTAPKL
LIYYNDQRPSGVPDRFSGSKSGTSASLAITGLQAEDEADYYCQSYDRYTHPALLFGTGTKVTVLGQPKAAP
SVTLFPPSSEELQANKATLVCLISDFYPGAVTVAWKADSSPVKAGVETTTPSKQSNNKYAASSYLSLTPEQ
WKSHRSYSCQVTHEGSTVEKTVAPTECS*

TABLE 3C
Complete Nucleotide Sequence of ABT-874 (J695) TEV
Expression Vector (SEQ ID NO: 37)

GAAGTTCCTATTCCGAAGTTCCTATTCTCTAGACGTTACATAACTTACGGTAAATGGCCCGCCTGGCTG
ACCGCCCAACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGTAC
ATGACCTTATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCTCCACCC
CATTGACGTCAATGGGAGTTTGTTTTGGCACCAAAATCAACGGGACTTTCCAAAATGTCGTAACAACT
CCGCCCCAATGACGCAAATGGGCAGGGAATTCGAGCTCGGTACTCGAGCGGTGTTCCGCGGTCCTCCT
CGTATAGAAACTCGGACCACTCTGAGACGAAGGCTCGCGTCCAGGCCAGCACGAAGGAGGCTAAGTG
GGAGGGGTAGCGGTCGTTGTCCACTAGGGGGTCCACTCGCTCCAGGGTGTGAAGACACATGTCGCCCT
CTTCGGCATCAAGGAAGGTGATTGGTTTATAGGTGTAGGCCACGTGACCGGGTGTTCCTGAAGGGGGG
CTATAAAAGGGGGTGGGGGCGCGTTCGTCCTCACTCTCTTCCGCATCGCTGTCTGCGAGGGCCAGCTG
TTGGGCTCGCGGTTGAGGACAAACTCTTCGCGGTCTTTCCAGTACTCTTGGATCGGAAACCCGTCGGCC
TCCGAACGGTACTCCGCCACCGAGGGACCTGAGCGAGTCCGCATCGACCGGATCGGAAAACCTCTCGA
CTGTTGGGGTGAGTACTCCCTCTCAAAAGCGGGCATGACTTCTGCGCTAAGATTGTCAGTTTCCAAAA
ACGAGGAGGATTTGATATTCACCTGGCCCGCGGTGATGCCTTTGAGGGTGGCCGCGTCCATCTGGTCA
GAAAAGACAATCTTTTTGTTGTCAAGCTTGAGGTGTGGCAGGCTTGAGATCTGGCCATACACTTGAGT
GACAATGACATCCACTTTGCCTTTCTCTCCACAGGTGTCCACTCCCAGGTCCAACCGGAATTGTACCCG
CGGCCAGAGCTTGCCCGGGCGCCACCATGGAGTTTGGGCTGAGCTGGCTTTTTCTTGTCGCGATTTTAA
AAGGTGTCCAGTGTCAGGTGCAGCTGGTGGAGTCTGGGGGAGGCGTGGTCCAGCCTGGGAGGTCCCTG
AGACTCTCCTGTGCAGCGTCTGGATTCACCTTCAGTAGCTATGGCATGCACTGGGTCCGCCAGGCTCCA
GGCAAGGGGCTGGAGTGGGTGGCATTTATACGGTATGATGGAAGTAATAAATACTATGCAGACTCCGT
GAAGGGCCGATTCACCATCTCCAGAGACAATTCCAAGAACACGCTGTATCTGCAGATGAACAGCCTGA
GAGCTGAGGACACGGCTGTGTATTACTGTAAGACCCATGGTAGCCATGACAACTGGGGCCAAGGGAC
AATGGTCACCGTCTCTTCAGCGTCGACCAAGGGCCCATCGGTCTTCCCCCTGGCACCCTCCTCCAAGAG
CACCTCTGGGGGCACAGCGGCCCTGGGCTGCCTGGTCAAGGACTACTTCCCCGAACCGGTGACGGTGT
CGTGGAACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTGCAACGT
GAATCACAAGCCCAGCAACACCAAGGTGGACAAGAAAGTTGAGCCCAAATCTTGTGACAAAACTCAC
ACATGCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGAAGA
CCCTGAGGTCAAGTTCAACTGGTACGTGGACGGCGTGGAGGTGCATAATGCCAAGACAAAGCCGCGG
GAGGAGCAGTACAACAGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGCGAGGAGATGACCAA
GAACCAGGTCAGCCTGACCTGCCTGGTCAAAGGCTTCTATCCCAGCGACATCGCCGTGGAGTGGGAGA
GCAATGGGCAGCCGGAGAACAACTACAAGACCACGCCTCCCGTGCTGGACTCCGACGGCTCCTTCTTC
CTCTACAGCAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTAGGGGTAAACGCGAACCAG
TTTATTTCCAGGGGAGCTTGTTTAAGGGGCCGCGTGATTATAACCCAATATCGAGTGCCATTTGTCATC
TAACGAATGAATCTGATGGGCACACAACATCGTTGTATGGTATTGGTTTTGGCCCTTTCATCATCACAA
ACAAGCATTTGTTTAGAAGAAATAATGGTACACTGTTAGTTCAATCACTACATGGTGTGTTCAAGGTA
AAGAATACCACAACTTTGCAACAACACCTCATTGATGGGAGGGACATGATGCTCATTCGCATGCCTAA
GGATTTCCCACCATTTCCTCAAAAGCTGAAATTCAGAGAGCCACAAAGGGAAGAGCGCATATGTCTTG
TGACAACCAACTTCCAAACTAAGAGCATGTCTAGCATGGTTTCAGATACTAGTTGCACATTCCCTTCAT
CTGATGGTATATTCTGGAAACATTGGATTCAGACCAAGGATGGGCACTGTGGTAGCCCGTTGGTGTCA
ACTAGAGATGGGTTTATTGTTGGTATACACTCAGCATCAAATTTCACCAACACAAACAATTATTTTACA
AGTGTGCCGAAAGACTTCATGGATTTATTGACAAATCAAGAGGCGCAGCAATGGGTTAGTGGTTGGCG
ATTGAATGCTGACTCAGTGTTATGGGGAGGCCACAAAGTTTTCATGAGCAAACCTGAAGAACCCTTTC
AGCCAGTCAAAGAAGCAACTCAACTCATGAGTGAATTAGTCTACTCGCAAGGGATGACTTGGACCCCA
CTCCTCTTCCTCACCCTCCTCCTCCACTGCACAGGAAGCTTATCCCAGTCTGTGCTGACTCAGCCCCCCT
CAGTGTCTGGGGCCCCCGGGCAGAGAGTCACCATCTCTTGTTCTGGAAGCAGATCCAACATCGGCAGT
AATACTGTAAAGTGGTATCAGCAGCTCCCAGGAACGGCCCCCAAACTCCTCATCTATTACAATGATCA
GCGGCCCTCAGGGGTCCCTGACCGATTCTCTGGATCCAAGTCTGGCACCTCAGCCTCCCTCGCCATCAC
TGGGCTCCAGGCTGAAGACGAGGCTGACTATTACTGCCAGTCATATGACAGATACACCCACCCCGCCC
TGCTCTTCGGAACTGGGACCAAGGTCACAGTACTAGGTCAGCCCAAGGCTGCCCCCTCGGTCACTCTG
TTCCCGCCCTCCTCTGAGGAGCTTCAAGCCAACAAGGCCACACTGGTGTGTCTCATAAGTGACTTCTAC
CCGGGAGCCGTGACAGTGGCCTGGAAGGCAGATAGCAGCCCCGTCAAGGCGGGAGTGGAGACCACCA
CACCCTCCAAACAAAGCAACAACAAGTACGCGGCCAGCAGCTACCTGAGCCTGACGCCTGAGCAGTG
GAAGTCCCACAGAAGCTACAGCTGCCAGGTCACGCATGAAGGGAGCACCGTGGAGAAGACAGTGGCC
CCTACAGAATGTTCATGAGCGGCCGCGTTTAAACTGAATGAGCGCGTCCATCCAGACATGATAAGATA
CATTGATGAGTTTGGACAAACCACAACTAGAATGCAGTGAAAAAAATGCTTTATTTGTGAAATTTGTG
ATGCTATTGCTTTATTTGTAACCATTATAAGCTGCAATAAACAAGTTAACAACAACAATTGCATTCATT
TTATGTTTCAGGTTCAGGGGGAGGTGTGGGAGGTTTTTTAAAGCAAGTAAAACCTCTACAAATGTGGT
ATGGCTGATTATGATCCGGCTGCCTCGCGCGTTTCGGTGATGACGGTGAAAACCTCTGACACATGCAG
CTCCCGGAGACGGTCACAGCTTGTCTGTAAGCGGATGCCGGGAGCAGACAAGCCCGTCAGGGCGCGT
CAGCGGGTGTTGGCGGGTGTCGGGGCGCAGCCATGACCGGTCGACGGCGCGCCTTTTTTTTTAATTTTT
ATTTTATTTTATTTTTGACGCGCCGAAGGCGCGATCTGAGCTCGGTACAGCTTGGCTGTGGAATGTGTG
TCAGTTAGGGTGTGGAAAGTCCCCAGGCTCCCCAGCAGGCAGAAGTATGCAAAGCATGCATCTCAATT
AGTCAGCAACCAGGTGTGGAAAGTCCCCAGGCTCCCCAGCAGGCAGAAGTATGCAAAGCATGCATCT
CAATTAGTCAGCAACCATAGTCCCGCCCCTAACTCCGCCCATCCCGCCCCTAACTCCGCCCAGTTCCGC
CCATTCTCCGCCCCATGGCTGACTAATTTTTTTTATTTATGCAGAGGCCGAGGCCGCCTCGGCCTCTGA
GCTATTCCAGAAGTAGTGAGGAGGCTTTTTTGGAGGCCTAGGCTTTTGCAAAAAGCTCCTCGAGGAAC
TGAAAAACCAGAAAGTTAACTGGTAAGTTTAGTCTTTTTGTCTTTTATTTCAGGTCCCGGATCCGGTGG
TGGTGCAAATCAAAGAACTGCTCCTCAGTGGATGTTGCCTTTACTTCTAGGCCTGTACGGAAGTGTTAC
TTCTGCTCTAAAAGCTGCGGAATTGTACCCGCGGCCTAATACGACTCACTATAGGGACTAGTATGGTTC
GACCATTGAACTGCATCGTCGCCGTGTCCCAAAATATGGGGATTGGCAAGAACGGAGACCTACCCTGG
CCTCCGCTCAGGAACGAGTTCAAGTACTTCCAAAGAATGACCACAACCTCTTCAGTGGAAGGTAAACA
GAATCTGGTGATTATGGGTAGGAAAACCTGGTTCTCCATTCCTGAGAAGAATCGACCTTTAAAGGACA
GAATTAATATAGTTCTCAGTAGAGAACTCAAAGAACCACCACGAGGAGCTCATTTTCTTGCCAAAAGT
TTAGATGATGCCTTAAGACTTATTGAACAACCGGAATTGGCAAGTAAAGTAGACATGGTTTGGATAGT
CGGAGGCAGTTCTGTTTACCAGGAAGCCATGAATCAACCAGGCCACCTCAGACTCTTTGTGACAAGGA
TCATGCAGGAATTTGAAAGTGACACGTTTTTCCCAGAAATTGATTTGGGGAAATATAAACTTCTCCCA
GAATACCCAGGCGTCCTCTCTGAGGTCCAGGAGGAAAAAGGCATCAAGTATAAGTTTGAAGTCTACGA
GAAGAAAGACTAAGCGGCCGAGCGCGCGGATCTGGAAACGGGAGATGGGGGAGGCTAACTGAAGCA
CGGAAGGAGACAATACCGGAAGGAACCCGCGCTATGACGGCAATAAAAAGACAGAATAAAACGCAC
GGGTGTTGGGTCGTTTGTTCATAAACGCGGGGTTCGGTCCCAGGGCTGGCACTCTGTCGATACCCCACC
GAGACCCCATTGGGGCCAATACGCCCGCGTTTCTTCCTTTTCCCCACCCCACCCCCCAAGTTCGGGTGA
AGGCCCAGGGCTCGCAGCCAACGTCGGGGCGGCAGGCCCTGCCATAGCCACTGGCCCCGTGGGTTAG
GGACGGGGTCCCCCATGGGGAATGGTTTATGGTTCGTGGGGGTTATTATTTTGGGCGTTGCGTGGGGT
CTGGAGATCCCCCGGGCTGCAGGAATTCCGTTACATTACTTACGGTAAATGGCCCGCCTGGCTGACCG
CCCAACGACCCCCGCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACATCAAGTGTATCATA
TGCCAAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGTGATGCGG
TTTTGGCAGTACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACTCCGC
CCCATTGACGCAAAAGGGCGGGAATTCGAGCTCGGTACTCGAGCGGTGTTCCGCGGTCCTCCTCGTAT
AGAAACTCGGACCACTCTGAGACGAAGGCTCGCGTCCAGGCCAGCACGAAGGAGGCTAAGTGGGAGG
GGTAGCGGTCGTTGTCCACTAGGGGGTCCACTCGCTCCAGGGTGTGAAGACACATGTCGCCCTCTTCG
GCATCAAGGAAGGTGATTGGTTTATAGGTGTAGGCCACGTGACCGGGTGTTCCTGAAGGGGGGCTATA
AAAGGGGGTGGGGGCGCGTTCGTCCTCACTCTCTTCCGCATCGCTGTCTGCGAGGGCCAGCTGTTGGG
CTCGCGGTTGAGGACAAACTCTTCGCGGTCTTTCCAGTACTCTTGGATCGGAAACCCGTCGGCCTCCGA
ACGGTACTCCGCCACCGAGGGACCTGAGCGAGTCCGCATCGACCGGATCGGAAAACCTCTCGACTGTT
GGGGTGAGTACTCCCTCTCAAAAGCGGGCATGACTTCTGCGCTAAGATTGTCAGTTTCCAAAAACGAG
GAGGATTTGATATTCACCTGGCCCGCGGTGATGCCTTTGAGGGTGGCCGCGTCCATCTGGTCAGAAAA
GACAATCTTTTTGTTGTCAAGCTTGAGGTGTGGCAGGCTTGAGATCTGGCCATACACTTGAGTGACAAT
GACATCCACTTTGCCTTTCTCTCCACAGGTGTCCACTCCCAGGTCCAACCGGAATTGTACCCGCGGCCA
GAGCTTGCGGGCGCCACCGCGGCCGCGGGGATCCAGACATGATAAGATACATTGATGAGTTTGGACA
AACCACAACTAGAATGCAGTGAAAAAAATGCTTTATTTGTGAAATTTGTGATGCTATTGCTTTATTTGT
AACCATTATAAGCTGCAATAAACAAGTTAACAACAACAATTGCATTCATTTTATGTTTCAGGTTCAGG
GGGAGGTGTGGGAGGTTTTTTCGGATCCTCTTGGCGTAATCATGGTCATAGCTGTTTCCTGTGTGAAAT
TGTTATCCGCTCACAATTCCACACAACATACGAGCCGGAAGCATAAAGTGTAAAGCCTGGGGTGCCTA
ATGAGTGAGCTAACTCACATTAATTGCGTTGCGCTCACTGCCCGCTTTCCAGTCGGGAAACCTGTCGTG
CCAGCTGCATTAATGAATCGGCCAACGCGCGGGGAAAGGCGGTTTGCGTATTGGGCGCTCTTCCGCTT
CCTCGCTCACTGACTCGCTGCGCTCGGTCGTTCGGCTGCGGCGAGCGGTATCAGCTCACTCAAAGGCG
GTAATACGGTTATCCACAGAATCAGGGGATAACGCAGGAAAGAACATGTGAGCAAAAGGCCAGCAAA
AGGCCAGGAACCGTAAAAAGGCCGCGTTGCTGGCGTTCTTCCATAGGCTCCGCCCCCCTGACGAGCAT
CACAAAAATCGACGCTCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAACAGGATTAGCA
GAGCGAGGTATGTAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGCAGAAAAAA
AGGATCTCAAGAAGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCCTTTTAATTAAAAATGAAGTT
TTAAATCAATCTAAAGTATATATGAGTAAACTTGGTCTGACAGTTACCAATGCTTAATCAGTGAGGCA
CCTATCTCAGCGATCTGTCTATTTCGTTCATCCATAGTTGCCTGACTCCCCGTCGTGTAGATAACTACG
ATACGGGAGGGCTTACCATCTGGCCCCAGTGCTGCAATGATACCGCGAGACCCACGCTCACCGGCTCC
AGATTTATCAGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCATTCTGA
GAATAGTGTATGCGGCGACCGAGTTGCTCTTGCCCGGCGTCAATACGGGATAATACCGCGCCACATAG
CAGAACTTTAAAAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTTATTGTCTCATGAG
CGGATACATATTTGAATGTATTTAGAAAAATAAACAAATAGGGGTTCCGCGCACATTTCCCCGAAAAG
TGCCACCTGACGTCTAAGAAACCATTATTATCATGACATTAACCTATAAAAATAGGCGTATCACGAGG
CCCTTTCGTCTCGCGCGTTTCGGTGATGACGGTGAAAACCTCTGACACATGCAGCTCCCGGAGACGGT
CACAGCTTGTCTGTAAGCGGATGCCGGGAGCAGACAAGCCCGTCAGGGCGCGTCAGCGGGTGTTGGC
GGGTGTCGGGGCTGGCTTAACTATGCGGCATCAGAGCAGATTGTACTGAGAGTGCACCATATGCGGTG
TGAAATACCGCACAGATGCGTAAGGAGAAAATACCGCATCAGGCGCCATTCGCCATTCAGGCTGCGC
AACTGTTGGGAAGGGCGATCGGTGCGGGCCTCTTCGCTATTACGCCAGCTGGCGAAAGGGGGATGTGC
TGCAAGGCGATTAAGTTGGGTAACGCCAGGGTTTTCCCAGTTACGACGTTGTAAAACGACGGCCAGTG
AATT

TABLE 4A
Nucleic Acid Sequence Encoding EL246 GG (Anti-E/L Selectin)
TEV Polyprotein (SEQ ID NO: 38)

ATGGAGTTTGGGCTGAGCTGGCTTTTTCTTGTCGCGATTTTAAAAGGTGTCCAGTGCGAGGTGCAGC
TGGTGCAGTCTGGAGCAGAGGTGAAAAAGCCCGGGGAGTCTCTGAAGATCTCCTGTAAGGGGTCCG
GATACGCATTCAGTAGTTCCTGGATCGGCTGGGTGCGCCAGATGCCCGGGAAAGGCCTGGAGTGG
ATGGGGCGGATTTATCCTGGAGATGGAGATACTAACTACAATGGGAAGTTCAAGGGCCAGGTCACC
ATCTCAGCCGACAAGTCCATCAGCACCGCCTACCTGCAGTGGAGCAGCCTGAAGGCTAGCGACACC
GCCATGTATTACTGTGCGAGAGCGCGCGTGGGATCCACGGTCTATGATGGTTACCTCTATGCAATG
GACTACTGGGGTCAAGGTACCTCAGTCACCGTCTCCTCAGCGTCGACCAAGGGCCCATCGGTCTTC
CCCCTGGCACCCTCCTCCAAGAGCACCTCTGGGGGCACAGCGGCCCTGGGCTGCCTGGTCAAGGA
CTACTTCCCCGAACCGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACCAAGGTGGACAAGAA
AGTTGAGCCCAAATCTTGTGACAAAACTCACACATGCCCACCGTGCCCAGCACCTGAAGCCGCGGG
GGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGA
CGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCT
CCAACAAAGCCCTCCCAGCCCCCATCGAGAAAACCATCTCCAAAGCCAAAGGGCAGCCCCGAGAAC
CACAGGTGTACACCCTGCCCCCATCCCGCGAGGAGATGACCAAGAACCAGGTCAGCCTGACCTGCC
TGGTCAAAGGCTTCTATCCCAGCGACATCGCCGTGGAGTGGGAGAGCAATGGGCAGCCGGAGAAC
AACTACAAGACCACGCCTCCCGTGCTGGACTCCGACGGCTCCTTCTTCCTCTACAGCAAGCTCACC
GTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTAGGGGTAAACGCGAACCAGTTTATTTCCAGGGGA
GCTTGTTTAAGGGGCCGCGTGATTATAACCCAATATCGAGTGCCATTTGTCATCTAACGAATGAATCT
GATGGGCACACAACATCGTTGTATGGTATTGGTTTTGGCCCTTTCATCATCACAAACAAGCATTTGTT
TAGAAGAAATAATGGTACACTGTTAGTTCAATCACTACATGGTGTGTTCAAGGTAAAGAATACCACAA
CTTTGCAACAACACCTCATTGATGGGAGGGACATGATGCTCATTCGCATGCCTAAGGATTTCCCACC
ATTTCCTCAAAAGCTGAAATTCAGAGAGCCACAAAGGGAAGAGCGCATATGTCTTGTGACAACCAAC
TTCCAAACTAAGAGCATGTCTAGCATGGTTTCAGATACTAGTTGCACATTCCCTTCATCTGATGGTAT
ATTCTGGAAACATTGGATTCAGACCAAGGATGGGCACTGTGGTAGCCCGTTGGTGTCAACTAGAGAT
GGGTTTATTGTTGGTATACACTCAGCATCAAATTTCACCAACACAAACAATTATTTTACAAGTGTGCCG
AAAGACTTCATGGATTTATTGACAAATCAAGAGGCGCAGCAATGGGTTAGTGGTTGGCGATTGAATG
CTGACTCAGTGTTATGGGGAGGCCACAAAGTTTTCATGAGCAAACCTGAAGAACCCTTTCAGCCAGT
CAAAGAAGCAACTCAACTCATGAGTGAATTAGTCTACTCGCAAGGGATGGACATGCGCGTGCCCGC
CCAGCTGCTGGGCCTGCTGCTGCTGTGGTTCCCCGGCTCGCGATGCGACATCGTGATGACCCAGTC
TCCAGACTCCCTGGCTGTGTCTCTGGGCGAGAGGGCCACCATCAACTGCAAGTCCAGTCAGAGCCT
TTCATATAGAAGCAATCAAAAGAACTCGTTGGCCTGGTACCAGCAGAAACCAGGACAGCCTCCTAAG
CTGCTCATTTACTGGGCTAGCACTAGGGAATCTGGGGTCCCTGACCGATTCAGTGGATCCGGGTCT
GGGACAGATTTCACTCTCACCATCAGCAGCCTGCAGGCTGAAGATGTGGCAGTTTATTACTGTCACC
AATATTATAGCTATCCGTACACGTTCGGAGGGGGGACCAAGGTGGAAATTAAACGTACGGTGGCTG
CACCATCTGTCTTCATCTTCCCGCCATCTGATGAGCAGTTGAAATCTGGAACTGCCTCTGTTGTGTGC
CTGCTGAATAACTTCTATCCCAGAGAGGCCAAAGTACAGTGGAAGGTGGATAACGCCCTCCAATCGG
GTAACTCCCAGGAGAGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTGA

TABLE 4B
Amino Acid Sequence of EL246 GG (Anti-E/L Selectin) TEV
Polyprotein (SEQ ID NO: 39)

MEFGLSWLFLVAILKGVQCEVQLVQSGAEVKKPGESLKISCKGSGYAFSSSWIGWVRQMPGKGLEWMG
RIYPGDGDTNYNGKFKGQVTISADKSISTAYLQWSSLKASDTAMYYCARARVGSTVYDGYLYAMDYWG
QGTSVTVSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSRGKREPVYF
QGSLFKGPRDYNPISSAICHLTNESDGHTTSLYGIGFGPFIITNKHLFRRNNGTLLVQSLHGVFKVKNTTTL
QQHLIDGRDMMLIRMPKDFPPFPQKLKFREPQREERICLVTTNFQTKSMSSMVSDTSCTFPSSDGIFWKH
WIQTKDGHCGSPLVSTRDGFIVGIHSASNFTNTNNYFTSVPKDFMDLLTNQEAQQWVSGWRLNADSVL
WGGHKVFMSKPEEPFQPVKEATQLMSELVYSQGMDMRVPAQLLGLLLLWFPGSRCDIVMTQSPDSLAV
SLGERATINCKSSQSLSYRSNQKNSLAWYQQKPGQPPKLLIYWASTRESGVPDRFSGSGSGTDFTLTISS
LQAEDVAVYYCHQYYSYPYTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC*

TABLE 4C
Complete Nucleotide Sequence for EL246 GG (Anti-E/L Selectin)
TEV Polyprotein Expression Vector (SEQ ID NO: 40)

GAAGTTCCTATTCCGAAGTTCCTATTCTCTAGACGTTACATAACTTACGGTAAATGGCCCGCCTGGCTG
ACCGCCCAACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGTAC
ATGACCTTATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCTCCACCC
CATTGACGTCAATGGGAGTTTGTTTTGGCACCAAAATCAACGGGACTTTCCAAAATGTCGTAACAACT
CCGCCCCAATGACGCAAATGGGCAGGGAATTCGAGCTCGGTACTCGAGCGGTGTTCCGCGGTCCTCCT
CGTATAGAAACTCGGACCACTCTGAGACGAAGGCTCGCGTCCAGGCCAGCACGAAGGAGGCTAAGTG
GGAGGGGTAGCGGTCGTTGTCCACTAGGGGGTCCACTCGCTCCAGGGTGTGAAGACACATGTCGCCCT
CTTCGGCATCAAGGAAGGTGATTGGTTTATAGGTGTAGGCCACGTGACCGGGTGTTCCTGAAGGGGGG
CTATAAAAGGGGGTGGGGGCGCGTTCGTCCTCACTCTCTTCCGCATCGCTGTCTGCGAGGGCCAGCTG
TTGGGCTCGCGGTTGAGGACAAACTCTTCGCGGTCTTTCCAGTACTCTTGGATCGGAAACCCGTCGGCC
TCCGAACGGTACTCCGCCACCGAGGGACCTGAGCGAGTCCGCATCGACCGGATCGGAAAACCTCTCGA
CTGTTGGGGTGAGTACTCCCTCTCAAAAGCGGGCATGACTTCTGCGCTAAGATTGTCAGTTTCCAAAA
ACGAGGAGGATTTGATATTCACCTGGCCCGCGGTGATGCCTTTGAGGGTGGCCGCGTCCATCTGGTCA
GAAAAGACAATCTTTTTGTTGTCAAGCTTGAGGTGTGGCAGGCTTGAGATCTGGCCATACACTTGAGT
GACAATGACATCCACTTTGCCTTTCTCTCCACAGGTGTCCACTCCCAGGTCCAACCGGAATTGTACCCG
CGGCCAGAGCTTGCCCGGGCGCCACCATGGAGTTTGGGCTGAGCTGGCTTTTTCTTGTCGCGATTTTAA
AAGGTGTCCAGTGCGAGGTGCAGCTGGTGCAGTCTGGAGCAGAGGTGAAAAAGCCCGGGGAGTCTCT
GAAGATCTCCTGTAAGGGGTCCGGATACGCATTCAGTAGTTCCTGGATCGGCTGGGTGCGCCAGATGC
CCGGGAAAGGCCTGGAGTGGATGGGGCGGATTTATCCTGGAGATGGAGATACTAACTACAATGGGAA
GTTCAAGGGCCAGGTCACCATCTCAGCCGACAAGTCCATCAGCACCGCCTACCTGCAGTGGAGCAGCC
TGAAGGCTAGCGACACCGCCATGTATTACTGTGCGAGAGCGCGCGTGGGATCCACGGTCTATGATGGT
TACCTCTATGCAATGGACTACTGGGGTCAAGGTACCTCAGTCACCGTCTCCTCAGCGTCGACCAAGGG
CCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCCCTCCA
GCAGCTTGGGCACCCAGACCTACATCTGCAACGTGAATCACAAGCCCAGCAACACCAAGGTGGACAA
GAAAGTTGAGCCCAAATCTTGTGACAAAACTCACACATGCCCACCGTGCCCAGCACCTGAAGCCGCGG
GGGGACCGTCAGTCTTCCTCTTCCCCCCAAAACCCAAGGACACCCTCATGATCTCCCGGACCCCTGAG
GTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCCCGAGAACCACAGGT
GTACACCCTGCCCCCATCCCGCGAGGAGATGACCAAGAACCAGGTCAGCCTGACCTGCCTGGTCAAAG
GCTTCTATCCCAGCGACATCGCCGTGGAGTGGGAGAGCAATGGGCAGCCGGAGAACAACTACAAGAC
CACGCCTCCCGTGCTGGACTCCGACGGCTCCTTCTTCCTCTACAGCAAGCTCACCGTGGACAAGAGCA
GGTGGCAGCAGGGGAACGTCTTCTCATGCTCCGTGATGCATGAGGCTCTGCACAACCACTACACGCAG
AAGAGCCTCTCCCTGTCTAGGGGTAAACGCGAACCAGTTTATTTCCAGGGGAGCTTGTTTAAGGGGCC
GCGTGATTATAACCCAATATCGAGTGCCATTTGTCATCTAACGAATGAATCTGATGGGCACACAACAT
CGTTGTATGGTATTGGTTTTGGCCCTTTCATCATCACAAACAAGCATTTGTTTAGAAGAAATAATGGTA
CACTGTTAGTTCAATCACTACATGGTGTGTTCAAGGTAAAGAATACCACAACTTTGCAACAACACCTC
ATTGATGGGAGGGACATGATGCTCATTCGCATGCCTAAGGATTTCCCACCATTTCCTCAAAAGCTGAA
ATTCAGAGAGCCACAAAGGGAAGAGCGCATATGTCTTGTGACAACCAACTTCCAAACTAAGAGCATG
TCTAGCATGGTTTCAGATACTAGTTGCACATTCCCTTCATCTGATGGTATATTCTGGAAACATTGGATT
CAGACCAAGGATGGGCACTGTGGTAGCCCGTTGGTGTCAACTAGAGATGGGTTTATTGTTGGTATACA
CTCAGCATCAAATTTCACCAACACAAACAATTATTTTACAAGTGTGCCGAAAGACTTCATGGATTTATT
GACAAATCAAGAGGCGCAGCAATGGGTTAGTGGTTGGCGATTGAATGCTGACTCAGTGTTATGGGGA
GGCCACAAAGTTTTCATGAGCAAACCTGAAGAACCCTTTCAGCCAGTCAAAGAAGCAACTCAACTCAT
GAGTGAATTAGTCTACTCGCAAGGGATGGACATGCGCGTGCCCGCCCAGCTGCTGGGCCTGCTGCTGC
TGTGGTTCCCCGGCTCGCGATGCGACATCGTGATGACCCAGTCTCCAGACTCCCTGGCTGTGTCTCTGG
GCGAGAGGGCCACCATCAACTGCAAGTCCAGTCAGAGCCTTTCATATAGAAGCAATCAAAAGAACTC
GTTGGCCTGGTACCAGCAGAAACCAGGACAGCCTCCTAAGCTGCTCATTTACTGGGCTAGCACTAGGG
AATCTGGGGTCCCTGACCGATTCAGTGGATCCGGGTCTGGGACAGATTTCACTCTCACCATCAGCAGC
CTGCAGGCTGAAGATGTGGCAGTTTATTACTGTCACCAATATTATAGCTATCCGTACACGTTCGGAGG
GGGGACCAAGGTGGAAATTAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCACAGAGCAGGACA
GCAAGGACAGCACCTACAGCCTCAGCAGCACCCTGACGCTGAGCAAAGCAGACTACGAGAAACACAA
AGTCTACGCCTGCGAAGTCACCCATCAGGGCCTGAGCTCGCCCGTCACAAAGAGCTTCAACAGGGGAG
AGTGTTGAGCGGCCGCGTTTAAACTGAATGAGCGCGTCCATCCAGACATGATAAGATACATTGATGAG
TTTGGACAAACCACAACTAGAATGCAGTGAAAAAAATGCTTTATTTGTGAAATTTGTGATGCTATTGCT
TTATTTGTAACCATTATAAGCTGCAATAAACAAGTTAACAACAACAATTGCATTCATTTTATGTTTCAG
GTTCAGGGGGAGGTGTGGGAGGTTTTTTAAAGCAAGTAAAACCTCTACAAATGTGGTATGGCTGATTA
TGATCCGGCTGCCTCGCGCGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGGGCGCGTCAGCGGGTGTT
GGCGGGTGTCGGGGCGCAGCCATGACCGGTCGACGGCGCGCCTTTTTTTTTAATTTTTATTTTATTTTAT
TTTTGACGCGCCGAAGGCGCGATCTGAGCTCGGTACAGCTTGGCTGTGGAATGTGTGTCAGTTAGGGT
GTGGAAAGTCCCCAGGCTCCCCAGCAGGCAGAAGTATGCAAAGCATGCATCTCAATTAGTCAGCAACC
AGGTGTGGAAAGTCCCCAGGCTCCCCAGCAGGCAGAAGTATGCAAAGCATGCATCTCAATTAGTCAGC
AACCATAGTCCCGCCCCTAACTCCGCCCATCCCGCCCCTAACTCCGCCCAGTTCCGCCCATTCTCCGCC
CCATGGCTGACTAATTTTTTTTATTTATGCAGAGGCCGAGGCCGCCTCGGCCTCTGAGCTATTCCAGAA
GTAGTGAGGAGGCTTTTTTGGAGGCCTAGGCTTTTGCAAAAAGCTCCTCGAGGAACTGAAAAACCAGA
AAGTTAACTGGTAAGTTTAGTCTTTTTGTCTTTTATTTCAGGTCCCGGATCCGGTGGTGGTGCAAATCA
AAGAACTGCTCCTCAGTGGATGTTGCCTTTACTTCTAGGCCTGTACGGAAGTGTTACTTCTGCTCTAAA
AGCTGCGGAATTGTACCCGCGGCCTAATACGACTCACTATAGGGACTAGTATGGTTCGACCATTGAAC
TGCATCGTCGCCGTGTCCCAAAATATGGGGATTGGCAAGAACGGAGACCTACCCTGGCCTCCGCTCAG
GAACGAGTTCAAGTACTTCCAAAGAATGACCACAACCTCTTCAGTGGAAGGTAAACAGAATCTGGTGA
TTATGGGTAGGAAAACCTGGTTCTCCATTCCTGAGAAGAATCGACCTTTAAAGGACAGAATTAATATA
GTTCTCAGTAGAGAACTCAAAGAACCACCACGAGGAGCTCATTTTCTTGCCAAAAGTTTAGATGATGC
CTTAAGACTTATTGAACAACCGGAATTGGCAAGTAAAGTAGACATGGTTTGGATAGTCGGAGGCAGTT
CTGTTTACCAGGAAGCCATGAATCAACCAGGCCACCTCAGACTCTTTGTGACAAGGATCATGCAGGAA
TTTGAAAGTGACACGTTTTTCCCAGAAATTGATTTGGGGAAATATAAACTTCTCCCAGAATACCCAGG
CGTCCTCTCTGAGGTCCAGGAGGAAAAAGGCATCAAGTATAAGTTTGAAGTCTACGAGAAGAAAGAC
TAAGCGGCCGAGCGCGCGGATCTGGAAACGGGAGATGGGGGAGGCTAACTGAAGCACGGAAGGAGA
CAATACCGGAAGGAACCCGCGCTATGACGGCAATAAAAAGACAGAATAAAACGCACGGGTGTTGGGT
CGTTTGTTCATAAACGCGGGGTTCGGTCCCAGGGCTGGCACTCTGTCGATACCCCACCGAGACCCCATT
GGGGCCAATACGCCCGCGTTTCTTCCTTTTCCCCACCCCACCCCCCAAGTTCGGGTGAAGGCCCAGGGC
TCGCAGCCAACGTCGGGGCGGCAGGCCCTGCCATAGCCACTGGCCCCGTGGGTTAGGGACGGGGTCCC
CCATGGGGAATGGTTTATGGTTCGTGGGGGTTATTATTTTGGGCGTTGCGTGGGGTCTGGAGATCCCCC
GGGCTGCAGGAATTCCGTTACATTACTTACGGTAAATGGCCCGCCTGGCTGACCGCCCAACGACCCCC
GCCCATTGACGTCAATAATGACGTATGTTCCCATAGTAACGCCAATAGGGACTTTCCATTGACGTCAAT
GGGTGGAGTATTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCTTATGGGACTTT
CCTACTTGGCAGTACATCTACGTATTAGTCATCGCTATTACCATGGTGATGCGGTTTTGGCAGTACATC
AATGGGCGTGGATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCCATTGACGCAAA
AGGGCGGGAATTCGAGCTCGGTACTCGAGCGGTGTTCCGCGGTCCTCCTCGTATAGAAACTCGGACCA
CTCTGAGACGAAGGCTCGCGTCCAGGCCAGCACGAAGGAGGCTAAGTGGGAGGGGTAGCGGTCGTTG
TCCACTAGGGGGTCCACTCGCTCCAGGGTGTGAAGACACATGTCGCCCTCTTCGGCATCAAGGAAGGT
GATTGGTTTATAGGTGTAGGCCACGTGACCGGGTGTTCCTGAAGGGGGGCTATAAAAGGGGGTGGGG
GCGCGTTCGTCCTCACTCTCTTCCGCATCGCTGTCTGCGAGGGCCAGCTGTTGGGCTCGCGGTTGAGGA
CAAACTCTTCGCGGTCTTTCCAGTACTCTTGGATCGGAAACCCGTCGGCCTCCGAACGGTACTCCGCCA
CCGAGGGACCTGAGCGAGTCCGCATCGACCGGATCGGAAAACCTCTCGACTGTTGGGGTGAGTACTCC
CTCTCAAAAGCGGGCATGACTTCTGCGCTAAGATTGTCAGTTTCCAAAAACGAGGAGGATTTGATATT
CACCTGGCCCGCGGTGATGCCTTTGAGGGTGGCCGCGTCCATCTGGTCAGAAAAGACAATCTTTTTGTT
GTCAAGCTTGAGGTGTGGCAGGCTTGAGATCTGGCCATACACTTGAGTGACAATGACATCCACTTTGC
CTTTCTCTCCACAGGTGTCCACTCCCAGGTCCAACCGGAATTGTACCCGCGGCCAGAGCTTGCGGGCG
CCACCGCGGCCGCGGGGATCCAGACATGATAAGATACATTGATGAGTTTGGACAAACCACAACTAGA
ATGCAGTGAAAAAAATGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGGTGTGGGA
GGTTTTTTCGGATCCTCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCGTGCCAGCTGCATTAA
TGAATCGGCCAACGCGCGGGGAAAGGCGGTTTGCGTATTGGGCGCTCTTCCGCTTCCTCGCTCACTGA
CTCGCTGCGCTCGGTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAAGGCCAGGAACC
GTAAAAAGGCCGCGTTGCTGGCGTTCTTCCATAGGCTCCGCCCCCCTGACGAGCATCACAAAAATCGA
CGCTCAAGTCAGAGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGTCTTGAGTCCAAC
CCGGTAAGACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACGTTAAGGGATTTTGG
TCATGAGATTATCAAAAAGGATCTTCACCTAGATCCCTTTTAATTAAAAATGAAGTTTTAAATCAATCT
AAAGTATATATGAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGTTGCCA
TTGCTACAGGCATCGTGGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCTGAGAATAGTGTATGC
GGCGACCGAGTTGCTCTTGCCCGGCGTCAATACGGGATAATACCGCGCCACATAGCAGAACTTTAAAA
GTGCTCATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTTCTGGGTG
AGCAAAAACAGGAAGGCAAAATGCCGCAAAAAAGGGAATAAGGGCGACACGGAAATGTTGAATACT
CATACTCTTCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACCTGACGT
CTAAGAAACCATTATTATCATGACATTAACCTATAAAAATAGGCGTATCACGAGGCCCTTTCGTCTCGC
GCGTTTCGGTGATGACGGTGAAAACCTCTGACACATGCAGCTCCCGGAGACGGTCACAGCTTGTCTGT
AAGCGGATGCCGGGAGCAGACAAGCCCGTCAGGGCGCGTCAGCGGGTGTTGGCGGGTGTCGGGGCTG
GCTTAACTATGCGGCATCAGAGCAGATTGTACTGAGAGTGCACCATATGCGGTGTGAAATACCGCACA
GATGCGTAAGGAGAAAATACCGCATCAGGCGCCATTCGCCATTCAGGCTGCGCAACTGTTGGGAAGG
GCGATCGGTGCGGGCCTCTTCGCTATTACGCCAGCTGGCGAAAGGGGGATGTGCTGCAAGGCGATTAA
GTTGGGTAACGCCAGGGTTTTCCCAGTTACGACGTTGTAAAACGACGGCCAGTGAATT

TABLE 5A
Coding Sequence for ABT-325 TEV Polyprotein (SEQ ID NO: 41)

ATGGAGTTTGGGCTGAGCTGGCTTTTCCTTGTCGCGATTTTAAAAGGTGTCCAGTGTGAGGTGCAGCTGGTGCAGTCTGGAAC
AGAGGTGAAAAAACCCGGGGAGTCTCTGAAGATCTCCTGTAAGGGTTCTGGATACACTGTTACCAGTTACTGGATCGGCTGGG
TGCGCCAGATGCCCGGGAAAGGCCTGGAGTGGATGGGATTCATCTATCCTGGTGACTCTGAAACCAGATACAGTCCGACCTTC
CAAGGCCAGGTCACCATCTCAGCCGACAAGTCCTTCAATACCGCCTTCCTGCAGTGGAGCAGTCTAAAGGCCTCGGACACCGC
CATGTATTACTGTGCGCGAGTCGGCAGTGGCTGGTACCCTTATACTTTTGATATCTGGGGCCAAGGGACAATGGTCACCGTCTC
TTCAGCGTCGACCAAGGGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATCTTGTGACAAAACTCACACATGC
CCACCGTGCCCAGCACCTGAAGCCGCGGGGGGACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCCTCCCAGCCCCCATCGAGAAAACCAT
CTCCAAAGCCAAAGGGCAGCCCCGAGAACCACAGGTGTACACCCTGCCCCCATCCCGCGAGGAGATGACCAAGAACCAGGTC
AGCCTGACCTGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAGCAGGTGGCA
GCAGGGGAACGTCTTCTCATGCTCCGTGATGCATGAGGCTCTGCACAACCACTACACGCAGAAGAGCCTCTCCCTGTCTAGGG
GTAAACGCGAACCAGTTTATTTCCAGGGGAGCTTGTTTAAGGGGCCGCGTGATTATAACCCAATATCGAGTGCCATTTGTCATC
TAACGAATGAATCTGATGGGCACACAACATCGTTGTATGGTATTGGTTTTGGCCCTTTCATCATCACAAACAAGCATTTGTTTAG
AAGAAATAATGGTACACTGTTAGTTCAATCACTACATGGTGTGTTCAAGGTAAAGAATACCACAACTTTGCAACAACACCTCATT
GATGGGAGGGACATGATGCTCATTCGCATGCCTAAGGATTTCCCACCATTTCCTCAAAAGCTGAAATTCAGAGAGCCACAAAGG
GAAGAGCGCATATGTCTTGTGACAACCAACTTCCAAACTAAGAGCATGTCTAGCATGGTTTCAGATACTAGTTGCACATTCCCTT
CATCTGATGGTATATTCTGGAAACATTGGATTCAGACCAAGGATGGGCACTGTGGTAGCCCGTTGGTGTCAACTAGAGATGGGT
TTATTGTTGGTATACACTCAGCATCAAATTTCACCAACACAAACAATTATTTTACAAGTGTGCCGAAAGACTTCATGGATTTATTG
ACAAATCAAGAGGCGCAGCAATGGGTTAGTGGTTGGCGATTGAATGCTGACTCAGTGTTATGGGGAGGCCACAAAGTTTTCAT
GAGCAAACCTGAAGAACCCTTTCAGCCAGTCAAAGAAGCAACTCAACTCATGAGTGAATTAGTCTACTCGCAAGGGATGGAAGC
CCCAGCGCAGCTTCTCTTCCTCCTGCTACTCTGGCTCCCAGATACCACTGGAGAAATAGTGATGACGCAGTCTCCAGCCACCCT
GTCTGTGTCTCCAGGGGAAAGAGCCACCCTCTCCTGCAGGGCCAGTGAGAGTATTAGCAGCAACTTAGCCTGGTACCAGCAGA
AACCTGGCCAGGCTCCCAGGCTCTTCATCTATACTGCATCCACCAGGGCCACTGATATCCCAGCCAGGTTCAGTGGCAGTGGG
TCTGGGACAGAGTTCACTCTCACCATCAGCAGCCTGCAGTCTGAAGATTTTGCAGTTTATTACTGTCAGCAGTATAATAACTGGC
CTTCGATCACCTTCGGCCAAGGGACACGACTGGAGATTAAACGAACTGTGGCTGCACCATCTGTCTTCATCTTCCCGCCATCTG
ATGAGCAGTTGAAATCTGGAACTGCTAGCGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGCGAAGTCACCCATCAGGGCCTGAGCTCGCCC
GTCACAAAGAGCTTCAACAGGGGAGAGTGTTGA

TABLE 5B
ABT-325 TEV Polyprotein Amino Acid Sequence (SEQ ID NO: 42)

MEFGLSWLFLVAILKGVQCEVQLVQSGTEVKKPGESLKISCKGSGYTVTSYWIGWVRQMPGKGLEWMGFIYPGDSETRYSPTFQGQ
VTISADKSFNTAFLQWSSLKASDTAMYYCARVGSGWYPYTFDIWGQGTMVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSRGKREPVYFQGSLFKGPRDYNPISSAICHLTNESDGHTTSLYGIGFGPFIITNK
HLFRRNNGTLLVQSLHGVFKVKNTTTLQQHLIDGRDMMLIRMPKDFPPFPQKLKFREPQREERICLVTTNFQTKSMSSMVSDTSCTF
PSSDGIFWKHWIQTKDGHCGSPLVSTRDGFIVGIHSASNFTNTNNYFTSVPKDFMDLLTNQEAQQWVSGWRLNADSVLWGGHKVF
MSKPEEPFQPVKEATQLMSELVYSQGMEAPAQLLFLLLLWLPDTTGEIVMTQSPATLSVSPGERATLSCRASESISSNLAWYQQKPG
QAPRLFIYTASTRATDIPARFSGSGSGTEFTLTISSLQSEDFAVYYCQQYNNWPSITFGQGTRLEIKRTVAAPSVFIFPPSDEQLKSGTA
SVVCLLNNFYPREAKVQWKVDNALQSGNSQESVTEQDSKDSTYSLSSTLTLSKADYEKHKVYACEVTHQGLSSPVTKSFNRGEC*

TABLE 5C
Nucleotide Sequence of Complete ABT-325 TEV Polyprotein
Expression Vector (SEQ ID NO: 43)

GAAGTTCCTATTCCGAAGTTCCTATTCTCTAGACGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGGATTTCCAAGTCTCCACCCCATTGACGT
CAATGGGAGTTTGTTTTGGCACCAAAATCAACGGGACTTTCCAAAATGTCGTAACAACTCCGCCCCAATGACGCAAATGGGCAG
GGAATTCGAGCTCGGTACTCGAGCGGTGTTCCGCGGTCCTCCTCGTATAGAAACTCGGACCACTCTGAGACGAAGGCTCGCGT
CCAGGCCAGCACGAAGGAGGCTAAGTGGGAGGGGTAGCGGTCGTTGTCCACTAGGGGGTCCACTCGCTCCAGGGTGTGAAG
ACACATGTCGCCCTCTTCGGCATCAAGGAAGGTGATTGGTTTATAGGTGTAGGCCACGTGACCGGGTGTTCCTGAAGGGGGGC
TATAAAAGGGGGTGGGGGCGCGTTCGTCCTCACTCTCTTCCGCATCGCTGTCTGCGAGGGCCAGCTGTTGGGCTCGCGGTTG
AGGACAAACTCTTCGCGGTCTTTCCAGTACTCTTGGATCGGAAACCCGTCGGCCTCCGAACGGTACTCCGCCACCGAGGGACC
TGAGCGAGTCCGCATCGACCGGATCGGAAAACCTCTCGACTGTTGGGGTGAGTACTCCCTCTCAAAAGCGGGCATGACTTCTG
CGCTAAGATTGTCAGTTTCCAAAAACGAGGAGGATTTGATATTCACCTGGCCCGCGGTGATGCCTTTGAGGGTGGCCGCGTCC
ATCTGGTCAGAAAAGACAATCTTTTTGTTGTCAAGCTTGAGGTGTGGCAGGCTTGAGATCTGGCCATACACTTGAGTGACAATG
ACATCCACTTTGCCTTTCTCTCCACAGGTGTCCACTCCCAGGTCCAACCGGAATTGTACCCGCGGCCAGAGCTTGCCCGGGCG
CCACCATGGAGTTTGGGCTGAGCTGGCTTTTCCTTGTCGCGATTTTAAAAGGTGTCCAGTGTGAGGTGCAGCTGGTGCAGTCT
GGAACAGAGGTGAAAAAACCCGGGGAGTCTCTGAAGATCTCCTGTAAGGGTTCTGGATACACTGTTACCAGTTACTGGATCGG
CTGGGTGCGCCAGATGCCCGGGAAAGGCCTGGAGTGGATGGGATTCATCTATCCTGGTGACTCTGAAACCAGATACAGTCCG
ACCTTCCAAGGCCAGGTCACCATCTCAGCCGACAAGTCCTTCAATACCGCCTTCCTGCAGTGGAGCAGTCTAAAGGCCTCGGA
CACCGCCATGTATTACTGTGCGCGAGTCGGCAGTGGCTGGTACCCTTATACTTTTGATATCTGGGGCCAAGGGACAATGGTCA
CCGTCTCTTCAGCGTCGACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCAGCGGCGTGCAC
ACCTTCCCGGCTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCCTGCCCCCATCCCGCGAGGAGATGACCAAGA
ACCAGGTCAGCCTGACCTGCCTGGTCAAAGGCTTCTATCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTACAGCAAGCTCACCGTGGACAAGAGCA
GGTGGCAGCAGGGGAACGTCTTCTCATGCTCCGTGATGCATGAGGCTCTGCACAACCACTACACGCAGAAGAGCCTCTCCCTG
TCTAGGGGTAAACGCGAACCAGTTTATTTCCAGGGGAGCTTGTTTAAGGGGCCGCGTGATTATAACCCAATATCGAGTGCCATT
TGTCATCTAACGAATGAATCTGATGGGCACACAACATCGTTGTATGGTATTGGTTTTGGCCCTTTCATCATCACAAACAAGCATT
TGTTTAGAAGAAATAATGGTACACTGTTAGTTCAATCACTACATGGTGTGTTCAAGGTAAAGAATACCACAACTTTGCAACAACA
CCTCATTGATGGGAGGGACATGATGCTCATTCGCATGCCTAAGGATTTCCCACCATTTCCTCAAAAGCTGAAATTCAGAGAGCC
ACAAAGGGAAGAGCGCATATGTCTTGTGACAACCAACTTCCAAACTAAGAGCATGTCTAGCATGGTTTCAGATACTAGTTGCAC
ATTCCCTTCATCTGATGGTATATTCTGGAAACATTGGATTCAGACCAAGGATGGGCACTGTGGTAGCCCGTTGGTGTCAACTAG
AGATGGGTTTATTGTTGGTATACACTCAGCATCAAATTTCACCAACACAAACAATTATTTTACAAGTGTGCCGAAAGACTTCATGG
ATTTATTGACAAATCAAGAGGCGCAGCAATGGGTTAGTGGTTGGCGATTGAATGCTGACTCAGTGTTATGGGGAGGCCACAAA
GTTTTCATGAGCAAACCTGAAGAACCCTTTCAGCCAGTCAAAGAAGCAACTCAACTCATGAGTGAATTAGTCTACTCGCAAGGG
ATGGAAGCCCCAGCGCAGCTTCTCTTCCTCCTGCTACTCTGGCTCCCAGATACCACTGGAGAAATAGTGATGACGCAGTCTCC
AGCCACCCTGTCTGTGTCTCCAGGGGAAAGAGCCACCCTCTCCTGCAGGGCCAGTGAGAGTATTAGCAGCAACTTAGCCTGGT
ACCAGCAGAAACCTGGCCAGGCTCCCAGGCTCTTCATCTATACTGCATCCACCAGGGCCACTGATATCCCAGCCAGGTTCAGT
GGCAGTGGGTCTGGGACAGAGTTCACTCTCACCATCAGCAGCCTGCAGTCTGAAGATTTTGCAGTTTATTACTGTCAGCAGTAT
AATAACTGGCCTTCGATCACCTTCGGCCAAGGGACACGACTGGAGATTAAACGAACTGTGGCTGCACCATCTGTCTTCATCTTC
CCGCCATCTGATGAGCAGTTGAAATCTGGAACTGCTAGCGTTGTGTGCCTGCTGAATAACTTCTATCCCAGAGAGGCCAAAGTA
CAGTGGAAGGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATCAGGGCCT
GAGCTCGCCCGTCACAAAGAGCTTCAACAGGGGAGAGTGTTGAGCGGCCGCGTTTAAACTGAATGAGCGCGTCCATCCAGAC
ATGATAAGATACATTGATGAGTTTGGACAAACCACAACTAGAATGCAGTGAAAAAAATGCTTTATTTGTGAAATTTGTGATGCTAT
TGCTTTATTTGTAACCATTATAAGCTGCAATAAACAAGTTAACAACAACAATTGCATTCATTTTATGTTTCAGGTTCAGGGGGAGG
TGTGGGAGGTTTTTTAAAGCAAGTAAAACCTCTACAAATGTGGTATGGCTGATTATGATCCGGCTGCCTCGCGCGTTTCGGTGA
TGACGGTGAAAACCTCTGACACATGCAGCTCCCGGAGACGGTCACAGCTTGTCTGTAAGCGGATGCCGGGAGCAGACAAGCC
CGTCAGGGCGCGTCAGCGGGTGTTGGCGGGTGTCGGGGCGCAGCCATGACCGGTCGACGGCGCGCCTTTTTTTTTAATTTTT
ATTTTATTTTATTTTTGACGCGCCGAAGGCGCGATCTGAGCTCGGTACAGCTTGGCTGTGGAATGTGTGTCAGTTAGGGTGTGG
AAAGTCCCCAGGCTCCCCAGCAGGCAGAAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATAGTCCCGCCCCTAACTCCGCCCATCCCG
CCCCTAACTCCGCCCAGTTCCGCCCATTCTCCGCCCCATGGCTGACTAATTTTTTTTATTTATGCAGAGGCCGAGGCCGCCTCG
GCCTCTGAGCTATTCCAGAAGTAGTGAGGAGGCTTTTTTGGAGGCCTAGGCTTTTGCAAAAAGCTCCTCGAGGAACTGAAAAAC
CAGAAAGTTAACTGGTAAGTTTAGTCTTTTTGTCTTTTATTTCAGGTCCCGGATCCGGTGGTGGTGCAAATCAAAGAACTGCTCC
TCAGTGGATGTTGCCTTTACTTCTAGGCCTGTACGGAAGTGTTACTTCTGCTCTAAAAGCTGCGGAATTGTACCCGCGGCCTAA
TACGACTCACTATAGGGACTAGTATGGTTCGACCATTGAACTGCATCGTCGCCGTGTCCCAAAATATGGGGATTGGCAAGAACG
GAGACCTACCCTGGCCTCCGCTCAGGAACGAGTTCAAGTACTTCCAAAGAATGACCACAACCTCTTCAGTGGAAGGTAAACAG
AATCTGGTGATTATGGGTAGGAAAACCTGGTTCTCCATTCCTGAGAAGAATCGACCTTTAAAGGACAGAATTAATATAGTTCTCA
GTAGAGAACTCAAAGAACCACCACGAGGAGCTCATTTTCTTGCCAAAAGTTTAGATGATGCCTTAAGACTTATTGAACAACCGGA
ATTGGCAAGTAAAGTAGACATGGTTTGGATAGTCGGAGGCAGTTCTGTTTACCAGGAAGCCATGAATCAACCAGGCCACCTCAG
ACTCTTTGTGACAAGGATCATGCAGGAATTTGAAAGTGACACGTTTTTCCCAGAAATTGATTTGGGGAAATATAAACTTCTCCCA
GAATACCCAGGCGTCCTCTCTGAGGTCCAGGAGGAAAAAGGCATCAAGTATAAGTTTGAAGTCTACGAGAAGAAAGACTAAGC
GGCCGAGCGCGCGGATCTGGAAACGGGAGATGGGGGAGGCTAACTGAAGCACGGAAGGAGACAATACCGGAAGGAACCCGC
GCTATGACGGCAATAAAAAGACAGAATAAAACGCACGGGTGTTGGGTCGTTTGTTCATAAACGCGGGGTTCGGTCCCAGGGCT
GGCACTCTGTCGATACCCCACCGAGACCCCATTGGGGCCAATACGCCCGCGTTTCTTCCTTTTCCCCACCCCACCCCCCAAGT
TCGGGTGAAGGCCCAGGGCTCGCAGCCAACGTCGGGGCGGCAGGCCCTGCCATAGCCACTGGCCCCGTGGGTTAGGGACG
GGGTCCCCCATGGGGAATGGTTTATGGTTCGTGGGGGTTATTATTTTGGGCGTTGCGTGGGGTCTGGAGATCCCCCGGGCTG
CAGGAATTCCGTTACATTACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAATGGCCCGCCTGGCATTATGCCCAGTACA
TGACCTTATGGGACTTTCCTACTTGGCAGTACATCTACGTATTAGTCATCGCTATTACCATGGTGATGCGGTTTTGGCAGTACAT
CAATGGGCGTGGATAGCGGTTTGACTCACGGGGATTTCCAAGTCTCCACCCCATTGACGTCAATGGGAGTTTGTTTTGGCACCA
AAATCAACGGGACTTTCCAAAATGTCGTAACAACTCCGCCCCATTGACGCAAAAGGGCGGGAATTCGAGCTCGGTACTCGAGC
GGTGTTCCGCGGTCCTCCTCGTATAGAAACTCGGACCACTCTGAGACGAAGGCTCGCGTCCAGGCCAGCACGAAGGAGGCTA
AGTGGGAGGGGTAGCGGTCGTTGTCCACTAGGGGGTCCACTCGCTCCAGGGTGTGAAGACACATGTCGCCCTCTTCGGCATC
AAGGAAGGTGATTGGTTTATAGGTGTAGGCCACGTGACCGGGTGTTCCTGAAGGGGGGCTATAAAAGGGGGTGGGGGCGCGT
TCGTCCTCACTCTCTTCCGCATCGCTGTCTGCGAGGGCCAGCTGTTGGGCTCGCGGTTGAGGACAAACTCTTCGCGGTCTTTC
CAGTACTCTTGGATCGGAAACCCGTCGGCCTCCGAACGGTACTCCGCCACCGAGGGACCTGAGCGAGTCCGCATCGACCGGA
TCGGAAAACCTCTCGACTGTTGGGGTGAGTACTCCCTCTCAAAAGCGGGCATGACTTCTGCGCTAAGATTGTCAGTTTCCAAAA
ACGAGGAGGATTTGATATTCACCTGGCCCGCGGTGATGCCTTTGAGGGTGGCCGCGTCCATCTGGTCAGAAAAGACAATCTTT
TTGTTGTCAAGCTTGAGGTGTGGCAGGCTTGAGATCTGGCCATACACTTGAGTGACAATGACATCCACTTTGCCTTTCTCTCCA
CAGGTGTCCACTCCCAGGTCCAACCGGAATTGTACCCGCGGCCAGAGCTTGCGGGCGCCACCGCGGCCGCGGGGATCCAGA
CATGATAAGATACATTGATGAGTTTGGACAAACCACAACTAGAATGCAGTGAAAAAAATGCTTTATTTGTGAAATTTGTGATGCTA
TTGCTTTATTTGTAACCATTATAAGCTGCAATAAACAAGTTAACAACAACAATTGCATTCATTTTATGTTTCAGGTTCAGGGGGAG
GTGTGGGAGGTTTTTTCGGATCCTCTTGGCGTAATCATGGTCATAGCTGTTTCCTGTGTGAAATTGTTATCCGCTCACAATTCCA
CACAACATACGAGCCGGAAGCATAAAGTGTAAAGCCTGGGGTGCCTAATGAGTGAGCTAACTCACATTAATTGCGTTGCGCTCA
CTGCCCGCTTTCCAGTCGGGAAACCTGTCGTGCCAGCTGCATTAATGAATCGGCCAACGCGCGGGGAAAGGCGGTTTGCGTA
TTGGGCGCTCTTCCGCTTCCTCGCTCACTGACTCGCTGCGCTCGGTCGTTCGGCTGCGGCGAGCGGTATCAGCTCACTCAAAG
GCGGTAATACGGTTATCCACAGAATCAGGGGATAACGCAGGAAAGAACATGTGAGCAAAAGGCCAGCAAAAGGCCAGGAACC
GTAAAAAGGCCGCGTTGCTGGCGTTCTTCCATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTGGTAGCGGTGGTTTTTTTGTTTGCAAGCAG
CAGATTACGCGCAGAAAAAAAGGATCTCAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCCTTTTAATTAAAAATGAAGTTTTAAATCAATCTAA
AGTATATATGAGTAAACTTGGTCTGACAGTTACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACCATCTGGCCCCAGTGCTGCAATGATACCGC
GAGACCCACGCTCACCGGCTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTTAATAGTTTGCGCAACGTTGTT
GCCATTGCTACAGGCATCGTGGTGTCACGCTCGTCGTTTGGTATGGCTTCATTCAGCTCCGGTTCCCAACGATCAAGGCGAGTT
ACATGATCCCCCATGTTGTGCAAAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTTTTCTGTGACTGGTGAGTACTCAA
CCAAGTCATTCTGAGAATAGTGTATGCGGCGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAAGGATCTTACCGCTGTTGAGATCCAGTTCG
ATGTAACCCACTCGTGCACCCAACTGATCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCCGCGCACATTTCCCCGAAAA
GTGCCACCTGACGTCTAAGAAACCATTATTATCATGACATTAACCTATAAAAATAGGCGTATCACGAGGCCCTTTCGTCTCGCGC
GTTTCGGTGATGACGGTGAAAACCTCTGACACATGCAGCTCCCGGAGACGGTCACAGCTTGTCTGTAAGCGGATGCCGGGAG
CAGACAAGCCCGTCAGGGCGCGTCAGCGGGTGTTGGCGGGTGTCGGGGCTGGCTTAACTATGCGGCATCAGAGCAGATTGTA
CTGAGAGTGCACCATATGCGGTGTGAAATACCGCACAGATGCGTAAGGAGAAAATACCGCATCAGGCGCCATTCGCCATTCAG
GCTGCGCAACTGTTGGGAAGGGCGATCGGTGCGGGCCTCTTCGCTATTACGCCAGCTGGCGAAAGGGGGATGTGCTGCAAG
GCGATTAAGTTGGGTAACGCCAGGGTTTTCCCAGTTACGACGTTGTAAAACGACGGCCAGTGAATT

TABLE 6A
Coding Sequence for D2E7 LC-LC-HC Polyprotein Construct
(SEQ ID NO: 29)

ATGGACATGCGCGTGCCCGCCCAGCTGCTGGGCCTGCTGCTGCTGTGGTTCCCCGGCTCGCGATGCGACATCCAGATGACCC
AGTCTCCATCCTCCCTGTCTGCATCTGTAGGGGACAGAGTCACCATCACTTGTCGGGCAAGTCAGGGCATCAGAAATTACTTAG
CCTGGTATCAGCAAAAACCAGGGAAAGCCCCTAAGCTCCTGATCTATGCTGCATCCACTTTGCAATCAGGGGTCCCATCTCGGT
TCAGTGGCAGTGGATCTGGGACAGATTTCACTCTCACCATCAGCAGCCTACAGCCTGAAGATGTTGCAACTTATTACTGTCAAA
GGTATAACCGTGCACCGTATACTTTTGGCCAGGGGACCAAGGTGGAAATCAAACGTACGGTGGCTGCACCATCTGTCTTCATCT
TCCCGCCATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAAGGTGTAAGAGACTTCTCAAGTTGGCAGGAGACGTTGAGTCCAACCC
TGGGCCCATGGACATGCGCGTGCCCGCCCAGCTGCTGGGCCTGCTGCTGCTGTGGTTCCCCGGCTCGCGATGCGACATCCA
GATGACCCAGTCTCCATCCTCCCTGTCTGCATCTGTAGGGGACAGAGTCACCATCACTTGTCGGGCAAGTCAGGGCATCAGAA
ATTACTTAGCCTGGTATCAGCAAAAACCAGGGAAAGCCCCTAAGCTCCTGATCTATGCTGCATCCACTTTGCAATCAGGGGTCC
CATCTCGGTTCAGTGGCAGTGGATCTGGGACAGATTTCACTCTCACCATCAGCAGCCTACAGCCTGAAGATGTTGCAACTTATT
ACTGTCAAAGGTATAACCGTGCACCGTATACTTTTGGCCAGGGGACCAAGGTGGAAATCAAACGTACGGTGGCTGCACCATCT
GTCTTCATCTTCCCGCCATCTGATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAAGGTGTAAGAGACTTCTCAAGTTGGCAGGAGACGTTGA
GTCCAACCCTGGGCCCATGGAGTTTGGGCTGAGCTGGCTTTTTCTTGTCGCGATTTTAAAAGGTGTCCAGTGTGAGGTGCAGC
TGGTGGAGTCTGGGGGAGGCTTGGTACAGCCCGGCAGGTCCCTGAGACTCTCCTGTGCGGCCTCTGGATTCACCTTTGATGAT
TATGCCATGCACTGGGTCCGGCAAGCTCCAGGGAAGGGCCTGGAATGGGTCTCAGCTATCACTTGGAATAGTGGTCACATAGA
CTATGCGGACTCTGTGGAGGGCCGATTCACCATCTCCAGAGACAACGCCAAGAACTCCCTGTATCTGCAAATGAACAGTCTGA
GAGCTGAGGATACGGCCGTATATTACTGTGCGAAAGTCTCGTACCTTAGCACCGCGTCCTCCCTTGACTATTGGGGCCAAGGT
ACCCTGGTCACCGTCTCGAGTGCGTCGACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGTGCCCTCCAGCAG
CTTGGGCACCCAGACCTACATCTGCAACGTGAATCACAAGCCCAGCAACACCAAGGTGGACAAGAAAGTTGAGCCCAAATCTT
GTGACAAAACTCACACATGCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCGGGAGGAGCAGTACAACAGCACGTACCGTGT
GGTCAGCGTCCTCACCGTCCTGCACCAGGACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCACAGGTGTACACCCTGCCCCCATCCCGGGATG
AGCTGACCAAGAACCAGGTCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTCCTCTACAGCAAGCTCACCG
TGGACAAGAGCAGGTGGCAGCAGGGGAACGTCTTCTCATGCTCCGTGATGCATGAGGCTCTGCACAACCACTACACGCAGAA
GAGCCTCTCCCTGTCTCCGGGTAAATGA

TABLE 6B
D2E7 LC-LC-HC Polyprotein Amino Acid Sequence (SEQ ID NO: 30)

MDMRVPAQLLGLLLLWFPGSRCDIQMTQSPSSLSASVGDRVTITCRASQGIRNYLAWYQQKPGKAPKLLIYAASTLQSGVPSRFSGS
GSGTDFTLTISSLQPEDVATYYCQRYNRAPYTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGRCKRLLKLAGDVESNPGPMDMRVPAQL
LGLLLLWFPGSRCDIQMTQSPSSLSASVGDRVTITCRASQGIRNYLAVVYQQKPGKAPKLLIYAASTLQSGVPSRFSGSGSGTDFTLTI
SSLQPEDVATYYCQRYNRAPYTFGQGTKVEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGRCKRLLKLAGDVESNPGPMEFGLSWLFLVAILKGVQC
EVQLVESGGGLVQPGRSLRLSCAASGFTFDDYAMHVVVRQAPGKGLEVVVSAITWNSGHIDYADSVEGRFTISRDNAKNSLYLQMNS
LRAEDTAVYYCAKVSYLSTASSLDYWGQGTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGK*

TABLE 6C
Complete Nucleotide Sequence of the D2E7 LC-LC-HC
Polyprotein Expression Vector DNA Sequence(SEQ ID NO: 31)

GAAGTTCCTATTCCGAAGTTCCTATTCTCTAGACGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGGATTTCCAAGTCTCCACCCCATTGACGT
CAATGGGAGTTTGTTTTGGCACCAAAATCAACGGGACTTTCCAAAATGTCGTAACAACTCCGCCCCAATGACGCAAATGGGCAG
GGAATTCGAGCTCGGTACTCGAGCGGTGTTCCGCGGTCCTCCTCGTATAGAAACTCGGACCACTCTGAGACGAAGGCTCGCGT
CCAGGCCAGCACGAAGGAGGCTAAGTGGGAGGGGTAGCGGTCGTTGTCCACTAGGGGGTCCACTCGCTCCAGGGTGTGAAG
ACACATGTCGCCCTCTTCGGCATCAAGGAAGGTGATTGGTTTATAGGTGTAGGCCACGTGACCGGGTGTTCCTGAAGGGGGGC
TATAAAAGGGGGTGGGGGCGCGTTCGTCCTCACTCTCTTCCGCATCGCTGTCTGCGAGGGCCAGCTGTTGGGCTCGCGGTTG
AGGACAAACTCTTCGCGGTCTTTCCAGTACTCTTGGATCGGAAACCCGTCGGCCTCCGAACGGTACTCCGCCACCGAGGGACC
TGAGCGAGTCCGCATCGACCGGATCGGAAAACCTCTCGACTGTTGGGGTGAGTACTCCCTCTCAAAAGCGGGCATGACTTCTG
CGCTAAGATTGTCAGTTTCCAAAAACGAGGAGGATTTGATATTCACCTGGCCCGCGGTGATGCCTTTGAGGGTGGCCGCGTCC
ATCTGGTCAGAAAAGACAATCTTTTTGTTGTCAAGCTTGAGGTGTGGCAGGCTTGAGATCTGGCCATACACTTGAGTGACAATG
ACATCCACTTTGCCTTTCTCTCCACAGGTGTCCACTCCCAGGTCCAACCGGAATTGTACCCGCGGCCAGAGCTTGCCCGGGCG
CCACCATGGACATGCGCGTGCCCGCCCAGCTGCTGGGCCTGCTGCTGCTGTGGTTCCCCGGCTCGCGATGCGACATCCAGAT
GACCCAGTCTCCATCCTCCCTGTCTGCATCTGTAGGGGACAGAGTCACCATCACTTGTCGGGCAAGTCAGGGCATCAGAAATT
ACTTAGCCTGGTATCAGCAAAAACCAGGGAAAGCCCCTAAGCTCCTGATCTATGCTGCATCCACTTTGCAATCAGGGGTCCCAT
CTCGGTTCAGTGGCAGTGGATCTGGGACAGATTTCACTCTCACCATCAGCAGCCTACAGCCTGAAGATGTTGCAACTTATTACT
GTCAAAGGTATAACCGTGCACCGTATACTTTTGGCCAGGGGACCAAGGTGGAAATCAAACGTACGGTGGCTGCACCATCTGTC
TTCATCTTCCCGCCATCTGATGAGCAGTTGAAATCTGGAACTGCCTCTGTTGTGTGCCTGCTGAATAACTTCTATCCCAGAGAG
GCCAAAGTACAGTGGAAGGTGGATAACGCCCTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAAGGTGTAAGAGACTTCTCAAGTTGGCAGGAGACGTTGAGT
CCAACCCTGGGCCCATGGACATGCGCGTGCCCGCCCAGCTGCTGGGCCTGCTGCTGCTGTGGTTCCCCGGCTCGCGATGCG
ACATCCAGATGACCCAGTCTCCATCCTCCCTGTCTGCATCTGTAGGGGACAGAGTCACCATCACTTGTCGGGCAAGTCAGGGC
ATCAGAAATTACTTAGCCTGGTATCAGCAAAAACCAGGGAAAGCCCCTAAGCTCCTGATCTATGCTGCATCCACTTTGCAATCA
GGGGTCCCATCTCGGTTCAGTGGCAGTGGATCTGGGACAGATTTCACTCTCACCATCAGCAGCCTACAGCCTGAAGATGTTGC
AACTTATTACTGTCAAAGGTATAACCGTGCACCGTATACTTTTGGCCAGGGGACCAAGGTGGAAATCAAACGTACGGTGGCTGC
ACCATCTGTCTTCATCTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCACAGAGCAGGACA
GCAAGGACAGCACCTACAGCCTCAGCAGCACCCTGACGCTGAGCAAAGCAGACTACGAGAAACACAAAGTCTACGCCTGCGA
AGTCACCCATCAGGGCCTGAGCTCGCCCGTCACAAAGAGCTTCAACAGGGGAAGGTGTAAGAGACTTCTCAAGTTGGCAGGA
GACGTTGAGTCCAACCCTGGGCCCATGGAGTTTGGGCTGAGCTGGCTTTTTCTTGTCGCGATTTTAAAAGGTGTCCAGTGTGAG
GTGCAGCTGGTGGAGTCTGGGGGAGGCTTGGTACAGCCCGGCAGGTCCCTGAGACTCTCCTGTGCGGCCTCTGGATTCACCT
TTGATGATTATGCCATGCACTGGGTCCGGCAAGCTCCAGGGAAGGGCCTGGAATGGGTCTCAGCTATCACTTGGAATAGTGGT
CACATAGACTATGCGGACTCTGTGGAGGGCCGATTCACCATCTCCAGAGACAACGCCAAGAACTCCCTGTATCTGCAAATGAA
CAGTCTGAGAGCTGAGGATACGGCCGTATATTACTGTGCGAAAGTCTCGTACCTTAGCACCGCGTCCTCCCTTGACTATTGGG
GCCAAGGTACCCTGGTCACCGTCTCGAGTGCGTCGACCAAGGGCCCATCGGTCTTCCCCCTGGCACCCTCCTCCAAGAGCAC
CTCTGGGGGCACAGCGGCCCTGGGCTGCCTGGTCAAGGACTACTTCCCCGAACCGGTGACGGTGTCGTGGAACTCAGGCGC
CCTGACCAGCGGCGTGCACACCTTCCCGGCTGTCCTACAGTCCTCAGGACTCTACTCCCTCAGCAGCGTGGTGACCGTGCCCT
CCAGCAGCTTGGGCACCCAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCAGTCTTCCTCTTCCCCCC
AAAACCCAAGGACACCCTCATGATCTCCCGGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCTATCCCAGCGACATCGCCGTGGAGTGGG
AGAGCAATGGGCAGCCGGAGAACAACTACAAGACCACGCCTCCCGTGCTGGACTCCGACGGCTCCTTCTTCCTCTACAGCAAG
CTCACCGTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTAAATGAGAATTAGTCTACTCGCAAGGGGCGGCCGCGTTTAAACTGAATGAGCGCG
TCCATCCAGACATGATAAGATACATTGATGAGTTTGGACAAACCACAACTAGAATGCAGTGAAAAAAATGCTTTATTTGTGAAATT
TGTGATGCTATTGCTTTATTTGTAACCATTATAAGCTGCAATAAACAAGTTAACAACAACAATTGCATTCATTTTATGTTTCAGGTT
CAGGGGGAGGTGTGGGAGGTTTTTTAAAGCAAGTAAAACCTCTACAAATGTGGTATGGCTGATTATGATCCGGCTGCCTCGCG
CGTTTCGGTGATGACGGTGAAAACCTCTGACACATGCAGCTCCCGGAGACGGTCACAGCTTGTCTGTAAGCGGATGCCGGGA
GCAGACAAGCCCGTCAGGGCGCGTCAGCGGGTGTTGGCGGGTGTCGGGGCGCAGCCATGACCGGTCGACGGCGCGCCTTTT
TTTTTAATTTTTATTTTATTTTATTTTTGACGCGCCGAAGGCGCGATCTGAGCTCGGTACAGCTTGGCTGTGGAATGTGTGTCAGT
TAGGGTGTGGAAAGTCCCCAGGCTCCCCAGCAGGCAGAAGTATGCAAAGCATGCATCTCAATTAGTCAGCAACCAGGTGTGGA
AAGTCCCCAGGCTCCCCAGCAGGCAGAAGTATGCAAAGCATGCATCTCAATTAGTCAGCAACCATAGTCCCGCCCCTAACTCC
GCCCATCCCGCCCCTAACTCCGCCCAGTTCCGCCCATTCTCCGCCCCATGGCTGACTAATTTTTTTTATTTATGCAGAGGCCGA
GGCCGCCTCGGCCTCTGAGCTATTCCAGAAGTAGTGAGGAGGCTTTTTTGGAGGCCTAGGCTTTTGCAAAAAGCTCCTCGAGG
AACTGAAAAACCAGAAAGTTAACTGGTAAGTTTAGTCTTTTTGTCTTTTATTTCAGGTCCCGGATCCGGTGGTGGTGCAAATCAA
AGAACTGCTCCTCAGTGGATGTTGCCTTTACTTCTAGGCCTGTACGGAAGTGTTACTTCTGCTCTAAAAGCTGCGGAATTGTACC
CGCGGCCTAATACGACTCACTATAGGGACTAGTATGGTTCGACCATTGAACTGCATCGTCGCCGTGTCCCAAAATATGGGGATT
GGCAAGAACGGAGACCTACCCTGGCCTCCGCTCAGGAACGAGTTCAAGTACTTCCAAAGAATGACCACAACCTCTTCAGTGGA
AGGTAAACAGAATCTGGTGATTATGGGTAGGAAAACCTGGTTCTCCATTCCTGAGAAGAATCGACCTTTAAAGGACAGAATTAAT
ATAGTTCTCAGTAGAGAACTCAAAGAACCACCACGAGGAGCTCATTTTCTTGCCAAAAGTTTAGATGATGCCTTAAGACTTATTG
AACAACCGGAATTGGCAAGTAAAGTAGACATGGTTTGGATAGTCGGAGGCAGTTCTGTTTACCAGGAAGCCATGAATCAACCAG
GCCACCTCAGACTCTTTGTGACAAGGATCATGCAGGAATTTGAAAGTGACACGTTTTTCCCAGAAATTGATTTGGGGAAATATAA
ACTTCTCCCAGAATACCCAGGCGTCCTCTCTGAGGTCCAGGAGGAAAAAGGCATCAAGTATAAGTTTGAAGTCTACGAGAAGAA
AGACTAAGCGGCCGAGCGCGCGGATCTGGAAACGGGAGATGGGGGAGGCTAACTGAAGCACGGAAGGAGACAATACCGGAA
GGAACCCGCGCTATGACGGCAATAAAAAGACAGAATAAAACGCACGGGTGTTGGGTCGTTTGTTCATAAACGCGGGGTTCGGT
CCCAGGGCTGGCACTCTGTCGATACCCCACCGAGACCCCATTGGGGCCAATACGCCCGCGTTTCTTCCTTTTCCCCACCCCAC
CCCCCAAGTTCGGGTGAAGGCCCAGGGCTCGCAGCCAACGTCGGGGCGGCAGGCCCTGCCATAGCCACTGGCCCCGTGGGT
TAGGGACGGGGTCCCCCATGGGGAATGGTTTATGGTTCGTGGGGGTTATTATTTTGGGCGTTGCGTGGGGTCTGGAGATCCCC
CGGGCTGCAGGAATTCCGTTACATTACTTACGGTAAATGGCCCGCCTGGCTGACCGCCCAACGACCCCCGCCCATTGACGTCA
ATAATGACGTATGTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTGGCATTATGCCC
AGTACATGACCTTATGGGACTTTCCTACTTGGCAGTACATCTACGTATTAGTCATCGCTATTACCATGGTGATGCGGTTTTGGCA
GTACATCAATGGGCGTGGATAGCGGTTTGACTCACGGGGATTTCCAAGTCTCCACCCCATTGACGTCAATGGGAGTTTGTTTTG
GCACCAAAATCAACGGGACTTTCCAAAATGTCGTAACAACTCCGCCCCATTGACGCAAAAGGGCGGGAATTCGAGCTCGGTAC
TCGAGCGGTGTTCCGCGGTCCTCCTCGTATAGAAACTCGGACCACTCTGAGACGAAGGCTCGCGTCCAGGCCAGCACGAAGG
AGGCTAAGTGGGAGGGGTAGCGGTCGTTGTCCACTAGGGGGTCCACTCGCTCCAGGGTGTGAAGACACATGTCGCCCTCTTC
GGCATCAAGGAAGGTGATTGGTTTATAGGTGTAGGCCACGTGACCGGGTGTTCCTGAAGGGGGGCTATAAAAGGGGGTGGGG
GCGCGTTCGTCCTCACTCTCTTCCGCATCGCTGTCTGCGAGGGCCAGCTGTTGGGCTCGCGGTTGAGGACAAACTCTTCGCG
GTCTTTCCAGTACTCTTGGATCGGAAACCCGTCGGCCTCCGAACGGTACTCCGCCACCGAGGGACCTGAGCGAGTCCGCATC
GACCGGATCGGAAAACCTCTCGACTGTTGGGGTGAGTACTCCCTCTCAAAAGCGGGCATGACTTCTGCGCTAAGATTGTCAGT
TTCCAAAAACGAGGAGGATTTGATATTCACCTGGCCCGCGGTGATGCCTTTGAGGGTGGCCGCGTCCATCTGGTCAGAAAAGA
CAATCTTTTTGTTGTCAAGCTTGAGGTGTGGCAGGCTTGAGATCTGGCCATACACTTGAGTGACAATGACATCCACTTTGCCTTT
CTCTCCACAGGTGTCCACTCCCAGGTCCAACCGGAATTGTACCCGCGGCCAGAGCTTGCGGGCGCCACCGCGGCCGCGGGG
ATCCAGACATGATAAGATACATTGATGAGTTTGGACAAACCACAACTAGAATGCAGTGAAAAAAATGCTTTATTTGTGAAATTTGT
GATGCTATTGCTTTATTTGTAACCATTATAAGCTGCAATAAACAAGTTAACAACAACAATTGCATTCATTTTATGTTTCAGGTTCAG
GGGGAGGTGTGGGAGGTTTTTTCGGATCCTCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATGAGTGAGCTAACTCACATTAATTGCGTT
GCGCTCACTGCCCGCTTTCCAGTCGGGAAACCTGTCGTGCCAGCTGCATTAATGAATCGGCCAACGCGCGGGGAAAGGCGGT
TTGCGTATTGGGCGCTCTTCCGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAGCAAAAGGCCAGCAAAAGGCCA
GGAACCGTAAAAAGGCCGCGTTGCTGGCGTTCTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGAACAGTATTTGGTATCTGCGCTCTGCTG
AAGCCAGTTACCTTCGGAAAAAGAGTTGGTAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTTTTCTACGGGGTCTGACGCTCAGTGGAACGAA
AACTCACGTTAAGGGATTTTGGTCATGAGATTATCAAAAAGGATCTTCACCTAGATCCCTTTTAATTAAAAATGAAGTTTTAAATC
AATCTAAAGTATATATGAGTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGCCCCAGTGCTGCAATGA
TACCGCGAGACCCACGCTCACCGGCTCCAGATTTATCAGCAATAAACCAGCCAGCCGGAAGGGCCGAGCGCAGAAGTGGTCC
TGCAACTTTATCCGCCTCCATCCAGTCTATTAATTGTTGCCGGGAAGCTAGAGTAAGTAGTTCGCCAGTTAATAGTTTGCGCAAC
GTTGTTGCCATTGCTACAGGCATCGTGGTGTCACGCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGGTCCTCCGATCGTTGTCAGAAGTAAGTTGGCCGCA
GTGTTATCACTCATGGTTATGGCAGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCAGCGTTTCTGGGTGAGCAAAAACAGGAA
GGCAAAATGCCGCAAAAAAGGGAATAAGGGCGACACGGAAATGTTGAATACTCATACTCTTCCTTTTTCAATATTATTGAAGCAT
TTATCAGGGTTATTGTCTCATGAGCGGATACATATTTGAATGTATTTAGAAAAATAAACAAATAGGGGTTCCGCGCACATTTCCC
CGAAAAGTGCCACCTGACGTCTAAGAAACCATTATTATCATGACATTAACCTATAAAAATAGGCGTATCACGAGGCCCTTTCGTC
TCGCGCGTTTCGGTGATGACGGTGAAAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGCTTAACTATGCGGCATCAGAGCAG
ATTGTACTGAGAGTGCACCATATGCGGTGTGAAATACCGCACAGATGCGTAAGGAGAAAATACCGCATCAGGCGCCATTCGCC
ATTCAGGCTGCGCAACTGTTGGGAAGGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTTACGACGTTGTAAAACGACGGCCAGTGAATT

### Example 4

### Expression of Antibody as Polyprotein with Internal Cleavable Signal Peptide Construct

Further embodiments are created of coding sequences, expression vectors, and methods for the expression of an antibody. A primary expression construct comprises a polyprotein with an internal cleavable signal peptide, so that expression and subsequent cleavage results in the formation of a multi-chain (e.g., two-chain) antibody molecule.

TABLE 7A
Coding Sequence for D2E7 internal cleavable signal peptide construct (SEQ ID NO: 45)

atggagtttgggctgagctggctttttcttgtcgcgattttaaaaggtgtccagtgtgaggtgcagctggtggagtctgggggaggcttggtac
agcccggcaggtccctgagactctcctgtgcggcctctggattcacctttgatgattatgccatgcactgggtccggcaagctccagggaag
ggcctggaatgggtctcagctatcacttggaatagtggtcacatagactatgcggactctgtggagggccgattcaccatctccagagacaa
cgccaagaactccctgtatctgcaaatgaacagtctgagagctgaggatacggccgtatattactgtgcgaaagtctcgtaccttagcaccg
cgtcctcccttgactattggggccaaggtaccctggtcaccgtctcgagtgcgtcgaccaagggcccatcggtcttccccctggcaccctcc
tccaagagcacctctgggggcacagcggccctgggctgcctggtcaaggactacttccccgaaccggtgacggtgtcgtggaactcagg
cgccctgaccagcggcgtgcacaccttcccggctgtcctacagtcctcaggactctactccctcagcagcgtggtgaccgtgccctccagc
agcttgggcacccagacctacatctgcaacgtgaatcacaagcccagcaacaccaaggtggacaagaaagttgagcccaaatcttgtgac
aaaactcacacatgcccaccgtgcccagcacctgaactcctggggggaccgtcagtcttcctcttccccccaaaacccaaggacaccctca
tgatctcccggacccctgaggtcacatgcgtggtggtggacgtgagccacgaagaccctgaggtcaagttcaactggtacgtggacggcg
tggaggtgcataatgccaagacaaagccgcgggaggagcagtacaacagcacgtaccgtgtggtcagcgtcctcaccgtcctgcaccag
gactggctgaatggcaaggagtacaagtgcaaggtctccaacaaagccctcccagcccccatcgagaaaaccatctccaaagccaaagg
gcagccccgagaaccacaggtgtacaccctgcccccatcccgggatgagctgaccaagaaccaggtcagcctgacctgcctggtcaaag
gcttctatcccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtgatgcatgagg
ctctgcacaaccactacacgcagaagagcctctccctgtctaggggtaaacgcatgggacgaatggcaatgaaatggttagttgttataatat
gtttctctataacaagtcaacctgcttctgctatggacatgcgcgtgcccgcccagctgctgggcctgctgctgctgtggttccccggctcgc
gatgcgacatccagatgacccagtctccatcctccctgtctgcatctgtaggggacagagtcaccatcacttgtcgggcaagtcagggcatc
agaaattacttagcctggtatcagcaaaaaccagggaaagcccctaagctcctgatctatgctgcatccactttgcaatcaggggtcccatct
cggttcagtggcagtggatctgggacagatttcactctcaccatcagcagcctacagcctgaagatgttgcaacttattactgtcaaaggtata
accgtgcaccgtatacttttggccaggggaccaaggtggaaatcaaacgtacggtggctgcaccatctgtcttcatcttcccgccatctgatg
agcagttgaaatctggaactgcctctgttgtgtgcctgctgaataacttctatcccagagaggccaaagtacagtggaaggtggataacgcc
ctccaatcgggtaactcccaggagagtgtcacagagcaggacagcaaggacagcacctacagcctcagcagcaccctgacgctgagca
aagcagactacgagaaacacaaagtctacgcctgcgaagtcacccatcagggcctgagctcgcccgtcacaaagagcttcaacagggga
gagtgttga

TABLE 7B
Amino Acid Sequence of the D2E7 Internal Cleavable Signal
Peptide Polyprotein (SEQ ID NO: 46)

MEFGLSWLFLVAILKGVQCEVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAKVSYLSTASSLDYWGQGTLVTV
SSASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSRGKRMGRMAMKWLVVIIC
FSITSQPASAMDMRVPAQLLGLLLLWFPGSRCDIQMTQSPSSLSASVGDRVTITCRASQGIRNYLAWYQQ
KPGKAPKLLIYAASTLQSGVPSRFSGSGSGTDFTLTISSLQPEDVATYYCQRYNRAPYTFGQGTKVEIKRT
VAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC*

TABLE 7C
Complete D2E7 Internal Cleavable Signal Peptide Polyprotein
Expression Vector DNA Sequence (SEQ ID NO: 47)

gaagttcctattccgaagttcctattctctagacgttacataacttacggtaaatggcccgcctggctgaccgcccaacgacc
cccgcccattgacgtcaataatgacgtatgttcccatagtaacgccaatagggactttccattgacgtcaatgggtggagtat
ttacggtaaactgcccacttggcagtacatcaagtgtatcatatgccaagtacgccccctattgacgtcaatgacggtaaat
ggcccgcctggcattatgcccagtacatgaccttatgggactttcctacttggcagtacatctacgtattagtcatcgctattac
catggtgatgcggttttggcagtacatcaatgggcgtggatagcggtttgactcacggggatttccaagtctccaccccattg
acgtcaatgggagtttgttttggcaccaaaatcaacgggactttccaaaatgtcgtaacaactccgccccaatgacgcaaa
tgggcagggaattcgagctcggtactcgagcggtgttccgcggtcctcctcgtatagaaactcggaccactctgagacga
aggctcgcgtccaggccagcacgaaggaggctaagtgggaggggtagcggtcgttgtccactagggggtccactcgct
ccagggtgtgaagacacatgtcgccctcttcggcatcaaggaaggtgattggtttataggtgtaggccacgtgaccgggtg
ttcctgaaggggggctataaaagggggtgggggcgcgttcgtcctcactctcttccgcatcgctgtctgcgagggccagct
gttgggctcgcggttgaggacaaactcttcgcggtctttccagtactcttggatcggaaacccgtcggcctccgaacggtact
ccgccaccgagggacctgagcgagtccgcatcgaccggatcggaaaacctctcgactgttggggtgagtactccctctc
aaaagcgggcatgacttctgcgctaagattgtcagtttccaaaaacgaggaggatttgatattcacctggcccgcggtgat
gcctttgagggtggccgcgtccatctggtcagaaaagacaatctttttgttgtcaagcttgaggtgtggcaggcttgagatctg
gccatacacttgagtgacaatgacatccactttgcctttctctccacaggtgtccactcccaggtccaaccggaattgtaccc
gcggccagagcttgcccgggcgccaccatggagtttgggctgagctggctttttcttgtcgcgattttaaaaggtgtccagtgt
gaggtgcagctggtggagtctgggggaggcttggtacagcccggcaggtccctgagactctcctgtgcggcctctggattc
acctttgatgattatgccatgcactgggtccggcaagctccagggaagggcctggaatgggtctcagctatcacttggaata
gtggtcacatagactatgcggactctgtggagggccgattcaccatctccagagacaacgccaagaactccctgtatctgc
aaatgaacagtctgagagctgaggatacggccgtatattactgtgcgaaagtctcgtaccttagcaccgcgtcctcccttga
ctattggggccaaggtaccctggtcaccgtctcgagtgcgtcgaccaagggcccatcggtcttccccctggcaccctcctc
caagagcacctctgggggcacagcggccctgggctgcctggtcaaggactacttccccgaaccggtgacggtgtcgtgg
aactcaggcgccctgaccagcggcgtgcacaccttcccggctgtcctacagtcctcaggactctactccctcagcagcgt
ggtgaccgtgccctccagcagcttgggcacccagacctacatctgcaacgtgaatcacaagcccagcaacaccaaggt
ggacaagaaagttgagcccaaatcttgtgacaaaactcacacatgcccaccgtgcccagcacctgaactcctgggggg
accgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaa
agccgcgggaggagcagtacaacagcacgtaccgtgtggtcagcgtcctcaccgtcctgcaccaggactggctgaatg
gcaaggagtacaagtgcaaggtctccaacaaagccctcccagcccccatcgagaaaaccatctccaaagccaaagg
gcagccccgagaaccacaggtgtacaccctgcccccatcccgggatgagctgaccaagaaccaggtcagcctgacct
gcctggtcaaaggcttctatcccagcgacatcgccgtggagtgggagagcaatgggcagccggagaacaactacaag
accacgcctcccgtgctggactccgacggctccttcttcctctacagcaagctcaccgtggacaagagcaggtggcagca
ggggaacgtcttctcatgctccgtgatgcatgaggctctgcacaaccactacacgcagaagagcctctccctgtctagggg
taaacgcatgggacgaatggcaatgaaatggttagttgttataatatgtttctctataacaagtcaacctgcttctgctatggac
atgcgcgtgcccgcccagctgctgggcctgctgctgctgtggttccccggctcgcgatgcgacatccagatgacccagtct
ccatcctccctgtctgcatctgtaggggacagagtcaccatcacttgtcgggcaagtcagggcatcagaaattacttagcct
ggtatcagcaaaaaccagggaaagcccctaagctcctgatctatgctgcatccactttgcaatcaggggtcccatctcggtt
cagtggcagtggatctgggacagatttcactctcaccatcagcagcctacagcctgaagatgttgcaacttattactgtcaa
aggtataaccgtgcaccgtatacttttggccaggggaccaaggtggaaatcaaacgtacggtggctgcaccatctgtcttc
atcttcccgccatctgatgagcagttgaaatctggaactgcctctgttgtgtgcctgctgaataacttctatcccagagaggcc
aaagtacagtggaaggtggataacgccctccaatcgggtaactcccaggagagtgtcacagagcaggacagcaagg
acagcacctacagcctcagcagcaccctgacgctgagcaaagcagactacgagaaacacaaagtctacgcctgcga
agtcacccatcagggcctgagctcgcccgtcacaaagagcttcaacaggggagagtgttgagcggccgcgtttaaactg
aatgagcgcgtccatccagacatgataagatacattgatgagtttggacaaaccacaactagaatgcagtgaaaaaaat
gctttatttgtgaaatttgtgatgctattgctttatttgtaaccattataagctgcaataaacaagttaacaacaacaattgcattc
attttatgtttcaggttcagggggaggtgtgggaggttttttaaagcaagtaaaacctctacaaatgtggtatggctgattatga
tccggctgcctcgcgcgtttcggtgatgacggtgaaaacctctgacacatgcagctcccggagacggtcacagcttgtctgt
aagcggatgccgggagcagacaagcccgtcagggcgcgtcagcgggtgttggcgggtgtcggggcgcagccatgac
cggtcgacggcgcgcctttttttttaatttttattttattttatttttgacgcgccgaaggcgcgatctgagctcggtacagcttggct
gtggaatgtgtgtcagttagggtgtggaaagtccccaggctccccagcaggcagaagtatgcaaagcatgcatctcaatt
agtcagcaaccaggtgtggaaagtccccaggctccccagcaggcagaagtatgcaaagcatgcatctcaattagtcag
caaccatagtcccgcccctaactccgcccatcccgcccctaactccgcccagttccgcccattctccgccccatggctgac
taattttttttatttatgcagaggccgaggccgcctcggcctctgagctattccagaagtagtgaggaggcttttttggaggcct
aggcttttgcaaaaagctcctcgaggaactgaaaaaccagaaagttaactggtaagtttagtctttttgtcttttatttcaggtcc
cggatccggtggtggtgcaaatcaaagaactgctcctcagtggatgttgcctttacttctaggcctgtacggaagtgttacttct
gctctaaaagctgcggaattgtacccgcggcctaatacgactcactatagggactagtatggttcgaccattgaactgcatc
gtcgccgtgtcccaaaatatggggattggcaagaacggagacctaccctggcctccgctcaggaacgagttcaagtactt
ccaaagaatgaccacaacctcttcagtggaaggtaaacagaatctggtgattatgggtaggaaaacctggttctccattcc
tgagaagaatcgacctttaaaggacagaattaatatagttctcagtagagaactcaaagaaccaccacgaggagctcatt
ttcttgccaaaagtttagatgatgccttaagacttattgaacaaccggaattggcaagtaaagtagacatggtttggatagtc
ggaggcagttctgtttaccaggaagccatgaatcaaccaggccacctcagactctttgtgacaaggatcatgcaggaattt
gaaagtgacacgtttttcccagaaattgatttggggaaatataaacttctcccagaatacccaggcgtcctctctgaggtcca
ggaggaaaaaggcatcaagtataagtttgaagtctacgagaagaaagactaagcggccgagcgcgcggatctggaa
acgggagatgggggaggctaactgaagcacggaaggagacaataccggaaggaacccgcgctatgacggcaataa
aaagacagaataaaacgcacgggtgttgggtcgtttgttcataaacgcggggttcggtcccagggctggcactctgtcgat
accccaccgagaccccattggggccaatacgcccgcgtttcttccttttccccaccccaccccccaagttcgggtgaaggc
ccagggctcgcagccaacgtcggggcggcaggccctgccatagccactggccccgtgggttagggacggggtccccc
atggggaatggtttatggttcgtgggggttattattttgggcgttgcgtggggtctggagatcccccgggctgcaggaattccg
ttacattacttacggtaaatggcccgcctggctgaccgcccaacgacccccgcccattgacgtcaataatgacgtatgttcc
catagtaacgccaatagggactttccattgacgtcaatgggtggagtatttacggtaaactgcccacttggcagtacatcaa
gtgtatcatatgccaagtacgccccctattgacgtcaatgacggtaaatggcccgcctggcattatgcccagtacatgacctt
atgggactttcctacttggcagtacatctacgtattagtcatcgctattaccatggtgatgcggttttggcagtacatcaatggg
cgtggatagcggtttgactcacggggatttccaagtctccaccccattgacgtcaatgggagtttgttttggcaccaaaatca
acgggactttccaaaatgtcgtaacaactccgccccattgacgcaaaagggcgggaattcgagctcggtactcgagcgg
tgttccgcggtcctcctcgtatagaaactcggaccactctgagacgaaggctcgcgtccaggccagcacgaaggaggct
aagtgggaggggtagcggtcgttgtccactagggggtccactcgctccagggtgtgaagacacatgtcgccctcttcggc
atcaaggaaggtgattggtttataggtgtaggccacgtgaccgggtgttcctgaaggggggctataaaagggggtggggg
cgcgttcgtcctcactctcttccgcatcgctgtctgcgagggccagctgttgggctcgcggttgaggacaaactcttcgcggt
ctttccagtactcttggatcggaaacccgtcggcctccgaacggtactccgccaccgagggacctgagcgagtccgcatc
gaccggatcggaaaacctctcgactgttggggtgagtactccctctcaaaagcgggcatgacttctgcgctaagattgtca
gtttccaaaaacgaggaggatttgatattcacctggcccgcggtgatgcctttgagggtggccgcgtccatctggtcagaaa
agacaatctttttgttgtcaagcttgaggtgtggcaggcttgagatctggccatacacttgagtgacaatgacatccactttgc
ctttctctccacaggtgtccactcccaggtccaaccggaattgtacccgcggccagagcttgcgggcgccaccgcggccg
cggggatccagacatgataagatacattgatgagtttggacaaaccacaactagaatgcagtgaaaaaaatgctttatttg
tgaaatttgtgatgctattgctttatttgtaaccattataagctgcaataaacaagttaacaacaacaattgcattcattttatgttt
caggttcagggggaggtgtgggaggttttttcggatcctcttggcgtaatcatggtcatagctgtttcctgtgtgaaattgttatcc
gctcacaattccacacaacatacgagccggaagcataaagtgtaaagcctggggtgcctaatgagtgagctaactcaca
ttaattgcgttgcgctcactgcccgctttccagtcgggaaacctgtcgtgccagctgcattaatgaatcggccaacgcgcgg
ggaaaggcggtttgcgtattgggcgctcttccgcttcctcgctcactgactcgctgcgctcggtcgttcggctgcggcgagcg
gtatcagctcactcaaaggcggtaatacggttatccacagaatcaggggataacgcaggaaagaacatgtgagcaaaa
ggccagcaaaaggccaggaaccgtaaaaaggccgcgttgctggcgttcttccataggctccgcccccctgacgagcat
cacaaaaatcgacgctcaagtcagaggtggcgaaacccgacaggactataaagataccaggcgtttccccctggaag
ctccctcgtgcgctctcctgttccgaccctgccgcttaccggatacctgtccgcctttctcccttcgggaagcgtggcgctttctc
atagctcacgctgtaggtatctcagttcggtgtaggtcgttcgctccaagctgggctgtgtgcacgaaccccccgttcagccc
gaccgctgcgccttatccggtaactatcgtcttgagtccaacccggtaagacacgacttatcgccactggcagcagccact
ggtaacaggattagcagagcgaggtatgtaggcggtgctacagagttcttgaagtggtggcctaactacggctacactag
aagaacagtatttggtatctgcgctctgctgaagccagttaccttcggaaaaagagttggtagctcttgatccggcaaacaa
accaccgctggtagcggtggtttttttgtttgcaagcagcagattacgcgcagaaaaaaaggatctcaagaagatcctttga
tcttttctacggggtctgacgctcagtggaacgaaaactcacgttaagggattttggtcatgagattatcaaaaaggatcttca
cctagatcccttttaattaaaaatgaagttttaaatcaatctaaagtatatatgagtaaacttggtctgacagttaccaatgctta
atcagtgaggcacctatctcagcgatctgtctatttcgttcatccatagttgcctgactccccgtcgtgtagataactacgatac
gggagggcttaccatctggccccagtgctgcaatgataccgcgagacccacgctcaccggctccagatttatcagcaata
aaccagccagccggaagggccgagcgcagaagtggtcctgcaactttatccgcctccatccagtctattaattgttgccgg
gaagctagagtaagtagttcgccagttaatagtttgcgcaacgttgttgccattgctacaggcatcgtggtgtcacgctcgtc
gtttggtatggcttcattcagctccggttcccaacgatcaaggcgagttacatgatcccccatgttgtgcaaaaaagcggtta
gctccttcggtcctccgatcgttgtcagaagtaagttggccgcagtgttatcactcatggttatggcagcactgcataattctctt
actgtcatgccatccgtaagatgcttttctgtgactggtgagtactcaaccaagtcattctgagaatagtgtatgcggcgaccg
agttgctcttgcccggcgtcaatacgggataataccgcgccacatagcagaactttaaaagtgctcatcattggaaaacgtt
cttcggggcgaaaactctcaaggatcttaccgctgttgagatccagttcgatgtaacccactcgtgcacccaactgatcttca
gcatcttttactttcaccagcgtttctgggtgagcaaaaacaggaaggcaaaatgccgcaaaaaagggaataagggcga
cacggaaatgttgaatactcatactcttcctttttcaatattattgaagcatttatcagggttattgtctcatgagcggatacatattt
gaatgtatttagaaaaataaacaaataggggttccgcgcacatttccccgaaaagtgccacctgacgtctaagaaaccat
tattatcatgacattaacctataaaaataggcgtatcacgaggccctttcgtctcgcgcgtttcggtgatgacggtgaaaacct
ctgacacatgcagctcccggagacggtcacagcttgtctgtaagcggatgccgggagcagacaagcccgtcagggcgc
gtcagcgggtgttggcgggtgtcggggctggcttaactatgcggcatcagagcagattgtactgagagtgcaccatatgcg
gtgtgaaataccgcacagatgcgtaaggagaaaataccgcatcaggcgccattcgccattcaggctgcgcaactgttgg
gaagggcgatcggtgcgggcctcttcgctattacgccagctggcgaaagggggatgtgctgcaaggcgattaagttgggt
aacgccagggttttcccagttacgacgttgtaaaacgacggccagtgaatt

Materials and Methods:

Transfection of described constructs into 293-6E cells is carried out as follows. The cells used are HEK293-6E cells in exponential growth phase (0.8 to 1.5×10⁶ cells/ml), which cells have been passaged in culture less than 30 times; the cultures are inoculated into fresh growth medium to a concentration of 3×10⁵ cells/ml, every three or four days. Growth medium is FreeStyle™ 293 Expression Medium (GIBCO™ Cat. No. 12338-018, Invitrogen, Carlsbad, Calif.) supplemented with Geneticin (G418) 25 ug/ml (GIBCO™ Cat. No. 10131-027) and 0.1% Pluronic F-68 (surfactant, GIBCO™ Cat. No. 24040-032). Transfection Medium is FreeStyle™ 293 Expression Medium (GIBCO™ Cat. No. 12338-018) with a final concentration of 10 mM HEPES Buffer Solution ml (GIBCO™ Cat. No. 15630-080). For transfection, the vector DNA of choice is added to achieve a concentration of 1 μg (Heavy Chain+Light Chain)/ml Subject to change based on optimization experiments. PEI (polyethylenimine), linear, 25 kDa, 1 mg/ml sterile stock solution, pH 7.0 (Polysciences, Inc., Warrington, Pa.) is added as a transfection mediator, with a DNA:PEI ratio of 1:2. The Feeding Medium used is Tryptone N1 Medium (TN1 powder from Organotechnie France, Cat No. 19554, available through TekniScience Inc. Tel#1-800-267-9799). 5% w/v stock solution in FreeStyle™ 293 Expression Medium is added to a final concentration of 0.5%. Standard laboratory equipment is generally used. A Cedex Cell Counting System is employed (Innovatis, Bielefeld, Germany).

Each small-scale transfection is carried out in a 125 ml Erlenmeyer flask as follows. An aliquot of 20 ml of fresh culture medium is inoculated with 1×10⁶ cells/ml of viable cells. (Note: For larger volumes, culture should be 20-25% of nominal capacity of vessel, e.g. 100 ml culture in 500 ml flask). Cultures are then placed in a 37° C. incubator with a humidified atmosphere of 5% CO₂ with 130 rpm rotation speed.

The DNA-PEI complex preparation is made by warming transfection medium to 37° C. in a water bath, thawing at room temperature frozen PEI stock and DNA solutions (stored at −20° C.). The amounts of DNA and PEI used are based on the total volume of culture being transfected. A 20 ml culture with 2.5 ml DNA/PEI complex and 2.5 ml Tn1 requires a total of 25 μg DNA and 50 μg PEI. DNA:PEI complexes (e.g., for ten transfections) are formed by combining a 12.5 ml of transfection medium to tube A to which has been added a solution containing the DNA vector of choice to a final concentration of 10 μg/ml and 12.5 ml of transfection medium to PEI has been added (20 μg/ml, final conc.). The PEI mixture is mixed by vortexing about 10 seconds prior to mixing with the DNA solution. After combining the PEI and DNA mixtures, the combination is mixed by vortexing for 10 seconds. Then the mixture is allowed to stand at room temperature for 15 minutes (but not more than 20 minutes). 2.5 ml of the DNA:PEI complex solution is added per 20 ml HEK-6E cells. The 5% TN1 supplement is added to a final concentration of 0.5% to each flask about 20 to 24 hours after transfection.

Cell density and viability are determined on day 4 and day 7. Cell pellets are collected from 2 ml aliquot of culture) for Western analysis and Northern Blot analysis on day 4. Pellets are frozen at −80° C. until analyzed. Cells are harvested by centrifugation at 1000 rpm (10 min) 7 days after transfection, and supernatants are filtered using pre-filter papers and a Corning 0.22 μm CA Filter system. Supernatant samples are also stored at 80° C. until analyzed, for example using ELISA assays.

For Northern Blot Analysis, total RNA is isolated from transiently transfected 293-6E cells as follows. Frozen cell pellets are thawed on ice. RNA is purified using the Qiagen Rneasy Mini Kit (Qiagen, cat. #74104), according to the manufacturer's instructions.

Formaldehyde/agarose gel preparation is as follows. 2 grams of agarose (Ambion, cat. #9040) is boiled in 161.3 ml distilled water. 4 ml 1M MOPS (Morpholinopropanesulfonic acid) PH 7.0, 1 ml 1M NaOAc, 0.4 ml 0.5 M EDTA are added and the mixture is cooled to 60° C. Then 33.3 ml 37% Formaldehyde (J. T. Baker, cat #2106-01) is added, and the molten agarose solution is mixed gently. The gel is poured and allowed to solidify in a fume hood.

Running buffer is prepared by mixing 30 ml 1M MOPS, pH 7.0, 7 ml 1M NaOAc, 3 ml 0.5M EDTA and DEPC (diethylpyrocarbonate) treated dH₂O to 1.5.

RNA samples are prepared by mixing 3 parts formaldehyde load dye (Ambion, cat. #8552) with 1 part RNA. 3 to 5 μg of RNA is run per lane. The RNA molecular weight markers used is from the 0.5-10 Kb RNA Ladder (Invitrogen, cat. #15623-200). Samples are heated at 65° C. for 5 minutes to denature and chill on ice. Then 0.5 μl 10 μg/μl Ethidium Bromide (Pierce, cat. #17898) is added to each sample. Each sample is spun briefly to pellet liquid.

Gel electrophoresis is carried out as follows. The formaldehyde/agarose gel is covered with running buffer, samples are loaded and then run at 150V for 2 hours in a fume hood. Bands are viewed using ultraviolet transillumination and photographed for a permanent record.

Capillary transfer is done by soaking the gel in several changes of DEPC-treated dH₂O for five minutes to remove formaldehyde. The gel is then soaked in 50 mM NaOH, 10 mM NaCl for 20 minutes at room temperature to further denature any double-stranded RNA. The gel is rinsed once in DEPC-treated dH₂O and then soaked in 20×SSC (175.3 g NaCl; 88.2 g Sodium Citrate; pH to ˜7.0 with 10M NaOH, volume adjusted to 1 L) for 20 minutes at room temperature to neutralize. Hybond-N+ membrane (Amersham Biosciences, cat #RPN303B) is soaked and cut to the same size as the gel, in DEPC-treated dH₂O to wet. 3M filter paper (Whatman cat#3030917) is cut to the same size as the gel and the membrane. The transfer system is assembled by placing a layer of 3M paper on a solid support over a reservoir of 20×SSC so that the paper wicks the 20×SSC through the layers to be assembled on top. The gel is placed on this wick, the Hybond-N+ membrane, 3 sheets of 3M paper cut to size, and a thick stack of Gel Blot Paper (Schleicher & Schuell, cat. #10427920). A flat support is placed on top of the stack, and weight is added (usually a liter bottle of water), if needed, to insure efficient capillary transfer. Plastic wrap is used to cover any of the reservoir exposed to air to prevent evaporation. The transfer is allowed to proceed overnight at room temperature. Then the transfer system is disassembled and the blot is soaked in 6×SSC to remove any agarose. The membrane is allowed to air dry and exposed to UV to crosslink the blot.

DNA probe templates are the coding region for heavy and light chain of D2E7. 100 ng of the desired template is labeled with Alkaline Phosphate using the AlkPhos Direct Labeling Reagents kit (alkaline phosphatase labeling system, Amersham Biosciences, cat. #RPN3680) according to the manufacturer's instructions. Prehybridization and hybridization steps were performed using the same kit as for labeling (contains hybridization buffer). Membranes were prehybridized for at least 1 hour at 65° C. in a hybridization oven, the probe was boiled and added directly to prehybridization buffer/blot. Hybridization took place overnight at 65° C. in a hybridization oven. The hybridization solution was decanted, and the membrane was washed briefly with 2×SSC to remove hybridization solution, then washed twice with 2×SSC, 0.1% SDS at 65° C. for 15 minutes each, and finally washed twice with 0.1×SSC, 0.1% SDS at 65° C. for 15 minutes each time. To visualize bands on the membrane, chemiluminescence was used. Blots were overlaid with CDP-Star Detection Reagent (alkaline phosphatase-dependent production of a photope from a 1,2-dioxetane substrate, Amersham Biosciences, cat. #RPN3682), for 5 minutes at room temperature. Excess reagent was drained from blots and they were then encased in plastic sheet protectors. Blots were exposed to Kodak Biomax MR film (x ray film, Kodak, cat. #8952855), starting for 10 seconds for up to 10 minutes. Films were developed using the Kodak M35A X-OMAT Processor (x ray developer/processor).

Cell pellet samples for western blotting were prepared as follows. For the analysis of intracellular antibody expression, cells were lysed in NP 40 Lysis buffer (50 mM Tris-HCl, pH 7.5, 150 mM NaCl, 1% NP40 (octylphenolpoly(ethyleneglycolether)), 5 mM BME, and protease inhibitors cocktail III), with incubation on ice for 10 min. The fractions for membranes and insoluble proteins are collected by centrifugation at 16,000 rpm for 30 min using a microcentrifuge. The supernatant, designated the soluble intracellular, or cytosolic fraction, was used for gel analysis, with the addition of SDS loading buffer with DTT. The pellets were suspended with equal volume of lysis buffer, and SDS gel loading buffer with DTT was added. Culture supernatant samples were prepared for western blotting as follows. Culture supernatants were either concentrated using Centricon Ultra (ultrafiltration device, Millipore), with a MW cut off of 30,000 daltons, or used directly for western blotting. For immunoblotting (western analysis), samples were resolved on NUPAGE 4-12% Bis-Tris (polyacrylamide) gels and transferred to PVDF membrane using standard methods. The membranes were incubated for 1 h in blocking solution (PBS with 0.05% Tween 20 (polyoxyethylene sorbitan monolaurate) and 5% dry milk), washed, incubated with polyclonal rabbit anti-human IgG/HRP or polyclonal rabbit anti-human kappa light chain/HRP, from DakoCytomation (Denmark), at 1:1000 dilution in PBST buffer, and then washed again in three changes of PBST at room temperature. ECL Plus Western Blotting Detection (chemiluminescent and chemifluorescent detection) System from GE/Amersham Biosciences (Piscataway, N.J.) was used for detection.

ELISA assays were carried out using standard methods, using Goat Anti-Human IgG, UNLB and Goat Anti-Human IgG/HRP from Southern Biotech (Birmingham, Ala.), 2% milk in PBS as blotting buffer, K-Blue (3,3′, 5,5′ tetramethylbenzidine and hydrogen peroxide (H₂O₂, Neogen, Lansing, Mich.) as substrate. Plates were read with Spectramax microplate reader at 650 nM primary wavelength and 490 nm reference wavelength.

The secreted antibody was affinity purified with standard methods using Protein A Agarose beads from Invitrogen (Carlsbad, Calif.), Immuno Pure (A) IgG Binding Buffer from Pierce, PBS, pH 7.4 as wash buffer, and 0.1 M Acetic Acid/150 mM NaCl, pH 3.5 as elution buffer (neutralized using 1 M Tris pH 9.5).

Determination of Intact Molecular Weight.

Intact molecular weights of the D2E7 samples produced from construct pTT3 HC-int-LC P.hori were analyzed by LC-MS. An 1100 capillary HPLC system (Agilent SN DE 14900659) with a protein microtrap (Michrom Bioresources, Inc. cat. 004/25109/03) was used to desalt and introduce samples into the Q Star Pulsar i mass spectrometer (Applied Biosystems, SN K1820202). To elute the samples, a gradient was run with buffer A (0.08% FA, 0.02% TFA in HPLC water) and buffer B (0.08% FA and 0.02% TFA in acetonitrile), at a flow rate of 50 μL/min, for 15 minutes.

Determination of Light Chain and Heavy Chain Molecular Weight.

Native D2E7 samples produced from construct pTT3 HC-int-LC P.hori were were analyzed by LC-MS. Reduction of the disulfide bonds that linked light chains and heavy chains together was conducted in 20 mM DTT at 37° C. for 30 minutes. An 1100 capillary HPLC system (Agilent SN DE 14900659) with a PLRP-S column (Michrom Bioresources, Inc. 8 μm, 4000 Å, 1.0×150 mm, P/N 901-00911-00) was used to separate light chains from heavy chains and introduce them into the Q Star Pulsar i mass spectrometer (Applied Biosystems, SN K1820202). The column was heated at 60° C. An HPLC gradient, which was run with buffer A (0.08% FA, 0.02% TFA in HPLC water) and buffer B (0.08% FA and 0.02% TFA in acetonitrile), at a flow rate of 50 μL/min, was run for 60 minutes to elute the samples.

Restriction endonucleases were from New England Biolabs (Beverly, Mass.). Custom oligonucleotides, DNA polymerases, DNA ligases, and E. coli strains used for cloning were from Invitrogen (Carlsbad, Calif.). Protease inhibitor cocktail III was from Calbiochem (La Jolla, Calif.). Qiagen (Valencia, Calif.) products were used for DNA isolation and purification.

### STATEMENTS REGARDING INCORPORATION BY REFERENCE AND VARIATIONS

All references mentioned throughout this application, for example patent documents including issued or granted patents or equivalents; patent application publications; unpublished patent applications; and non-patent literature documents or other source material; are hereby incorporated by reference herein in their entireties, as though individually incorporated by reference. In the event of any inconsistency between cited references and the disclosure of the present application, the disclosure herein takes precedence. Some references provided herein are incorporated by reference to provide information, e.g., details concerning sources of starting materials, additional starting materials, additional reagents, additional methods of synthesis, additional methods of analysis, additional biological materials, additional cells, and additional uses of the invention.

All patents and publications mentioned herein are indicative of the levels of skill of those skilled in the art to which the invention pertains. References cited herein can indicate the state of the art as of their publication or filing date, and it is intended that this information can be employed herein, if needed, to exclude specific embodiments that are in the qualifying prior art. For example, when compositions of matter are claimed herein, it should be understood that compounds known and available as qualifying prior art relative to Applicant's invention, including compounds for which an enabling disclosure is provided in the references cited herein, are not intended to be included in the composition of matter claims herein.

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Thus as used herein, comprising is synonymous with including, containing, having, or characterized by, and is inclusive or open-ended. As used herein, “consisting of” excludes any element, step, or ingredient, etc. not specified in the claim description. As used herein, “consisting essentially of” does not exclude materials or steps that do not materially affect the basic and novel characteristics of the claim (e.g., relating to the active ingredient). In each instance herein any of the terms “comprising”, “consisting essentially of” and “consisting of” may be replaced with either of the other two terms, thereby disclosing separate embodiments and/or scopes which are not necessarily coextensive. The invention illustratively described herein suitably may be practiced in the absence of any element or elements or limitation or limitations not specifically disclosed herein.

Whenever a range is disclosed herein, e.g., a temperature range, time range, composition or concentration range, or other value range, etc., all intermediate ranges and subranges as well as all individual values included in the ranges given are intended to be included in the disclosure. This invention is not to be limited by the embodiments disclosed, including any shown in the drawings or exemplified in the specification, which are given by way of example or illustration and not of limitation. It will be understood that any subranges or individual values in a range or subrange that are included in the description herein can be excluded from the claims herein.

The invention has been described with reference to various specific and/or preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be employed in the practice of the invention as broadly disclosed herein without resort to undue experimentation; this can extend, for example, to starting materials, biological materials, reagents, synthetic methods, purification methods, analytical methods, assay methods, and biological methods other than those specifically exemplified. All art-known functional equivalents of the foregoing (e.g., compositions, methods, devices, device elements, materials, procedures and techniques, etc.) described herein are intended to be encompassed by this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, preferred embodiments, and optional features, modification and variation of the concepts herein disclosed may be resorted to by those skilled in the art, and that such modifications and variations are considered to be within the scope of this invention as defined by the appended claims.

### ADDITIONAL REFERENCES

U.S. Pat. No. 6,258,562, U.S. Pat. No. 6,090,382; U.S. Pat. No. 6,455,275; EP1080206B1; WO 9960135; U.S. Pat. No. 5,912,167; U.S. Pat. No. 5,162,601; WO 199521249A1; U.S. Pat. No. 5,149,783; U.S. Pat. No. 5,955,072; U.S. Pat. No. 5,532,142; US 20040224391; U.S. Pat. No. 6,537,806; U.S. Pat. No. 5,846,767; US 20030099932; WO 9958663; US 20030157641; US 2003048306A2; U.S. Pat. No. 6,114,146; U.S. Pat. No. 6,060,273; U.S. Pat. No. 5,925,565; US 20040241821; WO 2003100021A2; WO 2003100022A2; US 20040265955; US 20050003482; US 20050042721; WO 2005017149; WO 2004113493; US 20050136035; WO 2004108893; U.S. Pat. No. 6,692,736; US 20050147962; U.S. Pat. No. 6,331,415; U.S. Pat. No. 6,632,637; US 20040063186; U.S. Pat. No. 7,026,526; U.S. Pat. No. 6,365,377; WO 2005123915; U.S. Pat. No. 5,665,567; WO 9741241A1; EP 0701616B1; US 20060010506; WO 2006048459; U.S. Pat. No. 6,852,510; WO 2005072129; U.S. Pat. No. 5,648,254; U.S. Pat. No. 6,908,751; US 20050221429; WO 2005071088; WO 2005108585; WO 2005085456; U.S. Pat. No. 7,029,876; U.S. Pat. No. 6,638,762; U.S. Pat. No. 6,544,780; U.S. Pat. No. 5,519,164; WO 2003031630; U.S. Pat. No. 6,294,353; WO 2005047512; U.S. Pat. No. 7,052,905; U.S. Pat. No. 7,018,833; US 20020034814; US 20040126883; US 20050002907; US 20050112095; US 20050214258; EP 0598029.
Mathys S et al., 1999, Gene 231(1-2):1-13, Characterization of a self-splicing mini-intein and its conversion into autocatalytic N- and C-terminal cleavage elements: facile production of protein building blocks for protein ligation.

We claim:

1. An expression vector for generating one or more recombinant protein products comprising a sORF insert; said sORF insert comprising a first nucleic acid sequence encoding a first polypeptide, a first intervening nucleic acid sequence encoding a first protein cleavage site, and a second nucleic acid sequence encoding a second polypeptide; wherein said intervening nucleic acid sequence encoding said first protein cleavage site is operably positioned between said first nucleic acid sequence and said second nucleic acid sequence; and wherein said expression vector is capable of expressing a sORF polypeptide cleavable at said first protein cleavage site.

2. The expression vector of claim 1 wherein said first protein cleavage site comprises a self-processing cleavage site.

3. The expression vector of claim 2 wherein said self-processing cleavage site comprises an intein segment or modified intein segment, wherein the modified intein segment permits cleavage but not complete ligation of said first polypeptide to said second polypeptide.

4. The expression vector of claim 2 wherein said self-processing cleavage site comprises a hedgehog segment or modified hedgehog segment, wherein the modified hedgehog segment permits cleavage of said first polypeptide from said second polypeptide.

5. The expression vector of claim 1 wherein the first polypeptide and second polypeptide are capable of multimeric assembly.

6. The expression vector of claim 1 wherein at least one of said first polypeptide and second polypeptide are capable of extracellular secretion.

7. The expression vector of claim 1 wherein at least one of said first polypeptide and second polypeptide are of mammalian origin.

8. The expression vector of claim 1 wherein at least one of said first polypeptide and second polypeptide comprises an immunoglobulin heavy chain or functional fragment thereof.

9. The expression vector of claim 1 wherein at least one of said first polypeptide and second polypeptide comprises an immunoglobulin light chain or functional fragment thereof.

10. The expression vector of claim 1 wherein said first polypeptide comprises an immunoglobulin heavy chain or functional fragment thereof and said second polypeptide comprises an immunoglobulin light chain or functional fragment thereof; and wherein said first and second polypeptides are in any order.

11. The expression vector of claim 1 wherein said first polypeptide and second polypeptide taken together are capable of associating in multimeric assembly to form a functional antibody or other antigen recognition molecule.

12. The expression vector of claim 1 wherein said first polypeptide is upstream of said second polypeptide.

13. The expression vector of claim 1 wherein said second polypeptide is upstream of said first polypeptide.

14. The expression vector of claim 1 further comprising a third nucleic acid sequence encoding a third polypeptide, wherein said third nucleic acid sequence is operably positioned after said second nucleic acid sequence; and wherein said third sequence may independently be the same or different from either of said first or second nucleic acid sequence.

15. The expression vector of claim 14 wherein at least two of said first, second, and third polypeptides taken together are capable of associating in multimeric assembly.

16. The expression vector of claim 1 further comprising a second intervening nucleic acid sequence encoding a second protein cleavage site, wherein said second intervening nucleic acid sequence is operably positioned after said first and said second nucleic acid sequence; and wherein said second intervening sequence may be the same or different from said first intervening nucleic acid sequence.

17. The expression vector of claim 1 further comprising a third nucleic acid sequence encoding a third polypeptide, and a second intervening nucleic acid sequence encoding a second protein cleavage site; wherein the second intervening nucleic acid sequence and third nucleic acid sequence, in that order, are operably positioned after said second nucleic acid sequence.

18. The expression vector of claim 14 wherein said third nucleic acid sequence encodes an immunoglobulin heavy chain, light chain, or respectively a functional fragment thereof.

19. The expression vector of claim 14 wherein said third nucleic acid sequence encodes an immunoglobulin light chain or functional fragment thereof.

20. The expression vector of claim 14 wherein said third nucleic acid sequence encodes an immunoglobulin heavy chain or functional fragment thereof.

21. The expression vector of claim 1 wherein said first intervening nucleic acid sequence encoding a first protein cleavage site comprises a signal peptide nucleic acid encoding a signal peptide cleavage site or modified signal peptide cleavage site sequence.

22. The expression vector of claim 1 further comprising a signal peptide nucleic acid sequence encoding a signal peptide cleavage site, operably positioned before said first nucleic acid sequence or said second nucleic acid sequence.

23. The expression vector of claim 1 further comprising two signal peptide nucleic acid sequences, each independently encoding a signal peptide cleavage site, wherein one signal peptide nucleic acid sequence is operably positioned before said first nucleic acid encoding said first polypeptide and the other signal peptide nucleic acid sequence is operably positioned before said second nucleic acid encoding said second polypeptide.

24. The expression vector of claim 21 wherein said signal peptide nucleic acid sequence encodes an immunoglobulin light chain signal peptide cleavage site or modified immunoglobulin light chain signal peptide cleavage site.

25. The expression vector of claim 24 wherein the signal peptide nucleic acid sequence encodes a modified or unmodified immunoglobulin light chain signal peptide cleavage site, and wherein said modified site is capable of effecting cleavage and increasing secretion of at least one of said first polypeptide, said second polypeptide, and an assembled molecule of said first and second polypeptides; and wherein a secretion level in the presence of said signal peptide site is about 10% greater to about 100-fold greater than a secretion level in the absence of said signal peptide site.

26. The expression vector of claim 1 wherein said intervening nucleic acid sequence encoding a first protein cleavage site comprises an intein or modified intein sequence selected from the group consisting of: a Pyrococcus horikoshii Pho Pol I sequence, a Saccharomyces cerevisiae VMA sequence, Synechocystis spp. Strain PCC6803 DnaE sequence, Mycobacterium xenopi GyrA sequence, Pyrococcus species GB-D DNA polymerase, A-type bacterial intein-like (BIL) domain, and B-type BIL.

27. The expression vector of claim 1 wherein said intervening nucleic acid sequence encoding a first protein cleavage site comprises a C-terminal auto-processing domain of a hedgehog family member, wherein the hedgehog family member is from Drosophila, mouse, human, or other insect or animal species.

28. The expression vector of claim 1 wherein said intervening nucleic acid sequence encoding a first protein cleavage site comprises a C-terminal auto-processing domain from a warthog, groundhog, or other hog-containing gene from a nematode, or Hoglet domain from a choanoflagellate.

29. The expression vector of claim 1 wherein said first and said second polypeptide comprise a functional antibody or other antigen recognition molecule; with an antigen specificity directed to binding an antigen selected from the group consisting of: tumor necrosis factor-a, erythropoietin receptor, RSV, EL/selectin, interleukin-1, interleukin-12, interleukin-13, interleukin-18, interleukin-23, CXCL-13, GLP-1R, and amyloid beta.

30. The expression vector of claim 1, wherein the first and second polypeptides comprise a pair of immunoglobulin chains from an antibody of D2E7, ABT-007, ABT-325, EL246, or ABT-874.

31. The expression vector of claim 1, wherein the first and second polypeptide are each independently selected from an immunoglobulin heavy chain or an immunoglobulin light chain segment from an analogous segment of D2E7, ABT-007, ABT-325, EL246, ABT-874, or other antibody.

32. The expression vector of claim 1, wherein said vector further comprises a promoter regulatory element for said sORF insert.

33. The expression vector according to claim 32, wherein said promoter regulatory element is inducible or constitutive.

34. The expression vector according to claim 32, wherein said promoter regulatory element is tissue specific.

35. The expression vector according to claim 32, wherein said promoter comprises an adenovirus major late promoter.

36. The expression vector according to claim 1, wherein said vector further comprises a nucleic acid encoding a protease capable of cleaving said first protein cleavage site.

37. The expression vector according to claim 36, wherein said nucleic acid encoding a protease is operably positioned within said sORF insert; said expression vector further comprising an additional nucleic acid encoding a second cleavage site located between said nucleic acid encoding a protease and at least one of said first nucleic acid and said second nucleic acid.

38. A host cell comprising a vector according to claim 1.

39. The host cell according to claim 38, wherein said host cell is a prokaryotic cell.

40. The host cell according to claim 39, wherein said host cell is Escherichia coli.

41. The host cell according to claim 38, wherein said host cell is a eukaryotic cell.

42. The host cell according to claim 41, wherein said eukaryotic cell is selected from the group consisting of a protist cell, animal cell, plant cell and fungal cell.

43. The host cell according to claim 42, wherein said eukaryotic cell is an animal cell selected from the group consisting of a mammalian cell, an avian cell, and an insect cell.

44. The host cell according to claim 43, wherein said host cell is a CHO cell or a dihydrofolate reductase-deficient CHO cell.

45. The host cell according to claim 43, wherein said host cell is a COS cell.

46. The host cell according to claim 42, wherein said host cell is a yeast cell.

47. The host cell according to claim 46, wherein said yeast cell is Saccharomyces cerevisiae.

48. The host cell according to claim 43, wherein said host cell is an insect Spodoptera frugiperda Sf9 cell.

49. The host cell according to claim 43, wherein said host cell is a human embryonic kidney cell.

50. A method for producing a recombinant polyprotein or a plurality of proteins, comprising culturing a host cell according to claim 38 in a culture medium under conditions sufficient to allow expression of a vector protein.

51. The method of claim 50 further comprising recovering and/or purifying said vector protein.

52. The method of claim 50 wherein said plurality of proteins are capable of multimeric assembly.

53. The method of claim 50 wherein the recombinant polyprotein or plurality of proteins are biologically functional and/or therapeutic.

54. A method for producing an immunoglobulin protein or functional fragment thereof, assembled antibody, or other antigen recognition molecule, comprising culturing a host cell according to claim 38 in a culture medium under conditions sufficient to produce an immunoglobulin protein or functional fragment thereof, assembled antibody, or other antigen recognition molecule.

55. A protein produced according to the method of claim 50.

56. A polyprotein produced according to the method of claim 50.

57. An assembled immunoglobulin; assembled other antigen recognition molecule; or individual immunoglobulin chain or functional fragment thereof produced according to the method of claim 50.

58. The immunoglobulin; other antigen recognition molecule; or individual immunoglobulin chain or functional fragment thereof according to claim 57, wherein there is a capability to effect or contribute to specific antigen binding to tumor necrosis factor-a, erythropoietin receptor, interleukin-18, EL/selectin or interleukin-12.

59. The immunoglobulin or functional fragment thereof according to claim 58, wherein the immunoglobulin is D2E7 or wherein the functional fragment is a fragment of D2E7.

60. A pharmaceutical composition comprising a protein according to claim 55, and a pharmaceutically acceptable carrier.

61. The expression vector of claim 1 wherein said first protein cleavage site comprises a cellular protease cleavage site or a viral protease cleavage site.

62. The expression vector according to claim 1 wherein said first protein cleavage site comprises a site recognized by furin; VP4 of IPNV; tobacco etch virus (TEV) protease; 3C protease of rhinovirus; PC5/6 protease; PACE protease, LPC/PC7 protease; enterokinase; Factor Xa protease; thrombin; genenase I; MMP protease; Nuclear inclusion protein a(N1a) of turnip mosaic potyvirus; NS2B/NS3 of Dengue type 4 flaviviruses, NS3 protease of yellow fever virus; ORF V of cauliflower mosaic virus; KEX2 protease; CB2; or 2A.

63. The expression vector of claim 1 wherein said first protein cleavage site is a viral internally cleavable signal peptide cleavage site.

64. The expression vector of claim 63 wherein said viral internally cleavable signal peptide cleavage site comprises a site from influenza C virus, hepatitis C virus, hantavirus, flavivirus, or rubella virus.

65. A method for expression of proteins of a two hybrid system, wherein said two hybrid system comprises a bait protein and a candidate prey protein, said method comprising the steps of: providing a host cell into which has been introduced an expression vector encoding a polyprotein comprising a bait protein portion and a candidate prey protein portion, said portions separated by a self-processing cleavage sequence, a signal peptide sequence or a protease cleavage site; and culturing the host cell under conditions which allow expression of the polyprotein and self processing or protease cleavage of the polyprotein.

66. The method of claim 65, wherein the polyprotein further comprises a cleavable component of a three hybrid system.

67. The expression vector according to claim 1 wherein said vector does not contain a 2A sequence.

68. The expression vector according to claim 1 wherein said first protein cleavage site comprises a FMDV 2A sequence; a 2A-like domain from other Picornaviridae, an insect virus, Type C rotavirus, trypanosome, or Thermatoga maritima.

69. An expression vector for expressing a recombinant protein, comprising a coding sequence for a polyprotein, wherein the polyprotein comprises at least a first and a second protein segment, wherein said protein segments are separated by a protein cleavage site therebetween, wherein the protein cleavage site comprises a self processing peptide cleavage sequence, a signal peptide cleavage sequence or a protease cleavage sequence; and wherein said coding sequence is expressible in a host cell and is cleaved within the host cell.

70. The expression vector of claim 1, wherein said intervening nucleic acid sequence additionally encodes a tag.


SEQUENCE LISTING (158 sequences)

SEQ ID NO: 1 - protein, 4 aa, Artificial
  Synthetic  cleavage recognition site for furin.
RXXR

SEQ ID NO: 2 - protein, 5 aa, Artificial
  Recognition sequence for VP4 of IPNV.
XXAXG

SEQ ID NO: 3 - protein, 7 aa, Artificial
  Recognition sequence for TEV protease.
EXXYXQG

SEQ ID NO: 4 - protein, 8 aa, Artificial
  recognition site for rhinovirus 3C protease
LEVLFQGP

SEQ ID NO: 5 - protein, 6 aa, Artificial
  Recognition sequence of PC5/6 protease, LPC/PC7 protease and enterokinase.
DDDDKX

SEQ ID NO: 6 - protein, 5 aa, Artificial
  Recognition sequence for Factor Xa protease.
IXGRX

SEQ ID NO: 7 - protein, 7 aa, Artificial
  Recognition sequence for thrombin.
LVGPRGS

SEQ ID NO: 8 - protein, 6 aa, Artificial
  Recognition sequence for genenase I.
PGAAHY

SEQ ID NO: 9 - protein, 7 aa, Artificial
  Recognition sequence for MMP protease, N1a of turnip mosaic potyvirus and KEX2 protease.
MYKREAD

SEQ ID NO: 10 - protein, 4 aa, Artificial
  Amino acid sequence of furin which targets protein to Trans Golgi Network.
EEDE

SEQ ID NO: 11 - protein, 24 aa, Artificial
  Internally cleavable signal peptide of influenza virus C.
MGRMAMKWLVVIICFSITSQPASA

SEQ ID NO: 12 - protein, 19 aa, Artificial
  FMDV 2A sequence
LLNFDLLKLAGDVESNPGP

SEQ ID NO: 13 - protein, 19 aa, Artificial
  FMDV 2A sequence.
TLNFDLLKLAGDVESNPGP

SEQ ID NO: 14 - protein, 14 aa, Artificial
  FDMV 2A sequence.
LLKLAGDVESNPGP

SEQ ID NO: 15 - protein, 20 aa, Artificial
  Variant of 2A sequence.
QLLNFDLLKLAGDVESNPGP

SEQ ID NO: 16 - protein, 19 aa, Artificial
  Variant of 2A sequence.
NFDLLKLAGDVESNPGPFF

SEQ ID NO: 17 - protein, 14 aa, Artificial
  Variant of 2A sequence.
LLKLAGDVESNPGP

SEQ ID NO: 18 - protein, 17 aa, Artificial
  Variant of 2A sequence.
NFDLLKLAGDVESNPGP

SEQ ID NO: 19 - protein, 24 aa, Artificial
  Variant of 2A sequence.
APVKQTLNFDLLKLAGDVESNPGP

SEQ ID NO: 20 - protein, 58 aa, Artificial
  Variant of 2A sequence.
VTELLYRMKRAETYCPRPLLAIHPTEARHKQKIVAPVKQTLNFDLLKLAGDVESNPGP

SEQ ID NO: 21 - protein, 10 aa, Artificial
  N-terminal sequence of D2E7 immunoglobulin heavy chain.
EVQLVESGGG

SEQ ID NO: 22 - protein, 10 aa, Artificial
  N-terminal sequence of D2E7 immunoglobulin light chain.
DIQMTQSPSS

SEQ ID NO: 23 - protein, 22 aa, Artificial
  D2E7 light chain signal sequence.
MDMRVPAQLLGLLLLWFPGSRC

SEQ ID NO: 24 - protein, 20 aa, Artificial
  D2E7 signal peptide sequence in Construct H.
MDMRVPAQLLGDEWFPGSRC

SEQ ID NO: 25 - protein, 15 aa, Artificial
  Amino acid sequence at end of intein and in start of light chain protein in Construct J.
MDMRVPAQWFPGSRC

SEQ ID NO: 26 - protein, 10 aa, Artificial
  N-terminal sequence of light chain in Construct H.
MDMRVPAQLL

SEQ ID NO: 27 - protein, 22 aa, Artificial
  Amino acid sequence following intein in Construct L.
MDMRVPAQLLGLLLLWFPGSGG

SEQ ID NO: 28 - protein, 10 aa, Artificial
  Signal peptidase cleavage site sequence.
LAGFATVAQA

SEQ ID NO: 29 - DNA, 2925 bp, Artificial
  Synthetic construct, D2E7 LC-LC-HC Polyprotein coding sequence.
atggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggaaggtgtaagagacttctcaagttggcaggagacgttgagtccaaccctgggcccatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggaaggtgtaagagacttctcaagttggcaggagacgttgagtccaaccctgggcccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - protein, 974 aa, Artificial [sequence omitted]

SEQ ID NO: 31 - DNA, 10323 bp, Artificial
  Synthetic construct, D2E7 LC-LC-HC Polyprotein Expression Vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggaaggtgtaagagacttctcaagttggcaggagacgttgagtccaaccctgggcccatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggaaggtgtaagagacttctcaagttggcaggagacgttgagtccaaccctgggcccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagaattagtctactcgcaaggggcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 32 - DNA, 2835 bp, Artificial
  Synthetic construct, coding seuqence for ABT-007 polyprotein.
atggagtttgggctgagctggctttttcttgtcgcgattttaaaaggtgtccagtgtcaggtgcagctgcaggagtcgggcccaggactggtgaagccttcggagaccctgtccctcacctgcactgtctctggtgcctccatcagtagttactactggagctggatccggcagcccccagggaagggactggagtggattgggtatatcgggggggaggggagcaccaactacaacccctccctcaagagtcgagtcaccatatcagtagacacgtccaagaaccagttctccctgaagctgaggtctgtgaccgctgcggacacggccgtgtattactgtgcgagagagcgactggggatcggggactactggggccagggaaccctggtcaccgtctcctcagcgtcgaccaagggcccatcggtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctgcagtcctcaggactctactccctcagcagcgtggtgaccgtgccctccagcaacttcggcacccagacctacacatgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatgcgcgtgcccgcccagctgctgggcctgctgctgctgtggttccccggctcgcgatgcgacatccagctgacccaatctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttaccctccgacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 33 - protein, 944 aa, Artificial [sequence omitted]

SEQ ID NO: 34 - DNA, 10212 bp, Artificial
  Synthetic construct, ABT-007 polyprotein expression vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggagtttgggctgagctggctttttcttgtcgcgattttaaaaggtgtccagtgtcaggtgcagctgcaggagtcgggcccaggactggtgaagccttcggagaccctgtccctcacctgcactgtctctggtgcctccatcagtagttactactggagctggatccggcagcccccagggaagggactggagtggattgggtatatcgggggggaggggagcaccaactacaacccctccctcaagagtcgagtcaccatatcagtagacacgtccaagaaccagttctccctgaagctgaggtctgtgaccgctgcggacacggccgtgtattactgtgcgagagagcgactggggatcggggactactggggccagggaaccctggtcaccgtctcctcagcgtcgaccaagggcccatcggtcttccccctggcgccctgctctagaagcacctccgagagcacagcggccctgggctgcctggtcaaggactacttccccgaaccggtgacggtgtcgtggaactcaggcgctctgaccagcggcgtgcacaccttcccagctgtcctgcagtcctcaggactctactccctcagcagcgtggtgaccgtgccctccagcaacttcggcacccagacctacacatgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatgcgcgtgcccgcccagctgctgggcctgctgctgctgtggttccccggctcgcgatgcgacatccagctgacccaatctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttaccctccgacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 35 - DNA, 2853 bp, Artificial
  Synthetic construct, sequence encoding ABT-874 (J695) TEV Polyprotein.
atggagtttgggctgagctggctttttcttgtcgcgattttaaaaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcatttatacggtatgatggaagtaataaatactatgcagactccgtgaagggccgattcaccatctccagagacaattccaagaacacgctgtatctgcagatgaacagcctgagagctgaggacacggctgtgtattactgtaagacccatggtagccatgacaactggggccaagggacaatggtcaccgtctcttcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatgacttggaccccactcctcttcctcaccctcctcctccactgcacaggaagcttatcccagtctgtgctgactcagcccccctcagtgtctggggcccccgggcagagagtcaccatctcttgttctggaagcagatccaacatcggcagtaatactgtaaagtggtatcagcagctcccaggaacggcccccaaactcctcatctattacaatgatcagcggccctcaggggtccctgaccgattctctggatccaagtctggcacctcagcctccctcgccatcactgggctccaggctgaagacgaggctgactattactgccagtcatatgacagatacacccaccccgccctgctcttcggaactgggaccaaggtcacagtactaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatga

SEQ ID NO: 36 - protein, 950 aa, Artificial [sequence omitted]

SEQ ID NO: 37 - DNA, 10230 bp, Artificial
  Synthetic construct, ABT-874 TEV polyprotein expression vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggagtttgggctgagctggctttttcttgtcgcgattttaaaaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcatttatacggtatgatggaagtaataaatactatgcagactccgtgaagggccgattcaccatctccagagacaattccaagaacacgctgtatctgcagatgaacagcctgagagctgaggacacggctgtgtattactgtaagacccatggtagccatgacaactggggccaagggacaatggtcaccgtctcttcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatgacttggaccccactcctcttcctcaccctcctcctccactgcacaggaagcttatcccagtctgtgctgactcagcccccctcagtgtctggggcccccgggcagagagtcaccatctcttgttctggaagcagatccaacatcggcagtaatactgtaaagtggtatcagcagctcccaggaacggcccccaaactcctcatctattacaatgatcagcggccctcaggggtccctgaccgattctctggatccaagtctggcacctcagcctccctcgccatcactgggctccaggctgaagacgaggctgactattactgccagtcatatgacagatacacccaccccgccctgctcttcggaactgggaccaaggtcacagtactaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatgagcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 38 - DNA, 2901 bp, Artificial
  Synthetic construct, sequence encoding EL246 GG TEV polyprotein.
atggagtttgggctgagctggctttttcttgtcgcgattttaaaaggtgtccagtgcgaggtgcagctggtgcagtctggagcagaggtgaaaaagcccggggagtctctgaagatctcctgtaaggggtccggatacgcattcagtagttcctggatcggctgggtgcgccagatgcccgggaaaggcctggagtggatggggcggatttatcctggagatggagatactaactacaatgggaagttcaagggccaggtcaccatctcagccgacaagtccatcagcaccgcctacctgcagtggagcagcctgaaggctagcgacaccgccatgtattactgtgcgagagcgcgcgtgggatccacggtctatgatggttacctctatgcaatggactactggggtcaaggtacctcagtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatggacatgcgcgtgcccgcccagctgctgggcctgctgctgctgtggttccccggctcgcgatgcgacatcgtgatgacccagtctccagactccctggctgtgtctctgggcgagagggccaccatcaactgcaagtccagtcagagcctttcatatagaagcaatcaaaagaactcgttggcctggtaccagcagaaaccaggacagcctcctaagctgctcatttactgggctagcactagggaatctggggtccctgaccgattcagtggatccgggtctgggacagatttcactctcaccatcagcagcctgcaggctgaagatgtggcagtttattactgtcaccaatattatagctatccgtacacgttcggaggggggaccaaggtggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 39 - protein, 966 aa, Artificial [sequence omitted]

SEQ ID NO: 40 - DNA, 10278 bp, Artificial
  Synthetic construct, EL246 GG TEV Polyprotein expression vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggagtttgggctgagctggctttttcttgtcgcgattttaaaaggtgtccagtgcgaggtgcagctggtgcagtctggagcagaggtgaaaaagcccggggagtctctgaagatctcctgtaaggggtccggatacgcattcagtagttcctggatcggctgggtgcgccagatgcccgggaaaggcctggagtggatggggcggatttatcctggagatggagatactaactacaatgggaagttcaagggccaggtcaccatctcagccgacaagtccatcagcaccgcctacctgcagtggagcagcctgaaggctagcgacaccgccatgtattactgtgcgagagcgcgcgtgggatccacggtctatgatggttacctctatgcaatggactactggggtcaaggtacctcagtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatggacatgcgcgtgcccgcccagctgctgggcctgctgctgctgtggttccccggctcgcgatgcgacatcgtgatgacccagtctccagactccctggctgtgtctctgggcgagagggccaccatcaactgcaagtccagtcagagcctttcatatagaagcaatcaaaagaactcgttggcctggtaccagcagaaaccaggacagcctcctaagctgctcatttactgggctagcactagggaatctggggtccctgaccgattcagtggatccgggtctgggacagatttcactctcaccatcagcagcctgcaggctgaagatgtggcagtttattactgtcaccaatattatagctatccgtacacgttcggaggggggaccaaggtggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 41 - DNA, 2865 bp, Artificial
  Synthetic construct, ABT-325 TEV polyprotein coding sequence.
atggagtttgggctgagctggcttttccttgtcgcgattttaaaaggtgtccagtgtgaggtgcagctggtgcagtctggaacagaggtgaaaaaacccggggagtctctgaagatctcctgtaagggttctggatacactgttaccagttactggatcggctgggtgcgccagatgcccgggaaaggcctggagtggatgggattcatctatcctggtgactctgaaaccagatacagtccgaccttccaaggccaggtcaccatctcagccgacaagtccttcaataccgccttcctgcagtggagcagtctaaaggcctcggacaccgccatgtattactgtgcgcgagtcggcagtggctggtacccttatacttttgatatctggggccaagggacaatggtcaccgtctcttcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatggaagccccagcgcagcttctcttcctcctgctactctggctcccagataccactggagaaatagtgatgacgcagtctccagccaccctgtctgtgtctccaggggaaagagccaccctctcctgcagggccagtgagagtattagcagcaacttagcctggtaccagcagaaacctggccaggctcccaggctcttcatctatactgcatccaccagggccactgatatcccagccaggttcagtggcagtgggtctgggacagagttcactctcaccatcagcagcctgcagtctgaagattttgcagtttattactgtcagcagtataataactggccttcgatcaccttcggccaagggacacgactggagattaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 42 - protein, 954 aa, Artificial
  Synthetic Construct
MEFGLSWLFLVAILKGVQCEVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSRGKREPVYFQGSLFKGPRDYNPISSAICHLTNESDGHTTSLYGIGFGPFIITNKHLFRRNNGTLLVQSLHGVFKVKNTTTLQQHLIDGRDMMLIRMPKDFPPFPQKLKFREPQREERICLVTTNFQTKSMSSMVSDTSCTFPSSDGIFWKHWIQTKDGHCGSPLVSTRDGFIVGIHSASNFTNTNNYFTSVPKDFMDLLTNQEAQQWVSGWRLNADSVLWGGHKVFMSKPEEPFQPVKEATQLMSELVYSQGMEAPAQLLFLLLLWLPDTTGEIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 10242 bp, Artificial
  Synthetic construct, ABT-325 TEV polyprotein expression vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggagtttgggctgagctggcttttccttgtcgcgattttaaaaggtgtccagtgtgaggtgcagctggtgcagtctggaacagaggtgaaaaaacccggggagtctctgaagatctcctgtaagggttctggatacactgttaccagttactggatcggctgggtgcgccagatgcccgggaaaggcctggagtggatgggattcatctatcctggtgactctgaaaccagatacagtccgaccttccaaggccaggtcaccatctcagccgacaagtccttcaataccgccttcctgcagtggagcagtctaaaggcctcggacaccgccatgtattactgtgcgcgagtcggcagtggctggtacccttatacttttgatatctggggccaagggacaatggtcaccgtctcttcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatggaagccccagcgcagcttctcttcctcctgctactctggctcccagataccactggagaaatagtgatgacgcagtctccagccaccctgtctgtgtctccaggggaaagagccaccctctcctgcagggccagtgagagtattagcagcaacttagcctggtaccagcagaaacctggccaggctcccaggctcttcatctatactgcatccaccagggccactgatatcccagccaggttcagtggcagtgggtctgggacagagttcactctcaccatcagcagcctgcagtctgaagattttgcagtttattactgtcagcagtataataactggccttcgatcaccttcggccaagggacacgactggagattaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 44 - DNA, 10245 bp, Artificial
  Synthetic construct, D2E7 TEV polyprotein expression vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcgaaccagtttatttccaggggagcttgtttaaggggccgcgtgattataacccaatatcgagtgccatttgtcatctaacgaatgaatctgatgggcacacaacatcgttgtatggtattggttttggccctttcatcatcacaaacaagcatttgtttagaagaaataatggtacactgttagttcaatcactacatggtgtgttcaaggtaaagaataccacaactttgcaacaacacctcattgatgggagggacatgatgctcattcgcatgcctaaggatttcccaccatttcctcaaaagctgaaattcagagagccacaaagggaagagcgcatatgtcttgtgacaaccaacttccaaactaagagcatgtctagcatggtttcagatactagttgcacattcccttcatctgatggtatattctggaaacattggattcagaccaaggatgggcactgtggtagcccgttggtgtcaactagagatgggtttattgttggtatacactcagcatcaaatttcaccaacacaaacaattattttacaagtgtgccgaaagacttcatggatttattgacaaatcaagaggcgcagcaatgggttagtggttggcgattgaatgctgactcagtgttatggggaggccacaaagttttcatgagcaaacctgaagaaccctttcagccagtcaaagaagcaactcaactcatgagtgaattagtctactcgcaagggatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 45 - DNA, 2196 bp, Artificial
  Synthetic construct, sequence encoding D2E7 internal cleavable signal peptide construct.
atggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcatgggacgaatggcaatgaaatggttagttgttataatatgtttctctataacaagtcaacctgcttctgctatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 46 - protein, 731 aa, Artificial
  Synthetic Construct
MEFGLSWLFLVAILKGVQCEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSRGKRMGRMAMKWLVVIICFSITSQPASAMDMRVPAQLLGLLLLWFPGSRCDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - DNA, 9573 bp, Artificial
  Synthetic construct, D2E7 internal cleavable signal peptide polyprotein expression vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctaggggtaaacgcatgggacgaatggcaatgaaatggttagttgttataatatgtttctctataacaagtcaacctgcttctgctatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 48 - DNA, 3252 bp, Artificial
  Synthetic construct, D2E7 intein fusion polyprotein coding sequence.
atggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaccattttaccggaagaatgggttccactaattaaaaacggtaaagttaagatattccgcattggggacttcgttgatggacttatgaaggcgaaccaaggaaaagtgaagaaaacgggggatacagaagttttagaagttgcaggaattcatgcgttttcctttgacaggaagtccaagaaggcccgtgtaatggcagtgaaagccgtgataagacaccgttattccggaaatgtttatagaatagtcttaaactctggtagaaaaataacaataacagaagggcatagcctatttgtctataggaacggggatctcgttgaggcaactggggaggatgtcaaaattggggatcttcttgcagttccaagatcagtaaacctaccagagaaaagggaacgcttgaatattgttgaacttcttctgaatctctcaccggaagagacagaagatataatacttacgattccagttaaaggcagaaagaacttcttcaagggaatgttgagaacattacgttggatttttggtgaggaaaagagagtaaggacagcgagccgctatctaagacaccttgaaaatctcggatacataaggttgaggaaaattggatacgacatcattgataaggaggggcttgagaaatatagaacgttgtacgagaaacttgttgatgttgtccgctataatggcaacaagagagagtatttagttgaatttaatgctgtccgggacgttatctcactaatgccagaggaagaactgaaggaatggcgtattggaactagaaatggattcagaatgggtacgttcgtagatattgatgaagattttgccaagcttggatacgatagcggagtctacagggtttatgtaaacgaggaacttaagtttacggaatacagaaagaaaaagaatgtatatcactctcacattgttccaaaggatattctcaaagaaacttttggtaaggtcttccagaaaaatataagttacaagaaatttagagagcttgtagaaaatggaaaacttgacagggagaaagccaaacgcattgagtggttacttaacggagatatagtcctagatagagtcgtagagattaagagagagtactatgatggttacgtttacgatctaagtgtcgatgaagatgagaatttccttgctggctttggattcctctatgcacataatgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 49 - protein, 1083 aa, Artificial
  Synthetic Construct
MEFGLSWLFLVAILKGVQCEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTILPEEWVPLIKNGKVKIFRIGDFVDGLMKANQGKVKKTGDTEVLEVAGIHAFSFDRKSKKARVMAVKAVIRHRYSGNVYRIVLNSGRKITITEGHSLFVYRNGDLVEATGEDVKIGDLLAVPRSVNLPEKRERLNIVELLLNLSPEETEDIILTIPVKGRKNFFKGMLRTLRWIFGEEKRVRTASRYLRHLENLGYIRLRKIGYDIIDKEGLEKYRTLYEKLVDVVRYNGNKREYLVEFNAVRDVISLMPEEELKEWRIGTRNGFRMGTFVDIDEDFAKLGYDSGVYRVYVNEELKFTEYRKKKNVYHSHIVPKDILKETFGKVFQKNISYKKFRELVENGKLDREKAKRIEWLLNGDIVLDRVVEIKREYYDGYVYDLSVDEDENFLAGFGFLYAHNDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - DNA, 10629 bp, Artificial
  Synthetic construct, D2E7 intein fusion protein expression vector.
gaagttcctattccgaagttcctattctctagacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccaatgacgcaaatgggcagggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcccgggcgccaccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaccattttaccggaagaatgggttccactaattaaaaacggtaaagttaagatattccgcattggggacttcgttgatggacttatgaaggcgaaccaaggaaaagtgaagaaaacgggggatacagaagttttagaagttgcaggaattcatgcgttttcctttgacaggaagtccaagaaggcccgtgtaatggcagtgaaagccgtgataagacaccgttattccggaaatgtttatagaatagtcttaaactctggtagaaaaataacaataacagaagggcatagcctatttgtctataggaacggggatctcgttgaggcaactggggaggatgtcaaaattggggatcttcttgcagttccaagatcagtaaacctaccagagaaaagggaacgcttgaatattgttgaacttcttctgaatctctcaccggaagagacagaagatataatacttacgattccagttaaaggcagaaagaacttcttcaagggaatgttgagaacattacgttggatttttggtgaggaaaagagagtaaggacagcgagccgctatctaagacaccttgaaaatctcggatacataaggttgaggaaaattggatacgacatcattgataaggaggggcttgagaaatatagaacgttgtacgagaaacttgttgatgttgtccgctataatggcaacaagagagagtatttagttgaatttaatgctgtccgggacgttatctcactaatgccagaggaagaactgaaggaatggcgtattggaactagaaatggattcagaatgggtacgttcgtagatattgatgaagattttgccaagcttggatacgatagcggagtctacagggtttatgtaaacgaggaacttaagtttacggaatacagaaagaaaaagaatgtatatcactctcacattgttccaaaggatattctcaaagaaacttttggtaaggtcttccagaaaaatataagttacaagaaatttagagagcttgtagaaaatggaaaacttgacagggagaaagccaaacgcattgagtggttacttaacggagatatagtcctagatagagtcgtagagattaagagagagtactatgatggttacgtttacgatctaagtgtcgatgaagatgagaatttccttgctggctttggattcctctatgcacataatgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgagcggccgcgtttaaactgaatgagcgcgtccatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgacggcgcgcctttttttttaatttttattttattttatttttgacgcgccgaaggcgcgatctgagctcggtacagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggcctaatacgactcactatagggactagtatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttagatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaagcggccgagcgcgcggatctggaaacgggagatgggggaggctaactgaagcacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggagatcccccgggctgcaggaattccgttacattacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaaagggcgggaattcgagctcggtactcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaaccggaattgtacccgcggccagagcttgcgggcgccaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggaaaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttcttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcccttttaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagttacgacgttgtaaaacgacggccagtgaatt

SEQ ID NO: 51 - protein, 547 aa, Pyrococcus sp.
NSILPEEWVPLIKNGKVKIFRIGDFVDGLMKANQGKVKKTGDTEVLEVAGIHAFSFDRKSKKARVMAVKAVIRHRYSGNVYRIVLNSGRKITITEGHSLFVYRNGDLVEATGEDVKIGDLLAVPRSVNLPEKRERLNIVELLLNLSPEETEDIILTIPVKGRKNFFKGMLRTLRWIFGEEKRVRTASRYLRHLENLGYIRLRKIGYDIIDKEGLEKYRTLYEKLVDVVRYNGNKREYLVEFNAVRDVISLMPEEELKEWRIGTRNGFRMGTFVDIDEDFAKLLGYYVSEGSARKWKNQTGGWSYTVRLYNENDEVLDDMEHLAKKFFGKVKRGKNYVEIPKKMAYIIFESLCGTLAENKRVPEVIFTSSKGVRWAFLEGYFIGDGDVHPSKRVRLSTKSELLVNGLVLLLNSLGVSAIKLGYDSGVYRVYVNEELKFTEYRKKKNVYHSHIVPKDILKETFGKVFQKNISYKKFRELVENGKLDREKAKRIEWLLNGDIVLDRVVEIKREYYDGYVYDLSVDEDENFLAGFGFLYAHNSYYGYYGYA

SEQ ID NO: 52 - DNA, 26 bp, Artificial
  Synthetic construct, oligonucleotide useful as primer.
agcattttaccagatgaatggctccc

SEQ ID NO: 53 - DNA, 27 bp, Artificial
  Synthetic construct, oligonucleotide useful as primer.
aacgaggaagttctcattatcctcaac

SEQ ID NO: 54 - DNA, 44 bp, Artificial
  Synthetic construct; oligonucleotide useful as a primer.
agcctctccctgtctccgggtaaaagcattttaccagatgaatg

SEQ ID NO: 55 - DNA, 42 bp, Artificial
  Synthetic construct  oligonucleotide useful as a primer.
gggcgggcacgcgcatgtccatgttgtgtgcgtaaagtagtc

SEQ ID NO: 56 - DNA, 47 bp, Artificial
  Synthetic construct  oligonucleotide useful as a primer.
agcctctccctgtctccgggtaaaaacagcattttaccagatgaatg

SEQ ID NO: 57 - DNA, 45 bp, Artificial
  Synthetic construct  oligonucleotide useful as a primer.
gggcgggcacgcgcatgtccatactgttgtgtgcgtaaagtagtc

SEQ ID NO: 58 - DNA, 53 bp, Artificial
  Synthetic construct  oligonucleotide useful as a primer.
agcctctccctgtctccgggtaaattagcaaacagcattttaccagatgaatg

SEQ ID NO: 59 - DNA, 51 bp, Artificial
  Synthetic construct  oligonucleotide useful as a primer.
gggcgggcacgcgcatgtccatgtaataactgttgtgtgcgtaaagtagtc

SEQ ID NO: 60 - DNA, 36 bp, Artificial
  Synthetic construct  oligonucleotide useful as a primer.
tgcccgggcgccaccatggagtttgggctgagctgg

SEQ ID NO: 61 - DNA, 36 bp, Artificial; identical to SEQ ID NO: 60
  Synthetic construct  oligonucleotide useful as a primer.

SEQ ID NO: 62 - DNA, 9460 bp, Artificial
  Synthetic construct  sequence of plasmid pTT3-HcintLC-p.hori
gcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttgggcgccaccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 63 - protein, 1166 aa, Artificial [sequence omitted]

SEQ ID NO: 64 - DNA, 1404 bp, Artificial
  Synthetic construct  partial coding sequence from pTT3-HcintLC1aa-p.hori
ccgggtaaaaacagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacagtatggacatg

SEQ ID NO: 65 - protein, 468 aa, Artificial
  Synthetic  partial amino acid sequence from pTT3-HcintLC1aa-p.hori, showing 4 amino acids upstream of the heavy chain and four amino acids downstream of the intein.
PGKNSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAHNSMDM

SEQ ID NO: 66 - DNA, 1416 bp, Artificial
  Synthetic construct  pTT3-HcintLC3aa-p.hori partial coding sequence.
ccgggtaaattagcaaacagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacagttattacatggacatg

SEQ ID NO: 67 - protein, 472 aa, Artificial
  Synthetic  pTT3-HcintLC3aa-p.hori partial amino acid sequence showing intein and flanking sequences.
PGKLANSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAHNSYYMDM

SEQ ID NO: 68 - DNA, 31 bp, Artificial
  Synthetic construct  oligonucleotide useful as a primer.
ggactactttacgcagccaacatggacatgc

SEQ ID NO: 69 - DNA, 31 bp, Artificial
  Synthetic construct  oligonucleotide useful as a primer.
gcatgtccatgttggctgcgtaaagtagtcc

SEQ ID NO: 70 - DNA, 34 bp, Artificial
  Synthetic construct  oligonucleotide useful as a primer.
ggactactttacgcagccaacagtatggacatgc

SEQ ID NO: 71 - DNA, 34 bp, Artificial
  Synthetic construct  oligonucleotide useful as a primer.
gcatgtccatactgttggctgcgtaaagtagtcc

SEQ ID NO: 72 - DNA, 18 bp, Artificial
  Synthetic  construct  oligonucleotide useful as a primer.
ggtgaggagaggaagagg

SEQ ID NO: 73 - DNA, 16 bp, Artificial
  Synthetic construct  oligonucleotide useful as a primer.
ccagaggtcgaggtcg

SEQ ID NO: 74 - DNA, 14 bp, Artificial
  Synthetic construct  oligonucleotide useful as a primer.
cggcgtggaggtgc

SEQ ID NO: 75 - DNA, 45 bp, Artificial
  Synthetic construct  oligonucleotide useful as a primer.
caacaattgggagccattcatctggtaaaatggttttacccggag

SEQ ID NO: 76 - DNA, 40 bp, Artificial
  Synthetic construct  oligonucleotide useful as a primer.
ccgcccagctgctgggcgacgagtggttccccggctcgcg

SEQ ID NO: 77 - DNA, 40 bp, Artificial
  Synthetic construct  oligonucleotide useful as a primer.
cgcgagccggggaaccactcgtcgcccagcagctgggcgg

SEQ ID NO: 78 - DNA, 15 bp, Artificial
  Synthetic construct  oligonucleotide useful as a primer.
tgagcggccgctcga

SEQ ID NO: 79 - DNA, 15 bp, Artificial
  Synthetic construct  oligonucleotide useful as a primer.
gttgtgtgcgtaaag

SEQ ID NO: 80 - DNA, 15 bp, Artificial
  Synthetic construct  oligonucleotide useful as a primer.
agcattttaccagat

SEQ ID NO: 81 - DNA, 15 bp, Artificial
  Synthetic construct  oligonucleotide useful as a primer.
ggtggcgcccaaact

SEQ ID NO: 82 - DNA, 30 bp, Artificial
  Synthetic construct  oligonucleotide useful as a primer.
ctttacgcacacaacatggacatgcgcgtg

SEQ ID NO: 83 - DNA, 27 bp, Artificial
  Synthetic construct  oligonucleotide useful as a primer.
tcgagcggccgctcaacactctcccct

SEQ ID NO: 84 - DNA, 30 bp, Artificial
  Synthetic construct  oligonucleotide useful as a primer.
agtttgggcgccaccatggagtttgggctg

SEQ ID NO: 85 - DNA, 30 bp, Artificial
  Synthetic construct  oligonucleotide useful as a primer.
atctggtaaaatgcttttacccggagacag

SEQ ID NO: 86 - DNA, 30 bp, Artificial
  Synthetic construct  oligonucleotide useful as a primer.
agtttgggcgccaccatggacatgcgcgtg

SEQ ID NO: 87 - DNA, 31 bp, Artificial
  Synthetic construct  oligonucleotide useful as a primer.
atctggtaaaatgctacactctcccctgttg

SEQ ID NO: 88 - DNA, 30 bp, Artificial
  Synthetic construct  oligonucleotide useful as a primer.
ctttacgcacacaacatggagtttgggctg

SEQ ID NO: 89 - DNA, 30 bp, Artificial
  Synthetic construct  oligonucleotide useful as a primer.
tcgagcggccgctcatttacccggagacag

SEQ ID NO: 90 - DNA, 14 bp, Artificial
  Synthetic construct  oligonucleotide useful as a primer.
cgccaagctctagc

SEQ ID NO: 91 - DNA, 14 bp, Artificial
  Synthetic construct  oligonucleotide useful as a primer.
ggtcgaggtcgggg

SEQ ID NO: 92 - DNA, 40 bp, Artificial
  Synthetic construct  oligonucleotide useful as a primer.
acatgcgcgtgcccgcccagtggttccccggctcgcgatg

SEQ ID NO: 93 - DNA, 40 bp, Artificial
  Synthetic construct  oligonucleotide useful as a primer.
catcgcgagccggggaaccactgggcgggcacgcgcatgt

SEQ ID NO: 94 - DNA, 30 bp, Artificial
  Synthetic construct  oligonucleotide useful as a primer.
ctttacgcacacaacgacatccagatgacc

SEQ ID NO: 95 - DNA, 30 bp, Artificial
  Synthetic construct  oligonucleotide useful as a primer.
ggtcatctggatgtcgttgtgtgcgtaaag

SEQ ID NO: 96 - DNA, 36 bp, Artificial
  Synthetic construct  oligonucleotide useful as a primer.
tggttccccggctcgggaggcgacatccagatgacc

SEQ ID NO: 97 - DNA, 36 bp, Artificial
  Synthetic construct  oligonucleotide useful as a primer.
ggtcatctggatgtcgcctcccgagccggggaacca

SEQ ID NO: 98 - DNA, 1464 bp, Artificial
  Synthetic construct  partial coding sequence of Construct A.
ccgggtaaaagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcagccaacatggacatgcgcgtgcccgcccagctgctgggcctgctgctgctgtggttccccggctcgcgatgcgacatccag

SEQ ID NO: 99 - protein, 488 aa, Artificial
  Synthetic  partial amino acid sequence of Construct A.
PGKSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAANMDMRVPAQLLGLLLLWFPGSRCDIQ

SEQ ID NO: 100 - DNA, 1467 bp, Artificial
  Synthetic construct  partial coding sequence of construct B.
ccgggtaaaagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcagccaacagtatggacatgcgcgtgcccgcccagctgctgggcctgctgctgctgtggttccccggctcgcgatgcgacatccag

SEQ ID NO: 101 - protein, 489 aa, Artificial
  Synthetic construct  partial amino acid sequence of construct A.
PGKSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAANSMDMRVPAQLLGLLLLWFPGSRCDIQ

SEQ ID NO: 102 - DNA, 1467 bp, Artificial
  Synthetic construct  partial coding sequence in construct E.
ccgggtaaaaccattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacagtatggacatgcgcgtgcccgcccagctgctgggcctgctgctgctgtggttccccggctcgcgatgcgacatccag

SEQ ID NO: 103 - protein, 489 aa, Artificial
  Synthetic construct  partial amino acid sequence from construct E.
PGKTILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAHNSMDMRVPAQLLGLLLLWFPGSRCDIQ

SEQ ID NO: 104 - DNA, 1458 bp, Artificial
  Synthetic construct  partial coding sequence from construct H.
ccgggtaaaagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacatggacatgcgcgtgcccgcccagctgctgggcgacgagtggttccccggctcgcgatgcgacatccag

SEQ ID NO: 105 - protein, 486 aa, Artificial
  Synthetic construct  partial amino acid sequence from construct H.
PGKSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAHNMDMRVPAQLLGDEWFPGSRCDIQ

SEQ ID NO: 106 - DNA, 1443 bp, Artificial
  Synthetic construct  partial coding sequence for construct J.
ccgggtaaaagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacatggacatgcgcgtgcccgcccagtggttccccggctcgcgatgcgacatccag

SEQ ID NO: 107 - protein, 481 aa, Artificial
  Synthetic construct  partial amino acid sequence from construct J.
PGKSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAHNMDMRVPAQWFPGSRCDIQ

SEQ ID NO: 108 - DNA, 1398 bp, Artificial
  Synthetic construct  partial coding sequence for construct K.
ccgggtaaaagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacgacatccag

SEQ ID NO: 109 - protein, 466 aa, Artificial
  Synthetic construct  partial amino acid sequence for construct K.
PGKSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAHNDIQ

SEQ ID NO: 110 - DNA, 1464 bp, Artificial
  Synthetic construct  partial coding sequence for construct L.
ccgggtaaaagcattttaccagatgaatggctcccaattgttgaaaatgaaaaagttcgattcgtaaaaattggagacttcatagatagggagattgaggaaaacgctgagagagtgaagagggatggtgaaactgaaattctagaggttaaagatcttaaagccctttccttcaatagagaaacaaaaaagagcgagctcaagaaggtaaaggccctaattagacaccgctattcagggaaggtttacagcattaaactaaagtcagggagaaggatcaaaataacctcaggtcatagtctgttctcagtaaaaaatggaaagctagttaaggtcaggggagatgaactcaagcctggtgatctcgttgtcgttccaggaaggttaaaacttccagaaagcaagcaagtgctaaatctcgttgaactactcctgaaattacccgaagaggagacatcgaacatcgtaatgatgatcccagttaaaggtagaaagaatttcttcaaagggatgctcaaaacattatactggatcttcggggagggagaaaggccaagaaccgcagggcgctatctcaagcatcttgaaagattaggatacgttaagctcaagagaagaggctgtgaagttctcgactgggagtcacttaagaggtacaggaagctttacgagaccctcattaagaacctgaaatataacggtaatagcagggcatacatggttgaatttaactctctcagggatgtagtgagcttaatgccaatagaagaacttaaggagtggataattggagaacctaggggtcctaagataggtaccttcattgatgtagatgattcatttgcaaagctcctaggttactacataagtagcggagatgtagagaaagatagggtgaagttccacagtaaagatcaaaacgttctcgaggatatagcgaaacttgccgagaagttatttggaaaggtgaggagaggaagaggatatattgaggtatcagggaaaattagccatgccatatttagagttttagcggaaggtaagagaattccagagttcatcttcacatccccaatggatattaaggtagccttccttaagggactcaacggtaatgctgaagaattaacgttctccactaagagtgagctattagttaaccagcttatccttctcctgaactccattggagtttcggatataaagattgaacatgagaaaggggtttacagagtttacataaataagaaggaatcctccaatggggatatagtacttgatagcgtcgaatctatcgaagttgaaaaatacgagggctacgtttatgatctaagtgttgaggataatgagaacttcctcgttggcttcggactactttacgcacacaacatggacatgcgcgtgcccgcccagctgctgggcctgctgctgctgtggttccccggctcgggaggcgacatccag

SEQ ID NO: 111 - protein, 488 aa, Artificial
  Synthetic construct  partial amino acid sequence of construct L.
PGKSILPDEWLPIVENEKVRFVKIGDFIDREIEENAERVKRDGETEILEVKDLKALSFNRETKKSELKKVKALIRHRYSGKVYSIKLKSGRRIKITSGHSLFSVKNGKLVKVRGDELKPGDLVVVPGRLKLPESKQVLNLVELLLKLPEEETSNIVMMIPVKGRKNFFKGMLKTLYWIFGEGERPRTAGRYLKHLERLGYVKLKRRGCEVLDWESLKRYRKLYETLIKNLKYNGNSRAYMVEFNSLRDVVSLMPIEELKEWIIGEPRGPKIGTFIDVDDSFAKLLGYYISSGDVEKDRVKFHSKDQNVLEDIAKLAEKLFGKVRRGRGYIEVSGKISHAIFRVLAEGKRIPEFIFTSPMDIKVAFLKGLNGNAEELTFSTKSELLVNQLILLLNSIGVSDIKIEHEKGVYRVYINKKESSNGDIVLDSVESIEVEKYEGYVYDLSVEDNENFLVGFGLLYAHNMDMRVPAQLLGLLLLWFPGSGGDIQ

SEQ ID NO: 112 - DNA, 26 bp, Artificial
  Synthetic construct  oligonucleotide useful as a primer.
tgctttgccaagggtaccaatgtttt

SEQ ID NO: 113 - DNA, 26 bp, Artificial
  Synthetic construct  oligonucleotide useful as a primer.
attatggacgacaacctggttggcaa

SEQ ID NO: 114 - DNA, 59 bp, Artificial
  Synthetic construct  oligonucleotide useful as a primer.
ccgcagaagagcctctccctgtctccgggtaaatgctttgccaagggtaccaatgtttt

SEQ ID NO: 115 - DNA, 62 bp, Artificial
  Synthetic construct  oligonucleotide useful as a primer.
ccgcagaagagcctctccctgtctccgggtaaagggtgctttgccaagggtaccaatgtttt

SEQ ID NO: 116 - DNA, 68 bp, Artificial
  Synthetic construct  oligonucleotide useful as a primer.
ccgcagaagagcctctccctgtctccgggtaaatatgtcgggtgctttgccaagggtaccaatgtttt

SEQ ID NO: 117 - DNA, 65 bp, Artificial
  Synthetic construct  oligonucleotide useful as a primer.
cagcaggcccagcagctgggcgggcacgcgcatgtccatattatggacgacaacctggttggcaa

SEQ ID NO: 118 - DNA, 68 bp, Artificial
  Synthetic construct  oligonucleotide useful as a primer.
cagcaggcccagcagctgggcgggcacgcgcatgtccatgcaattatggacgacaacctggttggcaa

SEQ ID NO: 119 - DNA, 74 bp, Artificial
  Synthetic construct  oligonucleotide useful as a primer.
cagcaggcccagcagctgggcgggcacgcgcatgtccatttctccgcaattatggacgacaacctggttggcaa

SEQ ID NO: 120 - DNA, 40 bp, Artificial
  Synthetic construct  oligonucleotide useful as a primer.
ccactacacgcagaagagcctctccctgtctccgggtaaa

SEQ ID NO: 121 - DNA, 40 bp, Artificial
  Synthetic construct  oligonucleotide useful as a primer.
gcagcaggcccagcagctgggcgggcacgcgcatgtccat

SEQ ID NO: 122 - DNA, 40 bp, Artificial
  Synthetic construct  oligonucleotide useful as a primer.
atggacatgcgcgtgcccgcccagctgctgggcctgctgc

SEQ ID NO: 123 - DNA, 41 bp, Artificial
  Synthetic construct  oligonucleotide useful as a primer.
tttacccggagacagggagaggctcttctgcgtgtagtggt

SEQ ID NO: 124 - DNA, 9442 bp, Artificial
  Synthetic construct  nucleotide sequence of plasmid pTT3-D2E7 Heavy Chain - intein - D2E7 Light Chain.
gcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttgggcgccaccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgctttgccaagggtaccaatgttttaatggcggatgggtctattgaatgtattgaaaacattgaggttggtaataaggtcatgggtaaagatggcagacctcgtgaggtaattaaattgcccagaggaagagaaactatgtacagcgtcgtgcagaaaagtcagcacagagcccacaaaagtgactcaagtcgtgaagtgccagaattactcaagtttacgtgtaatgcgacccatgagttggttgttagaacacctcgtagtgtccgccgtttgtctcgtaccattaagggtgtcgaatattttgaagttattacttttgagatgggccaaaagaaagcccccgacggtagaattgttgagcttgtcaaggaagtttcaaagagctacccaatatctgaggggcctgagagagccaacgaattagtagaatcctatagaaaggcttcaaataaagcttattttgagtggactattgaggccagagatctttctctgttgggttcccatgttcgtaaagctacctaccagacttacgctccaattctttatgagaatgaccactttttcgactacatgcaaaaaagtaagtttcatctcaccattgaaggtccaaaagtacttgcttatttacttggtttatggattggtgatggattgtctgacagggcaactttttcggttgattccagagatacttctttgatggaacgtgttactgaatatgctgaaaagttgaatttgtgcgccgagtataaggacagaaaagaaccacaagttgccaaaactgttaatttgtactctaaagttgtcagaggtaatggtattcgcaataatcttaatactgagaatccattatgggacgctattgttggcttaggattcttgaaggacggtgtcaaaaatattccttctttcttgtctacggacaatatcggtactcgtgaaacatttcttgctggtctaattgattctgatggctatgttactgatgagcatggtattaaagcaacaataaagacaattcatacttctgtcagagatggtttggtttcccttgctcgttctttaggcttagtagtctcggttaacgcagaacctgctaaggttgacatgaatggcaccaaacataaaattagttatgctatttatatgtctggtggagatgttttgcttaacgttctttcgaagtgtgccggctctaaaaaattcaggcctgctcccgccgctgcttttgcacgtgagtgccgcggattttatttcgagttacaagaattgaaggaagacgattattatgggattactttatctgatgattctgatcatcagtttttgcttgccaaccaggttgtcgtccataatatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 125 - DNA, 1386 bp, Artificial
  Synthetic construct  partial coding sequence in pTT3-HC-VMAint-LC-1aa.
ccgggtaaagggtgctttgccaagggtaccaatgttttaatggcggatgggtctattgaatgtattgaaaacattgaggttggtaataaggtcatgggtaaagatggcagacctcgtgaggtaattaaattgcccagaggaagagaaactatgtacagcgtcgtgcagaaaagtcagcacagagcccacaaaagtgactcaagtcgtgaagtgccagaattactcaagtttacgtgtaatgcgacccatgagttggttgttagaacacctcgtagtgtccgccgtttgtctcgtaccattaagggtgtcgaatattttgaagttattacttttgagatgggccaaaagaaagcccccgacggtagaattgttgagcttgtcaaggaagtttcaaagagctacccaatatctgaggggcctgagagagccaacgaattagtagaatcctatagaaaggcttcaaataaagcttattttgagtggactattgaggccagagatctttctctgttgggttcccatgttcgtaaagctacctaccagacttacgctccaattctttatgagaatgaccactttttcgactacatgcaaaaaagtaagtttcatctcaccattgaaggtccaaaagtacttgcttatttacttggtttatggattggtgatggattgtctgacagggcaactttttcggttgattccagagatacttctttgatggaacgtgttactgaatatgctgaaaagttgaatttgtgcgccgagtataaggacagaaaagaaccacaagttgccaaaactgttaatttgtactctaaagttgtcagaggtaatggtattcgcaataatcttaatactgagaatccattatgggacgctattgttggcttaggattcttgaaggacggtgtcaaaaatattccttctttcttgtctacggacaatatcggtactcgtgaaacatttcttgctggtctaattgattctgatggctatgttactgatgagcatggtattaaagcaacaataaagacaattcatacttctgtcagagatggtttggtttcccttgctcgttctttaggcttagtagtctcggttaacgcagaacctgctaaggttgacatgaatggcaccaaacataaaattagttatgctatttatatgtctggtggagatgttttgcttaacgttctttcgaagtgtgccggctctaaaaaattcaggcctgctcccgccgctgcttttgcacgtgagtgccgcggattttatttcgagttacaagaattgaaggaagacgattattatgggattactttatctgatgattctgatcatcagtttttgcttgccaaccaggttgtcgtccataattgcatggacatg

SEQ ID NO: 126 - DNA, 1398 bp, Artificial
  Synthetic construct  partial coding sequence from pTT3-HC-VMAint-LC-3aa.
ccgggtaaatatgtcgggtgctttgccaagggtaccaatgttttaatggcggatgggtctattgaatgtattgaaaacattgaggttggtaataaggtcatgggtaaagatggcagacctcgtgaggtaattaaattgcccagaggaagagaaactatgtacagcgtcgtgcagaaaagtcagcacagagcccacaaaagtgactcaagtcgtgaagtgccagaattactcaagtttacgtgtaatgcgacccatgagttggttgttagaacacctcgtagtgtccgccgtttgtctcgtaccattaagggtgtcgaatattttgaagttattacttttgagatgggccaaaagaaagcccccgacggtagaattgttgagcttgtcaaggaagtttcaaagagctacccaatatctgaggggcctgagagagccaacgaattagtagaatcctatagaaaggcttcaaataaagcttattttgagtggactattgaggccagagatctttctctgttgggttcccatgttcgtaaagctacctaccagacttacgctccaattctttatgagaatgaccactttttcgactacatgcaaaaaagtaagtttcatctcaccattgaaggtccaaaagtacttgcttatttacttggtttatggattggtgatggattgtctgacagggcaactttttcggttgattccagagatacttctttgatggaacgtgttactgaatatgctgaaaagttgaatttgtgcgccgagtataaggacagaaaagaaccacaagttgccaaaactgttaatttgtactctaaagttgtcagaggtaatggtattcgcaataatcttaatactgagaatccattatgggacgctattgttggcttaggattcttgaaggacggtgtcaaaaatattccttctttcttgtctacggacaatatcggtactcgtgaaacatttcttgctggtctaattgattctgatggctatgttactgatgagcatggtattaaagcaacaataaagacaattcatacttctgtcagagatggtttggtttcccttgctcgttctttaggcttagtagtctcggttaacgcagaacctgctaaggttgacatgaatggcaccaaacataaaattagttatgctatttatatgtctggtggagatgttttgcttaacgttctttcgaagtgtgccggctctaaaaaattcaggcctgctcccgccgctgcttttgcacgtgagtgccgcggattttatttcgagttacaagaattgaaggaagacgattattatgggattactttatctgatgattctgatcatcagtttttgcttgccaaccaggttgtcgtccataattgcggagaaatggacatg

SEQ ID NO: 127 - DNA, 1050 bp, Artificial
  Synthetic construct  engineered Synechococcus intein coding sequence.
gggcgaattgggtaccgaattctgcctgtccttcggcaccgagatcctgaccgtggagtacccgcttaacccatggcttaagacggacaggaagccgtggctctaggactggcacctcatcggccctctgcctatcggcaagatcgtgtccgaagagatcaactgctccgtgtactccgtgccgggagacggatagccgttctagcacaggcttctctagttgacgaggcacatgaggcaggaccctgagggccgggtgtatactcaggccatcgcccagtggcacgaccggggcgagcacctgggactcccggcccacatatgagtccggtagcgggtcaccgtgctggccccgctcgtggaggtgctggagtacgagctggaggacggctccgtgatccgggccacctccgaccaccgcctccacgacctcatgctcgacctcctgccgaggcactaggcccggtggaggctggtggcgtttctgaccaccgactatcagctgctggccatcgaggagatcttcgcccggcagctggacaaagactggtggctgatagtcgacgaccggtagctcctctagaagcgggccgtcgacctcctgctgaccctggagaacatcaagcagaccgaggaggccctggacaaccaccggctgccggacgactgggacctcttgtagttcgtctggctcctccgggacctgttggtggccgacggtttccctctgctggacgccggcaccatcaagatggtgaaggtgatcggcaggcggtccctaaagggagacgacctgcggccgtggtagttctaccacttccactagccgtccgccagggagggcgtgcagcggatcttcgacatcggcctgcctcaggaccacaactttctgctggccaacccgcacgtcgcctagaagctgtagccggacggagtcctggtgttgaaagacgaccggttcggcgccatcgccgccaacaagcttgagctccagcttttgttcccgccgcggtagcggcggttgttcgaactcgaggtcgaaaacaaggg

SEQ ID NO: 128 - protein, 159 aa, Artificial
  Synthetic  intein encoded by engineered Synechococcus sequence.
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIKMVKVIGRRSLGVQRIFDIGLPQDHNFLLANGAIAAN

SEQ ID NO: 129 - DNA, 61 bp, Artificial
  Synthetic construct  oligonucleotide useful as a primer.
ccactacacgcagaagagcctctccctgtctccgggtaaatgcctgtccttcggcaccgag

SEQ ID NO: 130 - DNA, 65 bp, Artificial
  Synthetic construct  oligonucleotide useful as a primer.
gcagcaggcccagcagctgggcgggcacgcgcatgtccatgttggcggcgatggcgccgttggcc

SEQ ID NO: 131 - DNA, 64 bp, Artificial
  Synthetic construct  oligonucleotide useful as a primer.
ccactacacgcagaagagcctctccctgtctccgggtaaatattgcctgtccttcggcaccgag

SEQ ID NO: 132 - DNA, 63 bp, Artificial
  Synthetic construct  oligonucleotide useful as a primer.
gcagcaggcccagcagctgggcgggcacgcgcatgtccatacagttggcggcgatggcgccgt

SEQ ID NO: 133 - DNA, 70 bp, Artificial
  Synthetic construct  oligonucleotide useful as a primer.
ccactacacgcagaagagcctctccctgtctccgggtaaagccgagtattgcctgtccttcggcaccgag

SEQ ID NO: 134 - DNA, 70 bp, Artificial; identical to SEQ ID NO: 133
  Synthetic construct  oligonucleotide useful as a primer.

SEQ ID NO: 135 - DNA, 8557 bp, Artificial
  Synthetic construct  nucleotide sequence of plasmid  pTT3-D2E7 Heavy Chain - Ssp-GA-intein - D2E7 Light Chain.
gcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttgggcgccaccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgcctgtccttcggcaccgagatcctgaccgtggagtacggccctctgcctatcggcaagatcgtgtccgaagagatcaactgctccgtgtactccgtggaccctgagggccgggtgtatactcaggccatcgcccagtggcacgaccggggcgagcaggaggtgctggagtacgagctggaggacggctccgtgatccgggccacctccgaccaccggtttctgaccaccgactatcagctgctggccatcgaggagatcttcgcccggcagctggacctgctgaccctggagaacatcaagcagaccgaggaggccctggacaaccaccggctgcctttccctctgctggacgccggcaccatcaagatggtgaaggtgatcggcaggcggtccctgggcgtgcagcggatcttcgacatcggcctgcctcaggaccacaactttctgctggccaacggcgccatcgccgccaacatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 136 - DNA, 501 bp, Artificial
  Synthetic construct  partial coding sequence from pTT3-HC-Ssp-GA-int-LC-1aa.
ccgggtaaatattgcctgtccttcggcaccgagatcctgaccgtggagtacggccctctgcctatcggcaagatcgtgtccgaagagatcaactgctccgtgtactccgtggaccctgagggccgggtgtatactcaggccatcgcccagtggcacgaccggggcgagcaggaggtgctggagtacgagctggaggacggctccgtgatccgggccacctccgaccaccggtttctgaccaccgactatcagctgctggccatcgaggagatcttcgcccggcagctggacctgctgaccctggagaacatcaagcagaccgaggaggccctggacaaccaccggctgcctttccctctgctggacgccggcaccatcaagatggtgaaggtgatcggcaggcggtccctgggcgtgcagcggatcttcgacatcggcctgcctcaggaccacaactttctgctggccaacggcgccatcgccgccaactgtatggacatg

SEQ ID NO: 137 - DNA, 513 bp, Artificial
  Synthetic construct::  relevant portion of coding sequence from plasmid pTT3-HC-Ssp-GA-int-LC-3aa.
ccgggtaaagccgagtattgcctgtccttcggcaccgagatcctgaccgtggagtacggccctctgcctatcggcaagatcgtgtccgaagagatcaactgctccgtgtactccgtggaccctgagggccgggtgtatactcaggccatcgcccagtggcacgaccggggcgagcaggaggtgctggagtacgagctggaggacggctccgtgatccgggccacctccgaccaccggtttctgaccaccgactatcagctgctggccatcgaggagatcttcgcccggcagctggacctgctgaccctggagaacatcaagcagaccgaggaggccctggacaaccaccggctgcctttccctctgctggacgccggcaccatcaagatggtgaaggtgatcggcaggcggtccctgggcgtgcagcggatcttcgacatcggcctgcctcaggaccacaactttctgctggccaacggcgccatcgccgccaactgtttcaacatggacatg

SEQ ID NO: 138 - protein, 11 aa, Pyrococcus sp.
RQRAIKILANS

SEQ ID NO: 139 - protein, 12 aa, Pyrococcus sp.
HNSYYGYYGYAK

SEQ ID NO: 140 - protein, 214 aa, Artificial
  Synthetic construct  partial amino acid sequence encompassing cleavage sites in Hedgehog-antibody constructs.
CFTPESTALLESGVRKPLGELSIGDRVLSMTANGQAVYSEVILFMDRNLEQMQNFVQLHTDGGAVLTVTPAHLVSVWQPESQKLTFVFADRIEEKNQVLVRDVETGELRPQRVVKVGSVRSKGVVAPLTREGTIVVNSVAASCYAVINSQSLAHWGLAPMRLLSTLEAWLPAKEQLHSSPKVVSSAQQQNGIHWYANALYKVKDYVLPQSWRHD

SEQ ID NO: 141 - protein, 40 aa, Artificial
  Variant of 2A sequence.
LLAIHPTEARHKQKIVAPVKQTLNFDLLKLAGDVESNPGP

SEQ ID NO: 142 - protein, 33 aa, Artificial
  Variant of 2A sequence.
EARHKQKIVAPVKQTLNFDLLKLAGDVESNPGP

SEQ ID NO: 143 - DNA, 20 bp, Artificial
  Synthetic construct  oligonucleotide useful as a primer.
atcgtggcgccagctctgcg

SEQ ID NO: 144 - DNA, 20 bp, Artificial
  Synthetic construct  oligonucleotide useful as a primer.
gcaactggcggccaccgagt

SEQ ID NO: 145 - DNA, 20 bp, Artificial
  Synthetic construct  oligonucleotide useful as a primer.
cgcatagcaactggcggcca

SEQ ID NO: 146 - DNA, 20 bp, Artificial
  Synthetic construct  oligonucleotide useful as a primer.
gttgtgggcggccaccgagt

SEQ ID NO: 147 - DNA, 60 bp, Artificial
  Synthetic construct  oligonucleotide useful as a primer.
ccactacacgcagaagagcctctccctgtctccgggtaaatgcttcacgccggagagcac

SEQ ID NO: 148 - DNA, 60 bp, Artificial
  Synthetic construct  oligonucleotide useful as a primer.
gcagcaggcccagcagctgggcgggcacgcgcatgtccatgcactggctgttgatcaccg

SEQ ID NO: 149 - DNA, 60 bp, Artificial
  Synthetic construct  oligonucleotide useful as a primer.
gcagcaggcccagcagctgggcgggcacgcgcatgtccatatcgtggcgccagctctgcg

SEQ ID NO: 150 - DNA, 60 bp, Artificial
  Synthetic construct  oligonucleotide useful as a primer.
gcagcaggcccagcagctgggcgggcacgcgcatgtccatgcaactggcggccaccgagt

SEQ ID NO: 151 - DNA, 60 bp, Artificial
  Synthetic construct  oligonucleotide useful as a primer.
gcagcaggcccagcagctgggcgggcacgcgcatgtccatcgcatagcaactggcggcca

SEQ ID NO: 152 - DNA, 60 bp, Artificial
  Synthetic construct  oligonucleotide useful as a primer.
gcagcaggcccagcagctgggcgggcacgcgcatgtccatgttgtgggcggccaccgagt

SEQ ID NO: 153 - DNA, 40 bp, Artificial; identical to SEQ ID NO: 122
  Synthetic construct  oligonucleotide useful as a primer.

SEQ ID NO: 154 - DNA, 41 bp, Artificial; identical to SEQ ID NO: 123
  Synthetic construct  oligonucleotide useful as a primer.

SEQ ID NO: 155 - DNA, 8533 bp, Artificial
  Synthetic construct  nucleotide sequence of plasmid  pTT3-D2E7 Heavy Chain - Hh-C17- D2E7 Light Chain.
gcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttgggcgccaccatggagtttgggctgagctggctttttcttgtcgcgattttaaaaggtgtccagtgtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcggatcgcatcgaggagaagaaccaggtgctcgtacgggatgtggagacgggcgagctgaggccccagcgagtcgtcaaggtgggcagtgtgcgcagtaagggcgtggtcgcgccgctgacccgcgagggcaccattgtggtcaactcggtggccgccagttgctatgcggtgatcaacagccagtcgatggacatgcgcgtgcccgcccagctgctgggcctgctgctgctgtggttccccggctcgcgatgcgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 156 - DNA, 447 bp, Artificial
  Synthetic construct  partial coding sequence of plasmid pTT3-HC-C17-sc-LC.
ccgggtaaatgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcggatcgcatcgaggagaagaaccaggtgctcgtacgggatgtggagacgggcgagctgaggccccagcgagtcgtcaaggtgggcagtgtgcgcagtaagggcgtggtcgcgccgctgacccgcgagggcaccattgtggtcaactcggtggccgccagttgcatggacatg

SEQ ID NO: 157 - DNA, 447 bp, Artificial
  Synthetic construct  partial coding sequence from plasmid pTT3-HC-C17-hn-LC.
ccgggtaaatgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcggatcgcatcgaggagaagaaccaggtgctcgtacgggatgtggagacgggcgagctgaggccccagcgagtcgtcaaggtgggcagtgtgcgcagtaagggcgtggtcgcgccgctgacccgcgagggcaccattgtggtcaactcggtggccgcccacaacatggacatg

SEQ ID NO: 158 - DNA, 660 bp, Artificial
  Synthetic construct  partial coding sequence from pTT3-HC-C25-Hint-LC.
ccgggtaaatgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcggatcgcatcgaggagaagaaccaggtgctcgtacgggatgtggagacgggcgagctgaggccccagcgagtcgtcaaggtgggcagtgtgcgcagtaagggcgtggtcgcgccgctgacccgcgagggcaccattgtggtcaactcggtggccgccagttgctatgcggtgatcaacagccagtcgctggcccactggggactggctcccatgcgcctgctgtccacgctggaggcgtggctgcccgccaaggagcagttgcacagttcgccgaaggtggtgagctcggcgcagcagcagaatggcatccattggtatgccaatgcgctctacaaggtcaaggactacgttctgccgcagagctggcgccacgatatggacatg